(19) United States
(12) Patent Application Publication
(10) Pub. No.: US 2024/0306492 A1
(43) Pub. Date: Sep. 12, 2024

(54) LIGHT EMITTING ELEMENT AND AMINE COMPOUND FOR THE SAME

(71) Applicant: Samsung Display Co., Ltd., Yongin-si (KR)

(72) Inventor: Ichinori TAKADA, Yokohama (JP)

(21) Appl. No.: 18/435,640

(22) Filed: Feb. 7, 2024

(30) Foreign Application Priority Data
Feb. 24, 2023 (KR) 10-2023-0025383

(51) Int. Cl. H10K 85/60; C09K 11/06; H10K 50/125; H10K 50/155; H10K 85/30

(52) U.S. Cl. CPC H10K 85/633; C09K 11/06; H10K 50/125; H10K 50/155; H10K 85/342; H10K 85/346; H10K 85/626; H10K 85/654; H10K 85/6572; H10K 85/6574; H10K 85/6576

(57) ABSTRACT

A light emitting element including a first electrode, a second electrode located on the first electrode, and at least one functional layer is provided. The functional layer includes an amine compound having substituents that may improve emission efficiency and element lifetime.

### CROSS-REFERENCE TO RELATED APPLICATION

This application claims priority to and the benefit of Korean Patent Application No. 10-2023-0025383, filed on Feb. 24, 2023, in the Korean Intellectual Property Office, the entire content of which is hereby incorporated by reference.

### BACKGROUND

### 1. Field

One or more aspects of embodiments of the present disclosure relate to a light emitting element and an amine compound utilized in the light emitting element.

### 2. Description of the Related Art

Recently, the development of an organic electroluminescence display device as an image display device is being actively conducted. The organic electroluminescence display device is so-called a display device including a “self-luminescent”-type or kind of light emitting element in which holes and electrons are injected from a first electrode and a second electrode (respectively). Subsequently, the holes and electrons recombine in an emission layer so that a light emitting material located in the emission layer emits light to achieve display (e.g., of an image).

Implementation of the organic electroluminescence device in a display device requires (or there is a desire for) the decrease of a driving voltage, and the increase of emission efficiency and lifetime. Therefore, the need exists for development of materials for a light emitting element, which is capable of stably (or suitably) achieving these required and/or desired properties. For example, in an effort to implement a light emitting element having relatively high efficiency and lifetime, the development of materials for a hole transport region having excellent or suitable hole transport properties is being pursued.

### 1 SUMMARY

One or more aspects of embodiments of the present disclosure are directed toward a light emitting element having improved emission efficiency and element lifetime.

One or more aspects of embodiments of the present disclosure are directed toward an amine compound which may improve the emission efficiency and element lifetime of a light emitting element.

A light emitting element of one or more embodiments includes: a first electrode; a second electrode provided on the first electrode; and at least one functional layer including an amine compound represented by Formula 1, and provided between the first electrode and the second electrode.

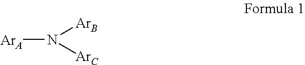

In Formula 1, Ar_{A} is represented by Formula A, Ar_{B} is represented by Formula B, and Ar_{C} is represented by Formula C.

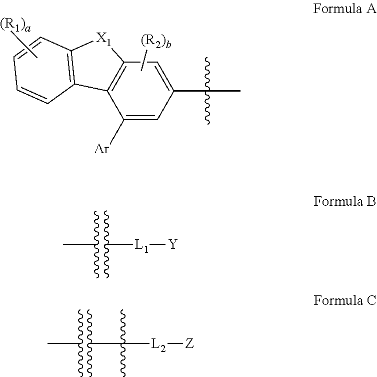

In Formula A to Formula C, X₁ is O or S, Ar is a substituted or unsubstituted aryl group with a total carbon number (i.e., number of carbon atoms) of 6 to 16, R₁ and R₂ may each independently be a hydrogen atom, a substituted or unsubstituted alkyl group of 1 to 10 carbon atoms, a substituted or unsubstituted aryl group of 6 to 30 ring-forming carbon atoms, or a substituted or unsubstituted heteroaryl group of 2 to 30 ring-forming carbon atoms, each of R₁ and R₂ excludes (e.g., does not include) a substituted or unsubstituted nitrogen-containing six-member heterocycle, “a” is an integer of 0 to 4, “b” is an integer of 0 to 2, L₁ and L₂ may each independently be a direct linkage, a substituted or unsubstituted arylene group of 6 to 30 ring-forming carbon atoms, Y and Z may each independently be a substituted or unsubstituted aryl group of 6 to 30 ring-forming carbon atoms, or a substituted or unsubstituted heteroaryl group of 2 to 30 ring-forming carbon atoms, at least one selected from among Y and Z is a substituted or unsubstituted aryl group of 10 to 30 ring-forming carbon atoms, or a substituted or unsubstituted heteroaryl group of 12 to 30 ring-forming carbon atoms, each of Y and Z excludes (e.g., does not include) a substituted or unsubstituted dimethylfluorenyl group, a substituted or unsubstituted fluoranthene group, or a halogen atom, when Y is a substituted or unsubstituted naphthyl group, L₁ is not a direct linkage, when Z is a substituted or unsubstituted naphthyl group, L₂ is not a direct linkage, when Y is a substituted or unsubstituted carbazole group, L₁ is a direct linkage or an unsubstituted phenylene group, when Z is a substituted or unsubstituted carbazole group, L₂ is a direct linkage or an unsubstituted phenylene group, when L₁ is a m-phenylene group, Y is not a substituted or unsubstituted 10-arylphenanthren-9-yl group, when L₂ is a m-phenylene group, Z is not a substituted or unsubstituted 10-arylphenanthren-9-yl group, [symbol] , [symbol] , [symbol] and are positions respectively bonded to the nitrogen atom of Formula 1, and Formula 1 includes a structure in which a hydrogen atom is optionally substituted by (e.g., with) a deuterium atom.

In one or more embodiments, at least one selected from among Y and Z may be represented by any one selected from among Formula 1a to Formula 1c.

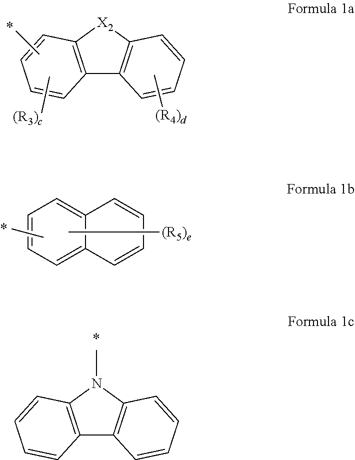

In Formula 1a to Formula 1c, X₂ is O, S, NRₐ, or CR_{b}R_{c}, R₃ to R₅, and Rₐ to R_{c} may each independently be a hydrogen atom, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring, “c” is an integer of 0 to 3, “d” is an integer of 0 to 4, “e” is an integer of 0 to 7, “*-” is a position where Y is bonded to L₁, or a position where Z is bonded to L₂, and Formula 1a to Formula 1c include structures in which a hydrogen atom is optionally substituted by (e.g., with) a deuterium atom.

In one or more embodiments, the amine compound represented by Formula 1 may be a monoamine compound.

In one or more embodiments, the amine compound represented by Formula 1 may be represented by Formula 2-1 or Formula 2-2.

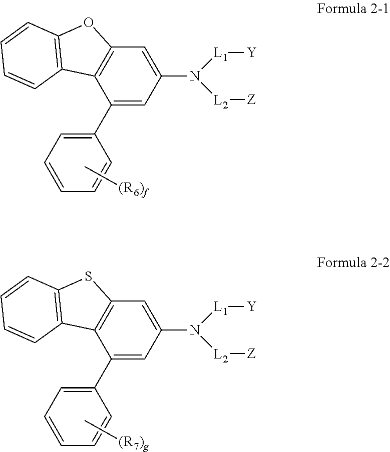

In Formula 2-1 and Formula 2-2, R₆ and R₇ may each independently be a hydrogen atom, a substituted or unsubstituted alkyl group of 1 to 10 carbon atoms, a substituted or unsubstituted alkenyl group of 2 to 10 carbon atoms, or a substituted or unsubstituted aryl group of 6 to 30 ring-forming carbon atoms, and/or combined with an adjacent group to form a ring, “f” and “g” may each independently be an integer of 0 to 5, L₁, L₂, Y and Z may each independently be as defined in Formula 1, and Formula 2-1 and Formula 2-2 include structures in which a hydrogen atom is optionally substituted by (e.g., with) a deuterium atom.

In one or more embodiments, R₁ and R₂ may both (e.g., simultaneously and respectively) be hydrogen atoms.

In one or more embodiments, L₁ and L₂ may each independently be a direct linkage, an unsubstituted phenylene group, or an unsubstituted biphenylene group.

In one or more embodiments, the at least one function layer may include: an emission layer; and a hole transport region provided between the first electrode and the emission layer, and the hole transport region may include the amine compound represented by Formula 1.

In one or more embodiments, the hole transport region may include: a hole injection layer provided on the first electrode; and a hole transport layer provided on the hole injection layer, and the hole transport layer may include the amine compound represented by Formula 1.

An amine compound of one or more embodiments is represented by Formula 1.

### BRIEF DESCRIPTION OF THE FIGURES

The accompanying drawings are included to provide a further understanding of the present disclosure and are incorporated in and constitute a part of this specification. The drawings illustrate example embodiments of the present disclosure and, together with the description, serve to explain principles of the present disclosure. In the drawings:

FIG. 1 is a plan view showing a display device according to one or more embodiments of the present disclosure;

FIG. 2 is a cross-sectional view showing a display device according to one or more embodiments of the present disclosure;

FIG. 3 is a cross-sectional view schematically showing a light emitting element of one or more embodiments of the present disclosure;

FIG. 4 is a cross-sectional view schematically showing a light emitting element of one or more embodiments of the present disclosure;

FIG. 5 is a cross-sectional view schematically showing a light emitting element of one or more embodiments of the present disclosure;

FIG. 6 is a cross-sectional view schematically showing a light emitting element of one or more embodiments of the present disclosure;

FIG. 7 is a cross-sectional view showing a display device according to one or more embodiments of the present disclosure;

FIG. 8 is a cross-sectional view showing a display device according to one or more embodiments of the present disclosure;

FIG. 9 is a cross-sectional view showing a display device according to one or more embodiments of the present disclosure;

FIG. 10 is a cross-sectional view showing a display device according to one or more embodiments of the present disclosure; and

FIG. 11 is a perspective view schematically showing an electronic device including a display device according to one or more embodiments of the present disclosure.

### DETAILED DESCRIPTION

The present disclosure may have one or more suitable modifications and may be embodied in different forms, and example embodiments will be explained in more detail with reference to the accompany drawings. The present disclosure may, however, be embodied in different forms and should not be construed as limited to the embodiments set forth herein. Rather, all modifications, equivalents, and substituents which are included in the spirit and technical scope of the present disclosure should be included in the present disclosure. Unless otherwise defined, all chemical names, technical and scientific terms, and terms defined in common dictionaries should be interpreted as having meanings consistent with the context of the related art, and should not be interpreted in an ideal or overly formal sense.

In describing the drawings, like reference numerals refer to like elements throughout, and duplicative descriptions thereof may not be provided. In the drawings, the dimensions of structures are exaggerated for clarity of illustration. It will be understood that, although the terms first, second, and/or the like may be utilized herein to describe one or more suitable elements, these elements should not be limited by these terms. These terms are only utilized to distinguish one element from another element. Thus, a first element could be termed a second element without departing from the teachings of the present disclosure. Similarly, a second element could be termed a first element. As utilized herein, the singular forms are intended to include the plural forms as well, unless the context clearly indicates otherwise.

In the application, it will be further understood that the terms “comprise,” “comprises,” “comprising,” “has,” “have,” “having,” “include,” “includes,” and/or “including” when utilized in this specification, specify the presence of stated features, numerals, steps, operations, elements, parts, or the combination thereof, but do not preclude the presence or addition of one or more other features, numerals, steps, operations, elements, parts, or the combination thereof.

As used herein, the terms “use,” “using,” and “used” may be considered synonymous with the terms “utilize,” “utilizing,” and “utilized,” respectively.

As used herein, expressions such as “at least one of,” “one of,” and “selected from,” when preceding a list of elements, modify the entire list of elements and do not modify the individual elements of the list. For example, the expressions “at least one of a to c,” “at least one of a, b or c,” and “at least one of a, b and/or c” may indicate only a, only b, only c, both (e.g., simultaneously) a and b, both (e.g., simultaneously) a and c, both (e.g., simultaneously) b and c, all of a, b, and c, or variations thereof.

As used herein, the term “and/or” includes any and all combinations of one or more of the associated listed items.

The term “may” will be understood to refer to “one or more embodiments of the present disclosure,” some of which include the described element and some of which exclude that element and/or include an alternate element. Similarly, alternative language such as “or” refers to “one or more embodiments of the present disclosure,” each including a corresponding listed item.

In the application, when a layer, a film, a region, a plate, and/or the like is referred to as being “on,” “connected to,” “coupled to,” or “above” another part, it can be “directly on, connected to, or coupled to,” the other part, or intervening layers may also be present. In contrast, when a layer, a film, a region, a plate, and/or the like is referred to as being “under” or “below” another part, it can be “directly under” the other part, or intervening layers may also be present. Also, when an element is referred to as being provided “on” another element, it can be provided under the other element.

Spatially relative terms, such as “beneath,” “below,” “lower,” “above,” “upper,” “bottom,” “top,” and the like, may be used herein for ease of description to describe one element or feature's relationship to another element(s) or feature(s) as illustrated in the drawings. It will be understood that the spatially relative terms are intended to encompass different orientations of the device in use or operation in addition to the orientation depicted in the drawings. For example, if the device in the drawings is turned over, elements described as “below” or “beneath” other elements or features would then be oriented “above” or “over” the other elements or features. Thus, the term “below” may encompass both an orientation of above and below. The device may be otherwise oriented (rotated 90 degrees or at other orientations), and the spatially relative descriptors used herein should be interpreted accordingly.

Unless otherwise defined, all terms (including technical and scientific terms) used herein have the same meaning as commonly understood by those of ordinary skill in the art to which the present disclosure pertains. It is also to be understood that terms defined in commonly used dictionaries should be interpreted as having meanings consistent with meanings in the context of the related art, unless expressly defined herein, and should not be interpreted in an ideal or overly formal sense.

In this context, “consisting essentially of” means that any additional components will not materially affect the chemical, physical, optical, or electrical properties of the semiconductor film.

Further, in this specification, the phrase “on a plane,” or “plan view,” means viewing a target portion from the top, and the phrase “on a cross-section” means viewing a cross-section formed by vertically cutting a target portion from the side.

### Definitions

In the description, the term “substituted or unsubstituted” corresponds to substituted or unsubstituted with at least one substituent selected from the group consisting of a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In some embodiments, each of the exemplified substituents may be substituted or unsubstituted. For example, a biphenyl group may be interpreted as an aryl group or a phenyl group substituted with a phenyl group.

In the description, the term “forming a ring via the combination with an adjacent group” may refer to forming a substituted or unsubstituted hydrocarbon ring, or a substituted or unsubstituted heterocycle via the combination with an adjacent group. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocycles or polycycles. In some embodiments, the ring formed via the combination with an adjacent group may be combined with another ring to form a spiro structure.

In the description, the term “adjacent group” may refer to a substituent substituted for an atom which is directly combined with an atom substituted with a corresponding substituent, another substituent substituted for an atom which is substituted with a corresponding substituent, or a substituent sterically positioned at the nearest position to a corresponding substituent. For example, in 1,2-dimethylbenzene, two methyl groups may be interpreted as “adjacent groups” to each other, and in 1,1-diethylcyclopentene, two ethyl groups may be interpreted as “adjacent groups” to each other. In some embodiments, in 4,5-dimethylphenanthrene, two methyl groups may be interpreted as “adjacent groups” to each other.

In the description, a halogen atom may be a fluorine atom, a chlorine atom, a bromine atom or an iodine atom.

In the description, an alkyl group may be a linear, branched, or cyclic type or kind. The carbon number of the alkyl group may be 1 to 50, 1 to 30, 1 to 20, 1 to 10, or 1 to 6. Examples of the alkyl group may include methyl, ethyl, n-propyl, isopropyl, n-butyl, s-butyl, t-butyl, i-butyl, 2-ethylbutyl, 3,3-dimethylbutyl, n-pentyl, i-pentyl, neopentyl, t-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-t-butylcyclohexyl, n-heptyl, 1-methylheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldocecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-henicosyl, n-docosyl, n-tricosyl, n-tetracosyl, n-pentacosyl, n-hexacosyl, n-heptacosyl, n-octacosyl, n-nonacosyl, n-triacontyl, and/or the like, without limitation.

In the description, an alkyl group may be a linear or branched type or kind. The carbon number of the alkyl group may be 1 to 50, 1 to 30, 1 to 20, 1 to 10, or 1 to 6. Examples of the alkyl group may include methyl, ethyl, n-propyl, isopropyl, n-butyl, s-butyl, t-butyl, i-butyl, 2-ethylbutyl, 3,3-dimethylbutyl, n-pentyl, i-pentyl, neopentyl, t-pentyl, 1-methylpentyl, 3-methylpentyl, 2-ethylpentyl, 4-methyl-2-pentyl, n-hexyl, 1-methylhexyl, 2-ethylhexyl, 2-butylhexyl, n-heptyl, 1-methylheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldocecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-henicosyl, n-docosyl, n-tricosyl, n-tetracosyl, n-pentacosyl, n-hexacosyl, n-heptacosyl, n-octacosyl, n-nonacosyl, n-triacontyl, and/or the like, without limitation.

In the description, a cycloalkyl group may refer to a ring-type or kind alkyl group. The carbon number of the cycloalkyl group may be 3 to 50, 3 to 30, 3 to 20, or 3 to 10. Examples of the cycloalkyl group include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group and/or the like, without limitation.

In the description, an alkenyl group refers to a hydrocarbon group including one or more carbon double bonds in the middle or at the terminal of an alkyl group having a carbon number of 2 or more. The alkenyl group may be a linear chain or a branched chain. The carbon number is not specifically limited, but may be 2 to 30, 2 to 20, or 2 to 10. Examples of the alkenyl group include a vinyl group, a 1-butenyl group, a 1-pentenyl group, a 1,3-butadienyl aryl group, a styrenyl group, a styrylvinyl group, and/or the like, without limitation.

In the description, an alkynyl group refers to a hydrocarbon group including one or more carbon triple bonds in the middle or at the terminal of an alkyl group having a carbon number of 2 or more. The alkynyl group may be a linear chain or a branched chain. The carbon number is not specifically limited, but may be 2 to 30, 2 to 20, or 2 to 10. Examples of the alkynyl group include an ethynyl group, a propionyl group, and/or the like, without limitation.

In the description, a hydrocarbon ring group refers to an optional functional group or substituent derived from an aliphatic hydrocarbon ring. A hydrocarbon ring group may be a saturated hydrocarbon ring group of 5 to 20 ring-forming carbon atoms.

In the description, an aryl group refers to an optional functional group or substituent derived from an aromatic hydrocarbon ring. The aryl group may be a monocyclic aryl group or a polycyclic aryl group. The carbon number for forming rings in the aryl group may be 6 to 30, 6 to 20, or 6 to 15. Examples of the aryl group may include phenyl, naphthyl, fluorenyl, anthracenyl, phenanthryl, biphenyl, terphenyl, quaterphenyl, quinquephenyl, sexiphenyl, triphenylenyl, pyrenyl, benzofluoranthenyl, chrysenyl, and/or the like, without limitation.

In the description, a fluorenyl group may be substituted, and two substituents may be combined with each other to form a spiro structure. Examples of a substituted fluorenyl group are as follows, but one or more embodiments of the present disclosure is not limited thereto.

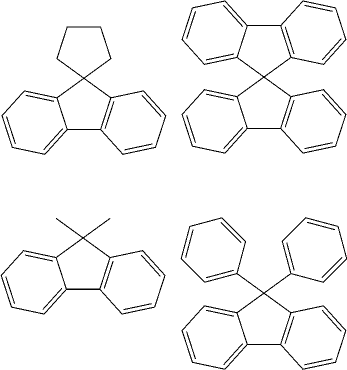

In the description, a heterocyclic group refers to an optional functional group or substituent derived from a ring including one or more among B, O, N, P, Si, and S as heteroatoms. The heterocyclic group includes an aliphatic heterocyclic group and an aromatic heterocyclic group. The aromatic heterocyclic group may be a heteroaryl group. The aliphatic heterocyclic group and the aromatic heterocyclic group may be a monocycle or a polycycle.

In the description, a heterocyclic group may include one or more among B, O, N, P, Si and S as heteroatoms. When the heterocyclic group includes two or more heteroatoms, two or more heteroatoms may be the same or different. The heterocyclic group may be a monocyclic heterocyclic group or polycyclic heterocyclic group and has the concept including a heteroaryl group. The carbon number for forming rings of the heterocyclic group may be 2 to 30, 2 to 20, or 2 to 10.

In the description, an aliphatic heterocyclic group may include one or more among B, O, N, P, Si, and S as heteroatoms. The number of ring-forming carbon atoms of the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, and/or the like, without limitation.

In the description, a heteroaryl group may include one or more among B, O, N, P, Si, and S as heteroatoms. When the heteroaryl group includes two or more heteroatoms, two or more heteroatoms may be the same or different. The heteroaryl group may be a monocyclic heterocyclic group or polycyclic heterocyclic group. The carbon number for forming rings of the heteroaryl group may be 2 to 30, 2 to 20, or 2 to 10. Examples of the heteroaryl group may include thiophene, furan, pyrrole, imidazole, pyridine, bipyridine, pyrimidine, triazine, triazole, acridyl, pyridazine, pyrazinyl, quinoline, quinazoline, quinoxaline, phenoxazine, phthalazine, pyrido pyrimidine, pyrido pyrazine, pyrazino pyrazine, isoquinoline, indole, carbazole, N-arylcarbazole, N-heteroarylcarbazole, N-alkylcarbazole, benzoxazole, benzimidazole, benzothiazole, benzocarbazole, benzothiophene, dibenzothiophene, thienothiophene, benzofuran, phenanthroline, thiazole, isooxazole, oxazole, oxadiazole, thiadiazole, phenothiazine, dibenzosilole, dibenzofuran, and/or the like, without limitation.

In the description, the same explanation on the described aryl group may be applied to an arylene group except that the arylene group is a divalent group. The same explanation on the described heteroaryl group may be applied to a heteroarylene group except that the heteroarylene group is a divalent group.

In the description, a silyl group includes an alkyl silyl group and an aryl silyl group. Examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and/or the like, without limitation.

In the description, the carbon number of an amino group is not specifically limited, but may be 1 to 30. The amino group may include an alkyl amino group, an aryl amino group, or a heteroaryl amino group. Examples of the amine group include a methylamino group, a dimethylamino group, a phenylamino group, a diphenylamino group, a naphthylamino group, a 9-methyl-anthracenylamino group, a triphenylamino group, and/or the like, without limitation.

In the description, the carbon number of a carbonyl group is not specifically limited, but the carbon number may be 1 to 40, 1 to 30, or 1 to 20. For example, the carbonyl group may have the structures, but is not limited thereto.

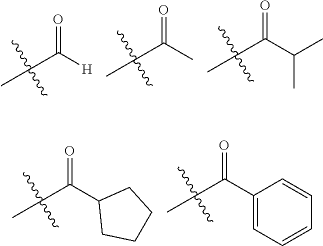

In the description, the carbon number of a sulfinyl group and sulfonyl group is not specifically limited, but may be 1 to 30. The sulfinyl group may include an alkyl sulfinyl group and an aryl sulfinyl group. The sulfonyl group may include an alkyl sulfonyl group and an aryl sulfonyl group.

In the description, a thio group may include an alkyl thio group and an aryl thio group. The thio group may refer to the defined alkyl group or aryl group combined with a sulfur atom. Examples of the thio group include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, and/or the like, without limitation.

In the description, an oxy group may refer to the defined alkyl group or aryl group which is combined with an oxygen atom. The oxy group may include an alkoxy group and an aryl oxy group. The alkoxy group may be a linear, branched or cyclic chain. The carbon number of the alkoxy group is not specifically limited but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, and/or the like However, one or more embodiments of the present disclosure is not limited thereto.

In the description, a boron group may refer to the defined alkyl group or aryl group combined with a boron atom. The boron group includes an alkyl boron group and an aryl boron group. Examples of the boron group include a dimethylboron group, a diethylboron group, a t-butylboron group, a diphenylboron group, a diphenylboron group, a phenylboron group, and/or the like, without limitation.

In the description, the alkenyl group may be a linear chain or a branched chain. The carbon number is not specifically limited, but may be 2 to 30, 2 to 20, or 2 to 10. Examples of the alkenyl group include a vinyl group, a 1-butenyl group, a 1-pentenyl group, a 1,3-butadienyl aryl group, a styrenyl group, a styrylvinyl group, and/or the like, without limitation.

In the description, the carbon number of an amine group is not specifically limited, but may be 1 to 30. The amine group may include an alkyl amine group and an aryl amine group. Examples of the amine group include a methylamine group, a dimethylamine group, a phenylamine group, a diphenylamine group, a naphthylamine group, a 9-methyl-anthracenylamine group, a triphenylamine group, and/or the like, without limitation.

In the description, alkyl groups in an alkylthio group, alkylsulfoxy group, alkylaryl group, alkylamino group, alkylboron group, alkyl silyl group, and alkyl amine group may be the same as the examples of the described alkyl group.

In the description, aryl groups in an aryloxy group, arylthio group, arylsulfoxy group, aryl amino group, arylboron group, and aryl silyl group may be the same as the examples of the described aryl group.

In the description, a direct linkage may refer to a single bond.

In some embodiments, in the description, “ [symbol] ”, and “ [symbol] ” refer to positions to be connected.

Hereinafter, the light emitting element of one or more embodiments will be explained referring to the drawings.

### Display Device

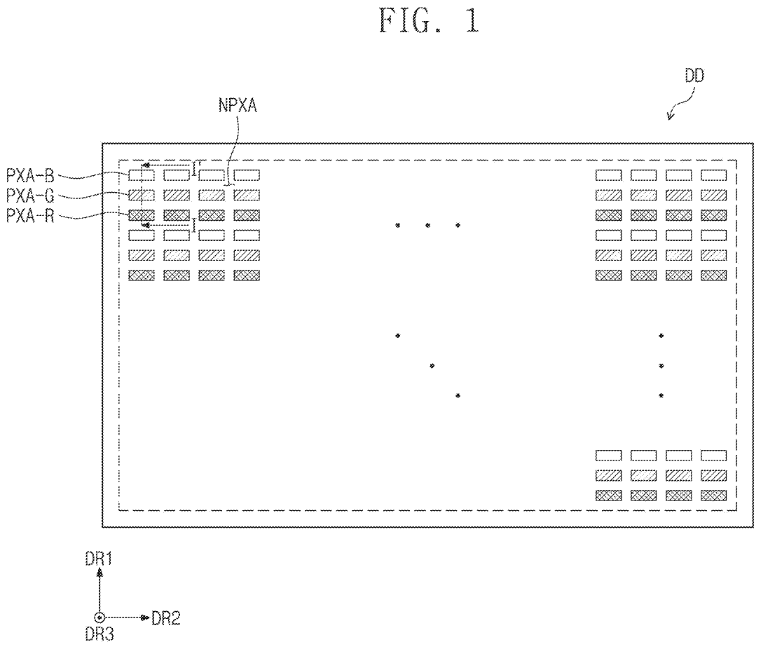

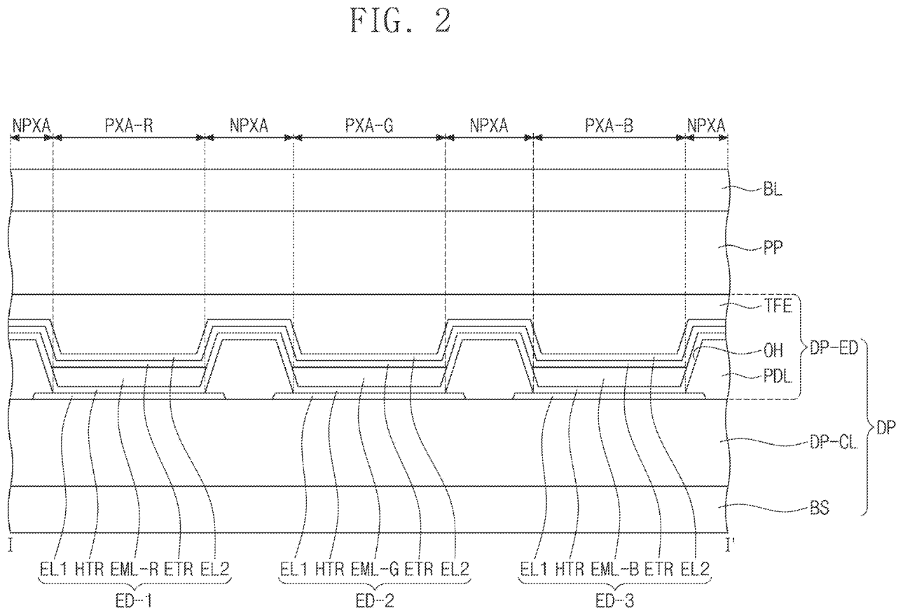

FIG. 1 is a plan view showing a display device DD according to one or more embodiments. FIG. 2 is a cross-sectional view showing a display device DD according to one or more embodiments. FIG. 2 is a cross-sectional view showing a part corresponding to line l-l′ of FIG. 1.

The display device DD may include a display panel DP and an optical layer PP provided on the display panel DP. The display panel DP may include light emitting elements ED-1, ED-2 and ED-3. The display device DD may include multiple light emitting elements ED-1, ED-2 and ED-3. The optical layer PP may be provided on the display panel DP and control reflected light by external light at the display panel DP. The optical layer PP may include, for example, a polarization layer or a color filter layer. In some embodiments, different from the drawings, the optical layer PP may not be provided in the display device DD of one or more embodiments.

On the optical layer PP, a base substrate BL may be provided. The base substrate BL may be a member providing a base surface where the optical layer PP is provided. The base substrate BL may be a glass substrate, a metal substrate, a plastic substrate, and/or the like However, one or more embodiments of the present disclosure is not limited thereto, and the base substrate BL may be an inorganic layer, an organic layer or a composite material layer. In some embodiments, different from the drawings, the base substrate BL may not be provided in one or more embodiments.

The display device DD according to one or more embodiments may further include a plugging layer. The plugging layer may be provided between a display element layer DP-ED and a base substrate BL. The plugging layer may be an organic layer. The plugging layer may include at least one selected from among an acrylic resin, a silicon-based resin and an epoxy-based resin.

The display panel DP may include a base layer BS, a circuit layer DP-CL provided on the base layer BS and a display element layer DP-ED. The display element layer DP-ED may include a pixel definition layer PDL, light emitting elements ED-1, ED-2 and ED-3 provided in the pixel definition layer PDL, and an encapsulating layer TFE provided on the light emitting elements ED-1, ED-2 and ED-3.

The base layer BS may be a member providing a base surface where the display element layer DP-ED is provided. The base layer BS may be a glass substrate, a metal substrate, a plastic substrate, and/or the like However, one or more embodiments of the present disclosure is not limited thereto, and the base layer BS may be an inorganic layer, an organic layer or a composite material layer.

In one or more embodiments, the circuit layer DP-CL is provided on the base layer BS, and the circuit layer DP-CL may include multiple transistors. Each of the transistors may include a control electrode, an input electrode, and an output electrode. For example, the circuit layer DP-CL may include switching transistors and driving transistors for driving the light emitting elements ED-1, ED-2 and ED-3 of the display element layer DP-ED.

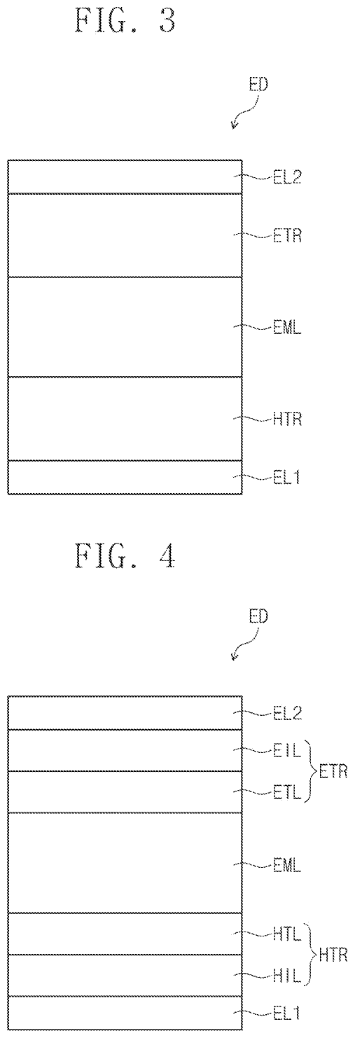

The light emitting elements ED-1, ED-2 and ED-3 may have the structures of the light emitting elements ED of embodiments according to FIG. 3 to FIG. 6, as described in more detail elsewhere herein. The light emitting elements ED-1, ED-2 and ED-3 may include a first electrode EL1, a hole transport region HTR, emission layers EML-R, EML-G and EML-B, an electron transport region ETR and a second electrode EL2.

In FIG. 2, shown is one or more embodiments where the emission layers EML-R, EML-G and EML-B of light emitting elements ED-1, ED-2 and ED-3 are provided in opening portions OH defined in a pixel definition layer PDL, and a hole transport region HTR, an electron transport region ETR and a second electrode EL2 are provided as common layers in all light emitting elements ED-1, ED-2 and ED-3. However, one or more embodiments of the present disclosure is not limited thereto. Different from FIG. 2, in one or more embodiments, the hole transport region HTR and the electron transport region ETR may be patterned and provided in the opening portions OH defined in the pixel definition layer PDL. For example, in one or more embodiments, the hole transport region HTR, the emission layers EML-R, EML-G and EML-B, and the electron transport region ETR of the light emitting elements ED-1, ED-2 and ED-3 may be patterned by an ink jet printing method and provided.

An encapsulating layer TFE may cover the light emitting elements ED-1, ED-2 and ED-3. The encapsulating layer TFE may encapsulate the display element layer DP-ED. The encapsulating layer TFE may be a thin film encapsulating layer. The encapsulating layer TFE may be one layer or a stacked layer of multiple layers. The encapsulating layer TFE includes at least one insulating layer. The encapsulating layer TFE according to one or more embodiments may include at least one inorganic layer (hereinafter, encapsulating inorganic layer). In some embodiments, the encapsulating layer TFE according to one or more embodiments may include at least one organic layer (hereinafter, encapsulating organic layer) and at least one encapsulating inorganic layer.

The encapsulating inorganic layer protects the display element layer DP-ED from moisture/oxygen, and the encapsulating organic layer protects the display element layer DP-ED from foreign materials such as dust particles. The encapsulating inorganic layer may include silicon nitride, silicon oxy nitride, silicon oxide, titanium oxide, or aluminum oxide, without specific limitation. The encapsulating organic layer may include an acrylic compound, an epoxy-based compound, and/or the like The encapsulating organic layer may include a photopolymerizable organic material, without specific limitation.

The encapsulating layer TFE may be provided on the second electrode EL2 and may be provided while filling the opening portion OH.

Referring to FIG. 1 and FIG. 2, the display device DD may include a non-luminous area NPXA and luminous areas PXA-R, PXA-G and PXA-B. The luminous areas PXA-R, PXA-G and PXA-B may be areas emitting light produced from the light emitting elements ED-1, ED-2 and ED-3, respectively. The luminous areas PXA-R, PXA-G and PXA-B may be separated from each other on a plane.

The luminous areas PXA-R, PXA-G and PXA-B may be areas separated by the pixel definition layer PDL. The non-luminous areas NPXA may be areas between neighboring luminous areas PXA-R, PXA-G and PXA-B and may be areas corresponding to the pixel definition layer PDL. In some embodiments, in the disclosure, each of the luminous areas PXA-R, PXA-G and PXA-B may correspond to each pixel. The pixel definition layer PDL may divide the light emitting elements ED-1, ED-2 and ED-3. The emission layers EML-R, EML-G and EML-B of the light emitting elements ED-1, ED-2 and ED-3 may be provided and divided in the opening portions OH defined in the pixel definition layer PDL.

The luminous areas PXA-R, PXA-G and PXA-B may be divided into multiple groups according to the color of light produced from the light emitting elements ED-1, ED-2 and ED-3. In the display device DD of one or more embodiments, shown in FIG. 1 and FIG. 2, three luminous areas PXA-R, PXA-G and PXA-B emitting red light, green light and blue light are illustrated as one or more embodiments. For example, the display device DD of one or more embodiments may include a red luminous area PXA-R, a green luminous area PXA-G and a blue luminous area PXA-B, which are separated from each other.

In the display device DD according to one or more embodiments, multiple light emitting elements ED-1, ED-2 and ED-3 may be to emit (e.g., configured to emit) light having different wavelength regions. For example, in one or more embodiments, the display device DD may include a first light emitting element ED-1 emitting red light, a second light emitting element ED-2 emitting green light, and a third light emitting element ED-3 emitting blue light. For example, each of the red luminous area PXA-R, the green luminous area PXA-G, and the blue luminous area PXA-B of the display device DD may correspond to the first light emitting element ED-1, the second light emitting element ED-2, and the third light emitting element ED-3.

However, one or more embodiments of the present disclosure is not limited thereto, and the first to third light emitting elements ED-1, ED-2 and ED-3 may be to emit (e.g., configured to emit) light in substantially the same wavelength region, or at least one thereof may be to emit (e.g., configured to emit) light in a different wavelength region. For example, all the first to third light emitting elements ED-1, ED-2 and ED-3 may be to emit (e.g., configured to emit) blue light.

The luminous areas PXA-R, PXA-G and PXA-B in the display device DD according to one or more embodiments may be arranged in a stripe shape. Referring to FIG. 1, multiple red luminous areas PXA-R may be arranged with each other along a second directional axis DR2, multiple green luminous areas PXA-G may be arranged with each other along a second directional axis DR2 and multiple blue luminous areas PXA-B may be arranged with each other along a second directional axis DR2. In some embodiments, the (one) red luminous area PXA-R, the (one) green luminous area PXA-G and the (one) blue luminous area PXA-B may be arranged with each other by turns along a first directional axis DR1.

In FIG. 1 and FIG. 2, the areas of the luminous areas PXA-R, PXA-G and PXA-B are shown similar, but one or more embodiments of the present disclosure is not limited thereto. The areas of the luminous areas PXA-R, PXA-G and PXA-B may be different from each other according to the wavelength region of light emitted. In some embodiments, the areas of the luminous areas PXA-R, PXA-G and PXA-B may refer to areas on a plane defined by the first directional axis DR1 and the second directional axis DR2.

In some embodiments, the arrangement type or kind of the luminous areas PXA-R, PXA-G and PXA-B is not limited to the configuration shown in FIG. 1, and the arrangement order of the red luminous areas PXA-R, the green luminous areas PXA-G and the blue luminous areas PXA-B may be provided in one or more suitable combinations according to the properties of display quality required for the display device DD. For example, the arrangement type or kind of the luminous areas PXA-R, PXA-G and PXA-B may be configured in a pentile (PENTILE®) arrangement form, or a diamond (Diamond Pixel®) arrangement form, (PENTILE® and Diamond Pixel® each is a registered trademark owned by Samsung Display Co., Ltd.).

In some embodiments, the areas of the luminous areas PXA-R, PXA-G and PXA-B may be different from each other. For example, in one or more embodiments, the area of the green luminous area PXA-G may be smaller than the area of the blue luminous area PXA-B, but one or more embodiments of the present disclosure is not limited thereto.

### Light Emitting Element

FIG. 3 to FIG. 6 are cross-sectional views schematically showing light emitting elements ED according to embodiments. The light emitting elements ED of embodiments may include a first electrode EL1, a hole transport region HTR, an emission layer EML, an electron transport region ETR, and a second electrode EL2, stacked in order.

As illustrated in FIG. 3, the light emitting element ED includes the first electrode EL1, a hole transport region HTR, a light emitting layer EML, an electron transport region ETR, and the second electrode EL2 which are sequentially laminated.

When compared with FIG. 3, FIG. 4 shows the cross-sectional view of a light emitting element ED of one or more embodiments, wherein a hole transport region HTR includes a hole injection layer HIL and a hole transport layer HTL, and an electron transport region ETR includes an electron injection layer EIL and an electron transport layer ETL.

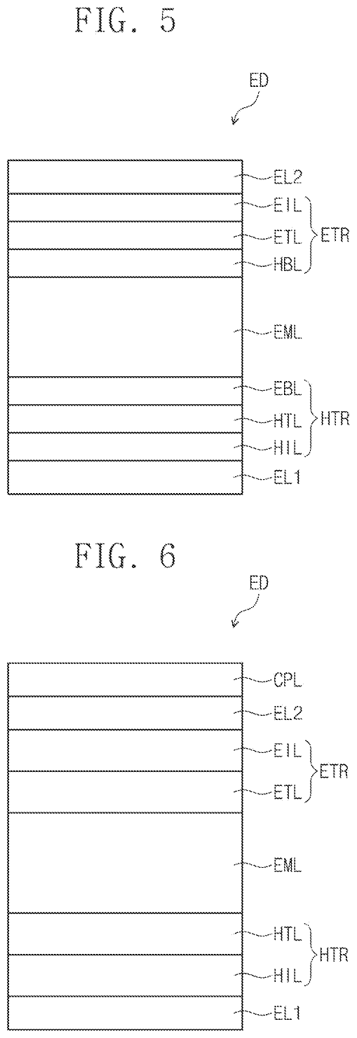

When compared with FIG. 3, FIG. 5 shows the cross-sectional view of a light emitting element ED of one or more embodiments, wherein a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL.

When compared with FIG. 4, FIG. 6 shows the cross-sectional view of a light emitting element ED of one or more embodiments, including a capping layer CPL provided on the second electrode EL2.

### Amine Compound

The light emitting element ED of one or more embodiments according to the present disclosure may include the amine compound of one or more embodiments in at least one functional layer provided between the first electrode EL1 and the second electrode EL2. The at least one functional layer may include a hole transport region HTR, an emission layer EML, and an electron transport region ETR. For example, the hole transport region HTR may include the amine compound of one or more embodiments. The hole transport region HTR of one or more embodiments may include a hole injection layer HIL provided on the first electrode and a hole transport layer HTL provided on the hole injection layer. The hole transport layer HTL may include the amine compound of one or more embodiments.

When the hole transport region HTR includes multiple layers, a layer adjacent to the emission layer EML may include the amine compound of one or more embodiments. For example, as shown in FIG. 5, when the hole transport region HTR includes a hole injection layer HIL, a hole transport layer HTL, and an electron blocking layer EBL, the electron blocking layer EBL adjacent to the emission layer EML may include the amine compound of one or more embodiments.

The amine compound of one or more embodiments according to the present disclosure may include a structure in which first to third substituents are bonded to the nitrogen atom of an amine. The first substituent may include a 1-aryldibenzofuran-3-yl group or a 1-aryldibenzothiophen-3-yl group. For example, position 3 of the first substituent may be directly bonded to the nitrogen atom of the amine. The second substituent and the third substituent may be substituted or unsubstituted aryl groups or heteroaryl groups. The second substituent and the third substituent may be directly bonded to the nitrogen atom of the amine, or bonded via a linker. In some embodiments, the amine compound of one or more embodiments may be a monoamine compound.

Due to the structure, the amine compound of one or more embodiments may have excellent or suitable hole transport capacity. Accordingly, the light emitting element ED including the amine compound of one or more embodiments in the hole transport region HTR may show improved charge balance, and the light emitting element ED may show relatively high emission efficiency and long-life characteristics.

The amine compound of one or more embodiments may be represented by Formula 1.

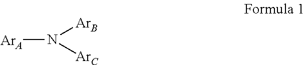

In Formula 1, Ar_{A} is represented by Formula A, Ar_{B} is represented by Formula B, and Ar_{C} is represented by Formula C.

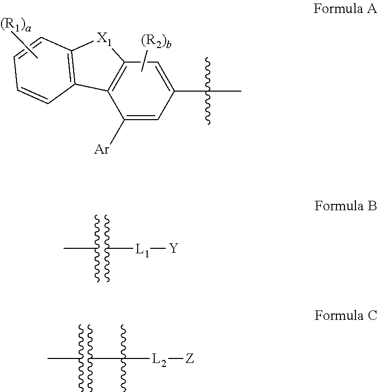

In Formula A to Formula C, X₁ is O, or S. For example, when X₁ is O, a first substituent may be a dibenzofuran group substituted with Ar. In some embodiments, when X₁ is S, a first substituent may be a dibenzothiophene group substituted with Ar. Here, the first substituent may refer to a substituent directly bonded to the nitrogen atom of an amine and including Ar.

Ar is a substituted or unsubstituted aryl group with a total carbon number (i.e., number of carbon atoms) of 6 to 16. For example, Ar may be a substituted or unsubstituted phenyl group, an unsubstituted naphthyl group, or an unsubstituted phenanthryl group. When Ar is a substituted phenyl group, Ar may be a phenyl group substituted with deuterium, a cyclohexane group, a phenyl group or a naphthyl group. Here, the total carbon number of 6 to 16 may refer to that the carbon number of the total substituents included in Ar is 6 to 16. For example, when Ar is a phenyl group substituted with one phenyl group, the total carbon number of Ar may be 12.

R₁ and R₂ may each independently be a hydrogen atom, a substituted or unsubstituted alkyl group of 1 to 10 carbon atoms, a substituted or unsubstituted aryl group of 6 to 30 ring-forming carbon atoms, or a substituted or unsubstituted heteroaryl group of 2 to 30 ring-forming carbon atoms. For example, R₁ and R₂ may be hydrogen atoms. R₁ and R₂ may be the same or different.

Each of R₁ and R₂ excludes (e.g., does not include) a substituted or unsubstituted nitrogen-containing six-member heterocycle. For example, each of R₁ and R₂ excludes (e.g., does not include) a triazine group.

“a” is an integer of 0 to 4. A case where “a” is 0, may be the same as a case where “a” is 4, and all R₁ are hydrogen atoms. When “a” is an integer of 2 or more, two or more R₁ may be the same, or at least one thereof may be different from the remainder.

“b” is an integer of 0 to 2. A case where “b” is 0, may be the same as a case where “b” is 2, and all R₂ are hydrogen atoms. When “b” is 2, two R₂ may be the same, or different.

L₁ and L₂ may each independently be a direct linkage, a substituted or unsubstituted arylene group of 6 to 30 ring-forming carbon atoms. For example, L₁ and L₂ may each independently be a direct linkage, a substituted or unsubstituted phenylene group, or an unsubstituted biphenylene group. When L₁ and L₂ are substituted phenylene groups, each of L₁ and L₂ may be a phenylene group substituted with a phenyl group. L₁ and L₂ may be the same or different.

Y and Z may each independently be a substituted or unsubstituted aryl group of 6 to 30 ring-forming carbon atoms, or a substituted or unsubstituted heteroaryl group of 2 to 30 ring-forming carbon atoms. For example, Y and Z may each independently be a substituted or unsubstituted phenyl group, an unsubstituted biphenyl group, an unsubstituted terphenyl group, a substituted or unsubstituted naphthyl group, a substituted or unsubstituted phenanthryl group, a substituted or unsubstituted carbazole group, a substituted or unsubstituted dibenzofuran group, a substituted or unsubstituted dibenzothiophene group, a fluorenyl group substituted with a phenyl group, an unsubstituted naphthobenzofuran group, or an unsubstituted naphthobenzothiophene group. When Y and Z are substituted phenyl groups, each of Y and Z may be a phenyl group substituted with a deuterium atom. When Y and Z are substituted naphthyl groups, each of Y and Z may be a naphthyl group substituted with a phenyl group. When Y and Z are substituted phenanthryl groups, each of Y and Z may be a phenanthryl group substituted with a phenyl group. When Y and Z are substituted carbazole groups, each of Y and Z may be a carbazole group substituted with a phenyl group. When Y and Z are substituted dibenzofuran groups, each of Y and Z may be a dibenzofuran group substituted with a deuterium or a phenyl group. When Y and Z are substituted dibenzothiophene groups, each of Y and Z may be a dibenzothiophene group substituted with a phenyl group. Y and Z may be the same or different.

At least one selected from among Y and Z may be a substituted or unsubstituted aryl group of 10 to 30 ring-forming carbon atoms, or a substituted or unsubstituted heteroaryl group of 12 to 30 ring-forming carbon atoms. For example, at least one selected from among Y and Z may be a substituted or unsubstituted naphthyl group, a substituted or unsubstituted phenanthryl group, a substituted or unsubstituted carbazole group, a substituted or unsubstituted dibenzofuran group, a substituted or unsubstituted dibenzothiophene group, a fluorenyl group substituted with a phenyl group, an unsubstituted naphthobenzofuran group, or an unsubstituted naphthobenzothiophene group. When Y and Z are substituted naphthyl groups, each of Y and Z may be a naphthyl group substituted with a phenyl group. When Y and Z are substituted phenanthryl groups, each of Y and Z may be a phenanthryl group substituted with a phenyl group. When Y and Z are substituted carbazole groups, each of Y and Z may be a carbazole group substituted with a phenyl group. When Y and Z are substituted dibenzofuran groups, each of Y and Z may be a dibenzofuran group substituted with a deuterium or a phenyl group. When Y and Z are substituted dibenzothiophene groups, each of Y and Z may be a dibenzothiophene group substituted with a phenyl group.

Each of Y and Z excludes (e.g., does not include) a substituted or unsubstituted dimethylfluorenyl group, a substituted or unsubstituted fluoranthene group, or a halogen atom.

When Y is a substituted or unsubstituted naphthyl group, L₁ is not a direct linkage. For example, when Y is a substituted or unsubstituted naphthyl group, L₁ may be a substituted or unsubstituted arylene group of 6 to 30 ring-forming carbon atoms. For example, when Y is an unsubstituted naphthyl group, L₁ may be an unsubstituted phenylene group, or an unsubstituted biphenylene group.

When Z is a substituted or unsubstituted naphthyl group, L₂ is not a direct linkage. For example, when Z is a substituted or unsubstituted naphthyl group, L₂ may be substituted or unsubstituted arylene group of 6 to 30 ring-forming carbon atoms. For example, when Z is an unsubstituted naphthyl group, L₂ may be an unsubstituted phenylene group, or an unsubstituted biphenylene group.

When L₁ is a m-phenylene group, Y is not a substituted or unsubstituted 10-arylphenanthren-9-yl group. Here, the 10-arylphenanthren-9-yl group is as follows.

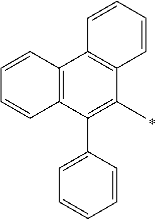 [SMILES: Cc1c(-c2ccccc2)c2ccccc2c2ccccc12]

Here, “*-” corresponds to a position where Y is bonded to L₁.

When L₂ is a m-phenylene group, Z is not a substituted or unsubstituted 10-arylphenanthren-9-yl group.

When Y is a substituted or unsubstituted carbazole group, L₁ is a direct linkage or an unsubstituted phenylene group, and when Z is a substituted or unsubstituted carbazole group, L₂ is a direct linkage or an unsubstituted phenylene group.

Formula 1 includes a structure where a hydrogen atom is optionally substituted by (e.g., with) a deuterium atom. For example, Formula 1 may have a structure not including a deuterium atom, or a structure in which some or all hydrogen atoms are substituted with deuterium atoms. For example, when R₁ and R₂ are hydrogen atoms, the hydrogen atoms may be unsubstituted with deuterium atoms, or some or all hydrogen atoms may be substituted with deuterium atoms.

Formula B and Formula C may correspond to a second substituent and a third substituent, respectively, in the description.

In one or more embodiments, at least one selected from among Y of Formula B and Z of Formula C may be represented by any one selected from among Formula 1a to Formula 1c. For example, both (e.g., simultaneously) Y and Z may be represented by any one among Formula 1a to Formula 1c, and any one among Y and Z may be represented by Formula 1a to Formula 1c.

Formula 1a to Formula 1c represent cases where at least one selected from among Y and Z is a substituted or unsubstituted aryl group of 10 to 30 ring-forming carbon atoms, or a substituted or unsubstituted heteroaryl group of 12 to 30 ring-forming carbon atoms.

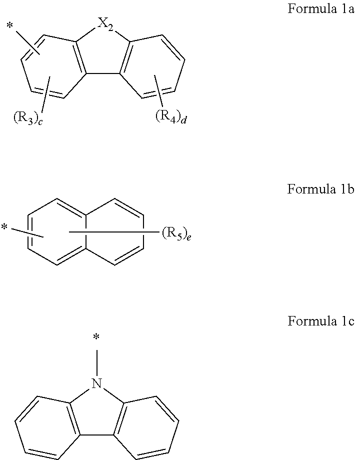

In Formula 1a, X₂ may be O, S, NRₐ, or CR_{b}R_{c}. For example, at least one selected from among Y of Formula B and Z of Formula C may be a substituted or unsubstituted dibenzofuran group, a substituted or unsubstituted dibenzothiophene group, a substituted or unsubstituted carbazole group, or a substituted or unsubstituted fluorenyl group.

In Formula 1a to Formula 1c, R₃ to R₅, and Rₐ to R_{c} may each independently be a hydrogen atom, a deuterium atom, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring.

R₃ to R₅ may each be the same, or at least one may be different from the remainder. For example, R₃ to R₅ may be hydrogen atoms or combined with adjacent groups to form hydrocarbon rings. R₃ to R₅ may be the same, or at least one may be different from the remainder.

“c” may be an integer of 0 to 3. A case where “c” is 0, may be the same as a case where “c” is 3, and all R₃ are hydrogen atoms. When “c” is an integer of 2 or more, two or more R₃ may be all the same, or at least one may be different from the remainder.

“d” may be an integer of 0 to 4. A case where “d” is 0, may be the same as a case where “d” is 4, and all R₄ are hydrogen atoms. When “d” is an integer of 2 or more, two or more R₄ may be all the same, or at least one may be different from the remainder.

“e” may be an integer of 0 to 7. A case where “e” is 0, may be the same as a case where “e” is 7, and all R₃ are hydrogen atoms. When “e” is an integer of 2 or more, two or more R₅ may be all the same, or at least one may be different from the remainder.

“*-” may be a position where Y is bonded to L₁ in Formula B, or a position where Z is bonded to L₂ in Formula C.

Formula 1a to Formula 1c include structures in which a hydrogen atom is optionally substituted by (e.g., with) a deuterium atom. For example, Formula 1a and Formula 1b may have structures not including a deuterium atom, or structures in which some or all hydrogen atoms are substituted with deuterium atoms. For example, all hydrogen atoms of R₃ and R₄ may be substituted with deuterium atoms.

In one or more embodiments, the amine compound represented by Formula 1 may be represented by Formula 2-1 or Formula 2-2.

Formula 2-1 and Formula 2-2 represent cases of Formula 1 where Ar is an unsubstituted phenyl group, and both (e.g., simultaneously) R₁ and R₂ are hydrogen atoms. In some embodiments, Formula 2-1 represents a case of Formula 1 where X₁ is O, and Formula 2-2 represents a case of Formula 1 where X₁ is S.

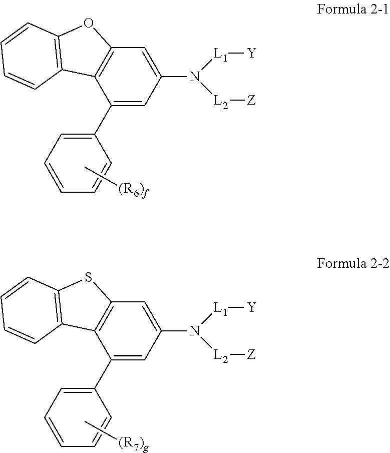

In Formula 2-1 and Formula 2-2, R₆ and R₇ may each independently be a hydrogen atom, a substituted or unsubstituted alkyl group of 1 to 10 carbon atoms, a substituted or unsubstituted alkenyl group of 2 to 10 carbon atoms, or a substituted or unsubstituted aryl group of 6 to 30 ring-forming carbon atoms, and/or combined with an adjacent group to form a ring. For example, R₆ and R₇ may each independently be a hydrogen atom, an unsubstituted cyclohexane group, an unsubstituted phenyl group, an unsubstituted naphthyl group, or an unsubstituted phenanthryl group.

“f” and “g” may each independently be an integer of 0 to 5. A case where “f” is 0, may be the same as a case where “f” is 5, and all R₆ are hydrogen atoms. When “f” is an integer of 2 or more, two or more R₆ may be the same, or at least one may be different from the remainder. A case where “g” is 0, may be the same as a case where “g” is 5, and all R₇ are hydrogen atoms. When “g” is an integer of 2 or more, two or more R₇ may be the same, or at least one may be different from the remainder.

L₁, L₂, Y and Z may each independently be as defined in Formula 1.

Formula 2-1 and Formula 2-2 may include structures in which a hydrogen atom is optionally substituted by (e.g., with) a deuterium atom. For example, when R₆ and R₇ are hydrogen atoms, the hydrogen atoms may be unsubstituted with deuterium atoms, or some or all hydrogen atoms may be substituted with deuterium atoms.

In one or more embodiments, both (e.g., simultaneously) R₁ and R₂ may be hydrogen atoms.

In one or more embodiments, L₁ and L₂ may each independently be a direct linkage, an unsubstituted phenylene group, or an unsubstituted biphenylene group.

In one or more embodiments, Ar_{A} may be any one selected from among a1 to a9, and b1 to b9:

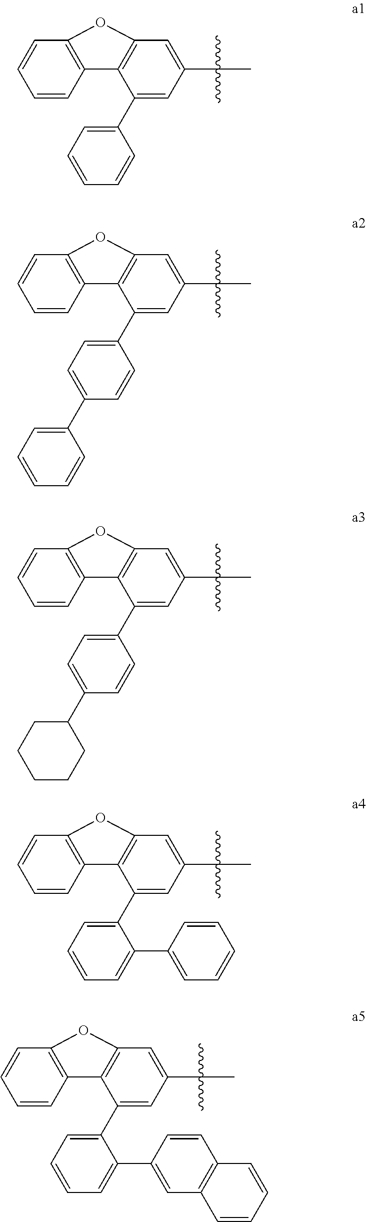 [SMILES: *c1cc(-c2ccccc2)c2c(c1)oc1ccccc12 ; *c1cc(-c2ccc(-c3ccccc3)cc2)c2c(c1)oc1ccccc12 ; *c1cc(-c2ccc(C3CCCCC3)cc2)c2c(c1)oc1ccccc12 ; *c1cc(-c2ccccc2-c2ccccc2)c2c(c1)oc1ccccc12 ; *c1cc(-c2ccccc2-c2ccc3ccccc3c2)c2c(c1)oc1ccccc12]

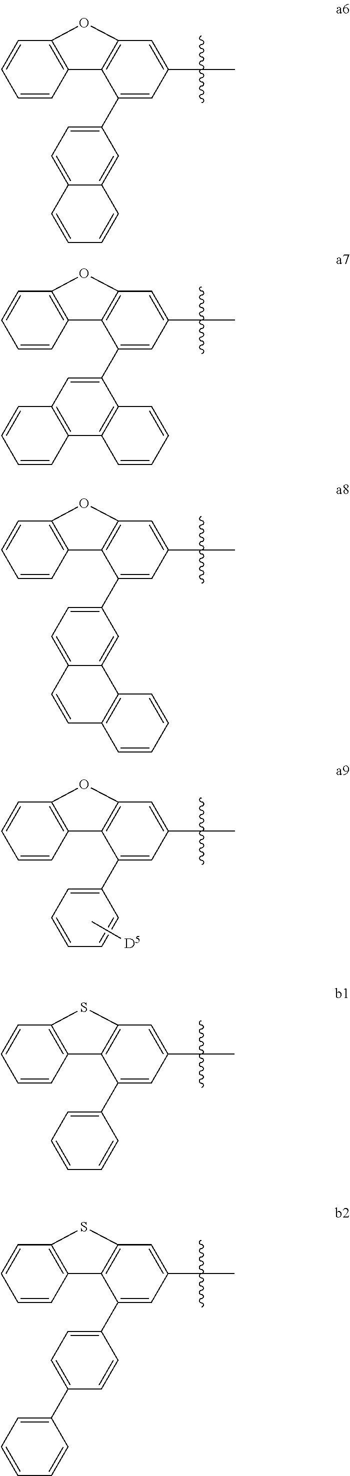

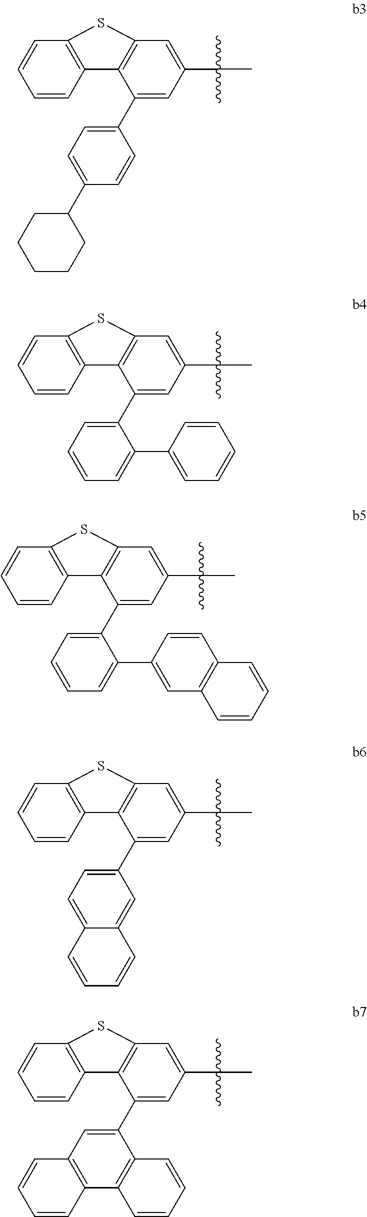

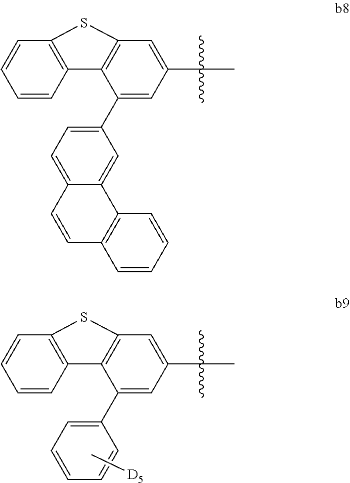

In a1 to a9, and b1 to b9, [symbol] is a position bonded to the nitrogen atom of Formula 1, and “D” is a deuterium atom.

In one or more embodiments, at least one selected from among Ar_{B} and Ar_{C} may be any one selected from among a1 and e1 to e67, and the remainder (e.g., when a remaining one selected from among Ar_{B} and Ar_{C} that is not a1 or e1 to e67) may be any one selected from among d1 to d10.

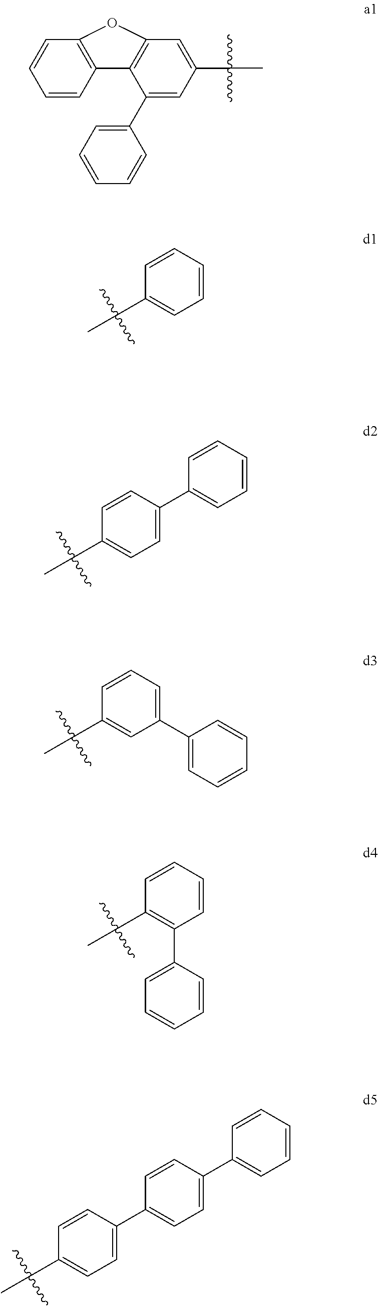 [SMILES: *c1cc(-c2ccccc2)c2c(c1)oc1ccccc12 ; *c1ccccc1 ; *c1ccc(-c2ccccc2)cc1 ; *c1cccc(-c2ccccc2)c1 ; *c1ccccc1-c1ccccc1 ; *c1ccc(-c2ccc(-c3ccccc3)cc2)cc1]

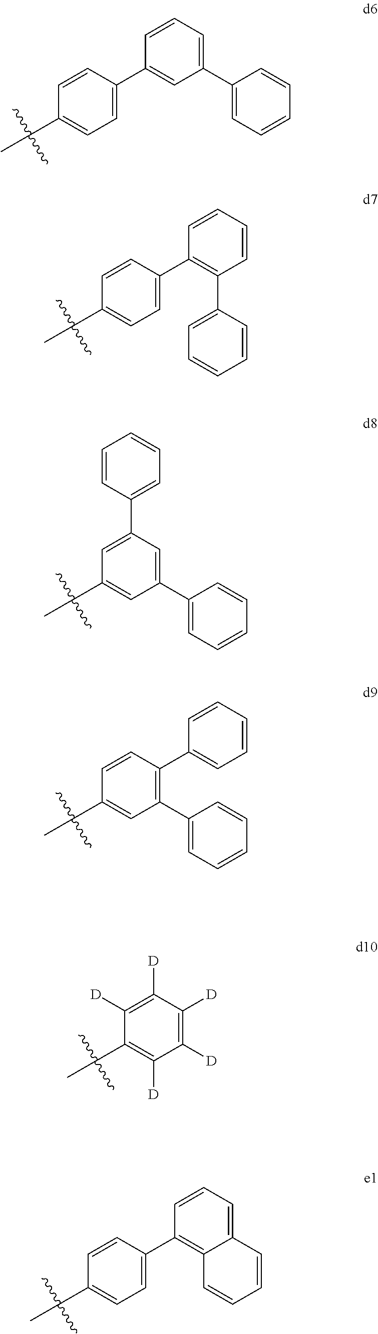 [SMILES: *c1ccc(-c2cccc(-c3ccccc3)c2)cc1 ; *c1ccc(-c2ccccc2-c2ccccc2)cc1 ; *c1cc(-c2ccccc2)cc(-c2ccccc2)c1 ; *c1ccc(-c2ccccc2)c(-c2ccccc2)c1 ; *c1c([2H])c([2H])c([2H])c([2H])c1[2H] ; *c1ccc(-c2cccc3ccccc23)cc1]

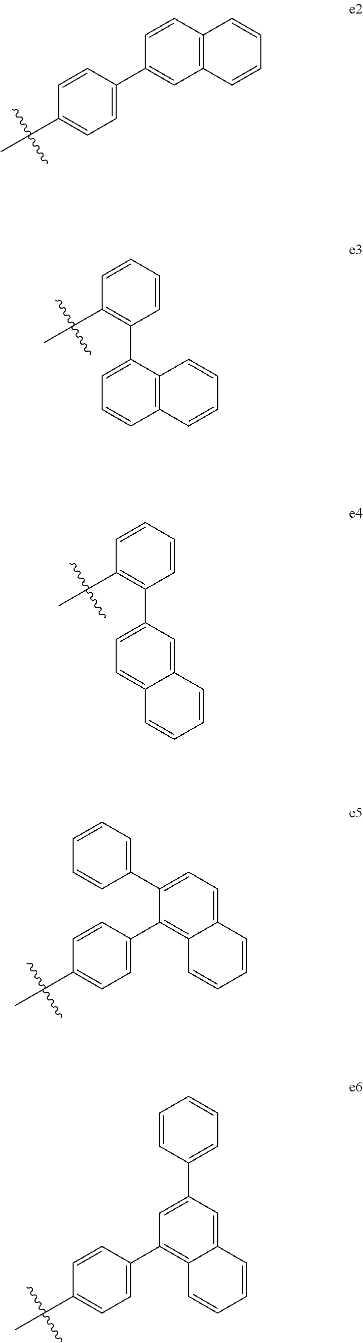 [SMILES: *c1ccc(-c2ccc3ccccc3c2)cc1 ; *c1ccccc1-c1cccc2ccccc12 ; *c1ccccc1-c1ccc2ccccc2c1 ; *c1ccc(-c2c(-c3ccccc3)ccc3ccccc23)cc1 ; *c1ccc(-c2cc(-c3ccccc3)cc3ccccc23)cc1]

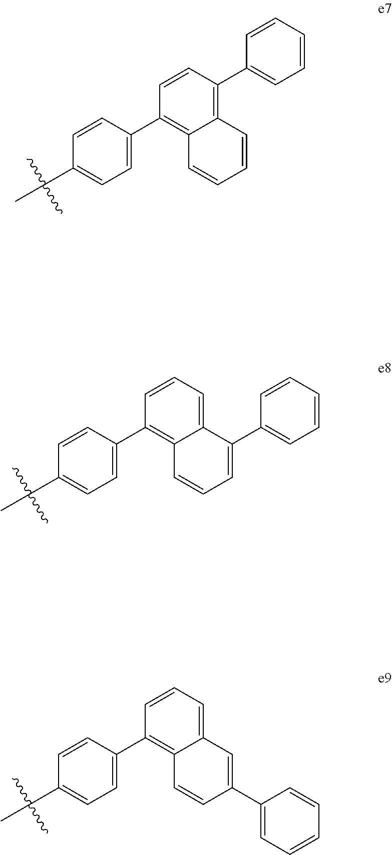 [SMILES: *c1ccc(-c2ccc(-c3ccccc3)c3ccccc23)cc1 ; *c1ccc(-c2cccc3c(-c4ccccc4)cccc23)cc1 ; *c1ccc(-c2cccc3cc(-c4ccccc4)ccc23)cc1]

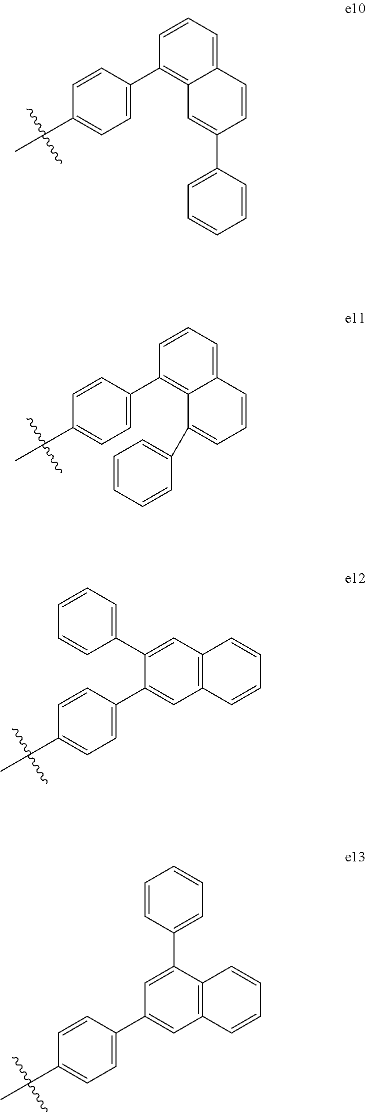 [SMILES: *c1ccc(-c2cccc3ccc(-c4ccccc4)cc23)cc1 ; *c1ccc(-c2cccc3cccc(-c4ccccc4)c23)cc1 ; *c1ccc(-c2cc3ccccc3cc2-c2ccccc2)cc1 ; *c1ccc(-c2cc(-c3ccccc3)c3ccccc3c2)cc1]

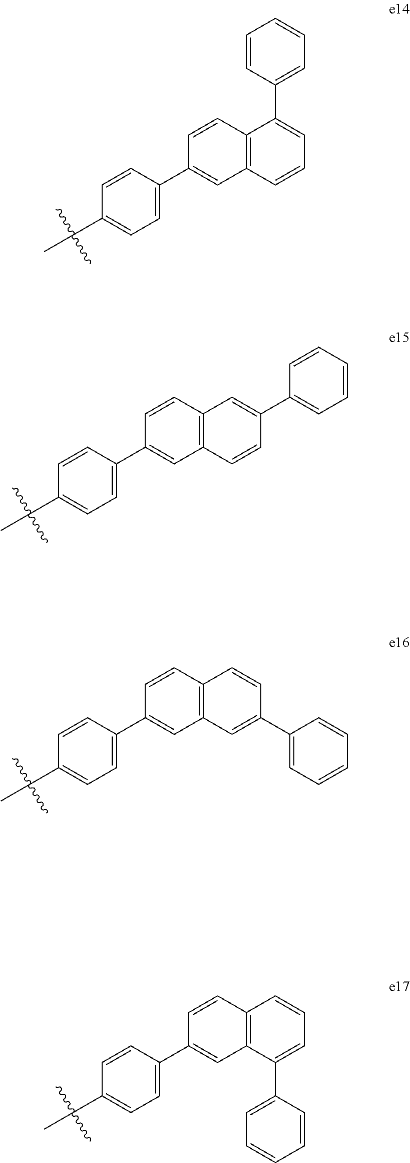 [SMILES: *c1ccc(-c2ccc3c(-c4ccccc4)cccc3c2)cc1 ; *c1ccc(-c2ccc3cc(-c4ccccc4)ccc3c2)cc1 ; *c1ccc(-c2ccc3ccc(-c4ccccc4)cc3c2)cc1 ; *c1ccc(-c2ccc3cccc(-c4ccccc4)c3c2)cc1]

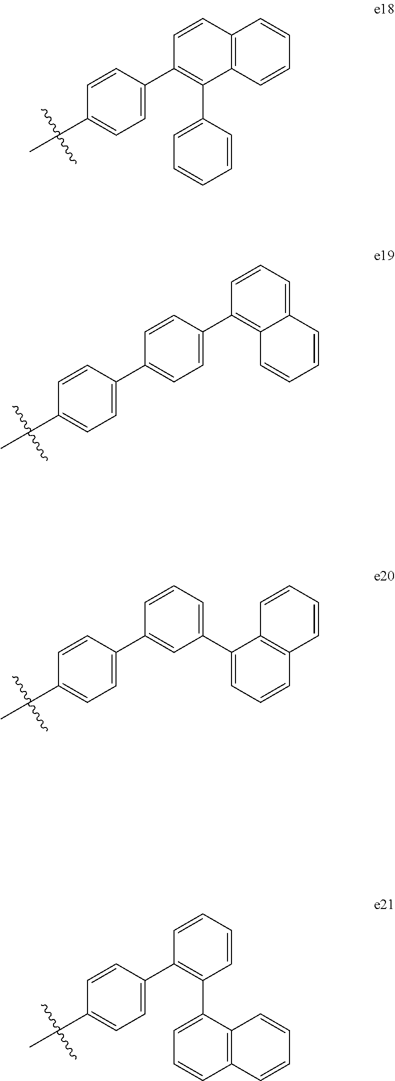 [SMILES: *c1ccc(-c2ccc3ccccc3c2-c2ccccc2)cc1 ; *c1ccc(-c2ccc(-c3cccc4ccccc34)cc2)cc1 ; *c1ccc(-c2cccc(-c3cccc4ccccc34)c2)cc1 ; *c1ccc(-c2ccccc2-c2cccc3ccccc23)cc1]

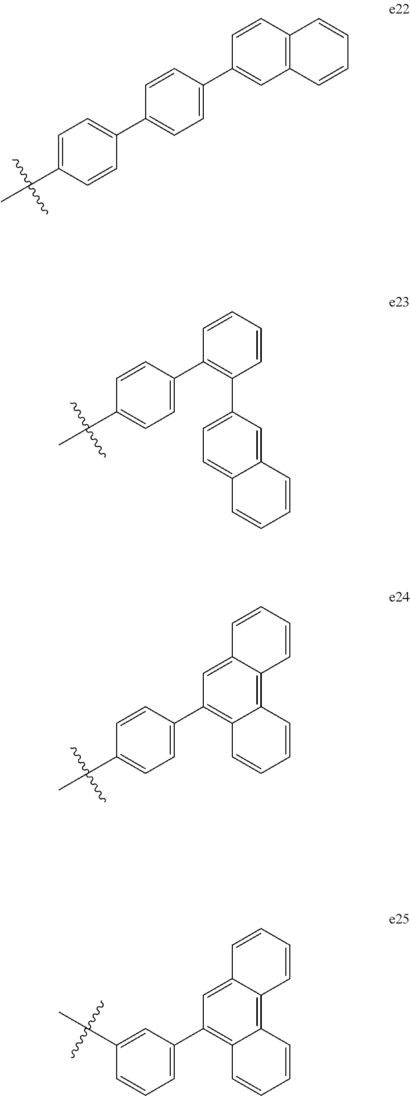 [SMILES: *c1ccc(-c2ccc(-c3ccc4ccccc4c3)cc2)cc1 ; *c1ccc(-c2ccccc2-c2ccc3ccccc3c2)cc1 ; *c1ccc(-c2cc3ccccc3c3ccccc23)cc1 ; *c1cccc(-c2cc3ccccc3c3ccccc23)c1]

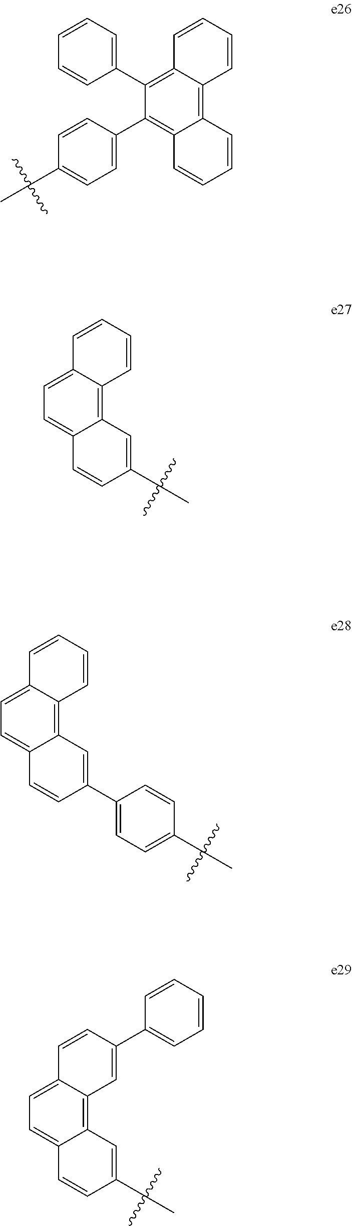 [SMILES: *c1ccc(-c2c(-c3ccccc3)c3ccccc3c3ccccc23)cc1 ; *c1ccc2ccc3ccccc3c2c1 ; *c1ccc(-c2ccc3ccc4ccccc4c3c2)cc1 ; *c1ccc2ccc3ccc(-c4ccccc4)cc3c2c1]

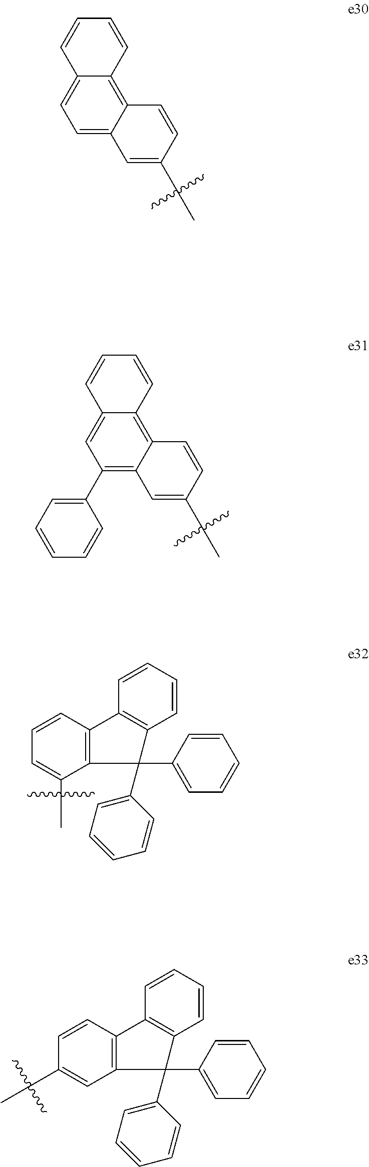 [SMILES: *c1ccc2c(ccc3ccccc32)c1 ; *c1ccc2c(c1)c(-c1ccccc1)cc1ccccc12 ; *c1cccc2c1C(c1ccccc1)(c1ccccc1)c1ccccc1-2 ; *c1ccc2c(c1)C(c1ccccc1)(c1ccccc1)c1ccccc1-2]

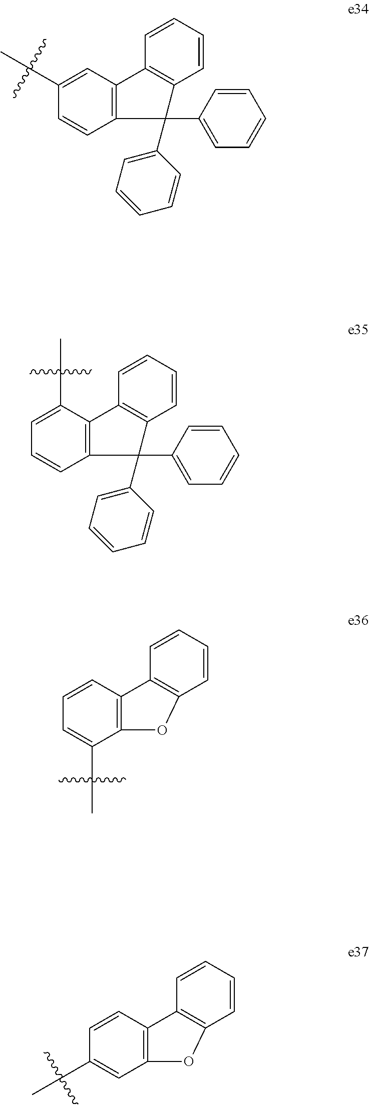 [SMILES: *c1ccc2c(c1)-c1ccccc1C2(c1ccccc1)c1ccccc1 ; *c1cccc2c1-c1ccccc1C2(c1ccccc1)c1ccccc1 ; *c1cccc2c1oc1ccccc12 ; *c1ccc2c(c1)oc1ccccc12]

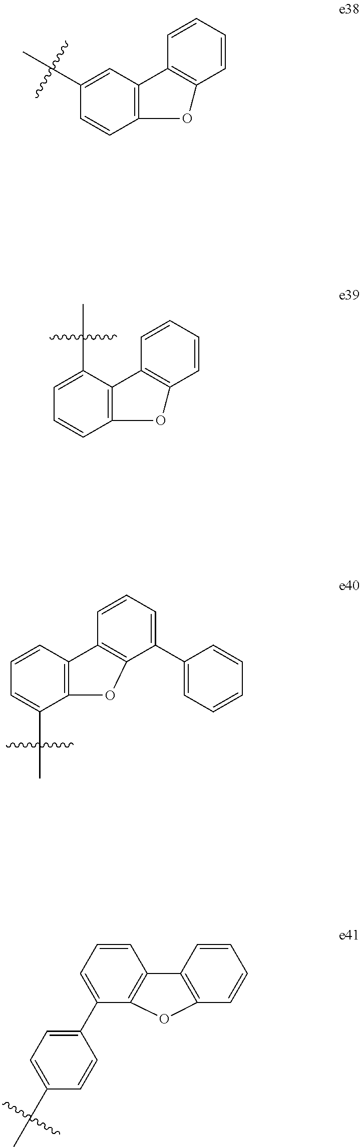 [SMILES: *c1ccc2oc3ccccc3c2c1 ; *c1cccc2oc3ccccc3c12 ; *c1cccc2c1oc1c(-c3ccccc3)cccc12 ; *c1ccc(-c2cccc3c2oc2ccccc23)cc1]

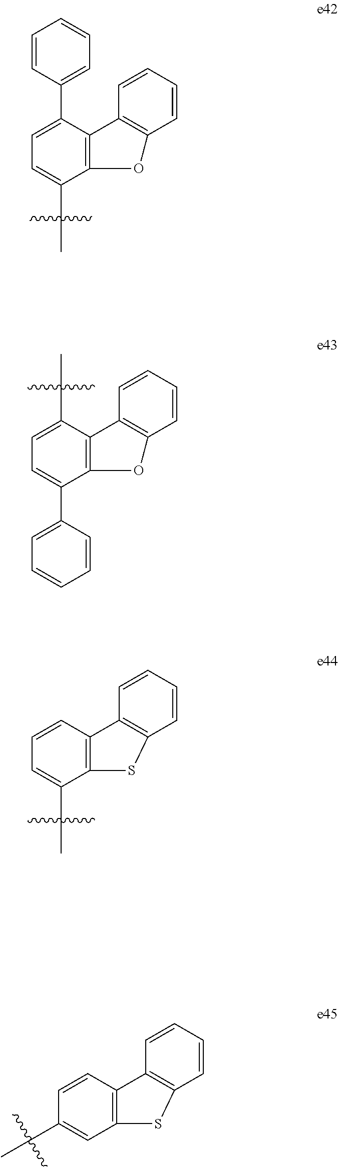 [SMILES: *c1ccc(-c2ccccc2)c2c1oc1ccccc12 ; *c1ccc(-c2ccccc2)c2oc3ccccc3c12 ; *c1cccc2c1sc1ccccc12 ; *c1ccc2c(c1)sc1ccccc12]

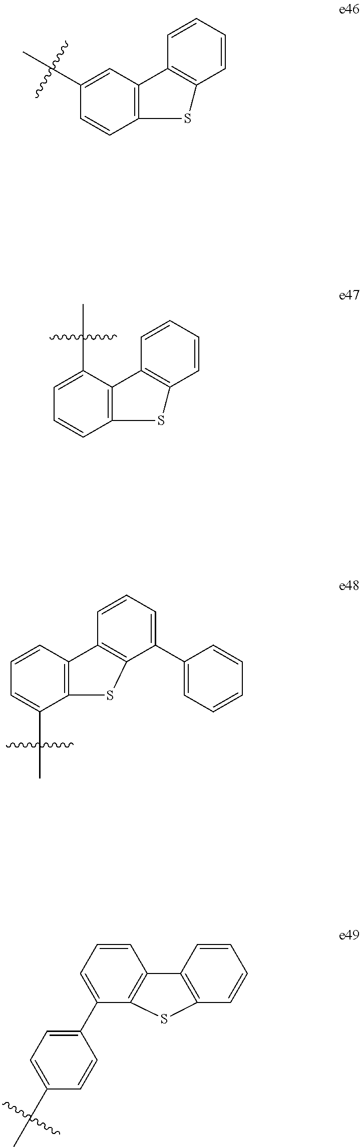 [SMILES: *c1ccc2sc3ccccc3c2c1 ; *c1cccc2sc3ccccc3c12 ; *c1cccc2c1sc1c(-c3ccccc3)cccc12 ; *c1ccc(-c2cccc3c2sc2ccccc23)cc1]

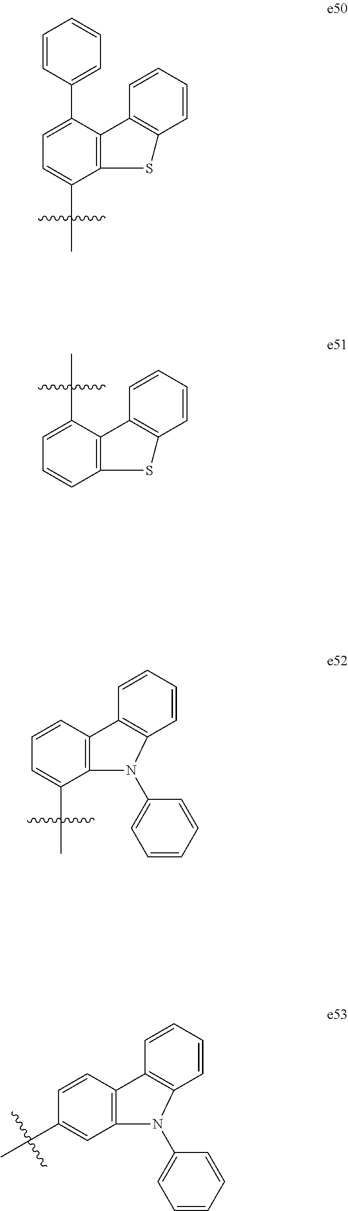 [SMILES: *c1ccc(-c2ccccc2)c2c1sc1ccccc12 ; *c1cccc2sc3ccccc3c12 ; *c1cccc2c3ccccc3n(-c3ccccc3)c12 ; *c1ccc2c3ccccc3n(-c3ccccc3)c2c1]

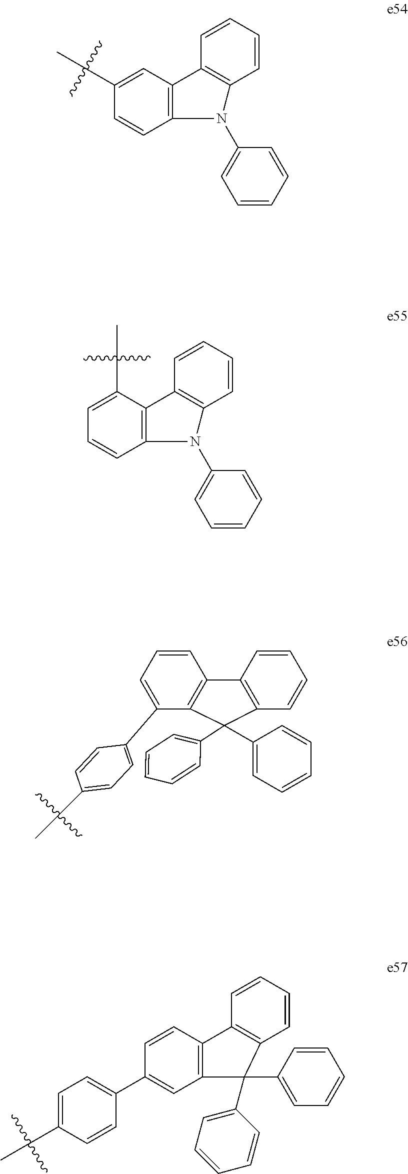 [SMILES: *c1ccc2c(c1)c1ccccc1n2-c1ccccc1 ; *c1cccc2c1c1ccccc1n2-c1ccccc1 ; *c1ccc(-c2cccc3c2C(c2ccccc2)(c2ccccc2)c2ccccc2-3)cc1 ; *c1ccc(-c2ccc3c(c2)C(c2ccccc2)(c2ccccc2)c2ccccc2-3)cc1]

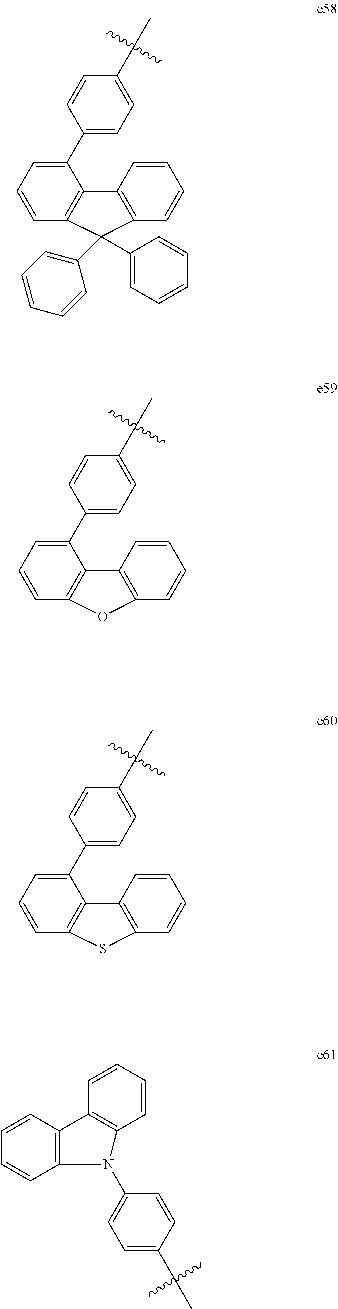 [SMILES: *c1ccc(-c2cccc3c2-c2ccccc2C3(c2ccccc2)c2ccccc2)cc1 ; *c1ccc(-c2cccc3oc4ccccc4c23)cc1 ; *c1ccc(-c2cccc3sc4ccccc4c23)cc1 ; *c1ccc(-n2c3ccccc3c3ccccc32)cc1]

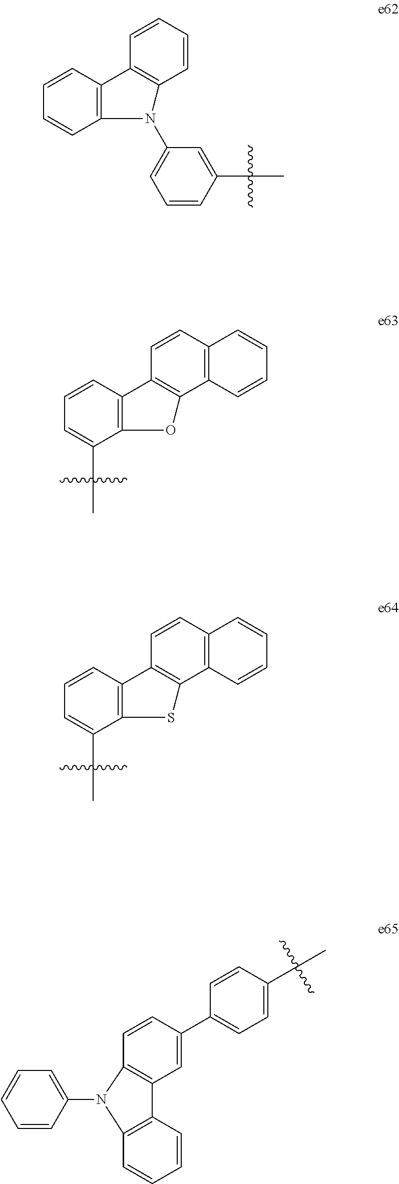 [SMILES: *c1cccc(-n2c3ccccc3c3ccccc32)c1 ; *c1cccc2c1oc1c3ccccc3ccc21 ; *c1cccc2c1sc1c3ccccc3ccc21 ; *c1ccc(-c2ccc3c(c2)c2ccccc2n3-c2ccccc2)cc1]

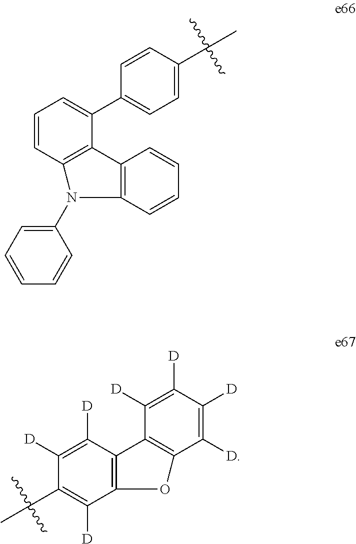

In a1, d1 to d10, and e1 to e67, [symbol] is a position bonded to the nitrogen atom of Formula 1, and “D” is a deuterium atom.

In one or more embodiments, the amine compound represented by Formula 1 may be any one selected from among the compounds in Compound Group 1 of Table 1 to Table 11. The light emitting element ED of one or more embodiments may include at least one selected from among the compounds in Table 1 to Table 11. Compound Group 1

TABLE 1

| Compound No. | Ar_{A} | Ar_{B} | Ar_{C} |
|---|---|---|---|
| 1 | a1 | e2 | e27 |
| 2 | a2 | e2 | e27 |
| 3 | a3 | e2 | e27 |
| 4 | a4 | e2 | e27 |
| 5 | a5 | e2 | e27 |
| 6 | a6 | e2 | e27 |
| 7 | a7 | e2 | e27 |
| 8 | a8 | e2 | e27 |
| 9 | a9 | e2 | e27 |
| 10 | b1 | e2 | e27 |
| 11 | b2 | e2 | e27 |
| 12 | b3 | e2 | e27 |
| 13 | b4 | e2 | e27 |
| 14 | b5 | e2 | e27 |
| 15 | b6 | e2 | e27 |
| 16 | b7 | e2 | e27 |
| 17 | b8 | e2 | e27 |
| 18 | b9 | e2 | e27 |
| 19 | a1 | d2 | a1 |
| 20 | a1 | d2 | e1 |
| 21 | a1 | d2 | e2 |
| 22 | a1 | d2 | e3 |
| 23 | a1 | d2 | e4 |
| 24 | a1 | d2 | e5 |
| 25 | a1 | d2 | e6 |
| 26 | a1 | d2 | e7 |
| 27 | a1 | d2 | e8 |
| 28 | a1 | d2 | e9 |
| 29 | a1 | d2 | e10 |
| 30 | a1 | d2 | e11 |
| 31 | a1 | d2 | e12 |
| 32 | a1 | d2 | e13 |
| 33 | a1 | d2 | e14 |
| 34 | a1 | d2 | e15 |
| 35 | a1 | d2 | e16 |
| 36 | a1 | d2 | e17 |
| 37 | a1 | d2 | e18 |
| 38 | a1 | d2 | e19 |
| 39 | a1 | d2 | e20 |
| 40 | a1 | d2 | e21 |
| 41 | a1 | d2 | e22 |
| 42 | a1 | d2 | e23 |
| 43 | a1 | d2 | e24 |
| 44 | a1 | d2 | e25 |
| 45 | a1 | d2 | e26 |
| 46 | a1 | d2 | e27 |
| 47 | a1 | d2 | e28 |
| 48 | a1 | d2 | e29 |
| 49 | a1 | d2 | e30 |
| 50 | a1 | d2 | e31 |
| 51 | a1 | d2 | e32 |
| 52 | a1 | d2 | e33 |
| 53 | a1 | d2 | e34 |
| 54 | a1 | d2 | e35 |
| 55 | a1 | d2 | e36 |
| 56 | a1 | d2 | e37 |
| 57 | a1 | d2 | e38 |
| 58 | a1 | d2 | e39 |
| 59 | a1 | d2 | e40 |
| 60 | a1 | d2 | e41 |
| 61 | a1 | d2 | e42 |
| 62 | a1 | d2 | e43 |
| 63 | a1 | d2 | e44 |
| 64 | a1 | d2 | e45 |
| 65 | a1 | d2 | e46 |
| 66 | a1 | d2 | e47 |
| 67 | a1 | d2 | e48 |
| 68 | a1 | d2 | e49 |
| 69 | a1 | d2 | e50 |
| 70 | a1 | d2 | e51 |
| 71 | a1 | d2 | e52 |
| 72 | a1 | d2 | e53 |
| 73 | a1 | d2 | e54 |
| 74 | a1 | d2 | e55 |
| 75 | a1 | d2 | e56 |
| 76 | a1 | d2 | e57 |
| 77 | a1 | d2 | e58 |
| 78 | a1 | d2 | e59 |
| 79 | a1 | d2 | e60 |
| 80 | a1 | d2 | e61 |
| 81 | a1 | d2 | e62 |
| 82 | a1 | d2 | e63 |
| 83 | a1 | d2 | e64 |
| 84 | a1 | d2 | e65 |
| 85 | a1 | d2 | e66 |
| 86 | a1 | d2 | e67 |
| 87 | a1 | d3 | a1 |
| 88 | a1 | d3 | e1 |
| 89 | a1 | d3 | e2 |
| 90 | a1 | d3 | e3 |
| 91 | a1 | d3 | e4 |
| 92 | a1 | d3 | e5 |
| 93 | a1 | d3 | e6 |
| 94 | a1 | d3 | e7 |
| 95 | a1 | d3 | e8 |
| 96 | a1 | d3 | e9 |
| 97 | a1 | d3 | e10 |
| 98 | a1 | d3 | e11 |
| 99 | a1 | d3 | e12 |
| 100 | a1 | d3 | e13 |
| 101 | a1 | d3 | e14 |
| 102 | a1 | d3 | e15 |
| 103 | a1 | d3 | e16 |
| 104 | a1 | d3 | e17 |
| 105 | a1 | d3 | e18 |
| 106 | a1 | d3 | e19 |
| 107 | a1 | d3 | e20 |
| 108 | a1 | d3 | e21 |
| 109 | a1 | d3 | e22 |
| 110 | a1 | d3 | e23 |
| 111 | a1 | d3 | e24 |
| 112 | a1 | d3 | e25 |
| 113 | a1 | d3 | e26 |
| 114 | a1 | d3 | e27 |
| 115 | a1 | d3 | e28 |
| 116 | a1 | d3 | e29 |
| 117 | a1 | d3 | e30 |
| 118 | a1 | d3 | e31 |
| 119 | a1 | d3 | e32 |
| 120 | a1 | d3 | e33 |
| 121 | a1 | d3 | e34 |
| 122 | a1 | d3 | e35 |
| 123 | a1 | d3 | e36 |
| 124 | a1 | d3 | e37 |
| 125 | a1 | d3 | e38 |
| 126 | a1 | d3 | e39 |
| 127 | a1 | d3 | e40 |
| 128 | a1 | d3 | e41 |
| 129 | a1 | d3 | e42 |
| 130 | a1 | d3 | e43 |
| 131 | a1 | d3 | e44 |
| 132 | a1 | d3 | e45 |
| 133 | a1 | d3 | e46 |
| 134 | a1 | d3 | e47 |
| 135 | a1 | d3 | e48 |
| 136 | a1 | d3 | e49 |
| 137 | a1 | d3 | e50 |
| 138 | a1 | d3 | e51 |
| 139 | a1 | d3 | e52 |
| 140 | a1 | d3 | e53 |
| 141 | a1 | d3 | e54 |
| 142 | a1 | d3 | e55 |
| 143 | a1 | d3 | e56 |
| 144 | a1 | d3 | e57 |
| 145 | a1 | d3 | e58 |
| 146 | a1 | d3 | e59 |
| 147 | a1 | d3 | e60 |
| 148 | a1 | d3 | e61 |
| 149 | a1 | d3 | e62 |
| 150 | a1 | d3 | e63 |
| 151 | a1 | d3 | e64 |
| 152 | a1 | d3 | e65 |
| 153 | a1 | d3 | e66 |
| 154 | a1 | d3 | e67 |
| 155 | a1 | d7 | a1 |
| 156 | a1 | d7 | e1 |
| 157 | a1 | d7 | e1 |
| 158 | a1 | d7 | e2 |
| 159 | a1 | d7 | e3 |
| 160 | a1 | d7 | e4 |
| 161 | a1 | d7 | e5 |
| 162 | a1 | d7 | e6 |
| 163 | a1 | d7 | e7 |
| 164 | a1 | d7 | e8 |
| 165 | a1 | d7 | e9 |
| 166 | a1 | d7 | e10 |
| 167 | a1 | d7 | e11 |
| 168 | a1 | d7 | e12 |
| 169 | a1 | d7 | e13 |
| 170 | a1 | d7 | e14 |
| 171 | a1 | d7 | e15 |
| 172 | a1 | d7 | e16 |
| 173 | a1 | d7 | e17 |
| 174 | a1 | d7 | e18 |
| 175 | a1 | d7 | e19 |
| 176 | a1 | d7 | e20 |
| 177 | a1 | d7 | e21 |
| 178 | a1 | d7 | e22 |
| 179 | a1 | d7 | e23 |
| 180 | a1 | d7 | e24 |
| 181 | a1 | d7 | e25 |
| 182 | a1 | d7 | e26 |
| 183 | a1 | d7 | e27 |
| 184 | a1 | d7 | e28 |
| 185 | a1 | d7 | e29 |
| 186 | a1 | d7 | e30 |
| 187 | a1 | d7 | e31 |
| 188 | a1 | d7 | e32 |
| 189 | a1 | d7 | e33 |
| 190 | a1 | d7 | e34 |
| 191 | a1 | d7 | e35 |
| 192 | a1 | d7 | e36 |
| 193 | a1 | d7 | e37 |
| 194 | a1 | d7 | e38 |
| 195 | a1 | d7 | e39 |
| 196 | a1 | d7 | e40 |
| 197 | a1 | d7 | e41 |
| 198 | a1 | d7 | e42 |
| 199 | a1 | d7 | e43 |
| 200 | a1 | d7 | e44 |

TABLE 2

| Compound No. | Ar_{A} | Ar_{B} | Ar_{C} |
|---|---|---|---|
| 201 | a1 | d7 | e45 |
| 202 | a1 | d7 | e46 |
| 203 | a1 | d7 | e47 |
| 204 | a1 | d7 | e48 |
| 205 | a1 | d7 | e49 |
| 206 | a1 | d7 | e50 |
| 207 | a1 | d7 | e51 |
| 208 | a1 | d7 | e52 |
| 209 | a1 | d7 | e53 |
| 210 | a1 | d7 | e54 |
| 211 | a1 | d7 | e55 |
| 212 | a1 | d7 | e56 |
| 213 | a1 | d7 | e57 |
| 214 | a1 | d7 | e58 |
| 215 | a1 | d7 | e59 |
| 216 | a1 | d7 | e60 |
| 217 | a1 | d7 | e61 |
| 218 | a1 | d7 | e62 |
| 219 | a1 | d7 | e63 |
| 220 | a1 | d7 | e64 |
| 221 | a1 | d7 | e65 |
| 222 | a1 | d7 | e66 |
| 223 | a1 | d7 | e67 |
| 224 | a1 | d9 | a1 |
| 225 | a1 | d9 | e1 |
| 226 | a1 | d9 | e1 |
| 227 | a1 | d9 | e2 |
| 228 | a1 | d9 | e3 |
| 229 | a1 | d9 | e4 |
| 230 | a1 | d9 | e5 |
| 231 | a1 | d9 | e6 |
| 232 | a1 | d9 | e7 |
| 233 | a1 | d9 | e8 |
| 234 | a1 | d9 | e9 |
| 235 | a1 | d9 | e10 |
| 236 | a1 | d9 | e11 |
| 237 | a1 | d9 | e12 |
| 238 | a1 | d9 | e13 |
| 239 | a1 | d9 | e14 |
| 240 | a1 | d9 | e15 |
| 241 | a1 | d9 | e16 |
| 242 | a1 | d9 | e17 |
| 243 | a1 | d9 | e18 |
| 244 | a1 | d9 | e19 |
| 245 | a1 | d9 | e20 |
| 246 | a1 | d9 | e21 |
| 247 | a1 | d9 | e22 |
| 248 | a1 | d9 | e23 |
| 249 | a1 | d9 | e24 |
| 250 | a1 | d9 | e25 |
| 251 | a1 | d9 | e26 |
| 252 | a1 | d9 | e27 |
| 253 | a1 | d9 | e28 |
| 254 | a1 | d9 | e29 |
| 255 | a1 | d9 | e30 |
| 256 | a1 | d9 | e31 |
| 257 | a1 | d9 | e32 |
| 258 | a1 | d9 | e33 |
| 259 | a1 | d9 | e34 |
| 260 | a1 | d9 | e35 |
| 261 | a1 | d9 | e36 |
| 262 | a1 | d9 | e37 |
| 263 | a1 | d9 | e38 |
| 264 | a1 | d9 | e39 |
| 265 | a1 | d9 | e40 |
| 266 | a1 | d9 | e41 |
| 267 | a1 | d9 | e42 |
| 268 | a1 | d9 | e43 |
| 269 | a1 | d9 | e44 |
| 270 | a1 | d9 | e45 |
| 271 | a1 | d9 | e46 |
| 272 | a1 | d9 | e47 |
| 273 | a1 | d9 | e48 |
| 274 | a1 | d9 | e49 |
| 275 | a1 | d9 | e50 |
| 276 | a1 | d9 | e51 |
| 277 | a1 | d9 | e52 |
| 278 | a1 | d9 | e53 |
| 279 | a1 | d9 | e54 |
| 280 | a1 | d9 | e55 |
| 281 | a1 | d9 | e56 |
| 282 | a1 | d9 | e57 |
| 283 | a1 | d9 | e58 |
| 284 | a1 | d9 | e59 |
| 285 | a1 | d9 | e60 |
| 286 | a1 | d9 | e61 |
| 287 | a1 | d9 | e62 |
| 288 | a1 | d9 | e63 |
| 289 | a1 | d9 | e64 |
| 290 | a1 | d9 | e65 |
| 291 | a1 | d9 | e66 |
| 292 | a1 | d9 | e67 |
| 293 | a1 | e1 | a1 |
| 294 | a1 | e1 | d1 |
| 295 | a1 | e1 | d1 |
| 296 | a1 | e1 | d4 |
| 297 | a1 | e1 | d5 |
| 298 | a1 | e1 | d6 |
| 299 | a1 | e1 | d8 |
| 300 | a1 | e1 | d10 |
| 301 | a1 | e1 | e1 |
| 302 | a1 | e1 | e1 |
| 303 | a1 | e1 | e2 |
| 304 | a1 | e1 | e3 |
| 305 | a1 | e1 | e4 |
| 306 | a1 | e1 | e5 |
| 307 | a1 | e1 | e6 |
| 308 | a1 | e1 | e7 |
| 309 | a1 | e1 | e8 |
| 310 | a1 | e1 | e9 |
| 311 | a1 | e1 | e10 |
| 312 | a1 | e1 | e11 |
| 313 | a1 | e1 | e12 |
| 314 | a1 | e1 | e13 |
| 315 | a1 | e1 | e14 |
| 316 | a1 | e1 | e15 |
| 317 | a1 | e1 | e16 |
| 318 | a1 | e1 | e17 |
| 319 | a1 | e1 | e18 |
| 320 | a1 | e1 | e19 |
| 321 | a1 | e1 | e20 |
| 322 | a1 | e1 | e21 |
| 323 | a1 | e1 | e22 |
| 324 | a1 | e1 | e23 |
| 325 | a1 | e1 | e24 |
| 326 | a1 | e1 | e25 |
| 327 | a1 | e1 | e26 |
| 328 | a1 | e1 | e27 |
| 329 | a1 | e1 | e28 |
| 330 | a1 | e1 | e29 |
| 331 | a1 | e1 | e30 |
| 332 | a1 | e1 | e31 |
| 333 | a1 | e1 | e32 |
| 334 | a1 | e1 | e33 |
| 335 | a1 | e1 | e34 |
| 336 | a1 | e1 | e35 |
| 337 | a1 | e1 | e36 |
| 338 | a1 | e1 | e37 |
| 339 | a1 | e1 | e38 |
| 340 | a1 | e1 | e39 |
| 341 | a1 | e1 | e40 |
| 342 | a1 | e1 | e41 |
| 343 | a1 | e1 | e42 |
| 344 | a1 | e1 | e43 |
| 345 | a1 | e1 | e44 |
| 346 | a1 | et | e45 |
| 347 | a1 | e1 | e46 |
| 348 | a1 | e1 | e47 |
| 349 | a1 | e1 | e48 |
| 350 | a1 | e1 | e49 |
| 351 | a1 | e1 | e50 |
| 352 | a1 | e1 | e51 |
| 353 | a1 | e1 | e52 |
| 354 | a1 | e1 | e53 |
| 355 | a1 | e1 | e54 |
| 356 | a1 | e1 | e55 |
| 357 | a1 | e1 | e56 |
| 358 | a1 | e1 | e57 |
| 359 | a1 | e1 | e58 |
| 360 | a1 | e1 | e59 |
| 361 | a1 | e1 | e60 |
| 362 | a1 | e1 | e61 |
| 363 | a1 | e1 | e62 |
| 364 | a1 | e1 | e63 |
| 365 | a1 | e1 | e64 |
| 366 | a1 | e1 | e65 |
| 367 | a1 | e1 | e66 |
| 368 | a1 | e1 | e67 |
| 369 | a1 | e2 | a1 |
| 370 | a1 | e2 | d1 |
| 371 | a1 | e2 | d1 |
| 372 | a1 | e2 | d4 |
| 373 | a1 | e2 | d5 |
| 374 | a1 | e2 | d6 |
| 375 | a1 | e2 | d8 |
| 376 | a1 | e2 | d10 |
| 377 | a1 | e2 | e2 |
| 378 | a1 | e2 | e3 |
| 379 | a1 | e2 | e4 |
| 380 | a1 | e2 | e5 |
| 381 | a1 | e2 | e6 |
| 382 | a1 | e2 | e7 |
| 383 | a1 | e2 | e8 |
| 384 | a1 | e2 | e9 |
| 385 | a1 | e2 | e10 |
| 386 | a1 | e2 | e11 |
| 387 | a1 | e2 | e12 |
| 388 | a1 | e2 | e13 |
| 389 | a1 | e2 | e14 |
| 390 | a1 | e2 | e15 |
| 391 | a1 | e2 | e16 |
| 392 | a1 | e2 | e17 |
| 393 | a1 | e2 | e18 |
| 394 | a1 | e2 | e19 |
| 395 | a1 | e2 | e20 |
| 396 | a1 | e2 | e21 |
| 397 | a1 | e2 | e22 |
| 398 | a1 | e2 | e23 |
| 399 | a1 | e2 | e24 |
| 400 | a1 | e2 | e25 |

TABLE 3

| Compound No. | Ar_{A} | Ar_{B} | Ar_{C} |
|---|---|---|---|
| 401 | a1 | e2 | e26 |
| 402 | a1 | e2 | e28 |
| 403 | a1 | e2 | e29 |
| 404 | a1 | e2 | e30 |
| 405 | a1 | e2 | e31 |
| 406 | a1 | e2 | e32 |
| 407 | a1 | e2 | e33 |
| 408 | a1 | e2 | e34 |
| 409 | a1 | e2 | e35 |
| 410 | a1 | e2 | e36 |
| 411 | a1 | e2 | e37 |
| 412 | a1 | e2 | e38 |
| 413 | a1 | e2 | e39 |
| 414 | a1 | e2 | e40 |
| 415 | a1 | e2 | e41 |
| 416 | a1 | e2 | e42 |
| 417 | a1 | e2 | e43 |
| 418 | a1 | e2 | e44 |
| 419 | a1 | e2 | e45 |
| 420 | a1 | e2 | e46 |
| 421 | a1 | e2 | e47 |
| 422 | a1 | e2 | e48 |
| 423 | a1 | e2 | e49 |
| 424 | a1 | e2 | e50 |
| 425 | a1 | e2 | e51 |
| 426 | a1 | e2 | e52 |
| 427 | a1 | e2 | e53 |
| 428 | a1 | e2 | e54 |
| 429 | a1 | e2 | e55 |
| 430 | a1 | e2 | e56 |
| 431 | a1 | e2 | e57 |
| 432 | a1 | e2 | e58 |
| 433 | a1 | e2 | e59 |
| 434 | a1 | e2 | e60 |
| 435 | a1 | e2 | e61 |
| 436 | a1 | e2 | e62 |
| 437 | a1 | e2 | e63 |
| 438 | a1 | e2 | e64 |
| 439 | a1 | e2 | e65 |
| 440 | a1 | e2 | e66 |
| 441 | a1 | e2 | e67 |
| 442 | a1 | e24 | a1 |
| 443 | a1 | e24 | d1 |
| 444 | a1 | e24 | d1 |
| 445 | a1 | e24 | d4 |
| 446 | a1 | e24 | d5 |
| 447 | a1 | e24 | d6 |
| 448 | a1 | e24 | d8 |
| 449 | a1 | e24 | d10 |
| 450 | a1 | e24 | e3 |
| 451 | a1 | e24 | e4 |
| 452 | a1 | e24 | e5 |
| 453 | a1 | e24 | e6 |
| 454 | a1 | e24 | e7 |
| 455 | a1 | e24 | e8 |
| 456 | a1 | e24 | e9 |
| 457 | a1 | e24 | e10 |
| 458 | a1 | e24 | e11 |
| 459 | a1 | e24 | e12 |
| 460 | a1 | e24 | e13 |
| 461 | a1 | e24 | e14 |
| 462 | a1 | e24 | e15 |
| 463 | a1 | e24 | e16 |
| 464 | a1 | e24 | e17 |
| 465 | a1 | e24 | e18 |
| 466 | a1 | e24 | e19 |
| 467 | a1 | e24 | e20 |
| 468 | a1 | e24 | e21 |
| 469 | a1 | e24 | e22 |
| 470 | a1 | e24 | e23 |
| 471 | a1 | e24 | e24 |
| 472 | a1 | e24 | e25 |
| 473 | a1 | e24 | e26 |
| 474 | a1 | e24 | e27 |
| 475 | a1 | e24 | e28 |
| 476 | a1 | e24 | e29 |
| 477 | a1 | e24 | e30 |
| 478 | a1 | e24 | e31 |
| 479 | a1 | e24 | e32 |
| 480 | a1 | e24 | e33 |
| 481 | a1 | e24 | e34 |
| 482 | a1 | e24 | e35 |
| 483 | a1 | e24 | e36 |
| 484 | a1 | e24 | e37 |
| 485 | a1 | e24 | e38 |
| 486 | a1 | e24 | e39 |
| 487 | a1 | e24 | e40 |
| 488 | a1 | e24 | e41 |
| 489 | a1 | e24 | e42 |
| 490 | a1 | e24 | e43 |
| 491 | a1 | e24 | e44 |
| 492 | a1 | e24 | e45 |
| 493 | a1 | e24 | e46 |
| 494 | a1 | e24 | e47 |
| 495 | a1 | e24 | e48 |
| 496 | a1 | e24 | e49 |
| 497 | a1 | e24 | e50 |
| 498 | a1 | e24 | e51 |
| 499 | a1 | e24 | e52 |
| 500 | a1 | e24 | e53 |
| 501 | a1 | e24 | e54 |
| 502 | a1 | e24 | e55 |
| 503 | a1 | e24 | e56 |
| 504 | a1 | e24 | e57 |
| 505 | a1 | e24 | e58 |
| 506 | a1 | e24 | e59 |
| 507 | a1 | e24 | e60 |
| 508 | a1 | e24 | e61 |
| 509 | a1 | e24 | e62 |
| 510 | a1 | e24 | e63 |
| 511 | a1 | e24 | e64 |
| 512 | a1 | e24 | e65 |
| 513 | a1 | e24 | e66 |
| 514 | a1 | e24 | e67 |
| 515 | a1 | e27 | a1 |
| 516 | a1 | e27 | d1 |
| 517 | a1 | e27 | d1 |
| 518 | a1 | e27 | d4 |
| 519 | a1 | e27 | d5 |
| 520 | a1 | e27 | d6 |
| 521 | a1 | e27 | d8 |
| 522 | a1 | e27 | d10 |
| 523 | a1 | e27 | e3 |
| 524 | a1 | e27 | e4 |
| 525 | a1 | e27 | e5 |
| 526 | a1 | e27 | e6 |
| 527 | a1 | e27 | e7 |
| 528 | a1 | e27 | e8 |
| 529 | a1 | e27 | e9 |
| 530 | a1 | e27 | e10 |
| 531 | a1 | e27 | e11 |
| 532 | a1 | e27 | e12 |
| 533 | a1 | e27 | e13 |
| 534 | a1 | e27 | e14 |
| 535 | a1 | e27 | e15 |
| 536 | a1 | e27 | e16 |
| 537 | a1 | e27 | e17 |
| 538 | a1 | e27 | e18 |
| 539 | a1 | e27 | e19 |
| 540 | a1 | e27 | e20 |
| 541 | a1 | e27 | e21 |
| 542 | a1 | e27 | e22 |
| 543 | a1 | e27 | e23 |
| 544 | a1 | e27 | e25 |
| 545 | a1 | e27 | e26 |
| 546 | a1 | e27 | e27 |
| 547 | a1 | e27 | e28 |
| 548 | a1 | e27 | e29 |
| 549 | a1 | e27 | e30 |
| 550 | a1 | e27 | e31 |
| 551 | a1 | e27 | e32 |
| 552 | a1 | e27 | e33 |
| 553 | a1 | e27 | e34 |
| 554 | a1 | e27 | e35 |
| 555 | a1 | e27 | e36 |
| 556 | a1 | e27 | e37 |
| 557 | a1 | e27 | e38 |
| 558 | a1 | e27 | e39 |
| 559 | a1 | e27 | e40 |
| 560 | a1 | e27 | e41 |
| 561 | a1 | e27 | e42 |
| 562 | a1 | e27 | e43 |
| 563 | a1 | e27 | e44 |
| 564 | a1 | e27 | e45 |
| 565 | a1 | e27 | e46 |
| 566 | a1 | e27 | e47 |
| 567 | a1 | e27 | e48 |
| 568 | a1 | e27 | e49 |
| 569 | a1 | e27 | e50 |
| 570 | a1 | e27 | e51 |
| 571 | a1 | e27 | e52 |
| 572 | a1 | e27 | e53 |
| 573 | a1 | e27 | e54 |
| 574 | a1 | e27 | e55 |
| 575 | a1 | e27 | e56 |
| 576 | a1 | e27 | e57 |
| 577 | a1 | e27 | e58 |
| 578 | a1 | e27 | e59 |
| 579 | a1 | e27 | e60 |
| 580 | a1 | e27 | e61 |
| 581 | a1 | e27 | e62 |
| 582 | a1 | e27 | e63 |
| 583 | a1 | e27 | e64 |
| 584 | a1 | e27 | e65 |
| 585 | a1 | e27 | e66 |
| 586 | a1 | e27 | e67 |
| 587 | a1 | e33 | a1 |
| 588 | a1 | e33 | d1 |
| 589 | a1 | e33 | d1 |
| 590 | a1 | e33 | d4 |
| 591 | a1 | e33 | d5 |
| 592 | a1 | e33 | d6 |
| 593 | a1 | e33 | d8 |
| 594 | a1 | e33 | d10 |
| 595 | a1 | e33 | e3 |
| 596 | a1 | e33 | e4 |
| 597 | a1 | e33 | e5 |
| 598 | a1 | e33 | e6 |
| 599 | a1 | e33 | e7 |
| 600 | a1 | e33 | e8 |

TABLE 4

| Compound No. | Ar_{A} | Ar_{B} | Ar_{C} |
|---|---|---|---|
| 801 | a1 | e41 | d4 |
| 802 | a1 | e41 | d5 |
| 803 | a1 | e41 | d6 |
| 804 | a1 | e41 | d8 |
| 805 | a1 | e41 | d10 |
| 806 | a1 | e41 | e3 |
| 807 | a1 | e41 | e4 |
| 808 | a1 | e41 | e5 |
| 809 | a1 | e41 | e6 |
| 810 | a1 | e41 | e7 |
| 811 | a1 | e41 | e8 |
| 812 | a1 | e41 | e9 |
| 813 | a1 | e41 | e10 |
| 814 | a1 | e41 | e11 |
| 815 | a1 | e41 | e12 |
| 816 | a1 | e41 | e13 |
| 817 | a1 | e41 | e14 |
| 818 | a1 | e41 | e15 |
| 819 | a1 | e41 | e16 |
| 820 | a1 | e41 | e17 |
| 821 | a1 | e41 | e18 |
| 822 | a1 | e41 | e19 |
| 823 | a1 | e41 | e20 |
| 824 | a1 | e41 | e21 |
| 825 | a1 | e41 | e22 |
| 826 | a1 | e41 | e23 |
| 827 | a1 | e41 | e25 |
| 828 | a1 | e41 | e26 |
| 829 | a1 | e41 | e28 |
| 830 | a1 | e41 | e29 |
| 831 | a1 | e41 | e30 |
| 832 | a1 | e41 | e31 |
| 833 | a1 | e41 | e32 |
| 834 | a1 | e41 | e34 |
| 835 | a1 | e41 | e35 |
| 836 | a1 | e41 | e38 |
| 837 | a1 | e41 | e39 |
| 838 | a1 | e41 | e40 |
| 839 | a1 | e41 | e41 |
| 840 | a1 | e41 | e42 |
| 841 | a1 | e41 | e43 |
| 842 | a1 | e41 | e44 |
| 843 | a1 | e41 | e45 |
| 844 | a1 | e41 | e46 |
| 845 | a1 | e41 | e47 |
| 846 | a1 | e41 | e48 |
| 847 | a1 | e41 | e49 |
| 848 | a1 | e41 | e50 |
| 849 | a1 | e41 | e51 |
| 850 | a1 | e41 | e52 |
| 851 | a1 | e41 | e53 |
| 852 | a1 | e41 | e54 |
| 853 | a1 | e41 | e55 |
| 854 | a1 | e41 | e56 |
| 855 | a1 | e41 | e57 |
| 856 | a1 | e41 | e58 |
| 857 | a1 | e41 | e59 |
| 858 | a1 | e41 | e60 |
| 859 | a1 | e41 | e61 |
| 860 | a1 | e41 | e62 |
| 861 | a1 | e41 | e63 |
| 862 | a1 | e41 | e64 |
| 863 | a1 | e41 | e65 |
| 864 | a1 | e41 | e66 |
| 865 | a1 | e41 | e67 |
| 866 | a1 | e59 | a1 |
| 867 | a1 | e59 | d1 |
| 868 | a1 | e59 | d1 |
| 869 | a1 | e59 | d4 |
| 870 | a1 | e59 | d5 |
| 871 | a1 | e59 | d6 |
| 872 | a1 | e59 | d8 |
| 873 | a1 | e59 | d10 |
| 874 | a1 | e59 | e3 |
| 875 | a1 | e59 | e4 |
| 876 | a1 | e59 | e5 |
| 877 | a1 | e59 | e6 |
| 878 | a1 | e59 | e7 |
| 879 | a1 | e59 | e8 |
| 880 | a1 | e59 | e9 |
| 881 | a1 | e59 | e10 |
| 882 | a1 | e59 | e11 |
| 883 | a1 | e59 | e12 |
| 884 | a1 | e59 | e13 |
| 885 | a1 | e59 | e14 |
| 886 | a1 | e59 | e15 |
| 887 | a1 | e59 | e16 |
| 888 | a1 | e59 | e17 |
| 889 | a1 | e59 | e18 |
| 890 | a1 | e59 | e19 |
| 891 | a1 | e59 | e20 |
| 892 | a1 | e59 | e21 |
| 893 | a1 | e59 | e22 |
| 894 | a1 | e59 | e23 |
| 895 | a1 | e59 | e25 |
| 896 | a1 | e59 | e26 |
| 897 | a1 | e59 | e28 |
| 898 | a1 | e59 | e29 |
| 899 | a1 | e59 | e30 |
| 900 | a1 | e59 | e31 |
| 901 | a1 | e59 | e32 |
| 902 | a1 | e59 | e34 |
| 903 | a1 | e59 | e35 |
| 904 | a1 | e59 | e38 |
| 905 | a1 | e59 | e39 |
| 906 | a1 | e59 | e40 |
| 907 | a1 | e59 | e42 |
| 908 | a1 | e59 | e43 |
| 909 | a1 | e59 | e44 |
| 910 | a1 | e59 | e45 |
| 911 | a1 | e59 | e46 |
| 912 | a1 | e59 | e47 |
| 913 | a1 | e59 | e48 |
| 914 | a1 | e59 | e49 |
| 915 | a1 | e59 | e50 |
| 916 | a1 | e59 | e51 |
| 917 | a1 | e59 | e52 |
| 918 | a1 | e59 | e53 |
| 919 | a1 | e59 | e54 |
| 920 | a1 | e59 | e55 |
| 921 | a1 | e59 | e56 |
| 922 | a1 | e59 | e57 |
| 923 | a1 | e59 | e58 |
| 924 | a1 | e59 | e59 |
| 925 | a1 | e59 | e60 |
| 926 | a1 | e59 | e61 |
| 927 | a1 | e59 | e62 |
| 928 | a1 | e59 | e63 |
| 929 | a1 | e59 | e64 |
| 930 | a1 | e59 | e65 |
| 931 | a1 | e59 | e66 |
| 932 | a1 | e59 | e67 |
| 933 | a1 | e62 | a1 |
| 934 | a1 | e62 | d1 |
| 935 | a1 | e62 | d1 |
| 936 | a1 | e62 | d5 |
| 937 | a1 | e62 | d6 |
| 938 | a1 | e62 | d8 |
| 939 | a1 | e62 | d10 |
| 940 | a1 | e62 | e5 |
| 941 | a1 | e62 | e6 |
| 942 | a1 | e62 | e7 |
| 943 | a1 | e62 | e8 |
| 944 | a1 | e62 | e9 |
| 945 | a1 | e62 | e10 |
| 946 | a1 | e62 | e11 |
| 947 | a1 | e62 | e12 |
| 948 | a1 | e62 | e13 |
| 949 | a1 | e62 | e14 |
| 950 | a1 | e62 | e15 |
| 951 | a1 | e62 | e16 |
| 952 | a1 | e62 | e17 |
| 953 | a1 | e62 | e18 |
| 954 | a1 | e62 | e19 |
| 955 | a1 | e62 | e20 |
| 956 | a1 | e62 | e21 |
| 957 | a1 | e62 | e22 |
| 958 | a1 | e62 | e23 |
| 959 | a1 | e62 | e25 |
| 960 | a1 | e62 | e26 |
| 961 | a1 | e62 | e28 |
| 962 | a1 | e62 | e29 |
| 963 | a1 | e62 | e30 |
| 964 | a1 | e62 | e31 |
| 965 | a1 | e62 | e32 |
| 966 | a1 | e62 | e34 |
| 967 | a1 | e62 | e35 |
| 968 | a1 | e62 | e38 |
| 969 | a1 | e62 | e39 |
| 970 | a1 | e62 | e40 |
| 971 | a1 | e62 | e42 |
| 972 | a1 | e62 | e43 |
| 973 | a1 | e62 | e44 |
| 974 | a1 | e62 | e45 |
| 975 | a1 | e62 | e46 |
| 976 | a1 | e62 | e47 |
| 977 | a1 | e62 | e48 |
| 978 | a1 | e62 | e49 |
| 979 | a1 | e62 | e50 |
| 980 | a1 | e62 | e51 |
| 981 | a1 | e62 | e52 |
| 982 | a1 | e62 | e53 |
| 983 | a1 | e62 | e54 |
| 984 | a1 | e62 | e55 |
| 985 | a1 | e62 | e56 |
| 986 | a1 | e62 | e57 |
| 987 | a1 | e62 | e58 |
| 988 | a1 | e62 | e60 |
| 989 | a1 | e62 | e61 |
| 990 | a1 | e62 | e62 |
| 991 | a1 | e62 | e63 |
| 992 | a1 | e62 | e64 |
| 993 | a1 | e62 | e65 |
| 994 | a1 | e62 | e66 |
| 995 | a1 | e62 | e67 |
| 996 | a1 | e63 | a1 |
| 997 | a1 | e63 | d1 |
| 998 | a1 | e63 | d1 |
| 999 | a1 | e63 | d4 |
| 1000 | a1 | e63 | d5 |

TABLE 5

| Compound No. | Ar_{A} | Ar_{B} | Ar_{C} |
|---|---|---|---|
| 1001 | a1 | e63 | d6 |
| 1002 | a1 | e63 | d8 |
| 1003 | a1 | e63 | d10 |
| 1004 | a1 | e63 | e3 |
| 1005 | a1 | e63 | e4 |
| 1006 | a1 | e63 | e5 |
| 1007 | a1 | e63 | e6 |
| 1008 | a1 | e63 | e7 |
| 1009 | a1 | e63 | e8 |
| 1010 | a1 | e63 | e9 |
| 1011 | a1 | e63 | e10 |
| 1012 | a1 | e63 | e11 |
| 1013 | a1 | e63 | e12 |
| 1014 | a1 | e63 | e13 |
| 1015 | a1 | e63 | e14 |
| 1016 | a1 | e63 | e15 |
| 1017 | a1 | e63 | e16 |
| 1018 | a1 | e63 | e17 |
| 1019 | a1 | e63 | e18 |
| 1020 | a1 | e63 | e19 |
| 1021 | a1 | e63 | e20 |
| 1022 | a1 | e63 | e21 |
| 1023 | a1 | e63 | e22 |
| 1024 | a1 | e63 | e23 |
| 1025 | a1 | e63 | e25 |
| 1026 | a1 | e63 | e26 |
| 1027 | a1 | e63 | e28 |
| 1028 | a1 | e63 | e29 |
| 1029 | a1 | e63 | e30 |
| 1030 | a1 | e63 | e31 |
| 1031 | a1 | e63 | e32 |
| 1032 | a1 | e63 | e34 |
| 1033 | a1 | e63 | e35 |
| 1034 | a1 | e63 | e38 |
| 1035 | a1 | e63 | e39 |
| 1036 | a1 | e63 | e40 |
| 1037 | a1 | e63 | e42 |
| 1038 | a1 | e63 | e43 |
| 1039 | a1 | e63 | e44 |
| 1040 | a1 | e63 | e45 |
| 1041 | a1 | e63 | e46 |
| 1042 | a1 | e63 | e47 |
| 1043 | a1 | e63 | e48 |
| 1044 | a1 | e63 | e49 |
| 1045 | a1 | e63 | e50 |
| 1046 | a1 | e63 | e51 |
| 1047 | a1 | e63 | e52 |
| 1048 | a1 | e63 | e53 |
| 1049 | a1 | e63 | e54 |
| 1050 | a1 | e63 | e55 |
| 1051 | a1 | e63 | e56 |
| 1052 | a1 | e63 | e57 |
| 1053 | a1 | e63 | e58 |
| 1054 | a1 | e63 | e60 |
| 1055 | a1 | e63 | e61 |
| 1056 | a1 | e63 | e63 |
| 1057 | a1 | e63 | e64 |
| 1058 | a1 | e63 | e65 |
| 1059 | a1 | e63 | e66 |
| 1060 | a1 | e63 | e67 |
| 1061 | a1 | e64 | a1 |
| 1062 | a1 | e64 | d1 |
| 1063 | a1 | e64 | d1 |
| 1064 | a1 | e64 | d4 |
| 1065 | a1 | e64 | d5 |
| 1066 | a1 | e64 | d6 |
| 1067 | a1 | e64 | d8 |
| 1068 | a1 | e64 | d10 |
| 1069 | a1 | e64 | e3 |
| 1070 | a1 | e64 | e4 |
| 1071 | a1 | e64 | e5 |
| 1072 | a1 | e64 | e6 |
| 1073 | a1 | e64 | e7 |
| 1074 | a1 | e64 | e8 |
| 1075 | a1 | e64 | e9 |
| 1076 | a1 | e64 | e10 |
| 1077 | a1 | e64 | e11 |
| 1078 | a1 | e64 | e12 |
| 1079 | a1 | e64 | e13 |
| 1080 | a1 | e64 | e14 |
| 1081 | a1 | e64 | e15 |
| 1082 | a1 | e64 | e16 |
| 1083 | a1 | e64 | e17 |
| 1084 | a1 | e64 | e18 |
| 1085 | a1 | e64 | e19 |
| 1086 | a1 | e64 | e20 |
| 1087 | a1 | e64 | e21 |
| 1088 | a1 | e64 | e22 |
| 1089 | a1 | e64 | e23 |
| 1090 | a1 | e64 | e25 |
| 1091 | a1 | e64 | e26 |
| 1092 | a1 | e64 | e28 |
| 1093 | a1 | e64 | e29 |
| 1094 | a1 | e64 | e30 |
| 1095 | a1 | e64 | e31 |
| 1096 | a1 | e64 | e32 |
| 1097 | a1 | e64 | e34 |
| 1098 | a1 | e64 | e35 |
| 1099 | a1 | e64 | e38 |
| 1100 | a1 | e64 | e39 |
| 1101 | a1 | e64 | e40 |
| 1102 | a1 | e64 | e42 |
| 1103 | a1 | e64 | e43 |
| 1104 | a1 | e64 | e44 |
| 1105 | a1 | e64 | e45 |
| 1106 | a1 | e64 | e46 |
| 1107 | a1 | e64 | e47 |
| 1108 | a1 | e64 | e48 |
| 1109 | a1 | e64 | e49 |
| 1110 | a1 | e64 | e50 |
| 1111 | a1 | e64 | e51 |
| 1112 | a1 | e64 | e52 |
| 1113 | a1 | e64 | e53 |
| 1114 | a1 | e64 | e54 |
| 1115 | a1 | e64 | e55 |
| 1116 | a1 | e64 | e56 |
| 1117 | a1 | e64 | e57 |
| 1118 | a1 | e64 | e58 |
| 1119 | a1 | e64 | e60 |
| 1120 | a1 | e64 | e61 |
| 1121 | a1 | e64 | e64 |
| 1122 | a1 | e64 | e65 |
| 1123 | a1 | e64 | e66 |
| 1124 | a1 | e64 | e67 |
| 1125 | a2 | d2 | a1 |
| 1126 | a2 | d2 | e1 |
| 1127 | a2 | d2 | e2 |
| 1128 | a2 | d2 | e3 |
| 1129 | a2 | d2 | e4 |
| 1130 | a2 | d2 | e5 |
| 1131 | a2 | d2 | e6 |
| 1132 | a2 | d2 | e7 |
| 1133 | a2 | d2 | e8 |
| 1134 | a2 | d2 | e9 |
| 1135 | a2 | d2 | e10 |
| 1136 | a2 | d2 | e11 |
| 1137 | a2 | d2 | e12 |
| 1138 | a2 | d2 | e13 |
| 1139 | a2 | d2 | e14 |
| 1140 | a2 | d2 | e15 |
| 1141 | a2 | d2 | e16 |
| 1142 | a2 | d2 | e17 |
| 1143 | a2 | d2 | e18 |
| 1144 | a2 | d2 | e19 |
| 1145 | a2 | d2 | e20 |
| 1146 | a2 | d2 | e21 |
| 1147 | a2 | d2 | e22 |
| 1148 | a2 | d2 | e23 |
| 1149 | a2 | d2 | e24 |
| 1150 | a2 | d2 | e25 |
| 1151 | a2 | d2 | e26 |
| 1152 | a2 | d2 | e27 |
| 1153 | a2 | d2 | e28 |
| 1154 | a2 | d2 | e29 |
| 1155 | a2 | d2 | e30 |
| 1156 | a2 | d2 | e31 |
| 1157 | a2 | d2 | e32 |
| 1158 | a2 | d2 | e33 |
| 1159 | a2 | d2 | e34 |
| 1160 | a2 | d2 | e35 |
| 1161 | a2 | d2 | e36 |
| 1162 | a2 | d2 | e37 |
| 1163 | a2 | d2 | e38 |
| 1164 | a2 | d2 | e39 |
| 1165 | a2 | d2 | e40 |
| 1166 | a2 | d2 | e41 |
| 1167 | a2 | d2 | e42 |
| 1168 | a2 | d2 | e43 |
| 1169 | a2 | d2 | e44 |
| 1170 | a2 | d2 | e45 |
| 1171 | a2 | d2 | e46 |
| 1172 | a2 | d2 | e47 |
| 1173 | a2 | d2 | e48 |
| 1174 | a2 | d2 | e49 |
| 1175 | a2 | d2 | e50 |
| 1176 | a2 | d2 | e51 |
| 1177 | a2 | d2 | e52 |
| 1178 | a2 | d2 | e53 |
| 1179 | a2 | d2 | e54 |
| 1180 | a2 | d2 | e55 |
| 1181 | a2 | d2 | e56 |
| 1182 | a2 | d2 | e57 |
| 1183 | a2 | d2 | e58 |
| 1184 | a2 | d2 | e59 |
| 1185 | a2 | d2 | e60 |
| 1186 | a2 | d2 | e61 |
| 1187 | a2 | d2 | e62 |
| 1188 | a2 | d2 | e63 |
| 1189 | a2 | d2 | e64 |
| 1190 | a2 | d2 | e65 |
| 1191 | a2 | d2 | e66 |
| 1192 | a2 | d2 | e67 |
| 1193 | a2 | d3 | a1 |
| 1194 | a2 | d3 | e1 |
| 1195 | a2 | d3 | e2 |
| 1196 | a2 | d3 | e3 |
| 1197 | a2 | d3 | e4 |
| 1198 | a2 | d3 | e5 |
| 1199 | a2 | d3 | e6 |
| 1200 | a2 | d3 | e7 |

TABLE 6

| Compound No. | Ar_{A} | Ar_{B} | Ar_{C} |
|---|---|---|---|
| 1201 | a2 | d3 | e8 |
| 1202 | a2 | d3 | e9 |
| 1203 | a2 | d3 | e10 |
| 1204 | a2 | d3 | e11 |
| 1205 | a2 | d3 | e12 |
| 1206 | a2 | d3 | e13 |
| 1207 | a2 | d3 | e14 |
| 1208 | a2 | d3 | e15 |
| 1209 | a2 | d3 | e16 |
| 1210 | a2 | d3 | e17 |
| 1211 | a2 | d3 | e18 |
| 1212 | a2 | d3 | e19 |
| 1213 | a2 | d3 | e20 |
| 1214 | a2 | d3 | e21 |
| 1215 | a2 | d3 | e22 |
| 1216 | a2 | d3 | e23 |
| 1217 | a2 | d3 | e24 |
| 1218 | a2 | d3 | e25 |
| 1219 | a2 | d3 | e26 |
| 1220 | a2 | d3 | e27 |
| 1221 | a2 | d3 | e28 |
| 1222 | a2 | d3 | e29 |
| 1223 | a2 | d3 | e30 |
| 1224 | a2 | d3 | e31 |
| 1225 | a2 | d3 | e32 |
| 1226 | a2 | d3 | e33 |
| 1227 | a2 | d3 | e34 |
| 1228 | a2 | d3 | e35 |
| 1229 | a2 | d3 | e36 |
| 1230 | a2 | d3 | e37 |
| 1231 | a2 | d3 | e38 |
| 1232 | a2 | d3 | e39 |
| 1233 | a2 | d3 | e40 |
| 1234 | a2 | d3 | e41 |
| 1235 | a2 | d3 | e42 |
| 1236 | a2 | d3 | e43 |
| 1237 | a2 | d3 | e44 |
| 1238 | a2 | d3 | e45 |
| 1239 | a2 | d3 | e46 |
| 1240 | a2 | d3 | e47 |
| 1241 | a2 | d3 | e48 |
| 1242 | a2 | d3 | e49 |
| 1243 | a2 | d3 | e50 |
| 1244 | a2 | d3 | e51 |
| 1245 | a2 | d3 | e52 |
| 1246 | a2 | d3 | e53 |
| 1247 | a2 | d3 | e54 |
| 1248 | a2 | d3 | e55 |
| 1249 | a2 | d3 | e56 |
| 1250 | a2 | d3 | e57 |
| 1251 | a2 | d3 | e58 |
| 1252 | a2 | d3 | e59 |
| 1253 | a2 | d3 | e60 |
| 1254 | a2 | d3 | e61 |
| 1255 | a2 | d3 | e62 |
| 1256 | a2 | d3 | e63 |
| 1257 | a2 | d3 | e64 |
| 1258 | a2 | d3 | e65 |
| 1259 | a2 | d3 | e66 |
| 1260 | a2 | d3 | e67 |
| 1261 | a2 | d7 | a1 |
| 1262 | a2 | d7 | e1 |
| 1263 | a2 | d7 | e1 |
| 1264 | a2 | d7 | e2 |
| 1265 | a2 | d7 | e3 |
| 1266 | a2 | d7 | e4 |
| 1267 | a2 | d7 | e5 |
| 1268 | a2 | d7 | e6 |
| 1269 | a2 | d7 | e7 |
| 1270 | a2 | d7 | e8 |
| 1271 | a2 | d7 | e9 |
| 1272 | a2 | d7 | e10 |
| 1273 | a2 | d7 | e11 |
| 1274 | a2 | d7 | e12 |
| 1275 | a2 | d7 | e13 |
| 1276 | a2 | d7 | e14 |
| 1277 | a2 | d7 | e15 |
| 1278 | a2 | d7 | e16 |
| 1279 | a2 | d7 | e17 |
| 1280 | a2 | d7 | e18 |
| 1281 | a2 | d7 | e19 |
| 1282 | a2 | d7 | e20 |
| 1283 | a2 | d7 | e21 |
| 1284 | a2 | d7 | e22 |
| 1285 | a2 | d7 | e23 |
| 1286 | a2 | d7 | e24 |
| 1287 | a2 | d7 | e25 |
| 1288 | a2 | d7 | e26 |
| 1289 | a2 | d7 | e27 |
| 1290 | a2 | d7 | e28 |
| 1291 | a2 | d7 | e29 |
| 1292 | a2 | d7 | e30 |
| 1293 | a2 | d7 | e31 |
| 1294 | a2 | d7 | e32 |
| 1295 | a2 | d7 | e33 |
| 1296 | a2 | d7 | e34 |
| 1297 | a2 | d7 | e35 |
| 1298 | a2 | d7 | e36 |
| 1299 | a2 | d7 | e37 |
| 1300 | a2 | d7 | e38 |
| 1301 | a2 | d7 | e39 |
| 1302 | a2 | d7 | e40 |
| 1303 | a2 | d7 | e41 |
| 1304 | a2 | d7 | e42 |
| 1305 | a2 | d7 | e43 |
| 1306 | a2 | d7 | e44 |
| 1307 | a2 | d7 | e45 |
| 1308 | a2 | d7 | e46 |
| 1309 | a2 | d7 | e47 |
| 1310 | a2 | d7 | e48 |
| 1311 | a2 | d7 | e49 |
| 1312 | a2 | d7 | e50 |
| 1313 | a2 | d7 | e51 |
| 1314 | a2 | d7 | e52 |
| 1315 | a2 | d7 | e53 |
| 1316 | a2 | d7 | e54 |
| 1317 | a2 | d7 | e55 |
| 1318 | a2 | d7 | e56 |
| 1319 | a2 | d7 | e57 |
| 1320 | a2 | d7 | e58 |
| 1321 | a2 | d7 | e59 |
| 1322 | a2 | d7 | e60 |
| 1323 | a2 | d7 | e61 |
| 1324 | a2 | d7 | e62 |
| 1325 | a2 | d7 | e63 |
| 1326 | a2 | d7 | e64 |
| 1327 | a2 | d7 | e65 |
| 1328 | a2 | d7 | e66 |
| 1329 | a2 | d7 | e67 |
| 1330 | a2 | d9 | a1 |
| 1331 | a2 | d9 | e1 |
| 1332 | a2 | d9 | e1 |
| 1333 | a2 | d9 | e2 |
| 1334 | a2 | d9 | e3 |
| 1335 | a2 | d9 | e4 |
| 1336 | a2 | d9 | e5 |
| 1337 | a2 | d9 | e6 |
| 1338 | a2 | d9 | e7 |
| 1339 | a2 | d9 | e8 |
| 1340 | a2 | d9 | e9 |
| 1341 | a2 | d9 | e10 |
| 1342 | a2 | d9 | e11 |
| 1343 | a2 | d9 | e12 |
| 1344 | a2 | d9 | e13 |
| 1345 | a2 | d9 | e14 |
| 1346 | a2 | d9 | e15 |
| 1347 | a2 | d9 | e16 |
| 1348 | a2 | d9 | e17 |
| 1349 | a2 | d9 | e18 |
| 1350 | a2 | d9 | e19 |
| 1351 | a2 | d9 | e20 |
| 1352 | a2 | d9 | e21 |
| 1353 | a2 | d9 | e22 |
| 1354 | a2 | d9 | e23 |
| 1355 | a2 | d9 | e24 |
| 1356 | a2 | d9 | e25 |
| 1357 | a2 | d9 | e26 |
| 1358 | a2 | d9 | e27 |
| 1359 | a2 | d9 | e28 |
| 1360 | a2 | d9 | e29 |
| 1361 | a2 | d9 | e30 |
| 1362 | a2 | d9 | e31 |
| 1363 | a2 | d9 | e32 |
| 1364 | a2 | d9 | e33 |
| 1365 | a2 | d9 | e34 |
| 1366 | a2 | d9 | e35 |
| 1367 | a2 | d9 | e36 |
| 1368 | a2 | d9 | e37 |
| 1369 | a2 | d9 | e38 |
| 1370 | a2 | d9 | e39 |
| 1371 | a2 | d9 | e40 |
| 1372 | a2 | d9 | e41 |
| 1373 | a2 | d9 | e42 |
| 1374 | a2 | d9 | e43 |
| 1375 | a2 | d9 | e44 |
| 1376 | a2 | d9 | e45 |
| 1377 | a2 | d9 | e46 |
| 1378 | a2 | d9 | e47 |
| 1379 | a2 | d9 | e48 |
| 1380 | a2 | d9 | e49 |
| 1381 | a2 | d9 | e50 |
| 1382 | a2 | d9 | e51 |
| 1383 | a2 | d9 | e52 |
| 1384 | a2 | d9 | e53 |
| 1385 | a2 | d9 | e54 |
| 1386 | a2 | d9 | e55 |
| 1387 | a2 | d9 | e56 |
| 1388 | a2 | d9 | e57 |
| 1389 | a2 | d9 | e58 |
| 1390 | a2 | d9 | e59 |
| 1391 | a2 | d9 | e60 |
| 1392 | a2 | d9 | e61 |
| 1393 | a2 | d9 | e62 |
| 1394 | a2 | d9 | e63 |
| 1395 | a2 | d9 | e64 |
| 1396 | a2 | d9 | e65 |
| 1397 | a2 | d9 | e66 |
| 1398 | a2 | d9 | e67 |
| 1399 | a2 | e1 | a1 |
| 1400 | a2 | e1 | d1 |

TABLE 7

| Compound No. | Ar_{A} | Ar_{B} | Ar_{C} |
|---|---|---|---|
| 1401 | a2 | e1 | d1 |
| 1402 | a2 | e1 | d4 |
| 1403 | a2 | e1 | d5 |
| 1404 | a2 | e1 | d6 |
| 1405 | a2 | e1 | d8 |
| 1406 | a2 | e1 | d10 |
| 1407 | a2 | e1 | e1 |
| 1408 | a2 | e1 | e1 |
| 1409 | a2 | e1 | e2 |
| 1410 | a2 | e1 | e3 |
| 1411 | a2 | e1 | e4 |
| 1412 | a2 | e1 | e5 |
| 1413 | a2 | e1 | e6 |
| 1414 | a2 | e1 | e7 |
| 1415 | a2 | e1 | e8 |
| 1416 | a2 | e1 | e9 |
| 1417 | a2 | e1 | e10 |
| 1418 | a2 | e1 | e11 |
| 1419 | a2 | e1 | e12 |
| 1420 | a2 | e1 | e13 |
| 1421 | a2 | e1 | e14 |
| 1422 | a2 | e1 | e15 |
| 1423 | a2 | e1 | e16 |
| 1424 | a2 | e1 | e17 |
| 1425 | a2 | e1 | e18 |
| 1426 | a2 | e1 | e19 |
| 1427 | a2 | e1 | e20 |
| 1428 | a2 | e1 | e21 |
| 1429 | a2 | e1 | e22 |
| 1430 | a2 | e1 | e23 |
| 1431 | a2 | e1 | e24 |
| 1432 | a2 | e1 | e25 |
| 1433 | a2 | e1 | e26 |
| 1434 | a2 | e1 | e27 |
| 1435 | a2 | e1 | e28 |
| 1436 | a2 | e1 | e29 |
| 1437 | a2 | e1 | e30 |
| 1438 | a2 | e1 | e31 |
| 1439 | a2 | e1 | e32 |
| 1440 | a2 | e1 | e33 |
| 1441 | a2 | e1 | e34 |
| 1442 | a2 | e1 | e35 |
| 1443 | a2 | e1 | e36 |
| 1444 | a2 | e1 | e37 |
| 1445 | a2 | e1 | e38 |
| 1446 | a2 | e1 | e39 |
| 1447 | a2 | e1 | e40 |
| 1448 | a2 | e1 | e41 |
| 1449 | a2 | e1 | e42 |
| 1450 | a2 | e1 | e43 |
| 1451 | a2 | e1 | e44 |
| 1452 | a2 | e1 | e45 |
| 1453 | a2 | e1 | e46 |
| 1454 | a2 | e1 | e47 |
| 1455 | a2 | e1 | e48 |
| 1456 | a2 | e1 | e49 |
| 1457 | a2 | e1 | e50 |
| 1458 | a2 | e1 | e51 |
| 1459 | a2 | e1 | e52 |
| 1460 | a2 | e1 | e53 |
| 1461 | a2 | e1 | e54 |
| 1462 | a2 | e1 | e55 |
| 1463 | a2 | e1 | e56 |
| 1464 | a2 | e1 | e57 |
| 1465 | a2 | e1 | e58 |
| 1466 | a2 | e1 | e59 |
| 1467 | a2 | e1 | e60 |
| 1468 | a2 | e1 | e61 |
| 1469 | a2 | e1 | e62 |
| 1470 | a2 | e1 | e63 |
| 1471 | a2 | e1 | e64 |
| 1472 | a2 | e1 | e65 |
| 1473 | a2 | e1 | e66 |
| 1474 | a2 | e1 | e67 |
| 1475 | a2 | e2 | a1 |
| 1476 | a2 | e2 | d1 |
| 1477 | a2 | e2 | d1 |
| 1478 | a2 | e2 | d4 |
| 1479 | a2 | e2 | d5 |
| 1480 | a2 | e2 | d6 |
| 1481 | a2 | e2 | d8 |
| 1482 | a2 | e2 | d10 |
| 1483 | a2 | e2 | e2 |
| 1484 | a2 | e2 | e3 |
| 1485 | a2 | e2 | e4 |
| 1486 | a2 | e2 | e5 |
| 1487 | a2 | e2 | e6 |
| 1488 | a2 | e2 | e7 |
| 1489 | a2 | e2 | e8 |
| 1490 | a2 | e2 | e9 |
| 1491 | a2 | e2 | e10 |
| 1492 | a2 | e2 | e11 |
| 1493 | a2 | e2 | e12 |
| 1494 | a2 | e2 | e13 |
| 1495 | a2 | e2 | e14 |
| 1496 | a2 | e2 | e15 |
| 1497 | a2 | e2 | e16 |
| 1498 | a2 | e2 | e17 |
| 1499 | a2 | e2 | e18 |
| 1500 | a2 | e2 | e19 |
| 1501 | a2 | e2 | e20 |
| 1502 | a2 | e2 | e21 |
| 1503 | a2 | e2 | e22 |
| 1504 | a2 | e2 | e23 |
| 1505 | a2 | e2 | e24 |
| 1506 | a2 | e2 | e25 |
| 1507 | a2 | e2 | e26 |
| 1508 | a2 | e2 | e28 |
| 1509 | a2 | e2 | e29 |
| 1510 | a2 | e2 | e30 |
| 1511 | a2 | e2 | e31 |
| 1512 | a2 | e2 | e32 |
| 1513 | a2 | e2 | e33 |
| 1514 | a2 | e2 | e34 |
| 1515 | a2 | e2 | e35 |
| 1516 | a2 | e2 | e36 |
| 1517 | a2 | e2 | e37 |
| 1518 | a2 | e2 | e38 |
| 1519 | a2 | e2 | e39 |
| 1520 | a2 | e2 | e40 |
| 1521 | a2 | e2 | e41 |
| 1522 | a2 | e2 | e42 |
| 1523 | a2 | e2 | e43 |
| 1524 | a2 | e2 | e44 |
| 1525 | a2 | e2 | e45 |
| 1526 | a2 | e2 | e46 |
| 1527 | a2 | e2 | e47 |
| 1528 | a2 | e2 | e48 |
| 1529 | a2 | e2 | e49 |
| 1530 | a2 | e2 | e50 |
| 1531 | a2 | e2 | e51 |
| 1532 | a2 | e2 | e52 |
| 1533 | a2 | e2 | e53 |
| 1534 | a2 | e2 | e54 |
| 1535 | a2 | e2 | e55 |
| 1536 | a2 | e2 | e56 |
| 1537 | a2 | e2 | e57 |
| 1538 | a2 | e2 | e58 |
| 1539 | a2 | e2 | e59 |
| 1540 | a2 | e2 | e60 |
| 1541 | a2 | e2 | e61 |
| 1542 | a2 | e2 | e62 |
| 1543 | a2 | e2 | e63 |
| 1544 | a2 | e2 | e64 |
| 1545 | a2 | e2 | e65 |
| 1546 | a2 | e2 | e66 |
| 1547 | a2 | e2 | e67 |
| 1548 | a2 | e24 | a1 |
| 1549 | a2 | e24 | d1 |
| 1550 | a2 | e24 | d1 |
| 1551 | a2 | e24 | d4 |
| 1552 | a2 | e24 | d5 |
| 1553 | a2 | e24 | d6 |
| 1554 | a2 | e24 | d8 |
| 1555 | a2 | e24 | d10 |
| 1556 | a2 | e24 | e3 |
| 1557 | a2 | e24 | e4 |
| 1558 | a2 | e24 | e5 |
| 1559 | a2 | e24 | e6 |
| 1560 | a2 | e24 | e7 |
| 1561 | a2 | e24 | e8 |
| 1562 | a2 | e24 | e9 |
| 1563 | a2 | e24 | e10 |
| 1564 | a2 | e24 | e11 |
| 1565 | a2 | e24 | e12 |
| 1566 | a2 | e24 | e13 |
| 1567 | a2 | e24 | e14 |
| 1568 | a2 | e24 | e15 |
| 1569 | a2 | e24 | e16 |
| 1570 | a2 | e24 | e17 |
| 1571 | a2 | e24 | e18 |
| 1572 | a2 | e24 | e19 |
| 1573 | a2 | e24 | e20 |
| 1574 | a2 | e24 | e21 |
| 1575 | a2 | e24 | e22 |
| 1576 | a2 | e24 | e23 |
| 1577 | a2 | e24 | e24 |
| 1578 | a2 | e24 | e25 |
| 1579 | a2 | e24 | e26 |
| 1580 | a2 | e24 | e27 |
| 1581 | a2 | e24 | e28 |
| 1582 | a2 | e24 | e29 |
| 1583 | a2 | e24 | e30 |
| 1584 | a2 | e24 | e31 |
| 1585 | a2 | e24 | e32 |
| 1586 | a2 | e24 | e33 |
| 1587 | a2 | e24 | e34 |
| 1588 | a2 | e24 | e35 |
| 1589 | a2 | e24 | e36 |
| 1590 | a2 | e24 | e37 |
| 1591 | a2 | e24 | e38 |
| 1592 | a2 | e24 | e39 |
| 1593 | a2 | e24 | e40 |
| 1594 | a2 | e24 | e41 |
| 1595 | a2 | e24 | e42 |
| 1596 | a2 | e24 | e43 |
| 1597 | a2 | e24 | e44 |
| 1598 | a2 | e24 | e45 |
| 1599 | a2 | e24 | e46 |
| 1600 | a2 | e24 | e47 |

TABLE 8

| Compound No. | Ar_{A} | Ar_{B} | Ar_{C} |
|---|---|---|---|
| 1601 | a2 | e24 | e48 |
| 1602 | a2 | e24 | e49 |
| 1603 | a2 | e24 | e50 |
| 1604 | a2 | e24 | e51 |
| 1605 | a2 | e24 | e52 |
| 1606 | a2 | e24 | e53 |
| 1607 | a2 | e24 | e54 |
| 1608 | a2 | e24 | e55 |
| 1609 | a2 | e24 | e56 |
| 1610 | a2 | e24 | e57 |
| 1611 | a2 | e24 | e58 |
| 1612 | a2 | e24 | e59 |
| 1613 | a2 | e24 | e60 |
| 1614 | a2 | e24 | e61 |
| 1615 | a2 | e24 | e62 |
| 1616 | a2 | e24 | e63 |
| 1617 | a2 | e24 | e64 |
| 1618 | a2 | e24 | e65 |
| 1619 | a2 | e24 | e66 |
| 1620 | a2 | e24 | e67 |
| 1621 | a2 | e27 | a1 |
| 1622 | a2 | e27 | d1 |
| 1623 | a2 | e27 | d1 |
| 1624 | a2 | e27 | d4 |
| 1625 | a2 | e27 | d5 |
| 1626 | a2 | e27 | d6 |
| 1627 | a2 | e27 | d8 |
| 1628 | a2 | e27 | d10 |
| 1629 | a2 | e27 | e3 |
| 1630 | a2 | e27 | e4 |
| 1631 | a2 | e27 | e5 |
| 1632 | a2 | e27 | e6 |
| 1633 | a2 | e27 | e7 |
| 1634 | a2 | e27 | e8 |
| 1635 | a2 | e27 | e9 |
| 1636 | a2 | e27 | e10 |
| 1637 | a2 | e27 | e11 |
| 1638 | a2 | e27 | e12 |
| 1639 | a2 | e27 | e13 |
| 1640 | a2 | e27 | e14 |
| 1641 | a2 | e27 | e15 |
| 1642 | a2 | e27 | e16 |
| 1643 | a2 | e27 | e17 |
| 1644 | a2 | e27 | e18 |
| 1645 | a2 | e27 | e19 |
| 1646 | a2 | e27 | e20 |
| 1647 | a2 | e27 | e21 |
| 1648 | a2 | e27 | e22 |
| 1649 | a2 | e27 | e23 |
| 1650 | a2 | e27 | e25 |
| 1651 | a2 | e27 | e26 |
| 1652 | a2 | e27 | e27 |
| 1653 | a2 | e27 | e28 |
| 1654 | a2 | e27 | e29 |
| 1655 | a2 | e27 | e30 |
| 1656 | a2 | e27 | e31 |
| 1657 | a2 | e27 | e32 |
| 1658 | a2 | e27 | e33 |
| 1659 | a2 | e27 | e34 |
| 1660 | a2 | e27 | e35 |
| 1661 | a2 | e27 | e36 |
| 1662 | a2 | e27 | e37 |
| 1663 | a2 | e27 | e38 |
| 1664 | a2 | e27 | e39 |
| 1665 | a2 | e27 | e40 |
| 1666 | a2 | e27 | e41 |
| 1667 | a2 | e27 | e42 |
| 1668 | a2 | e27 | e43 |
| 1669 | a2 | e27 | e44 |
| 1670 | a2 | e27 | e45 |
| 1671 | a2 | e27 | e46 |
| 1672 | a2 | e27 | e47 |
| 1673 | a2 | e27 | e48 |
| 1674 | a2 | e27 | e49 |
| 1675 | a2 | e27 | e50 |
| 1676 | a2 | e27 | e51 |
| 1677 | a2 | e27 | e52 |
| 1678 | a2 | e27 | e53 |
| 1679 | a2 | e27 | e54 |
| 1680 | a2 | e27 | e55 |
| 1681 | a2 | e27 | e56 |
| 1682 | a2 | e27 | e57 |
| 1683 | a2 | e27 | e58 |
| 1684 | a2 | e27 | e59 |
| 1685 | a2 | e27 | e60 |
| 1686 | a2 | e27 | e61 |
| 1687 | a2 | e27 | e62 |
| 1688 | a2 | e27 | e63 |
| 1689 | a2 | e27 | e64 |
| 1690 | a2 | e27 | e65 |
| 1691 | a2 | e27 | e66 |
| 1692 | a2 | e27 | e67 |
| 1693 | a2 | e33 | a1 |
| 1694 | a2 | e33 | d1 |
| 1695 | a2 | e33 | d1 |
| 1696 | a2 | e33 | d4 |
| 1697 | a2 | e33 | d5 |
| 1698 | a2 | e33 | d6 |
| 1699 | a2 | e33 | d8 |
| 1700 | a2 | e33 | d10 |
| 1701 | a2 | e33 | e3 |
| 1702 | a2 | e33 | e4 |
| 1703 | a2 | e33 | e5 |
| 1704 | a2 | e33 | e6 |
| 1705 | a2 | e33 | e7 |
| 1706 | a2 | e33 | e8 |
| 1707 | a2 | e33 | e9 |
| 1708 | a2 | e33 | e10 |
| 1709 | a2 | e33 | e11 |
| 1710 | a2 | e33 | e12 |
| 1711 | a2 | e33 | e13 |
| 1712 | a2 | e33 | e14 |
| 1713 | a2 | e33 | e15 |
| 1714 | a2 | e33 | e16 |
| 1715 | a2 | e33 | e17 |
| 1716 | a2 | e33 | e18 |
| 1717 | a2 | e33 | e19 |
| 1718 | a2 | e33 | e20 |
| 1719 | a2 | e33 | e21 |
| 1720 | a2 | e33 | e22 |
| 1721 | a2 | e33 | e23 |
| 1722 | a2 | e33 | e25 |
| 1723 | a2 | e33 | e26 |
| 1724 | a2 | e33 | e27 |
| 1725 | a2 | e33 | e28 |
| 1726 | a2 | e33 | e29 |
| 1727 | a2 | e33 | e30 |
| 1728 | a2 | e33 | e31 |
| 1729 | a2 | e33 | e32 |
| 1730 | a2 | e33 | e33 |
| 1731 | a2 | e33 | e34 |
| 1732 | a2 | e33 | e35 |
| 1733 | a2 | e33 | e36 |
| 1734 | a2 | e33 | e37 |
| 1735 | a2 | e33 | e38 |
| 1736 | a2 | e33 | e39 |
| 1737 | a2 | e33 | e40 |
| 1738 | a2 | e33 | e41 |
| 1739 | a2 | e33 | e42 |
| 1740 | a2 | e33 | e43 |
| 1741 | a2 | e33 | e44 |
| 1742 | a2 | e33 | e45 |
| 1743 | a2 | e33 | e46 |
| 1744 | a2 | e33 | e47 |
| 1745 | a2 | e33 | e48 |
| 1746 | a2 | e33 | e49 |
| 1747 | a2 | e33 | e50 |
| 1748 | a2 | e33 | e51 |
| 1749 | a2 | e33 | e52 |
| 1750 | a2 | e33 | e53 |
| 1751 | a2 | e33 | e54 |
| 1752 | a2 | e33 | e55 |
| 1753 | a2 | e33 | e56 |
| 1754 | a2 | e33 | e57 |
| 1755 | a2 | e33 | e58 |
| 1756 | a2 | e33 | e59 |
| 1757 | a2 | e33 | e60 |
| 1758 | a2 | e33 | e61 |
| 1759 | a2 | e33 | e62 |
| 1760 | a2 | e33 | e63 |
| 1761 | a2 | e33 | e64 |
| 1762 | a2 | e33 | e65 |
| 1763 | a2 | e33 | e66 |
| 1764 | a2 | e33 | e67 |
| 1765 | a2 | e36 | a1 |
| 1766 | a2 | e36 | d1 |
| 1767 | a2 | e36 | d1 |
| 1768 | a2 | e36 | d4 |
| 1769 | a2 | e36 | d5 |
| 1770 | a2 | e36 | d6 |
| 1771 | a2 | e36 | d8 |
| 1772 | a2 | e36 | d10 |
| 1773 | a2 | e36 | e3 |
| 1774 | a2 | e36 | e4 |
| 1775 | a2 | e36 | e5 |
| 1776 | a2 | e36 | e6 |
| 1777 | a2 | e36 | e7 |
| 1778 | a2 | e36 | e8 |
| 1779 | a2 | e36 | e9 |
| 1780 | a2 | e36 | e10 |
| 1781 | a2 | e36 | e11 |
| 1782 | a2 | e36 | e12 |
| 1783 | a2 | e36 | e13 |
| 1784 | a2 | e36 | e14 |
| 1785 | a2 | e36 | e15 |
| 1786 | a2 | e36 | e16 |
| 1787 | a2 | e36 | e17 |
| 1788 | a2 | e36 | e18 |
| 1789 | a2 | e36 | e19 |
| 1790 | a2 | e36 | e20 |
| 1791 | a2 | e36 | e21 |
| 1792 | a2 | e36 | e22 |
| 1793 | a2 | e36 | e23 |
| 1794 | a2 | e36 | e25 |
| 1795 | a2 | e36 | e26 |
| 1796 | a2 | e36 | e28 |
| 1797 | a2 | e36 | e29 |
| 1798 | a2 | e36 | e30 |
| 1799 | a2 | e36 | e31 |
| 1800 | a2 | e36 | e32 |

TABLE 9

| Compound No. | Ar_{A} | Ar_{B} | Ar_{C} |
|---|---|---|---|
| 1801 | a2 | e36 | e34 |
| 1802 | a2 | e36 | e35 |
| 1803 | a2 | e36 | e36 |
| 1804 | a2 | e36 | e37 |
| 1805 | a2 | e36 | e38 |
| 1806 | a2 | e36 | e39 |
| 1807 | a2 | e36 | e40 |
| 1808 | a2 | e36 | e41 |
| 1809 | a2 | e36 | e42 |
| 1810 | a2 | e36 | e43 |
| 1811 | a2 | e36 | e44 |
| 1812 | a2 | e36 | e45 |
| 1813 | a2 | e36 | e46 |
| 1814 | a2 | e36 | e47 |
| 1815 | a2 | e36 | e48 |
| 1816 | a2 | e36 | e49 |
| 1817 | a2 | e36 | e50 |
| 1818 | a2 | e36 | e51 |
| 1819 | a2 | e36 | e52 |
| 1820 | a2 | e36 | e53 |
| 1821 | a2 | e36 | e54 |
| 1822 | a2 | e36 | e55 |
| 1823 | a2 | e36 | e56 |
| 1824 | a2 | e36 | e57 |
| 1825 | a2 | e36 | e58 |
| 1826 | a2 | e36 | e59 |
| 1827 | a2 | e36 | e60 |
| 1828 | a2 | e36 | e61 |
| 1829 | a2 | e36 | e62 |
| 1830 | a2 | e36 | e63 |
| 1831 | a2 | e36 | e64 |
| 1832 | a2 | e36 | e65 |
| 1833 | a2 | e36 | e66 |
| 1834 | a2 | e36 | e67 |
| 1835 | a2 | e37 | a1 |
| 1836 | a2 | e37 | d1 |
| 1837 | a2 | e37 | d1 |
| 1838 | a2 | e37 | d4 |
| 1839 | a2 | e37 | d5 |
| 1840 | a2 | e37 | d6 |
| 1841 | a2 | e37 | d8 |
| 1842 | a2 | e37 | d10 |
| 1843 | a2 | e37 | e3 |
| 1844 | a2 | e37 | e4 |
| 1845 | a2 | e37 | e5 |
| 1846 | a2 | e37 | e6 |
| 1847 | a2 | e37 | e7 |
| 1848 | a2 | e37 | e8 |
| 1849 | a2 | e37 | e9 |
| 1850 | a2 | e37 | e10 |
| 1851 | a2 | e37 | e11 |
| 1852 | a2 | e37 | e12 |
| 1853 | a2 | e37 | e13 |
| 1854 | a2 | e37 | e14 |
| 1855 | a2 | e37 | e15 |
| 1856 | a2 | e37 | e16 |
| 1857 | a2 | e37 | e17 |
| 1858 | a2 | e37 | e18 |
| 1859 | a2 | e37 | e19 |
| 1860 | a2 | e37 | e20 |
| 1861 | a2 | e37 | e21 |
| 1862 | a2 | e37 | e22 |
| 1863 | a2 | e37 | e23 |
| 1864 | a2 | e37 | e25 |
| 1865 | a2 | e37 | e26 |
| 1866 | a2 | e37 | e28 |
| 1867 | a2 | e37 | e29 |
| 1868 | a2 | e37 | e30 |
| 1869 | a2 | e37 | e31 |
| 1870 | a2 | e37 | e32 |
| 1871 | a2 | e37 | e34 |
| 1872 | a2 | e37 | e35 |
| 1873 | a2 | e37 | e37 |
| 1874 | a2 | e37 | e38 |
| 1875 | a2 | e37 | e39 |
| 1876 | a2 | e37 | e40 |
| 1877 | a2 | e37 | e41 |
| 1878 | a2 | e37 | e42 |
| 1879 | a2 | e37 | e43 |
| 1880 | a2 | e37 | e44 |
| 1881 | a2 | e37 | e45 |
| 1882 | a2 | e37 | e46 |
| 1883 | a2 | e37 | e47 |
| 1884 | a2 | e37 | e48 |
| 1885 | a2 | e37 | e49 |
| 1886 | a2 | e37 | e50 |
| 1887 | a2 | e37 | e51 |
| 1888 | a2 | e37 | e52 |
| 1889 | a2 | e37 | e53 |
| 1890 | a2 | e37 | e54 |
| 1891 | a2 | e37 | e55 |
| 1892 | a2 | e37 | e56 |
| 1893 | a2 | e37 | e57 |
| 1894 | a2 | e37 | e58 |
| 1895 | a2 | e37 | e59 |
| 1896 | a2 | e37 | e60 |
| 1897 | a2 | e37 | e61 |
| 1898 | a2 | e37 | e62 |
| 1899 | a2 | e37 | e63 |
| 1900 | a2 | e37 | e64 |
| 1901 | a2 | e37 | e65 |
| 1902 | a2 | e37 | e66 |
| 1903 | a2 | e37 | e67 |
| 1904 | a2 | e41 | a1 |
| 1905 | a2 | e41 | d1 |
| 1906 | a2 | e41 | d1 |
| 1907 | a2 | e41 | d4 |
| 1908 | a2 | e41 | d5 |
| 1909 | a2 | e41 | d6 |
| 1910 | a2 | e41 | d8 |
| 1911 | a2 | e41 | d10 |
| 1912 | a2 | e41 | e3 |
| 1913 | a2 | e41 | e4 |
| 1914 | a2 | e41 | e5 |
| 1915 | a2 | e41 | e6 |
| 1916 | a2 | e41 | e7 |
| 1917 | a2 | e41 | e8 |
| 1918 | a2 | e41 | e9 |
| 1919 | a2 | e41 | e10 |
| 1920 | a2 | e41 | e11 |
| 1921 | a2 | e41 | e12 |
| 1922 | a2 | e41 | e13 |
| 1923 | a2 | e41 | e14 |
| 1924 | a2 | e41 | e15 |
| 1925 | a2 | e41 | e16 |
| 1926 | a2 | e41 | e17 |
| 1927 | a2 | e41 | e18 |
| 1928 | a2 | e41 | e19 |
| 1929 | a2 | e41 | e20 |
| 1930 | a2 | e41 | e21 |
| 1931 | a2 | e41 | e22 |
| 1932 | a2 | e41 | e23 |
| 1933 | a2 | e41 | e25 |
| 1934 | a2 | e41 | e26 |
| 1935 | a2 | e41 | e28 |
| 1936 | a2 | e41 | e29 |
| 1937 | a2 | e41 | e30 |
| 1938 | a2 | e41 | e31 |
| 1939 | a2 | e41 | e32 |
| 1940 | a2 | e41 | e34 |
| 1941 | a2 | e41 | e35 |
| 1942 | a2 | e41 | e38 |
| 1943 | a2 | e41 | e39 |
| 1944 | a2 | e41 | e40 |
| 1945 | a2 | e41 | e41 |
| 1946 | a2 | e41 | e42 |
| 1947 | a2 | e41 | e43 |
| 1948 | a2 | e41 | e44 |
| 1949 | a2 | e41 | e45 |
| 1950 | a2 | e41 | e46 |
| 1951 | a2 | e41 | e47 |
| 1952 | a2 | e41 | e48 |
| 1953 | a2 | e41 | e49 |
| 1954 | a2 | e41 | e50 |
| 1955 | a2 | e41 | e51 |
| 1956 | a2 | e41 | e52 |
| 1957 | a2 | e41 | e53 |
| 1958 | a2 | e41 | e54 |
| 1959 | a2 | e41 | e55 |
| 1960 | a2 | e41 | e56 |
| 1961 | a2 | e41 | e57 |
| 1962 | a2 | e41 | e58 |
| 1963 | a2 | e41 | e59 |
| 1964 | a2 | e41 | e60 |
| 1965 | a2 | e41 | e61 |
| 1966 | a2 | e41 | e62 |
| 1967 | a2 | e41 | e63 |
| 1968 | a2 | e41 | e64 |
| 1969 | a2 | e41 | e65 |
| 1970 | a2 | e41 | e66 |
| 1971 | a2 | e41 | e67 |
| 1972 | a2 | e59 | a1 |
| 1973 | a2 | e59 | d1 |
| 1974 | a2 | e59 | d1 |
| 1975 | a2 | e59 | d4 |
| 1976 | a2 | e59 | d5 |
| 1977 | a2 | e59 | d6 |
| 1978 | a2 | e59 | d8 |
| 1979 | a2 | e59 | d10 |
| 1980 | a2 | e59 | e3 |
| 1981 | a2 | e59 | e4 |
| 1982 | a2 | e59 | e5 |
| 1983 | a2 | e59 | e6 |
| 1984 | a2 | e59 | e7 |
| 1985 | a2 | e59 | e8 |
| 1986 | a2 | e59 | e9 |
| 1987 | a2 | e59 | e10 |
| 1988 | a2 | e59 | e11 |
| 1989 | a2 | e59 | e12 |
| 1990 | a2 | e59 | e13 |
| 1991 | a2 | e59 | e14 |
| 1992 | a2 | e59 | e15 |
| 1993 | a2 | e59 | e16 |
| 1994 | a2 | e59 | e17 |
| 1995 | a2 | e59 | e18 |
| 1996 | a2 | e59 | e19 |
| 1997 | a2 | e59 | e20 |
| 1998 | a2 | e59 | e21 |
| 1999 | a2 | e59 | e22 |
| 2000 | a2 | e59 | e23 |

TABLE 10

| Compound No. | Ar_{A} | Ar_{B} | Ar_{C} |
|---|---|---|---|
| 2001 | a2 | e59 | e25 |
| 2002 | a2 | e59 | e26 |
| 2003 | a2 | e59 | e28 |
| 2004 | a2 | e59 | e29 |
| 2005 | a2 | e59 | e30 |
| 2006 | a2 | e59 | e31 |
| 2007 | a2 | e59 | e32 |
| 2008 | a2 | e59 | e34 |
| 2009 | a2 | e59 | e35 |
| 2010 | a2 | e59 | e38 |
| 2011 | a2 | e59 | e39 |
| 2012 | a2 | e59 | e40 |
| 2013 | a2 | e59 | e42 |
| 2014 | a2 | e59 | e43 |
| 2015 | a2 | e59 | e44 |
| 2016 | a2 | e59 | e45 |
| 2017 | a2 | e59 | e46 |
| 2018 | a2 | e59 | e47 |
| 2019 | a2 | e59 | e48 |
| 2020 | a2 | e59 | e49 |
| 2021 | a2 | e59 | e50 |
| 2022 | a2 | e59 | e51 |
| 2023 | a2 | e59 | e52 |
| 2024 | a2 | e59 | e53 |
| 2025 | a2 | e59 | e54 |
| 2026 | a2 | e59 | e55 |
| 2027 | a2 | e59 | e56 |
| 2028 | a2 | e59 | e57 |
| 2029 | a2 | e59 | e58 |
| 2030 | a2 | e59 | e59 |
| 2031 | a2 | e59 | e60 |
| 2032 | a2 | e59 | e61 |
| 2033 | a2 | e59 | e62 |
| 2034 | a2 | e59 | e63 |
| 2035 | a2 | e59 | e64 |
| 2036 | a2 | e59 | e65 |
| 2037 | a2 | e59 | e66 |
| 2038 | a2 | e59 | e67 |
| 2039 | a2 | e62 | a1 |
| 2040 | a2 | e62 | d1 |
| 2041 | a2 | e62 | d1 |
| 2042 | a2 | e62 | d5 |
| 2043 | a2 | e62 | d6 |
| 2044 | a2 | e62 | d8 |
| 2045 | a2 | e62 | d10 |
| 2046 | a2 | e62 | e5 |
| 2047 | a2 | e62 | e6 |
| 2048 | a2 | e62 | e7 |
| 2049 | a2 | e62 | e8 |
| 2050 | a2 | e62 | e9 |
| 2051 | a2 | e62 | e10 |
| 2052 | a2 | e62 | e11 |
| 2053 | a2 | e62 | e12 |
| 2054 | a2 | e62 | e13 |
| 2055 | a2 | e62 | e14 |
| 2056 | a2 | e62 | e15 |
| 2057 | a2 | e62 | e16 |
| 2058 | a2 | e62 | e17 |
| 2059 | a2 | e62 | e18 |
| 2060 | a2 | e62 | e19 |
| 2061 | a2 | e62 | e20 |
| 2062 | a2 | e62 | e21 |
| 2063 | a2 | e62 | e22 |
| 2064 | a2 | e62 | e23 |
| 2065 | a2 | e62 | e25 |
| 2066 | a2 | e62 | e26 |
| 2067 | a2 | e62 | e28 |
| 2068 | a2 | e62 | e29 |
| 2069 | a2 | e62 | e30 |
| 2070 | a2 | e62 | e31 |
| 2071 | a2 | e62 | e32 |
| 2072 | a2 | e62 | e34 |
| 2073 | a2 | e62 | e35 |
| 2074 | a2 | e62 | e38 |
| 2075 | a2 | e62 | e39 |
| 2076 | a2 | e62 | e40 |
| 2077 | a2 | e62 | e42 |
| 2078 | a2 | e62 | e43 |
| 2079 | a2 | e62 | e44 |
| 2080 | a2 | e62 | e45 |
| 2081 | a2 | e62 | e46 |
| 2082 | a2 | e62 | e47 |
| 2083 | a2 | e62 | e48 |
| 2084 | a2 | e62 | e49 |
| 2085 | a2 | e62 | e50 |
| 2086 | a2 | e62 | e51 |
| 2087 | a2 | e62 | e52 |
| 2088 | a2 | e62 | e53 |
| 2089 | a2 | e62 | e54 |
| 2090 | a2 | e62 | e55 |
| 2091 | a2 | e62 | e56 |
| 2092 | a2 | e62 | e57 |
| 2093 | a2 | e62 | e58 |
| 2094 | a2 | e62 | e60 |
| 2095 | a2 | e62 | e61 |
| 2096 | a2 | e62 | e62 |
| 2097 | a2 | e62 | e63 |
| 2098 | a2 | e62 | e64 |
| 2099 | a2 | e62 | e65 |
| 2100 | a2 | e62 | e66 |
| 2101 | a2 | e62 | e67 |
| 2102 | a2 | e63 | a1 |
| 2103 | a2 | e63 | d1 |
| 2104 | a2 | e63 | d1 |
| 2105 | a2 | e63 | d4 |
| 2106 | a2 | e63 | d5 |
| 2107 | a2 | e63 | d6 |
| 2108 | a2 | e63 | d8 |
| 2109 | a2 | e63 | d10 |
| 2110 | a2 | e63 | e3 |
| 2111 | a2 | e63 | e4 |
| 2112 | a2 | e63 | e5 |
| 2113 | a2 | e63 | e6 |
| 2114 | a2 | e63 | e7 |
| 2115 | a2 | e63 | e8 |
| 2116 | a2 | e63 | e9 |
| 2117 | a2 | e63 | e10 |
| 2118 | a2 | e63 | e11 |
| 2119 | a2 | e63 | e12 |
| 2120 | a2 | e63 | e13 |
| 2121 | a2 | e63 | e14 |
| 2122 | a2 | e63 | e15 |
| 2123 | a2 | e63 | e16 |
| 2124 | a2 | e63 | e17 |
| 2125 | a2 | e63 | e18 |
| 2126 | a2 | e63 | e19 |
| 2127 | a2 | e63 | e20 |
| 2128 | a2 | e63 | e21 |
| 2129 | a2 | e63 | e22 |
| 2130 | a2 | e63 | e23 |
| 2131 | a2 | e63 | e25 |
| 2132 | a2 | e63 | e26 |
| 2133 | a2 | e63 | e28 |
| 2134 | a2 | e63 | e29 |
| 2135 | a2 | e63 | e30 |
| 2136 | a2 | e63 | e31 |
| 2137 | a2 | e63 | e32 |
| 2138 | a2 | e63 | e34 |
| 2139 | a2 | e63 | e35 |
| 2140 | a2 | e63 | e38 |
| 2141 | a2 | e63 | e39 |
| 2142 | a2 | e63 | e40 |
| 2143 | a2 | e63 | e42 |
| 2144 | a2 | e63 | e43 |
| 2145 | a2 | e63 | e44 |
| 2146 | a2 | e63 | e45 |
| 2147 | a2 | e63 | e46 |
| 2148 | a2 | e63 | e47 |
| 2149 | a2 | e63 | e48 |
| 2150 | a2 | e63 | e49 |
| 2151 | a2 | e63 | e50 |
| 2152 | a2 | e63 | e51 |
| 2153 | a2 | e63 | e52 |
| 2154 | a2 | e63 | e53 |
| 2155 | a2 | e63 | e54 |
| 2156 | a2 | e63 | e55 |
| 2157 | a2 | e63 | e56 |
| 2158 | a2 | e63 | e57 |
| 2159 | a2 | e63 | e58 |
| 2160 | a2 | e63 | e60 |
| 2161 | a2 | e63 | e61 |
| 2162 | a2 | e63 | e63 |
| 2163 | a2 | e63 | e64 |
| 2164 | a2 | e63 | e65 |
| 2165 | a2 | e63 | e66 |
| 2166 | a2 | e63 | e67 |
| 2167 | a2 | e64 | a1 |
| 2168 | a2 | e64 | d1 |
| 2169 | a2 | e64 | d1 |
| 2170 | a2 | e64 | d4 |
| 2171 | a2 | e64 | d5 |
| 2172 | a2 | e64 | d6 |
| 2173 | a2 | e64 | d8 |
| 2174 | a2 | e64 | d10 |
| 2175 | a2 | e64 | e3 |
| 2176 | a2 | e64 | e4 |
| 2177 | a2 | e64 | e5 |
| 2178 | a2 | e64 | e6 |
| 2179 | a2 | e64 | e7 |
| 2180 | a2 | e64 | e8 |
| 2181 | a2 | e64 | e9 |
| 2182 | a2 | e64 | e10 |
| 2183 | a2 | e64 | e11 |
| 2184 | a2 | e64 | e12 |
| 2185 | a2 | e64 | e13 |
| 2186 | a2 | e64 | e14 |
| 2187 | a2 | e64 | e15 |
| 2188 | a2 | e64 | e16 |
| 2189 | a2 | e64 | e17 |
| 2190 | a2 | e64 | e18 |
| 2191 | a2 | e64 | e19 |
| 2192 | a2 | e64 | e20 |
| 2193 | a2 | e64 | e21 |
| 2194 | a2 | e64 | e22 |
| 2195 | a2 | e64 | e23 |
| 2196 | a2 | e64 | e25 |
| 2197 | a2 | e64 | e26 |
| 2198 | a2 | e64 | e28 |
| 2199 | a2 | e64 | e29 |
| 2200 | a2 | e64 | e30 |

TABLE 11

| Compound | | | |
|---|---|---|---|
| No. | Ar_{A} | Ar_{B} | Ar_{C} |
| 2201 | a2 | e64 | e31 |
| 2202 | a2 | e64 | e32 |
| 2203 | a2 | e64 | e34 |
| 2204 | a2 | e64 | e35 |
| 2205 | a2 | e64 | e38 |
| 2206 | a2 | e64 | e39 |
| 2207 | a2 | e64 | e40 |
| 2208 | a2 | e64 | e42 |
| 2209 | a2 | e64 | e43 |
| 2210 | a2 | e64 | e44 |
| 2211 | a2 | e64 | e45 |
| 2212 | a2 | e64 | e46 |
| 2213 | a2 | e64 | e47 |
| 2214 | a2 | e64 | e48 |
| 2215 | a2 | e64 | e49 |
| 2216 | a2 | e64 | e50 |
| 2217 | a2 | e64 | e51 |
| 2218 | a2 | e64 | e52 |
| 2219 | a2 | e64 | e53 |
| 2220 | a2 | e64 | e54 |
| 2221 | a2 | e64 | e55 |
| 2222 | a2 | e64 | e56 |
| 2223 | a2 | e64 | e57 |
| 2224 | a2 | e64 | e58 |
| 2225 | a2 | e64 | e60 |
| 2226 | a2 | e64 | e61 |
| 2227 | a2 | e64 | e64 |
| 2228 | a2 | e64 | e65 |
| 2229 | a2 | e64 | e66 |
| 2230 | a2 | e64 | e67 |

The amine compound of one or more embodiments according to the present disclosure includes a 1-aryldibenzofuran-3-yl group or a 1-aryldibenzothiophen-3-yl group at the nitrogen atom of an amine (hereinafter, a first substituent). For example, position 3 of the dibenzofuran or dibenzothiophene of the first substituent is directly bonded to the nitrogen atom of the amine. The total carbon number of an aryl group bonded at position 1 of the dibenzofuran or dibenzothiophene of the first substituent is 6 to 16. The second substituent and the third substituent may be a substituted or unsubstituted aryl group or heteroaryl group. The second substituent and the third substituent may be directly bonded to the nitrogen atom of the amine or bonded via a linker. The amine compound of one or more embodiments may be a monoamine compound.

The amine compound of one or more embodiments according to the present disclosure, having the structure may have excellent or suitable hole transport capacity. The light emitting element ED of one or more embodiments according to the present disclosure includes the amine compound in a hole transport region HTR, and accordingly, the charge balance of the light emitting element ED may be improved, and the light emitting element ED may show relatively high emission efficiency and long lifetime. The amine compound of one or more embodiments according to the present disclosure may be included in a hole transport layer HTL and/or an electron blocking layer EBL.

The hole transport region HTR is provided on the first electrode EL1. The thickness of the hole transport region HTR may be, for example, about 50 Å to about 15,000 Å.

The hole transport region HTR may include at least one selected from among a hole injection layer HIL, a hole transport layer HTL, a buffer layer or an emission auxiliary layer, and an electron blocking layer EBL. At least one selected from among the hole injection layer HIL, the hole transport layer HTL and the electron blocking layer EBL may include the amine compound of one or more embodiments. For example, the hole transport layer HTL may include at least one amine compound of one or more embodiments.

The hole transport region HTR may have a single layer formed utilizing a single material, a single layer formed utilizing multiple different materials, or a multilayer structure including multiple layers formed utilizing multiple different materials.

For example, the hole transport region HTR may have the structure of a single layer of a hole injection layer HIL or a hole transport layer HTL, and may have a structure of a single layer formed utilizing a hole injection material and a hole transport material. In some embodiments, the hole transport region HTR may have a structure of a single layer formed utilizing multiple different materials, or a structure stacked from the first electrode EL1 of hole injection layer HIL/hole transport layer HTL, hole injection layer HIL/hole transport layer HTL/buffer layer, hole injection layer HIL/buffer layer, hole transport layer HTL/buffer layer, or hole injection layer HIL/hole transport layer HTLelectron blocking layer EBL, without limitation.

The hole transport region HTR may be formed utilizing one or more suitable methods such as a vacuum deposition method, a spin coating method, a cast method, a Langmuir-Blodgett (LB) method, an inkjet printing method, a laser printing method, and/or a laser induced thermal imaging (LITI) method.

The hole transport region HTR may further include the compounds explained herein. The hole transport region HTR may include a compound represented by Formula H-1.

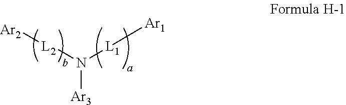

In Formula H-1, L₁ and L₂ may each independently be a direct linkage, a substituted or unsubstituted arylene group of 6 to 30 ring-forming carbon atoms, or a substituted or unsubstituted heteroarylene group of 2 to 30 ring-forming carbon atoms. In Formula H-1, “a” and “b” may each independently be an integer of 0 to 10. In some embodiments, when “a” or “b” is an integer of 2 or more, multiple L₁ and L₂ may each independently be a substituted or unsubstituted arylene group of 6 to 30 ring-forming carbon atoms, or a substituted or unsubstituted heteroarylene group of 2 to 30 ring-forming carbon atoms.

In Formula H-1, Ar₁ and Ar₂ may each independently be a substituted or unsubstituted aryl group of 6 to 30 ring-forming carbon atoms, or a substituted or unsubstituted heteroaryl group of 2 to 30 ring-forming carbon atoms. In some embodiments, in Formula H-1, Ar₃ may be a substituted or unsubstituted aryl group of 6 to 30 ring-forming carbon atoms.

The compound represented by Formula H-1 may be a monoamine compound. In some embodiments, the compound represented by Formula H-1 may be a diamine compound in which at least one selected from among Ar₁ to Ar₃ includes an amine group as a substituent. In some embodiments, the compound represented by Formula H-1 may be a carbazole-based compound in which at least one selected from among Ar₁ and Ar₂ includes a substituted or unsubstituted carbazole group, or a fluorene-based compound in which at least one selected from among Ar₁ and Ar₂ includes a substituted or unsubstituted fluorenyl group.

The compound represented by Formula H-1 may be represented by any one selected from among the compounds in Compound Group H-1. However, the compounds listed in Compound Group H-1 are only illustrations, and the compound represented by Formula H-1 is not limited to the compounds represented in Compound Group H-1.

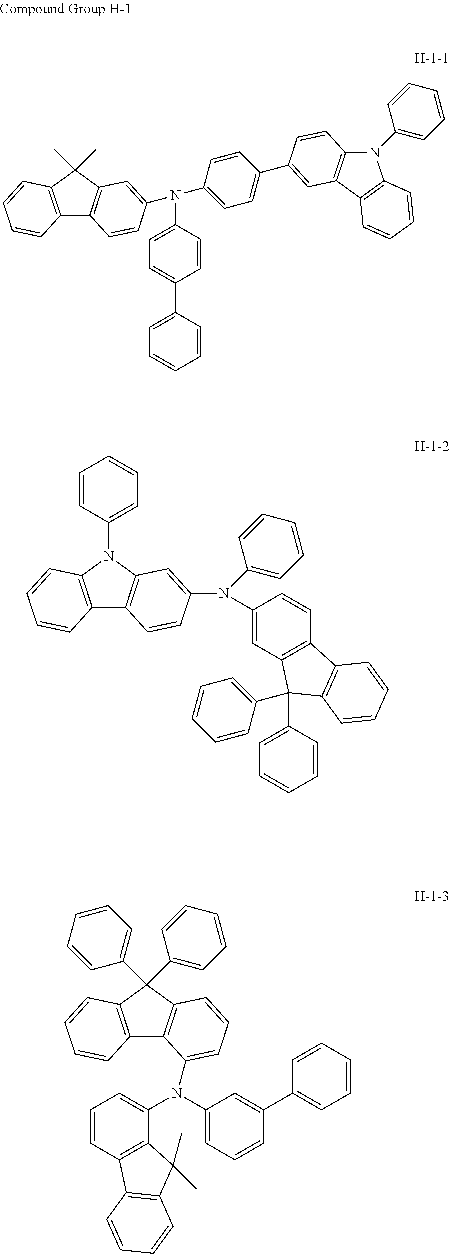 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21]

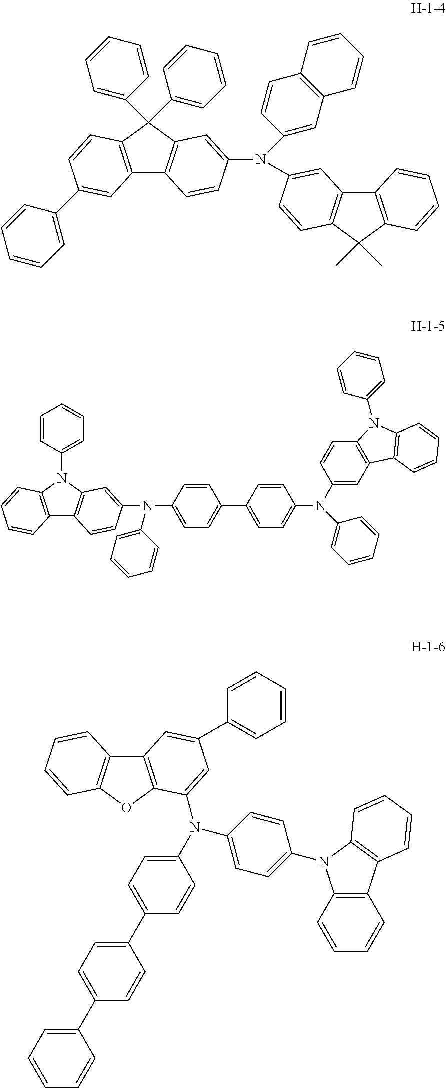 [SMILES: CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21 ; c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1]

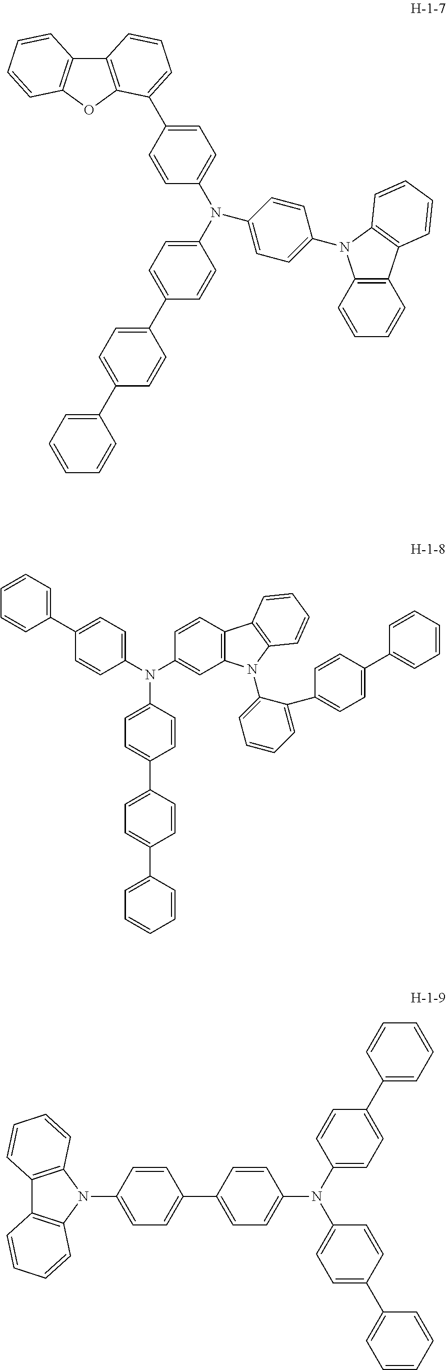 [SMILES: c1ccc(-c2ccc(-c3ccc(N(c4ccc(-c5cccc6c5oc5ccccc56)cc4)c4ccc(-n5c6ccccc6c6ccccc65)cc4)cc3)cc2)cc1 ; c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

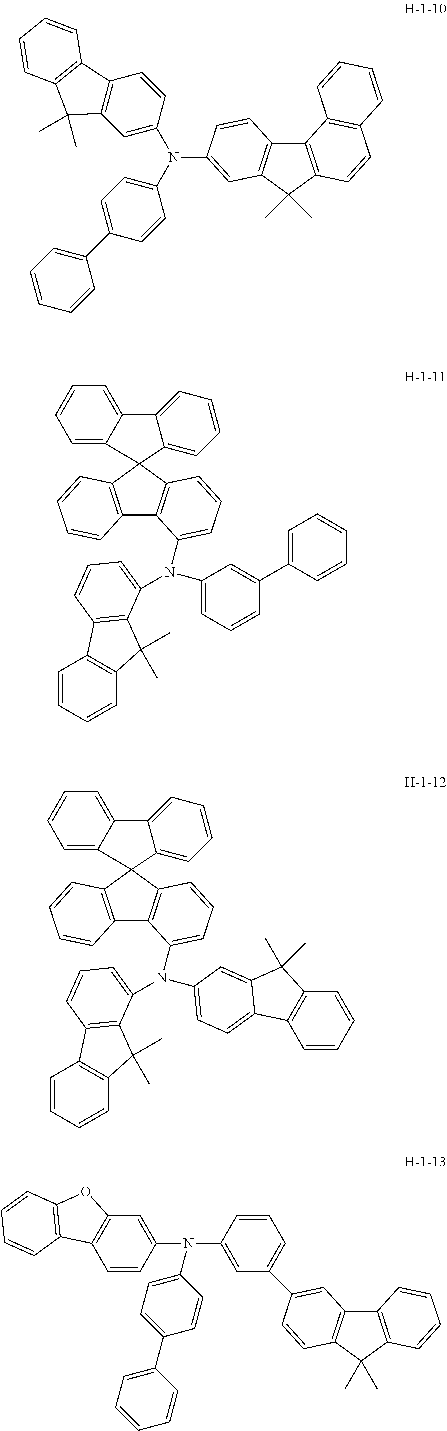 [SMILES: CC1(C)c2ccccc2-c2ccc(N(c3ccc(-c4ccccc4)cc3)c3ccc4c(c3)C(C)(C)c3ccc5ccccc5c3-4)cc21 ; CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

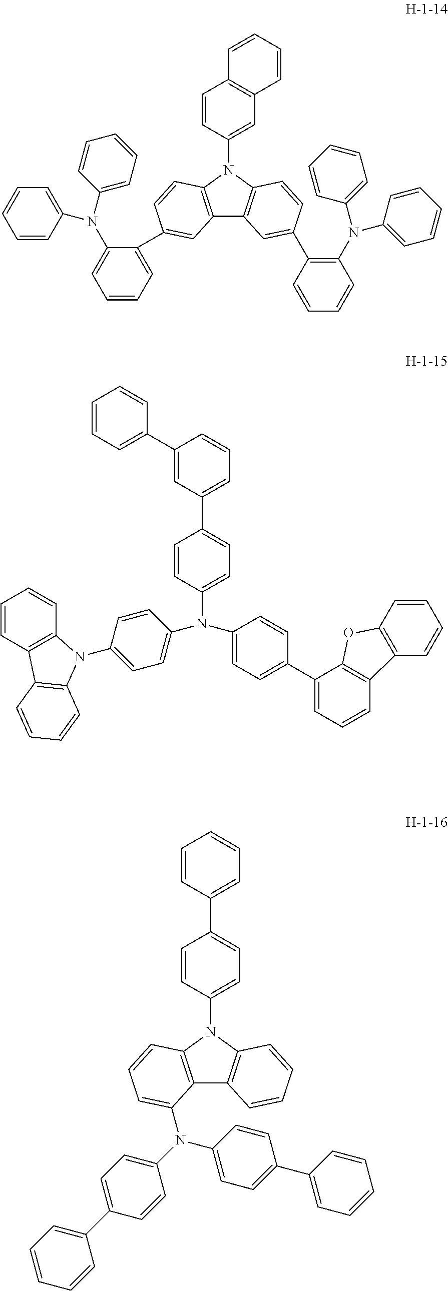 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

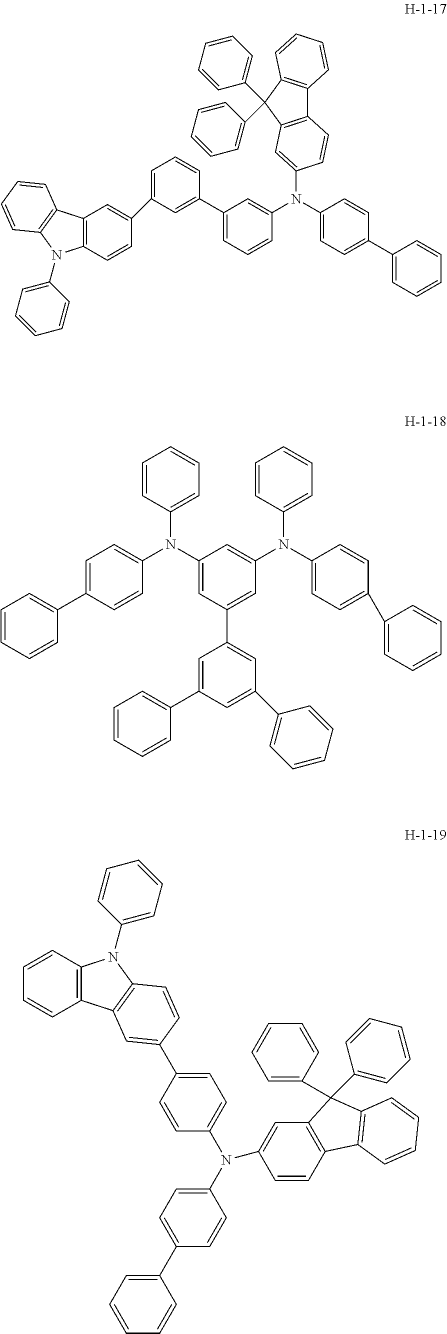 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may include a phthalocyanine compound such as copper phthalocyanine, N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino]triphenylamine (m-MTDATA), 4,4′,4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(1-naphthalene-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], and dipyrazino[2,3-f:2′,3′-h]quinoxaline-2,3,6,7,10,11-hexacarbonitrile (HAT-CN), and/or the like.

The hole transport region HTR may include carbazole derivatives such as N-phenyl carbazole and polyvinyl carbazole, fluorene-based derivatives, N, N′-bis(3-methylphenyl)-N, N′-diphenyl-[1,1′-biphenyl]-4,4′-diamine (TPD), triphenylamine-based derivatives such as 4,4′,4″-tris(N-carbazolyl)triphenylamine (TCTA), N,N′-di(naphthalene-1-yl)-N, N′-diphenyl-benzidine (NPB), 4,4′-cyclohexylidene bis[N,N-bis(4-methylphenyl)benzeneamine] (TAPC), 4,4′-bis[N, N′-(3-tolyl)amino]-3,3′-dimethylbiphenyl (HMTPD), 1,3-bis(N-carbazolyl)benzene, and/or the like.

In some embodiments, the hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), and/or the like.

The hole transport region HTR may include one or more of the compounds of the hole transport region in at least one selected from among a hole injection layer HIL, hole transport layer HTL, and electron blocking layer EBL. The thickness of the hole transport region HTR may be from about 100 Å to about 10,000 Å, for example, from about 100 Å to about 5,000 Å. When the hole transport region HTR includes a hole injection layer HIL, the thickness of the hole injection region HIL may be, for example, from about 30 Å to about 1,000 Å. When the hole transport region HTR includes a hole transport layer HTL, the thickness of the hole transport layer HTL may be from about 30 Å to about 1,000 Å. For example, when the hole transport region HTR includes an electron blocking layer EBL, the thickness of the electron blocking layer EBL may be from about 10 Å to about 1,000 Å. When the thicknesses of the hole transport region HTR, the hole injection layer HIL, the hole transport layer HTL and the electron blocking layer EBL satisfy the described ranges, satisfactory hole transport properties may be achieved without substantial increase of a driving voltage.

The hole transport region HTR may further include a charge generating material to increase conductivity in addition to the described materials. The charge generating material may be dispersed uniformly or non-uniformly in the hole transport region HTR. The charge generating material may be, for example, a p-dopant. The p-dopant may include at least one of metal halide compounds, quinone derivatives, metal oxides, and cyano group-containing compounds, without limitation. For example, the p-dopant may include metal halide compounds such as Cul and Rbl, quinone derivatives such as tetracyanoquinodimethane (TCNQ) and 2,3,5,6-tetrafluoro-7,7′,8,8-tetracyanoquinodimethane (F4-TCNQ), metal oxides such as tungsten oxide and molybdenum oxide, cyano group-containing compounds such as dipyrazino[2,3-f: 2′,3′-h] quinoxaline-2,3,6,7,10,11-hexacarbonitrile (HATCN) and 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), and/or the like, without limitation.

As described, the hole transport region HTR may further include a buffer layer in addition to the hole injection layer HIL, the hole transport layer HTL and the electron blocking layer EBL. The buffer layer may compensate for resonance distance according to the wavelength of light emitted from an emission layer EML and may increase emission efficiency. The materials included in the buffer layer may utilize materials which may be included in the hole transport region HTR. The electron blocking layer EBL is a layer playing the role of preventing or reducing electron injection from the electron transport region ETR to the hole transport region HTR.

The first electrode EL1 has conductivity (e.g., is a conductor). The first electrode EL1 may be formed utilizing a metal material, a metal alloy or a conductive compound. The first electrode EL1 may be an anode or a cathode. However, one or more embodiments of the present disclosure is not limited thereto. In some embodiments, the first electrode EL1 may be a pixel electrode. The first electrode EL1 may be a transmissive electrode, a transflective electrode, or a reflective electrode. The first electrode EL1 may include at least one selected among Ag, Mg, Cu, Al, Pt, Pd, Au, Ni, Nd, Ir, Cr, Li, Ca, LiF, Mo, Ti, W, In, Sn and Zn, compounds of two or more selected therefrom, mixtures of two or more selected therefrom, and/or oxides thereof.

When the first electrode EL1 is the transmissive electrode, the first electrode EL1 may include a transparent metal oxide such as indium tin oxide (ITO), indium zinc oxide (IZO), zinc oxide (ZnO), and/or indium tin zinc oxide (ITZO). When the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, compounds thereof, or mixtures thereof (for example, a mixture of Ag and Mg). Also, the first electrode EL1 may have a structure including multiple layers including a reflective layer or a transflective layer formed utilizing the described materials, and a transmissive conductive layer formed utilizing ITO, IZO, ZnO, or ITZO. For example, the first electrode EL1 may have a three-layer structure of ITO/Ag/ITO. However, one or more embodiments of the present disclosure is not limited thereto. The first electrode EL1 may include the described metal materials, combinations of two or more metal materials selected from the described metal materials, and/or oxides of the described metal materials. The thickness of the first electrode EL1 may be from about 700 Å to about 10,000 Å. For example, the thickness of the first electrode EL1 may be from about 1,000 Å to about 3,000 Å.

The emission layer EML is provided on the hole transport region HTR. The emission layer EML may have a thickness of, for example, about 100 Å to about 1,000 Å or about 100 Å to about 300 Å. The emission layer EML may have a single layer formed utilizing a single material, a single layer formed utilizing multiple different materials, or a multilayer structure having multiple layers formed utilizing multiple different materials.

In the light emitting element ED of one or more embodiments, the emission layer EML may include anthracene derivatives, pyrene derivatives, fluoranthene derivatives, chrysene derivatives, dihydrobenzanthracene derivatives, and/or triphenylene derivatives. For example, the emission layer EML may include anthracene derivatives or pyrene derivatives.

In the light emitting elements ED of embodiments, shown in FIG. 3 to FIG. 6, the emission layer EML may include a host and a dopant, and the emission layer EML may include a compound represented by Formula E-1. The compound represented by Formula E-1 may be utilized as a fluorescence host material.

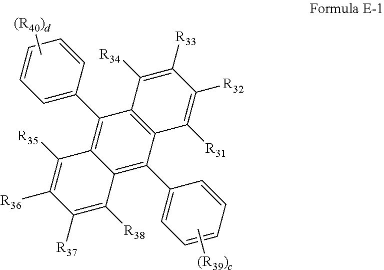

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. In some embodiments, R₃₁ to R₄₀ may be combined with an adjacent group to form a saturated hydrocarbon ring, an unsaturated hydrocarbon ring, a saturated heterocycle, or an unsaturated heterocycle.

In Formula E-1, “c” and “d” may each independently be an integer of 0 to 5.

Formula E-1 may be represented by any one among Compound E1 to Compound E19.

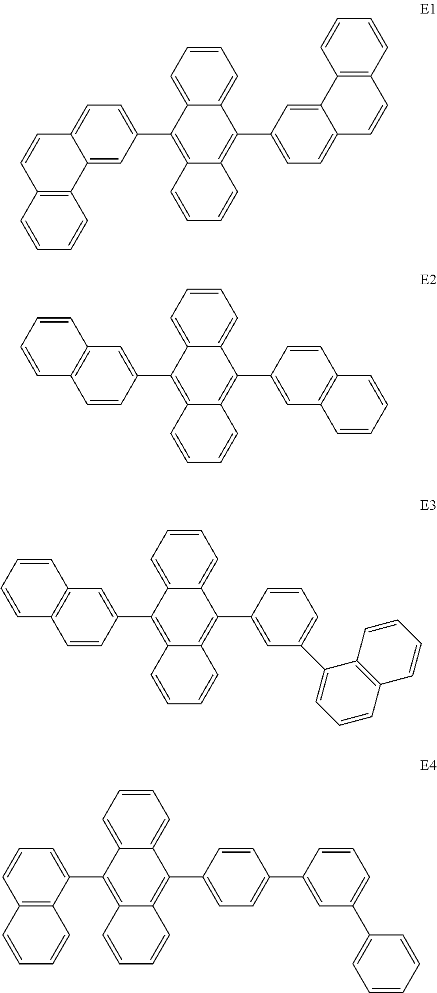

In one or more embodiments, the emission layer EML may include at least one selected from among a first compound represented by Formula E-1, a second compound represented by Formula HT-1, a third compound represented by Formula ET-1 and a fourth compound represented by Formula M-b.

In one or more embodiments, the second compound may be utilized as the hole transport host material of the emission layer EML.

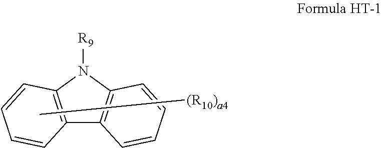

In Formula HT-1, a4 may be an integer of 0 to 8. When a4 is an integer of 2 or more, multiple R₁₀ may be the same, or at least one may be different. R₉ and R₁₀ may each independently be a hydrogen atom, a deuterium atom, a substituted or unsubstituted aryl group of 6 to 60 ring-forming carbon atoms, or a substituted or unsubstituted heteroaryl group of 2 to 60 ring-forming carbon atoms. For example, R₉ may be a substituted phenyl group, an unsubstituted dibenzofuran group, or a substituted fluorenyl group. R₁₀ may be a substituted or unsubstituted carbazole group.

The second compound may be represented by any one among the compounds in Compound Group 2. In Compound Group 2, D is a deuterium atom.

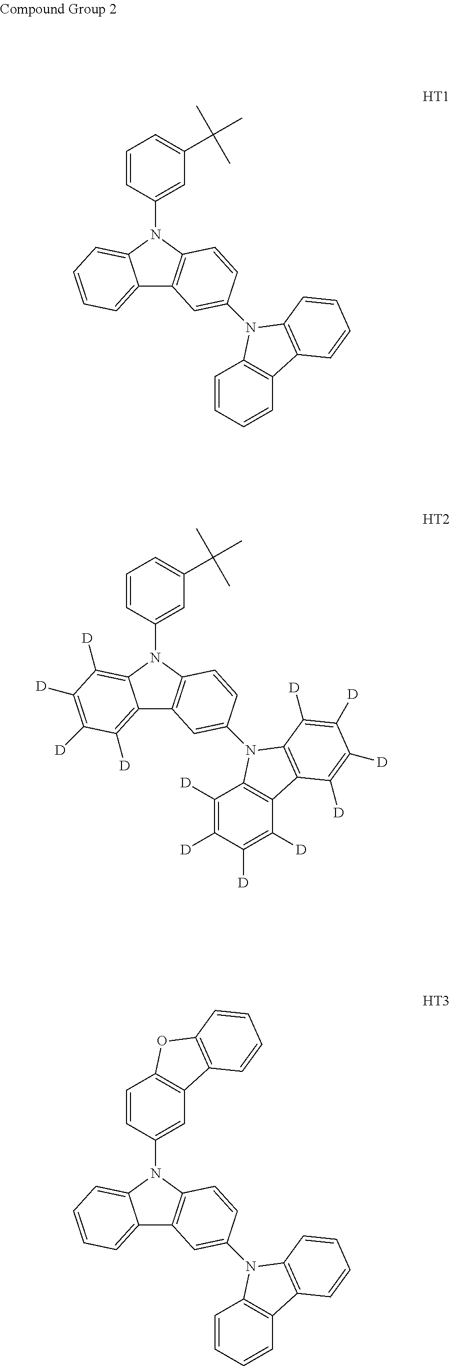 [SMILES: CC(C)(C)c1cccc(-n2c3ccccc3c3cc(-n4c5ccccc5c5ccccc54)ccc32)c1 ; [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1 ; c1ccc2c(c1)oc1ccc(-n3c4ccccc4c4cc(-n5c6ccccc6c6ccccc65)ccc43)cc12]

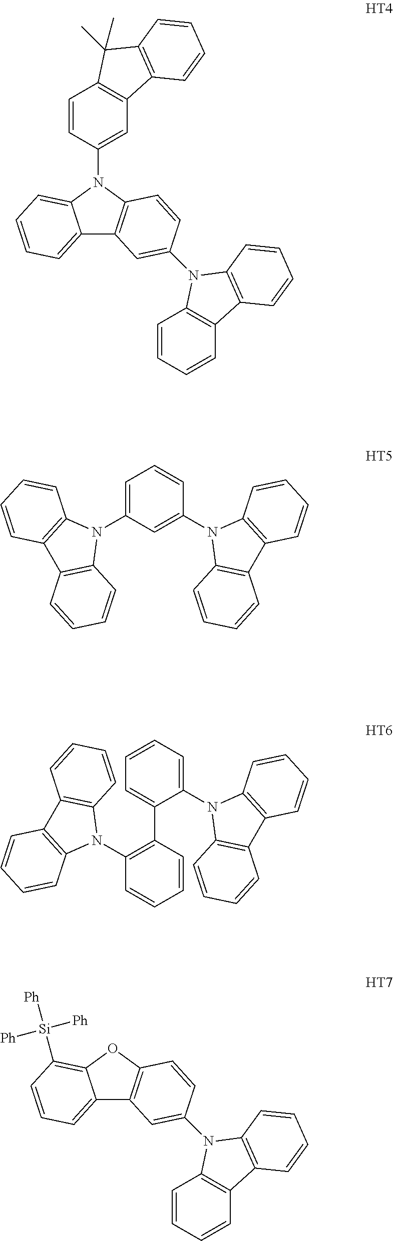 [SMILES: CC1(C)c2ccccc2-c2cc(-n3c4ccccc4c4cc(-n5c6ccccc6c6ccccc65)ccc43)ccc21 ; c1cc(-n2c3ccccc3c3ccccc32)cc(-n2c3ccccc3c3ccccc32)c1 ; c1ccc(-n2c3ccccc3c3ccccc32)c(-c2ccccc2-n2c3ccccc3c3ccccc32)c1 ; c1ccc([Si](c2ccccc2)(c2ccccc2)c2cccc3c2oc2ccc(-n4c5ccccc5c5ccccc54)cc23)cc1]

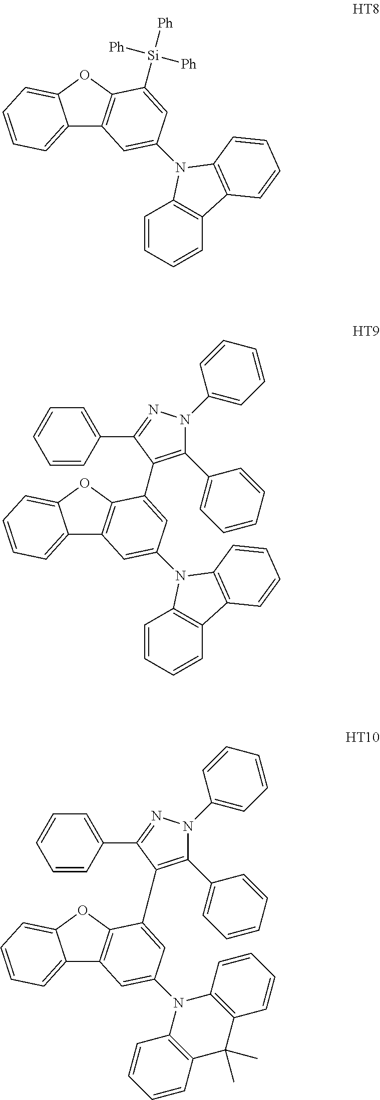 [SMILES: c1ccc([Si](c2ccccc2)(c2ccccc2)c2cc(-n3c4ccccc4c4ccccc43)cc3c2oc2ccccc23)cc1 ; c1ccc(-c2nn(-c3ccccc3)c(-c3ccccc3)c2-c2cc(-n3c4ccccc4c4ccccc43)cc3c2oc2ccccc23)cc1 ; CC1(C)c2ccccc2N(c2cc(-c3c(-c4ccccc4)nn(-c4ccccc4)c3-c3ccccc3)c3oc4ccccc4c3c2)c2ccccc21]

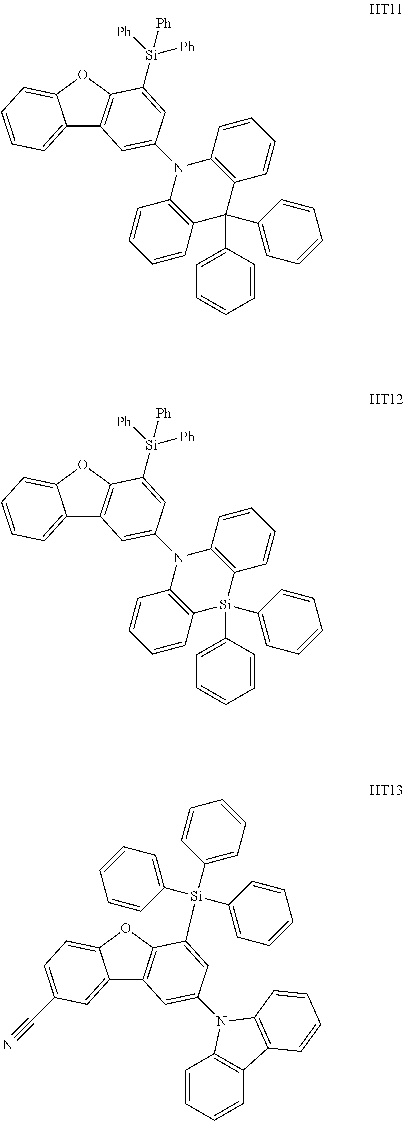 [SMILES: c1ccc(C2(c3ccccc3)c3ccccc3N(c3cc([Si](c4ccccc4)(c4ccccc4)c4ccccc4)c4oc5ccccc5c4c3)c3ccccc32)cc1 ; c1ccc([Si]2(c3ccccc3)c3ccccc3N(c3cc([Si](c4ccccc4)(c4ccccc4)c4ccccc4)c4oc5ccccc5c4c3)c3ccccc32)cc1 ; N#Cc1ccc2oc3c([Si](c4ccccc4)(c4ccccc4)c4ccccc4)cc(-n4c5ccccc5c5ccccc54)cc3c2c1]

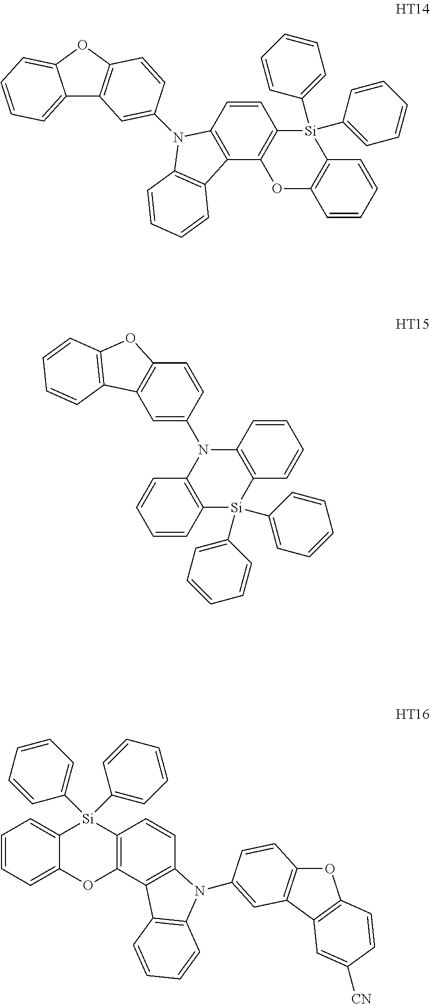 [SMILES: c1ccc([Si]2(c3ccccc3)c3ccccc3Oc3c2ccc2c3c3ccccc3n2-c2ccc3oc4ccccc4c3c2)cc1 ; c1ccc([Si]2(c3ccccc3)c3ccccc3N(c3ccc4oc5ccccc5c4c3)c3ccccc32)cc1 ; N#Cc1ccc2oc3ccc(-n4c5ccccc5c5c6c(ccc54)[Si](c4ccccc4)(c4ccccc4)c4ccccc4O6)cc3c2c1]

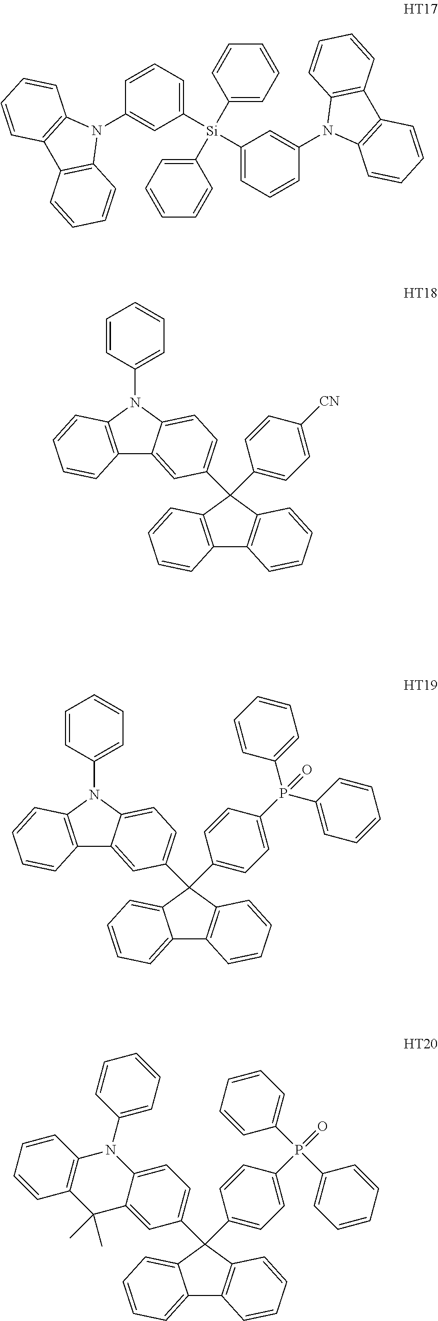 [SMILES: c1ccc([Si](c2ccccc2)(c2cccc(-n3c4ccccc4c4ccccc43)c2)c2cccc(-n3c4ccccc4c4ccccc43)c2)cc1 ; N#Cc1ccc(C2(c3ccc4c(c3)c3ccccc3n4-c3ccccc3)c3ccccc3-c3ccccc32)cc1 ; O=P(c1ccccc1)(c1ccccc1)c1ccc(C2(c3ccc4c(c3)c3ccccc3n4-c3ccccc3)c3ccccc3-c3ccccc32)cc1 ; CC1(C)c2ccccc2N(c2ccccc2)c2ccc(C3(c4ccc(P(=O)(c5ccccc5)c5ccccc5)cc4)c4ccccc4-c4ccccc43)cc21]

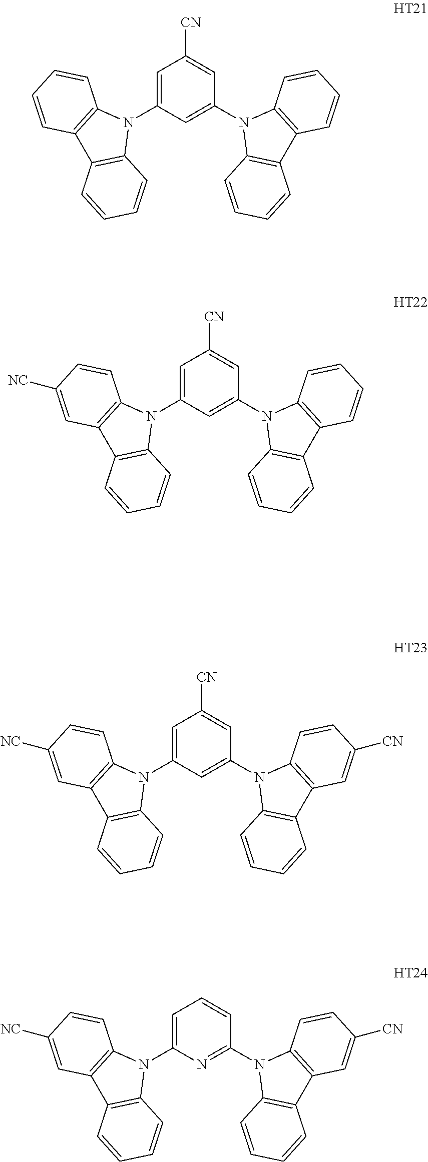 [SMILES: N#Cc1cc(-n2c3ccccc3c3ccccc32)cc(-n2c3ccccc3c3ccccc32)c1 ; N#Cc1cc(-n2c3ccccc3c3ccccc32)cc(-n2c3ccccc3c3cc(C#N)ccc32)c1 ; N#Cc1cc(-n2c3ccccc3c3cc(C#N)ccc32)cc(-n2c3ccccc3c3cc(C#N)ccc32)c1 ; N#Cc1ccc2c(c1)c1ccccc1n2-c1cccc(-n2c3ccccc3c3cc(C#N)ccc32)n1]

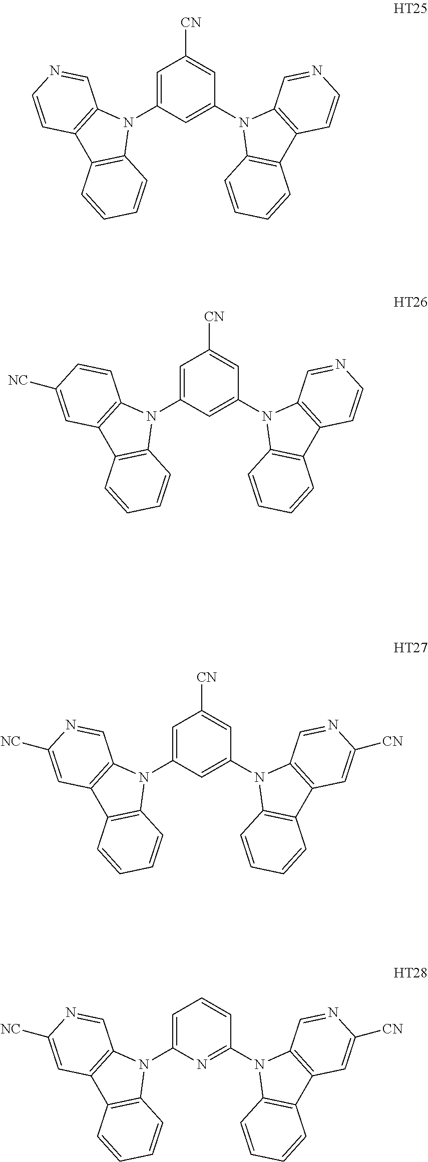 [SMILES: N#Cc1cc(-n2c3ccccc3c3ccncc32)cc(-n2c3ccccc3c3ccncc32)c1 ; N#Cc1cc(-n2c3ccccc3c3cc(C#N)ccc32)cc(-n2c3ccccc3c3ccncc32)c1 ; N#Cc1cc(-n2c3ccccc3c3cc(C#N)ncc32)cc(-n2c3ccccc3c3cc(C#N)ncc32)c1 ; N#Cc1cc2c3ccccc3n(-c3cccc(-n4c5ccccc5c5cc(C#N)ncc54)n3)c2cn1]

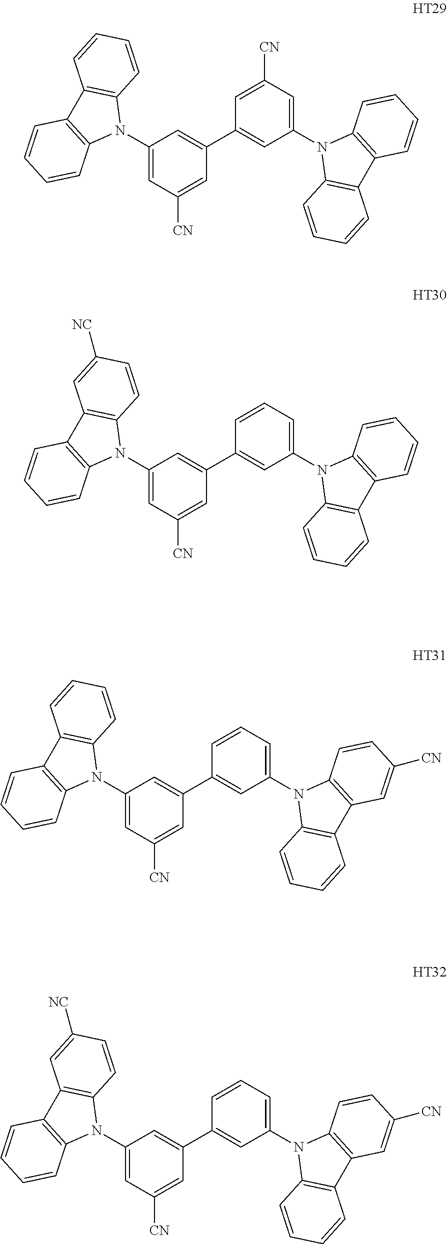 [SMILES: N#Cc1cc(-c2cc(C#N)cc(-n3c4ccccc4c4ccccc43)c2)cc(-n2c3ccccc3c3ccccc32)c1 ; N#Cc1cc(-c2cccc(-n3c4ccccc4c4ccccc43)c2)cc(-n2c3ccccc3c3cc(C#N)ccc32)c1 ; N#Cc1cc(-c2cccc(-n3c4ccccc4c4cc(C#N)ccc43)c2)cc(-n2c3ccccc3c3ccccc32)c1 ; N#Cc1cc(-c2cccc(-n3c4ccccc4c4cc(C#N)ccc43)c2)cc(-n2c3ccccc3c3cc(C#N)ccc32)c1]

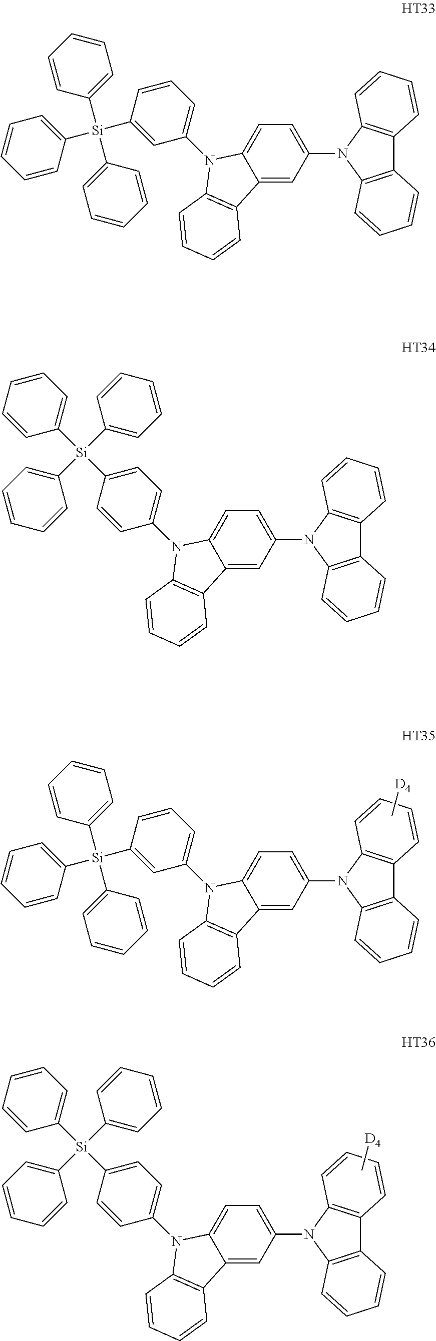

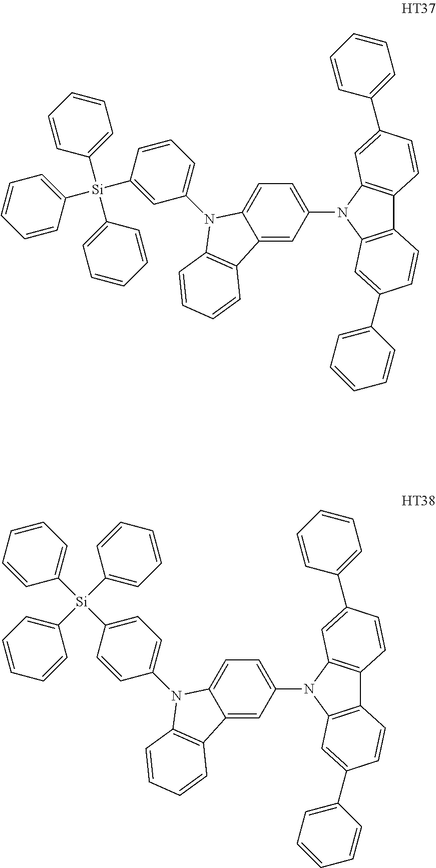 [SMILES: c1ccc(-c2ccc3c4ccc(-c5ccccc5)cc4n(-c4ccc5c(c4)c4ccccc4n5-c4cccc([Si](c5ccccc5)(c5ccccc5)c5ccccc5)c4)c3c2)cc1 ; c1ccc(-c2ccc3c4ccc(-c5ccccc5)cc4n(-c4ccc5c(c4)c4ccccc4n5-c4ccc([Si](c5ccccc5)(c5ccccc5)c5ccccc5)cc4)c3c2)cc1]

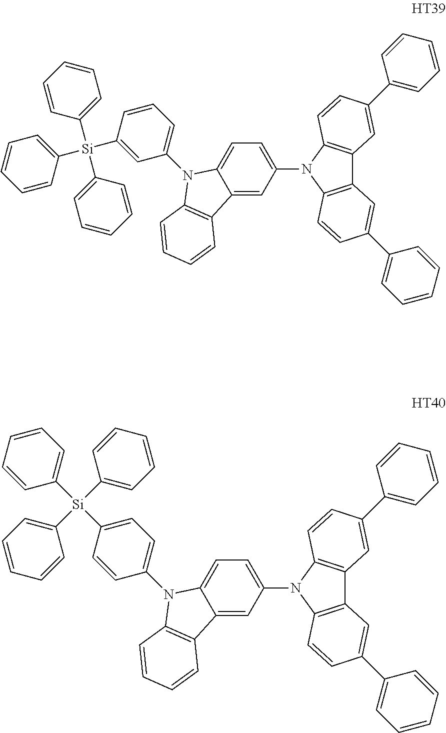 [SMILES: c1ccc(-c2ccc3c(c2)c2cc(-c4ccccc4)ccc2n3-c2ccc3c(c2)c2ccccc2n3-c2cccc([Si](c3ccccc3)(c3ccccc3)c3ccccc3)c2)cc1 ; c1ccc(-c2ccc3c(c2)c2cc(-c4ccccc4)ccc2n3-c2ccc3c(c2)c2ccccc2n3-c2ccc([Si](c3ccccc3)(c3ccccc3)c3ccccc3)cc2)cc1]

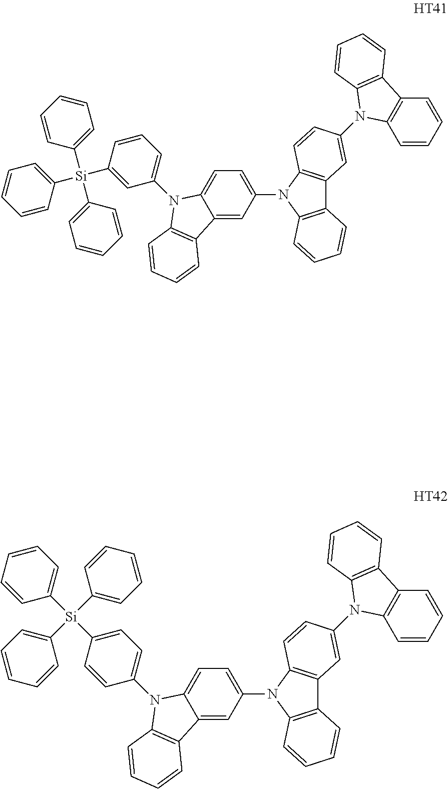 [SMILES: c1ccc([Si](c2ccccc2)(c2ccccc2)c2cccc(-n3c4ccccc4c4cc(-n5c6ccccc6c6cc(-n7c8ccccc8c8ccccc87)ccc65)ccc43)c2)cc1 ; c1ccc([Si](c2ccccc2)(c2ccccc2)c2ccc(-n3c4ccccc4c4cc(-n5c6ccccc6c6cc(-n7c8ccccc8c8ccccc87)ccc65)ccc43)cc2)cc1]

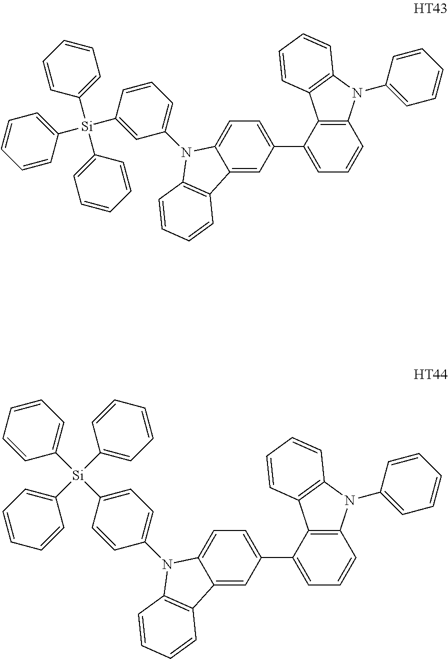 [SMILES: c1ccc(-n2c3ccccc3c3c(-c4ccc5c(c4)c4ccccc4n5-c4cccc([Si](c5ccccc5)(c5ccccc5)c5ccccc5)c4)cccc32)cc1 ; c1ccc(-n2c3ccccc3c3c(-c4ccc5c(c4)c4ccccc4n5-c4ccc([Si](c5ccccc5)(c5ccccc5)c5ccccc5)cc4)cccc32)cc1]

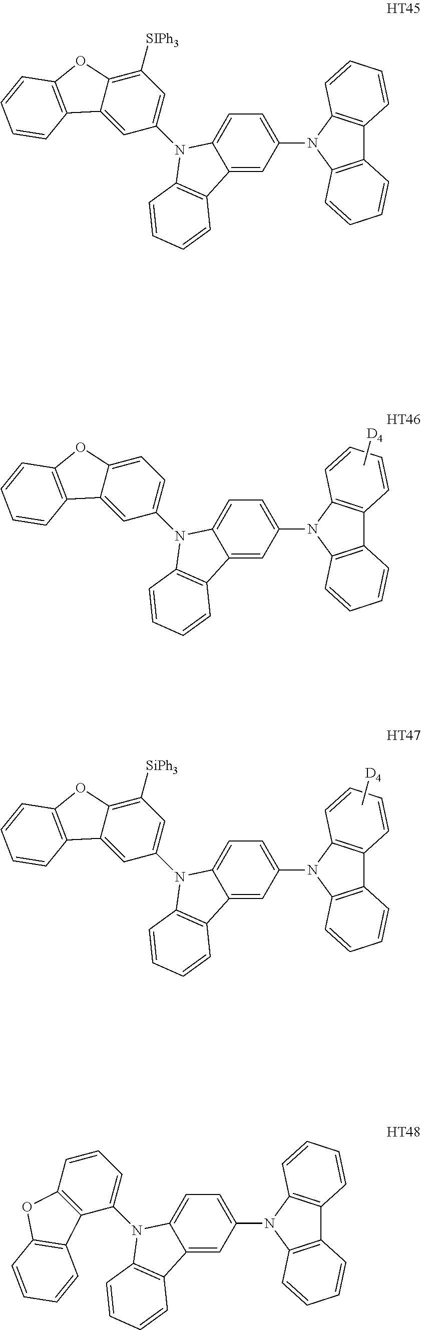

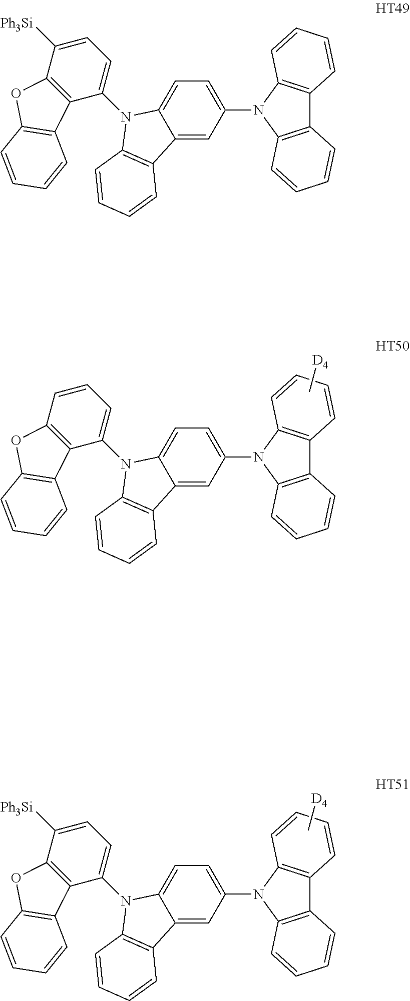

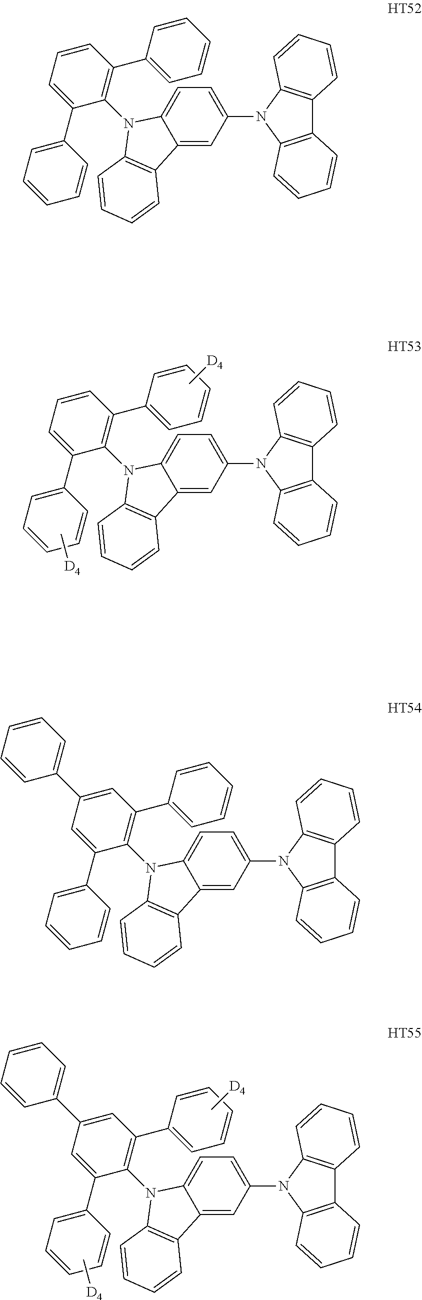

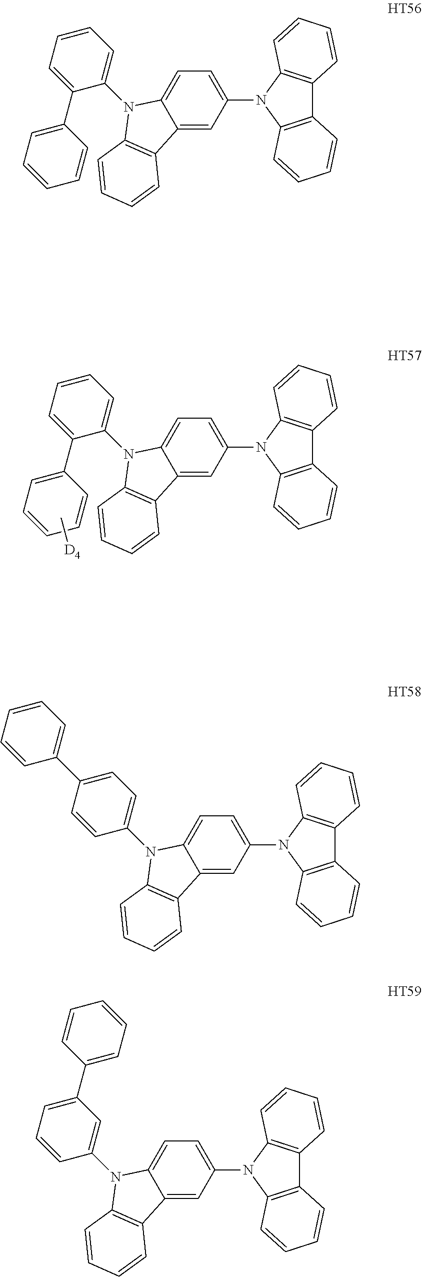

In one or more embodiments, the emission layer EML may include a third compound represented by Formula ET-1. For example, the third compound may be utilized as the electron transport host material of the emission layer EML.

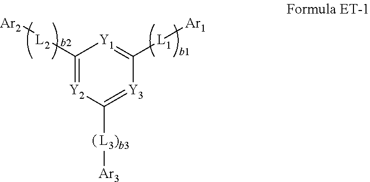

In Formula ET-1, at least one selected from among Y₁ to Y₃ may be N, and the remainder may be CRₐ, and Rₐ may be a hydrogen atom, a deuterium atom, a substituted or unsubstituted alkyl group of 1 to 20 carbon atoms, a substituted or unsubstituted aryl group of 6 to 60 ring-forming carbon atoms, or a substituted or unsubstituted heteroaryl group of 2 to 60 ring-forming carbon atoms.

b1 to b3 may each independently be an integer of 0 to 10. L₁ to L₃ may each independently be a direct linkage, a substituted or unsubstituted arylene group of 6 to 30 ring-forming carbon atoms, or a substituted or unsubstituted heteroarylene group of 2 to 30 ring-forming carbon atoms.

Ar₁ to Ar₃ may each independently be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, Ar₁ to Ar₃ may be substituted or unsubstituted phenyl groups, or substituted or unsubstituted carbazole groups.

The third compound may be represented by any one among the compounds in Compound Group 3. The light emitting element ED of one or more embodiments may include any one among the compounds in Compound Group 3. In Compound Group 3, D is a deuterium atom.

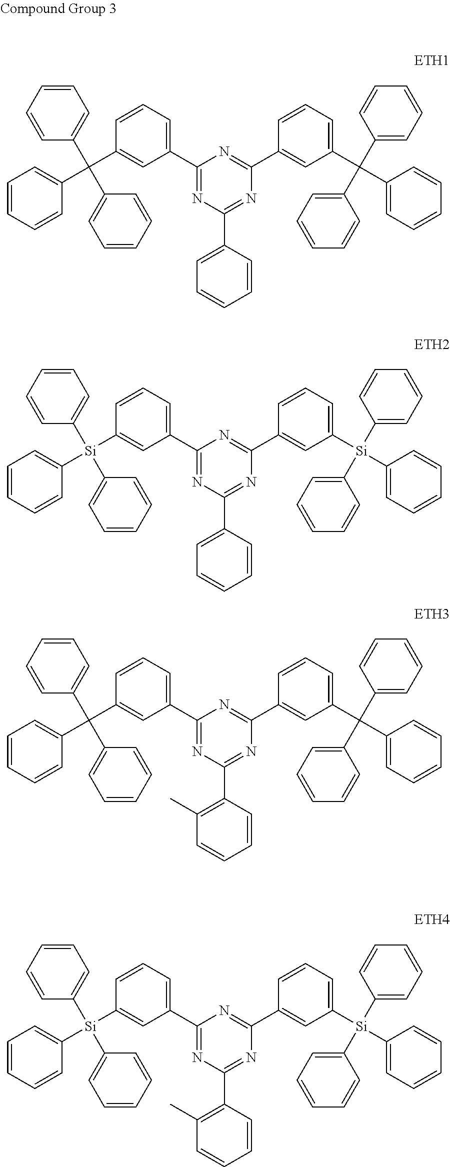 [SMILES: c1ccc(-c2nc(-c3cccc(C(c4ccccc4)(c4ccccc4)c4ccccc4)c3)nc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)nc(-c2cccc([Si](c3ccccc3)(c3ccccc3)c3ccccc3)c2)n1]

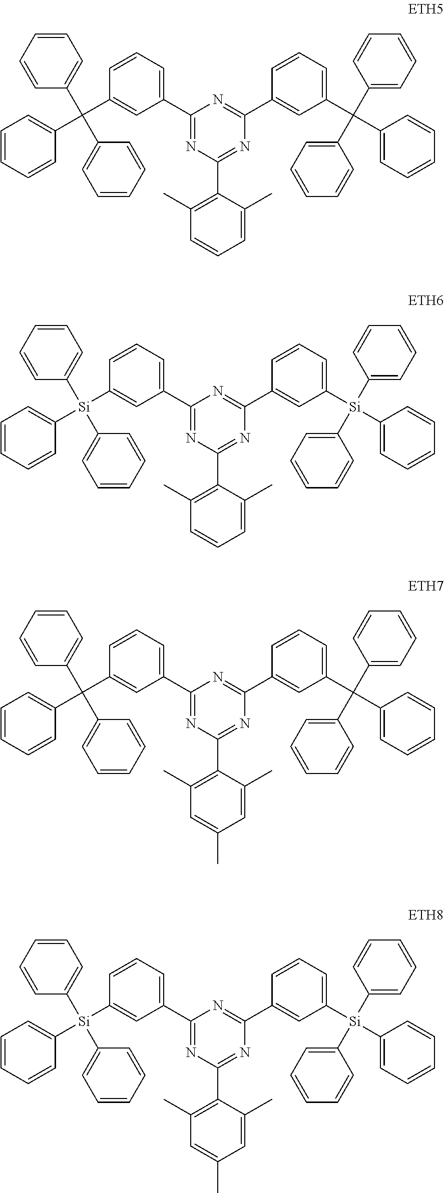 [SMILES: Cc1cccc(C)c1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)nc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)nc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)nc(-c3cccc([Si](c4ccccc4)(c4ccccc4)c4ccccc4)c3)n2)c(C)c1]

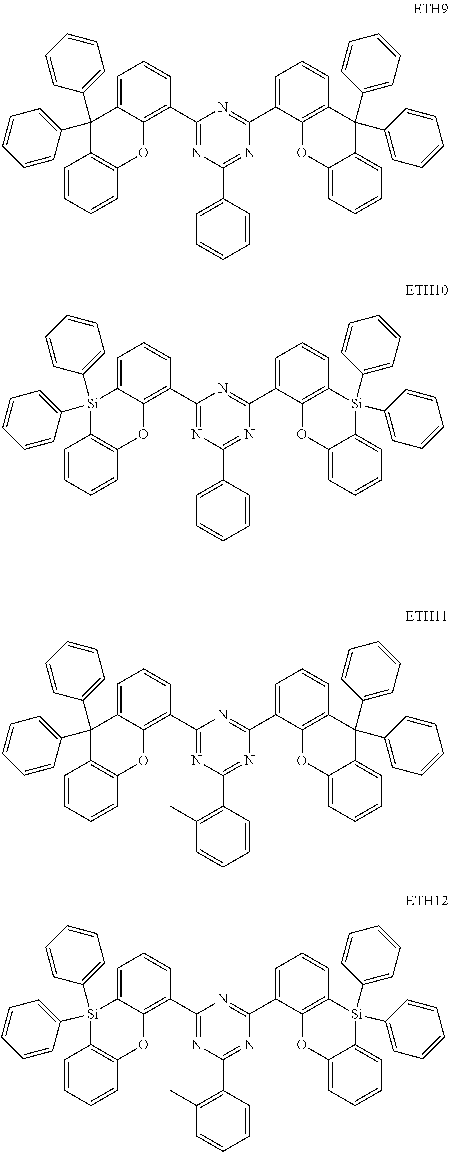 [SMILES: c1ccc(-c2nc(-c3cccc4c3Oc3ccccc3C4(c3ccccc3)c3ccccc3)nc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1]

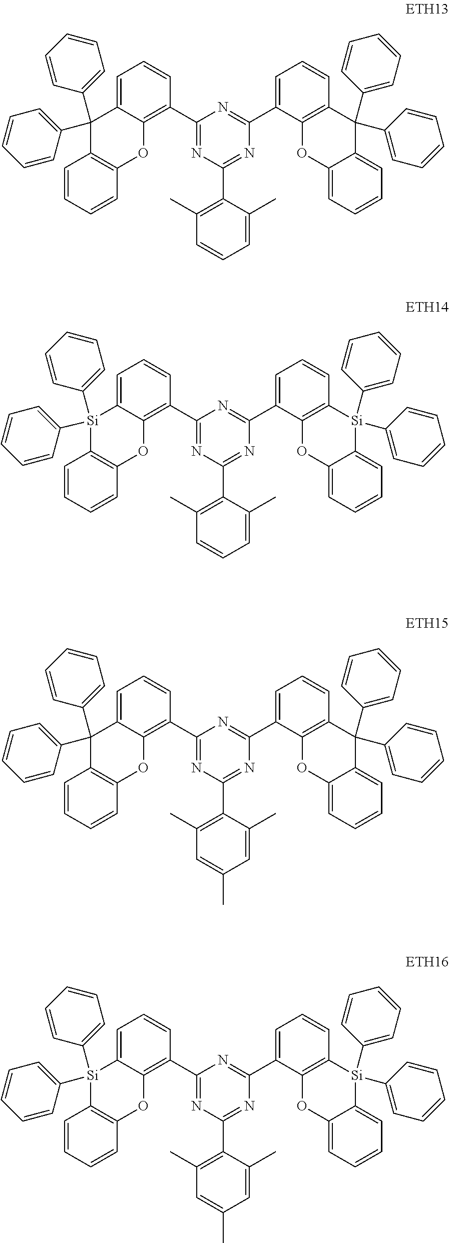 [SMILES: Cc1cccc(C)c1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)nc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)c(C)c1]

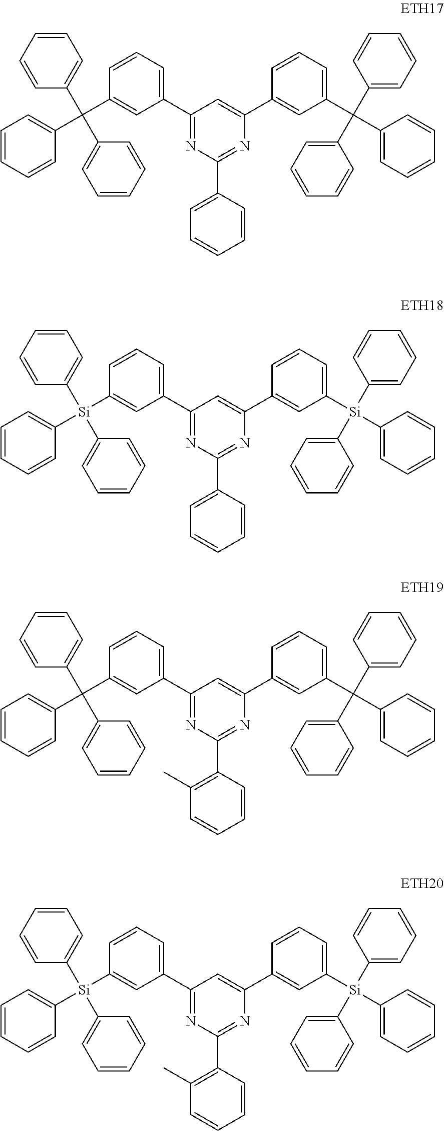 [SMILES: c1ccc(-c2nc(-c3cccc(C(c4ccccc4)(c4ccccc4)c4ccccc4)c3)cc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)cc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)cc(-c2cccc([Si](c3ccccc3)(c3ccccc3)c3ccccc3)c2)n1]

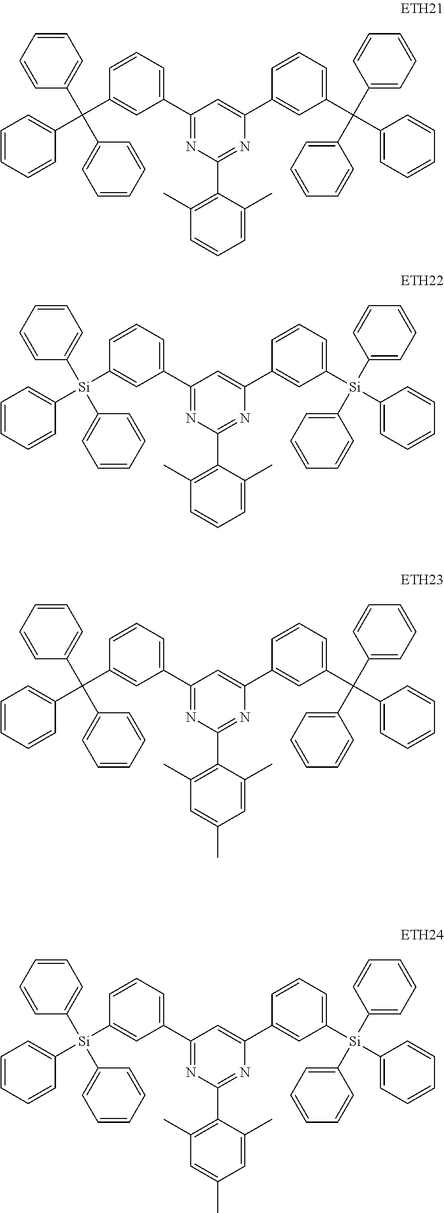 [SMILES: Cc1cccc(C)c1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)cc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)cc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)cc(-c3cccc([Si](c4ccccc4)(c4ccccc4)c4ccccc4)c3)n2)c(C)c1]

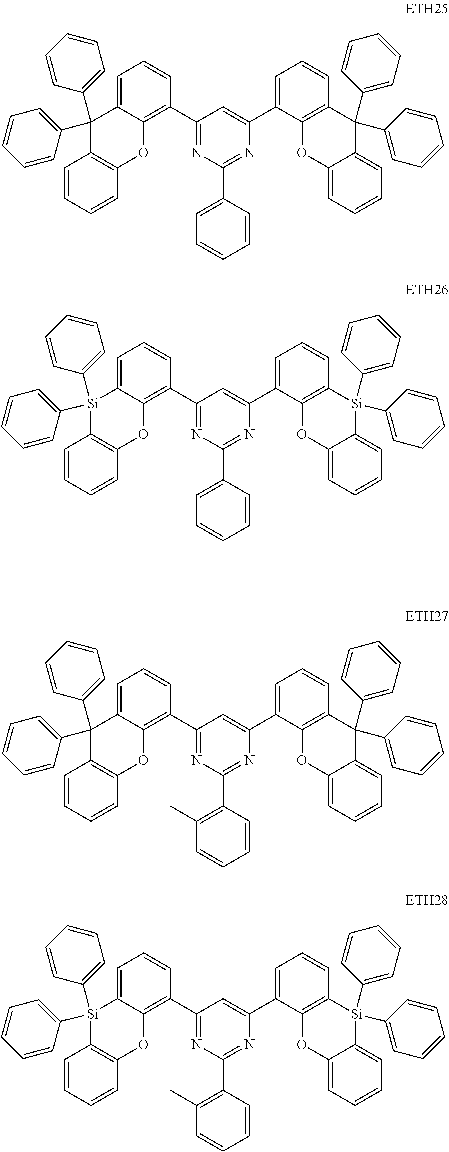 [SMILES: c1ccc(-c2nc(-c3cccc4c3Oc3ccccc3C4(c3ccccc3)c3ccccc3)cc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1]

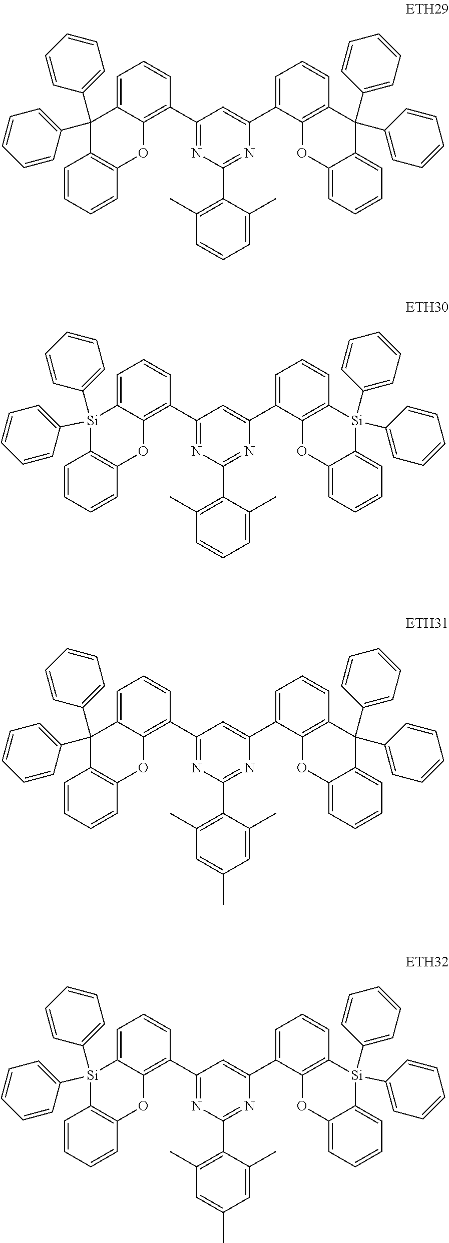 [SMILES: Cc1cccc(C)c1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)cc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)c(C)c1]

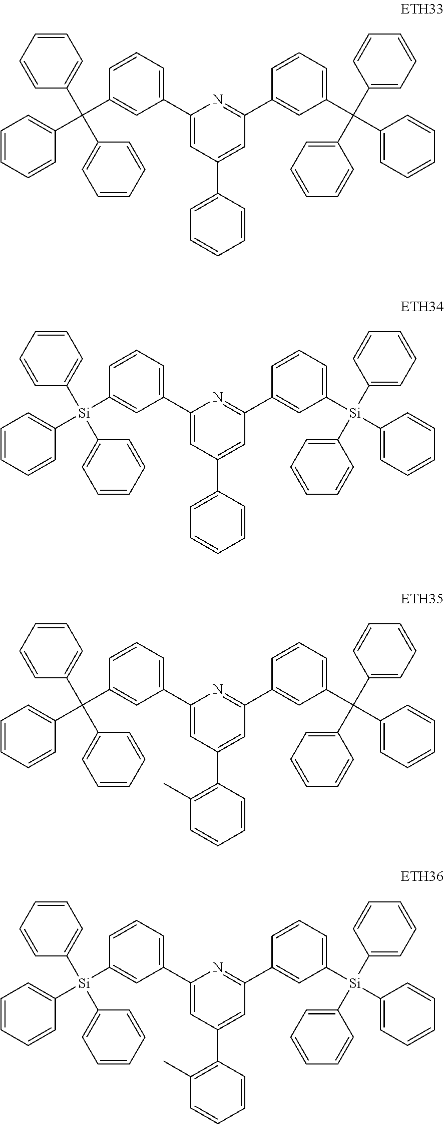 [SMILES: c1ccc(-c2cc(-c3cccc(C(c4ccccc4)(c4ccccc4)c4ccccc4)c3)nc(-c3cccc(C(c4ccccc4)(c4ccccc4)c4ccccc4)c3)c2)cc1 ; c1ccc(-c2cc(-c3cccc([Si](c4ccccc4)(c4ccccc4)c4ccccc4)c3)nc(-c3cccc([Si](c4ccccc4)(c4ccccc4)c4ccccc4)c3)c2)cc1 ; Cc1ccccc1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1ccccc1-c1cc(-c2cccc([Si](c3ccccc3)(c3ccccc3)c3ccccc3)c2)nc(-c2cccc([Si](c3ccccc3)(c3ccccc3)c3ccccc3)c2)c1]

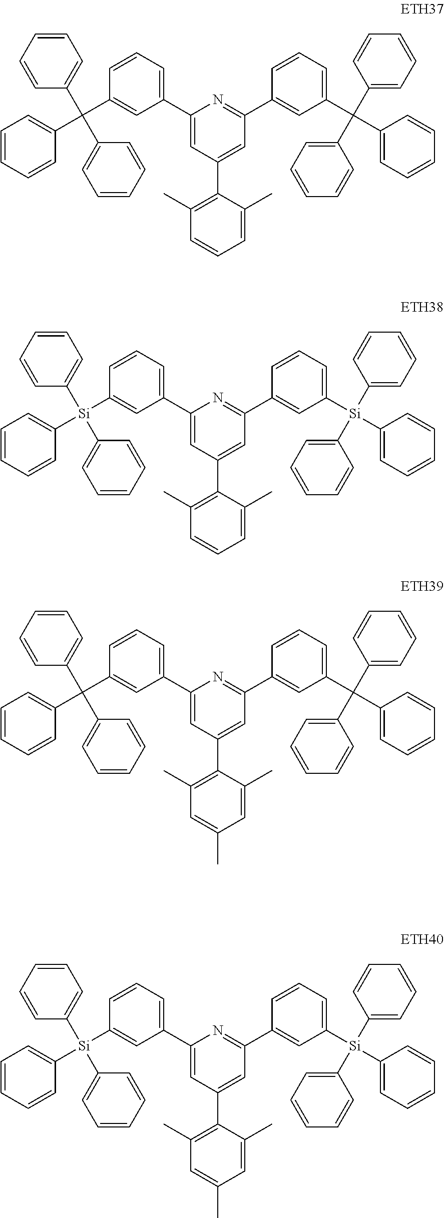 [SMILES: Cc1cccc(C)c1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1cccc(C)c1-c1cc(-c2cccc([Si](c3ccccc3)(c3ccccc3)c3ccccc3)c2)nc(-c2cccc([Si](c3ccccc3)(c3ccccc3)c3ccccc3)c2)c1 ; Cc1cc(C)c(-c2cc(-c3cccc(C(c4ccccc4)(c4ccccc4)c4ccccc4)c3)nc(-c3cccc(C(c4ccccc4)(c4ccccc4)c4ccccc4)c3)c2)c(C)c1 ; Cc1cc(C)c(-c2cc(-c3cccc([Si](c4ccccc4)(c4ccccc4)c4ccccc4)c3)nc(-c3cccc([Si](c4ccccc4)(c4ccccc4)c4ccccc4)c3)c2)c(C)c1]

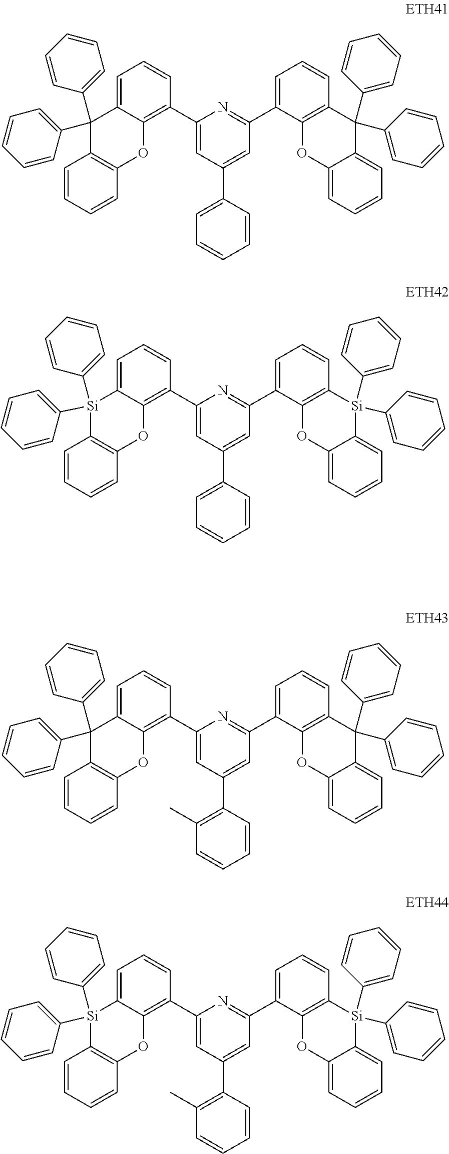 [SMILES: c1ccc(-c2cc(-c3cccc4c3Oc3ccccc3C4(c3ccccc3)c3ccccc3)nc(-c3cccc4c3Oc3ccccc3C4(c3ccccc3)c3ccccc3)c2)cc1 ; c1ccc(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)cc1 ; Cc1ccccc1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1ccccc1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1]

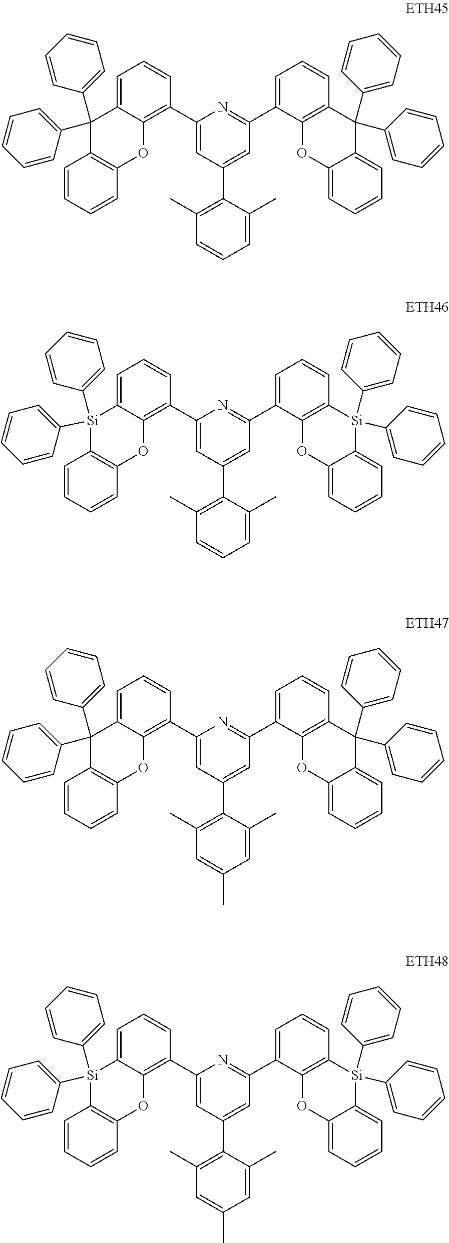 [SMILES: Cc1cccc(C)c1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1cccc(C)c1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1 ; Cc1cc(C)c(-c2cc(-c3cccc4c3Oc3ccccc3C4(c3ccccc3)c3ccccc3)nc(-c3cccc4c3Oc3ccccc3C4(c3ccccc3)c3ccccc3)c2)c(C)c1 ; Cc1cc(C)c(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)c(C)c1]

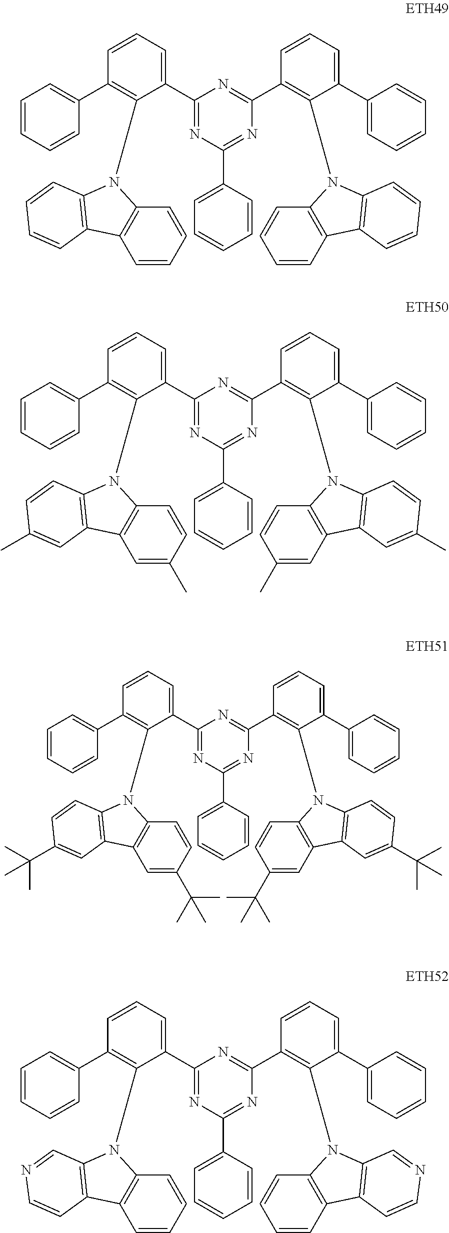

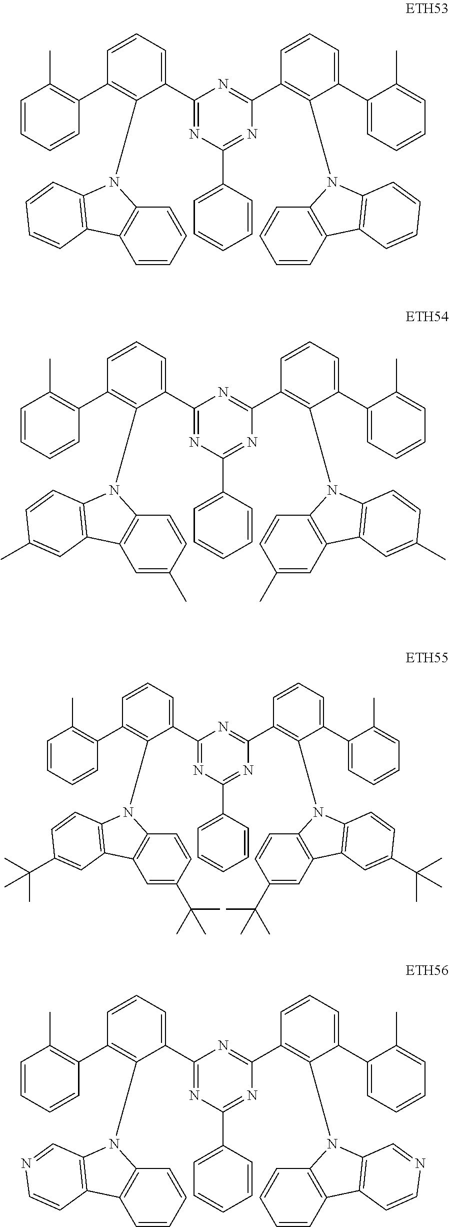

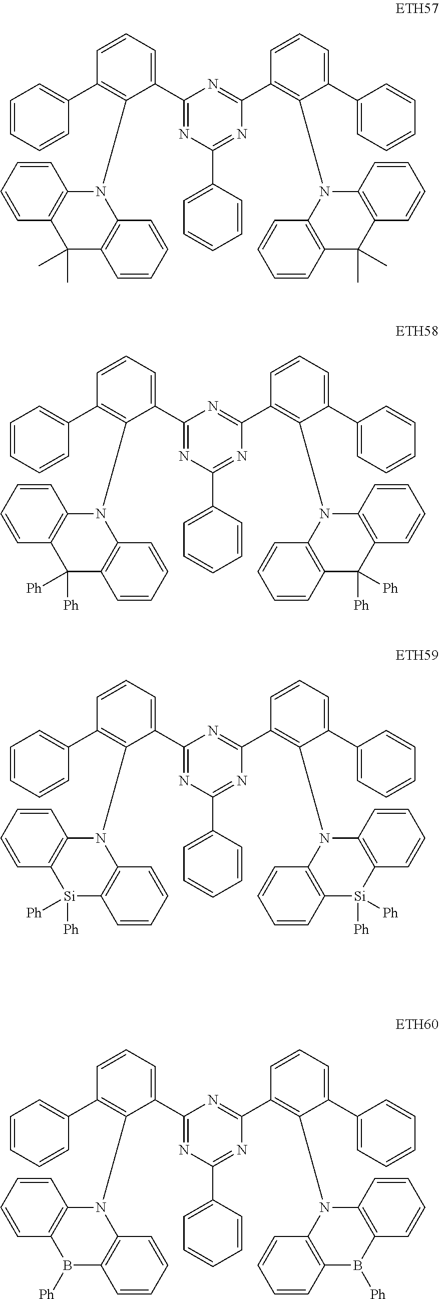

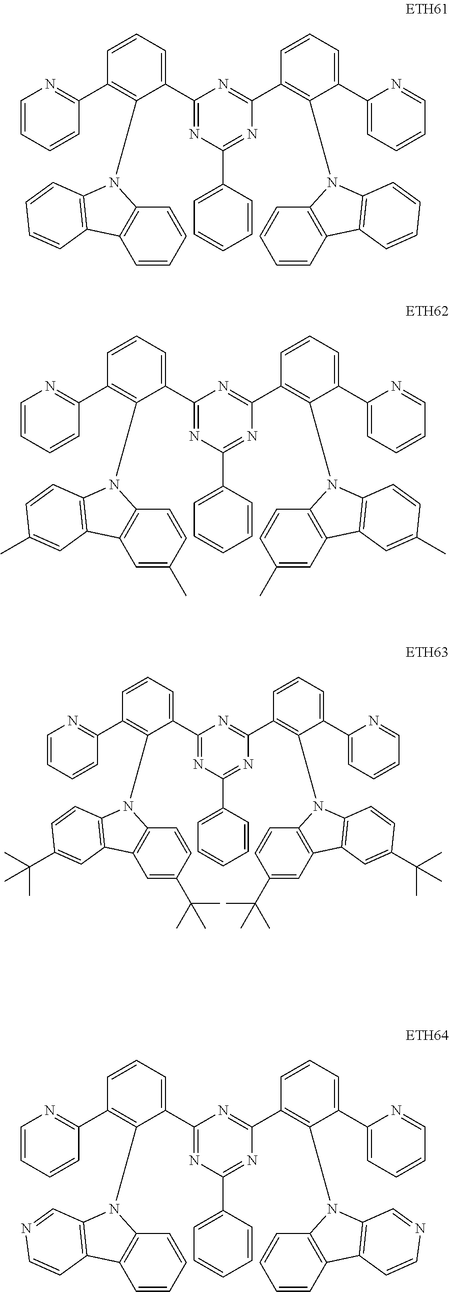

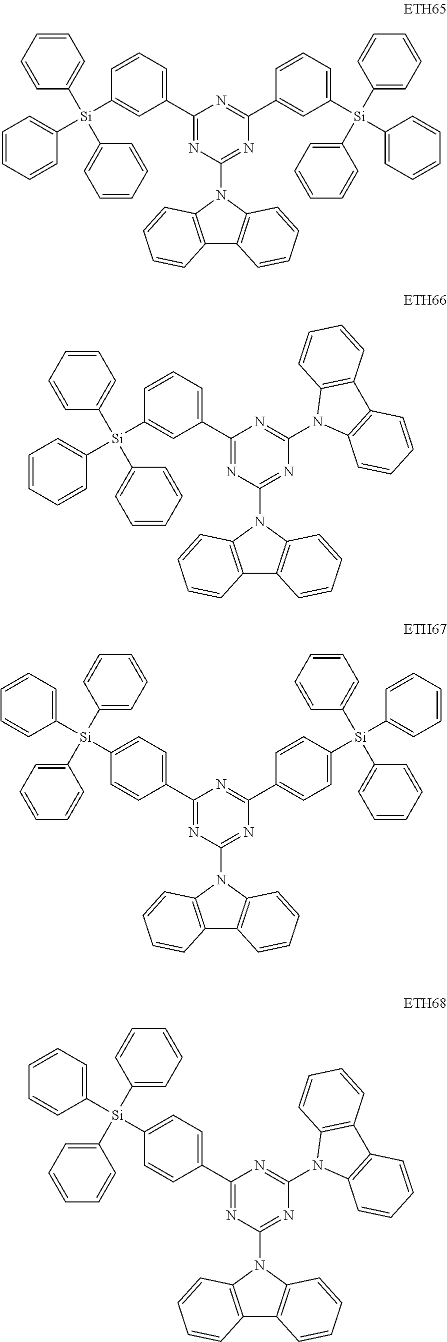 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5ccccc54)n3)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5ccccc54)n3)cc2)cc1 ; c1ccc([Si](c2ccccc2)(c2ccccc2)c2ccc(-c3nc(-n4c5ccccc5c5ccccc54)nc(-n4c5ccccc5c5ccccc54)n3)cc2)cc1]

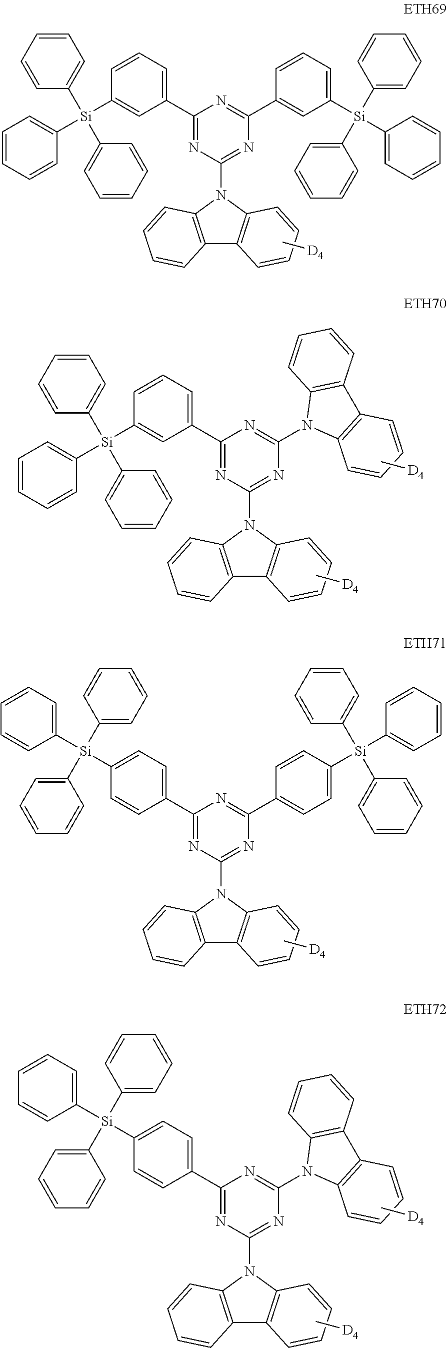

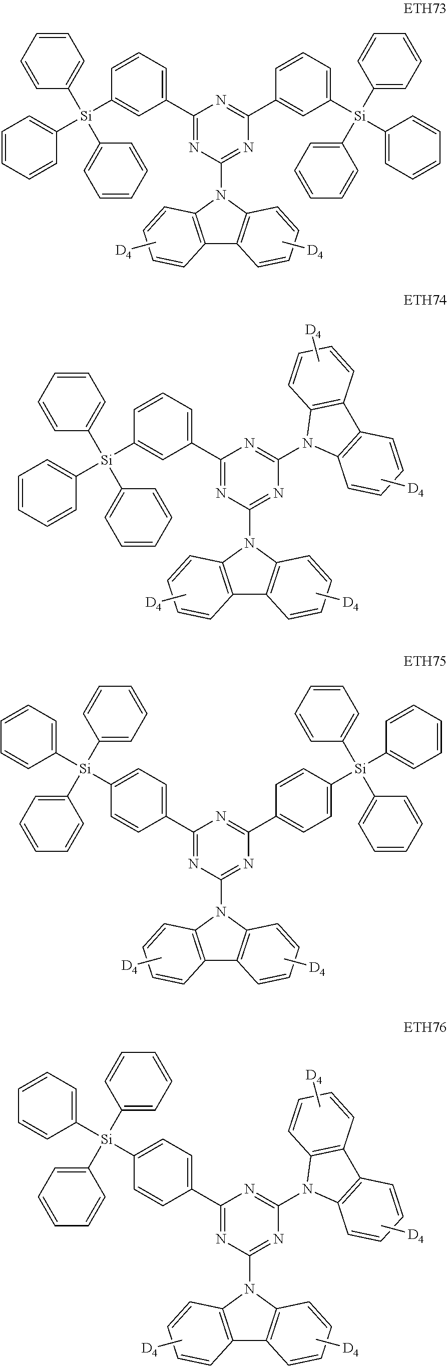

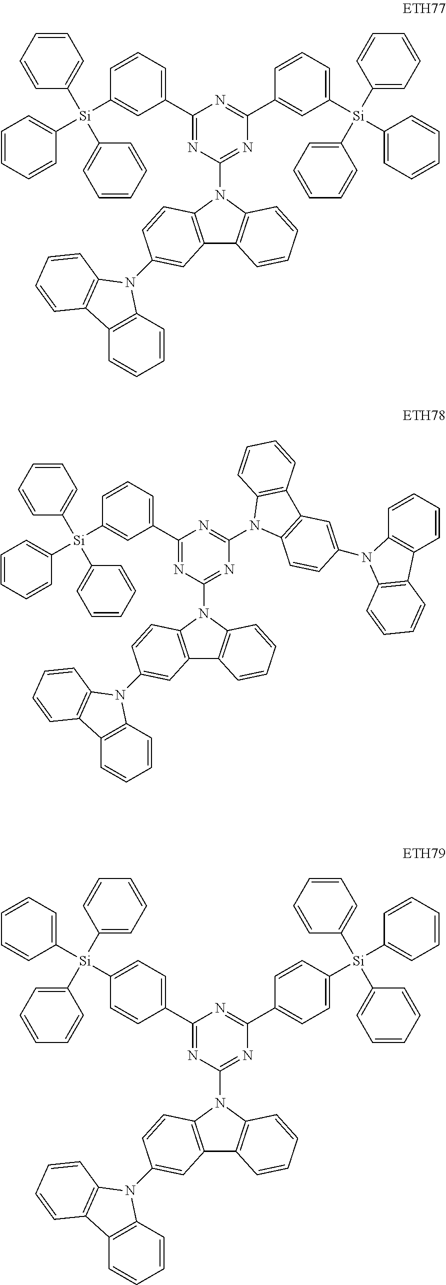 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5cc(-n6c7ccccc7c7ccccc76)ccc54)n3)c2)cc1 ; c1ccc([Si](c2ccccc2)(c2ccccc2)c2cccc(-c3nc(-n4c5ccccc5c5cc(-n6c7ccccc7c7ccccc76)ccc54)nc(-n4c5ccccc5c5cc(-n6c7ccccc7c7ccccc76)ccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5cc(-n6c7ccccc7c7ccccc76)ccc54)n3)cc2)cc1]

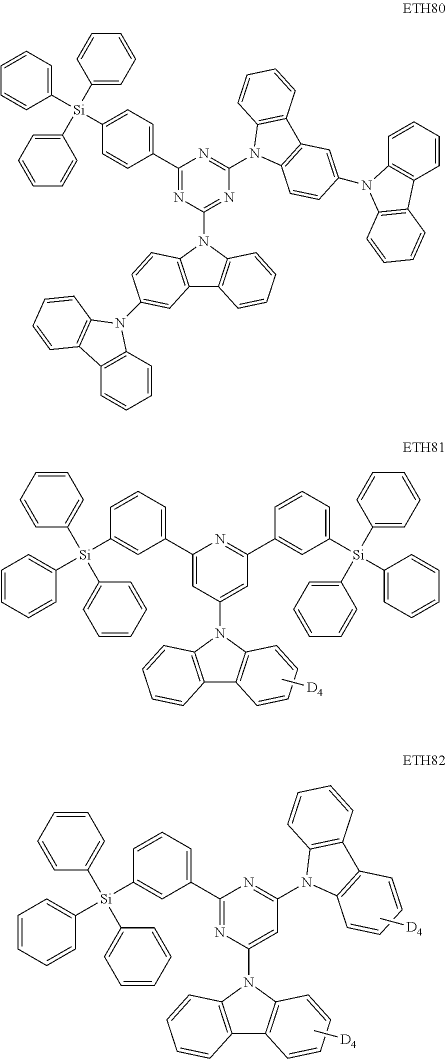

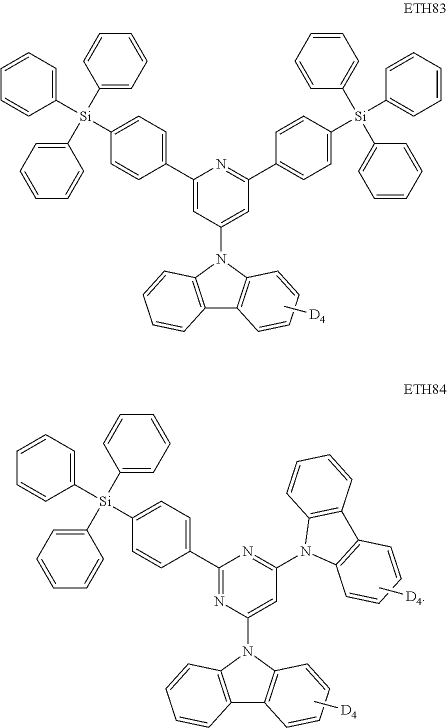

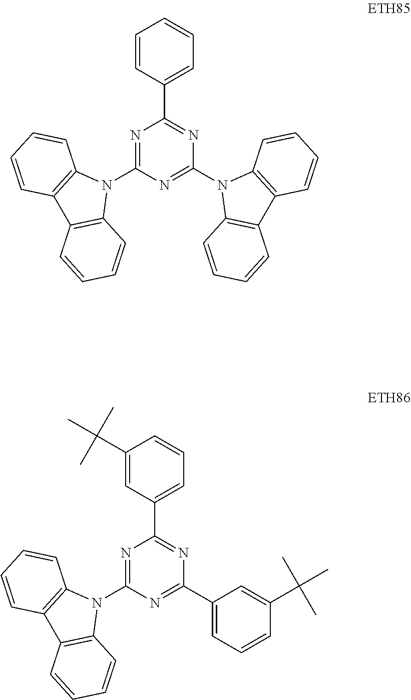 [SMILES: c1ccc(-c2nc(-n3c4ccccc4c4ccccc43)nc(-n3c4ccccc4c4ccccc43)n2)cc1 ; CC(C)(C)c1cccc(-c2nc(-c3cccc(C(C)(C)C)c3)nc(-n3c4ccccc4c4ccccc43)n2)c1]

In one or more embodiments, the emission layer EML may include a compound represented by Formula E-2a or Formula E-2b. The compound represented by Formula E-2a or Formula E-2b may be utilized as a phosphorescence host material.

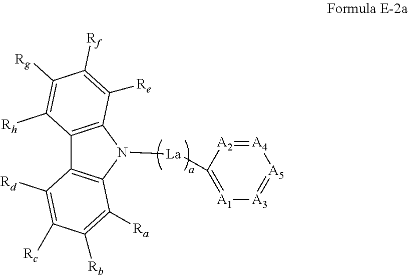

In Formula E-2a, “a” may be an integer of 0 to 10, Lₐ may be a direct linkage, a substituted or unsubstituted arylene group of 6 to 30 ring-forming carbon atoms, or a substituted or unsubstituted heteroarylene group of 2 to 30 ring-forming carbon atoms. In some embodiments, when “a” is an integer of 2 or more, multiple Lₐ may each independently be a substituted or unsubstituted arylene group of 6 to 30 ring-forming carbon atoms, or a substituted or unsubstituted heteroarylene group of 2 to 30 ring-forming carbon atoms.

In some embodiments, in Formula E-2a, A₁ to A₅ may each independently be N or CRi. Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. Rₐ to Rᵢ may be combined with an adjacent group to form a hydrocarbon ring or a heterocycle including N, O, S, and/or the like as a ring-forming atom.

In some embodiments, in Formula E-2a, two or three selected from among A₁ to A₅ may be N, and the remainder may be CRi.

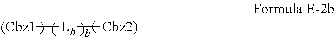

In Formula E-2b, Cbz1 and Cbz2 may each independently be an unsubstituted carbazole group, or a carbazole group substituted with an aryl group of 6 to 30 ring-forming carbon atoms. L_{b} may be a direct linkage, a substituted or unsubstituted arylene group of 6 to 30 ring-forming carbon atoms, or a substituted or unsubstituted heteroarylene group of 2 to 30 ring-forming carbon atoms. “b” is an integer of 0 to 10, and when “b” is an integer of 2 or more, multiple L_{b} may each independently be a substituted or unsubstituted arylene group of 6 to 30 ring-forming carbon atoms, or a substituted or unsubstituted heteroarylene group of 2 to 30 ring-forming carbon atoms.

The compound represented by Formula E-2a or Formula E-2b may be represented by any one among the compounds in Compound Group E-2. However, the compounds listed in Compound Group E-2 are only illustrations, and the compound represented by Formula E-2a or Formula E-2b is not limited to the compounds represented in Compound Group E-2.

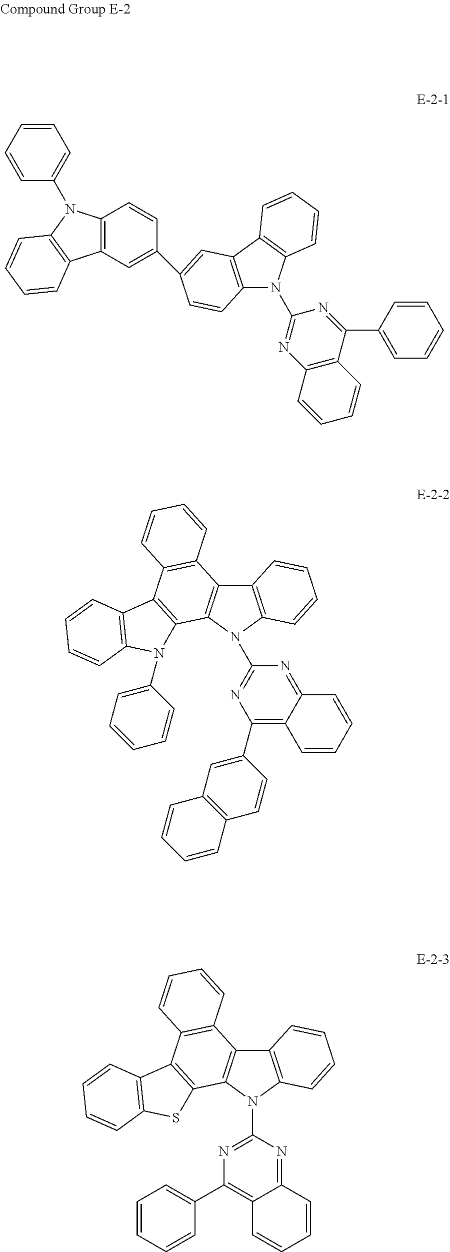 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

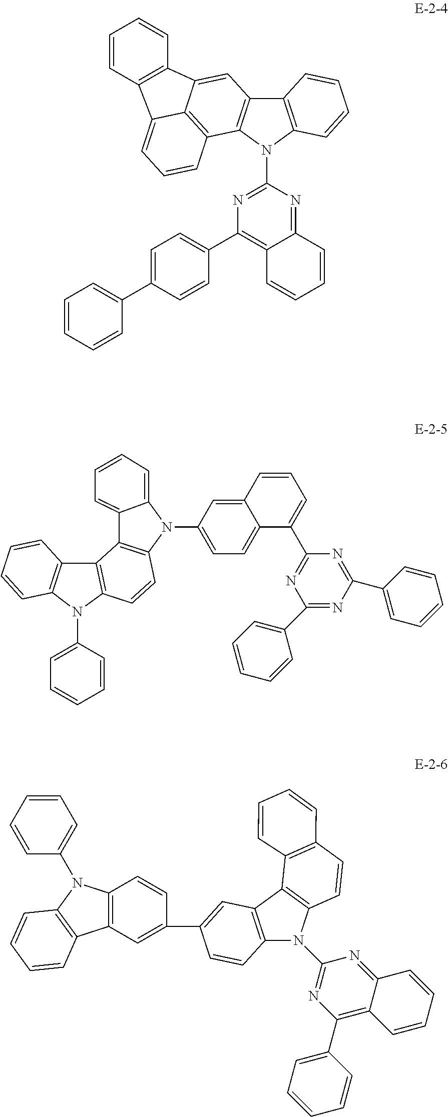 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

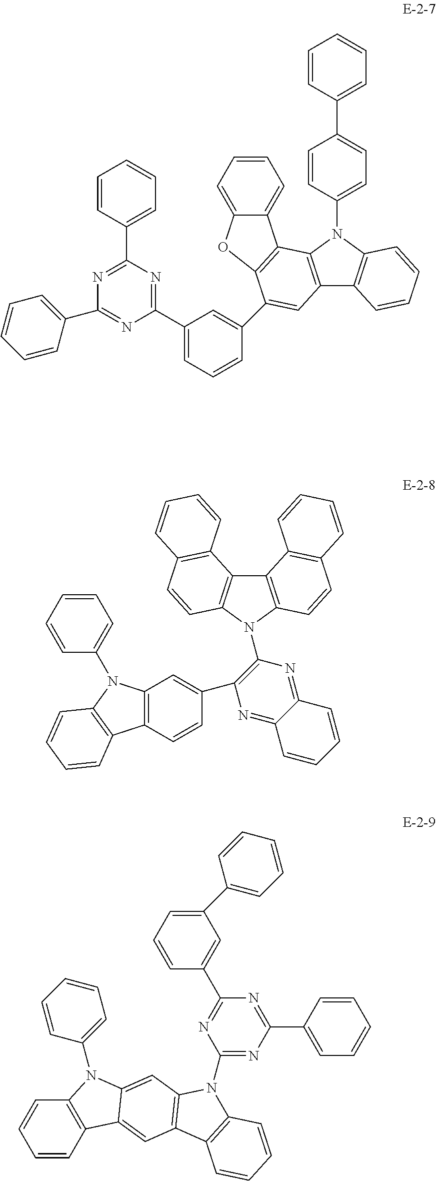 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1]

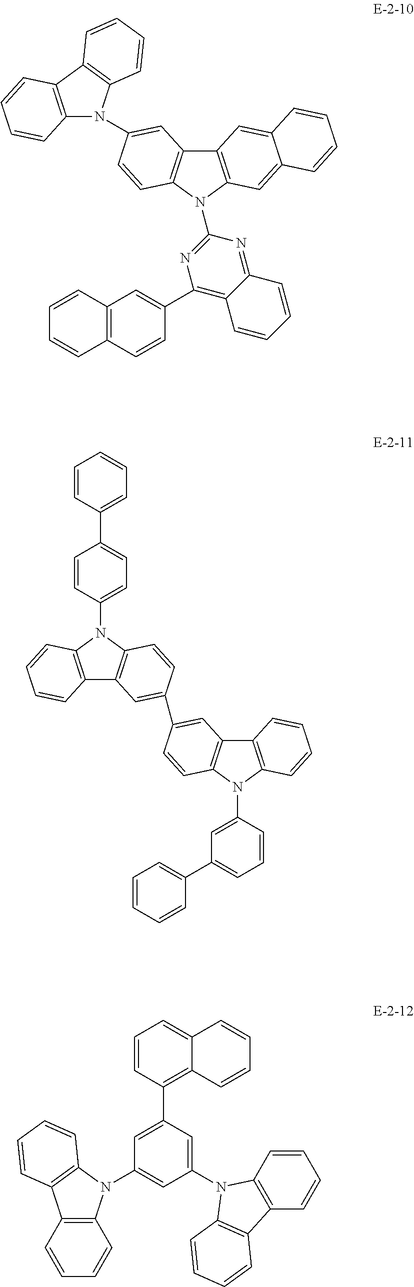 [SMILES: c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1]

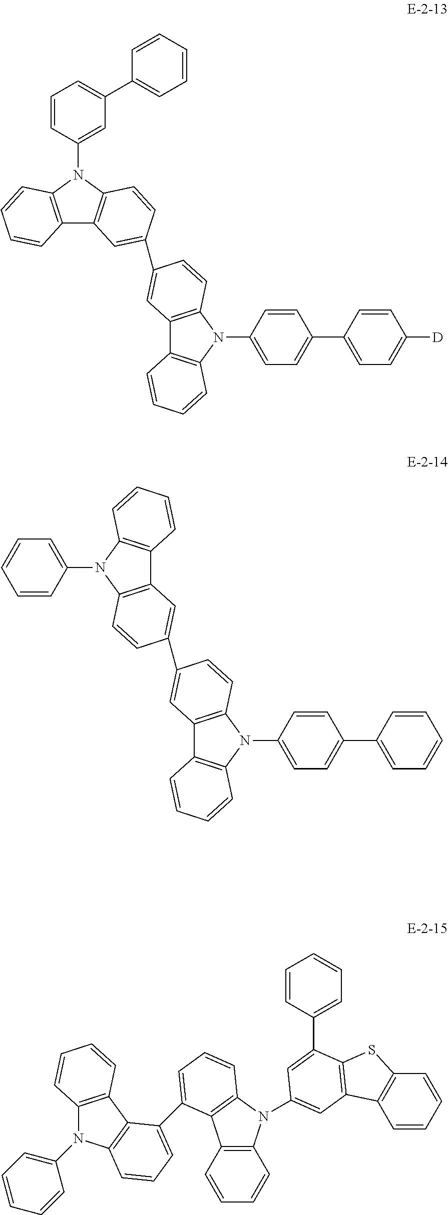 [SMILES: [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

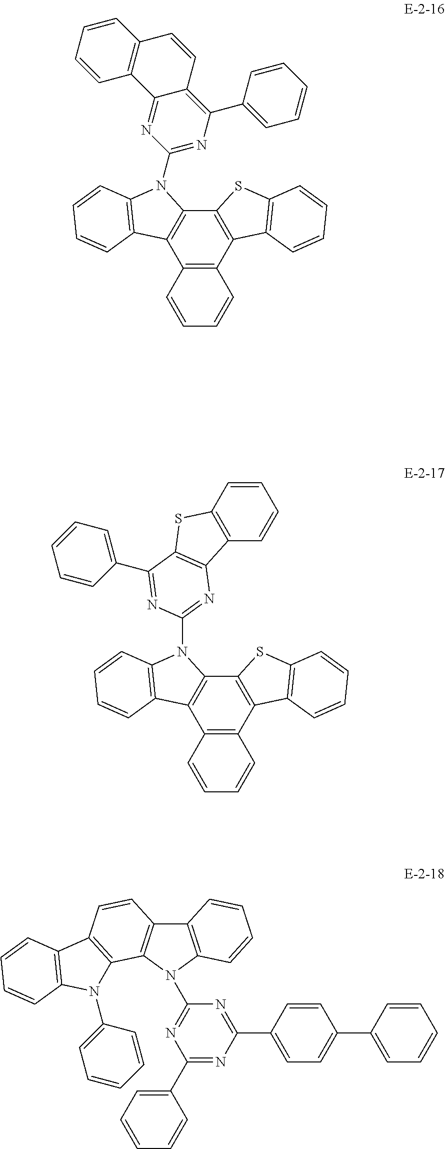 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1]

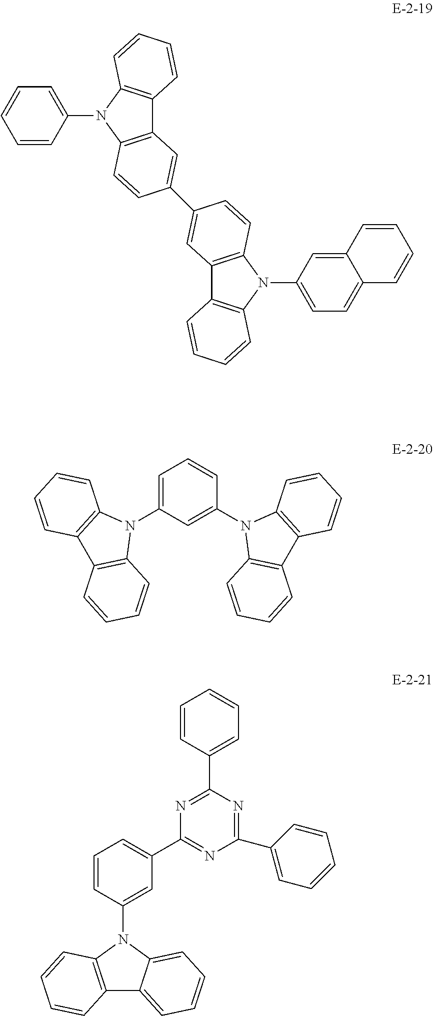 [SMILES: c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1]

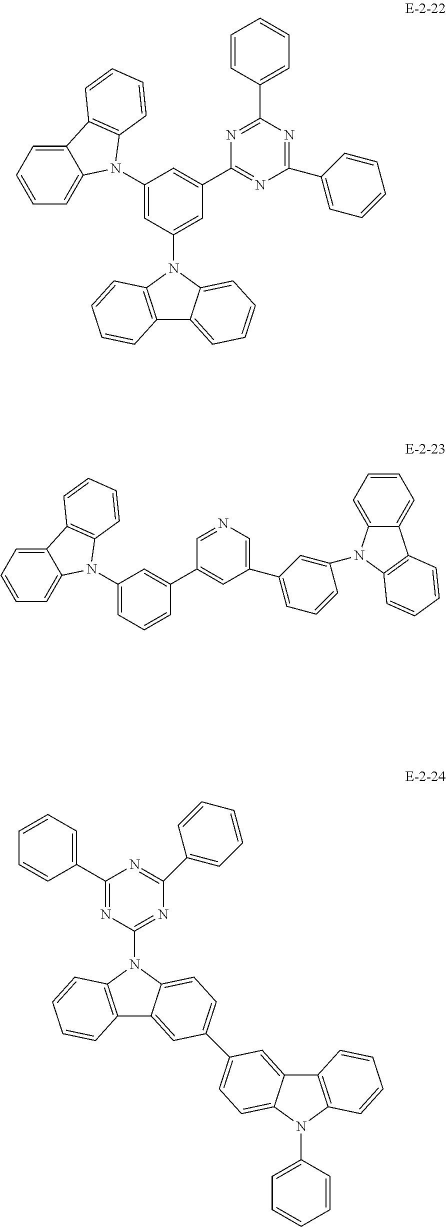 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a common material well-suitable in the art as a host material. For example, the emission layer EML may include as a host material, at least one of bis (4-(9H-carbazol-9-yl) phenyl) diphenylsilane (BCPDS), (4-(1-(4-(diphenylamino) phenyl) cyclohexyl) phenyl) diphenyl-phosphine oxide (POPCPA), bis[2-(diphenylphosphino)phenyl] ether oxide (DPEPO), 4,4′-bis(carbazol-9-yl)biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, one or more embodiments of the present disclosure is not limited thereto. For example, tris(8-hydroxyquinolino)aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO3), octaphenylcyclotetra siloxane (DPSiO4), and/or the like may be utilized as the host material.

The emission layer EML may include a compound represented by Formula M-a or Formula M-b. The compound represented by Formula M-a or Formula M-b may be utilized as a phosphorescence dopant material.

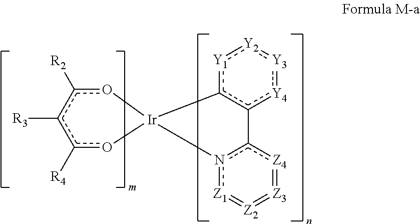

In Formula M-a, Y₁ to Y₄, and Z₁ to Z₄ may each independently be CR₁ or N, and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. In Formula M-a, “m” is 0 or 1, and “n” is 2 or 3. In Formula M-a, when “m” is 0, “n” is 3, and when “m” is 1, “n” is 2.

The compound represented by Formula M-a may be utilized as a phosphorescence dopant.

The compound represented by Formula M-a may be represented by any one selected from among Compounds M-a1 to M-a25. However, Compounds M-a1 to M-a25 are illustrations, and the compound represented by Formula M-a is not limited to the compounds represented by Compounds M-a1 to M-a25.

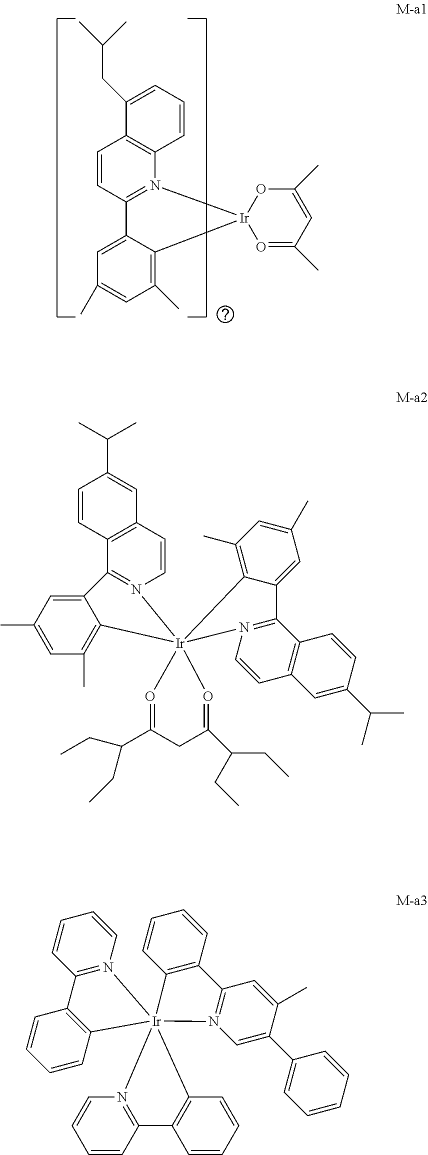

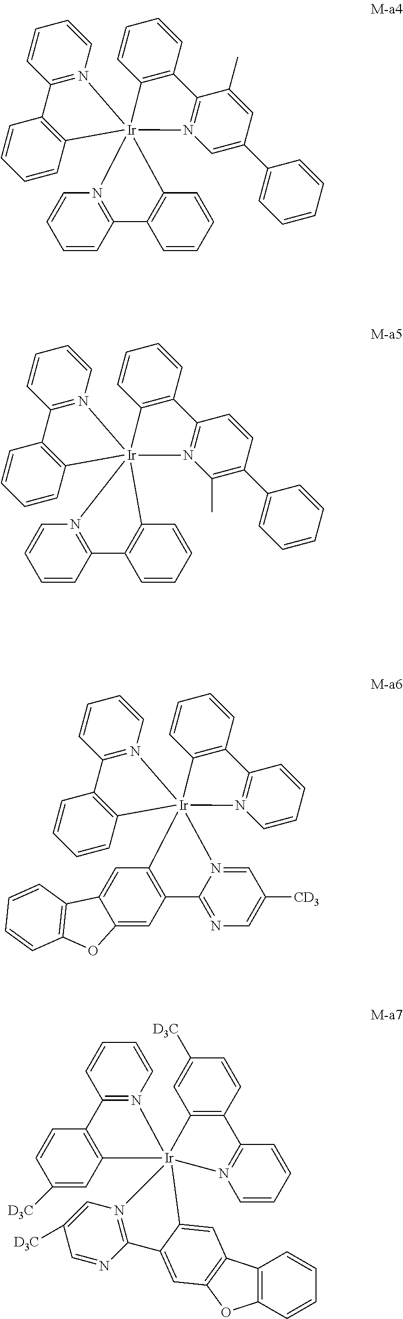

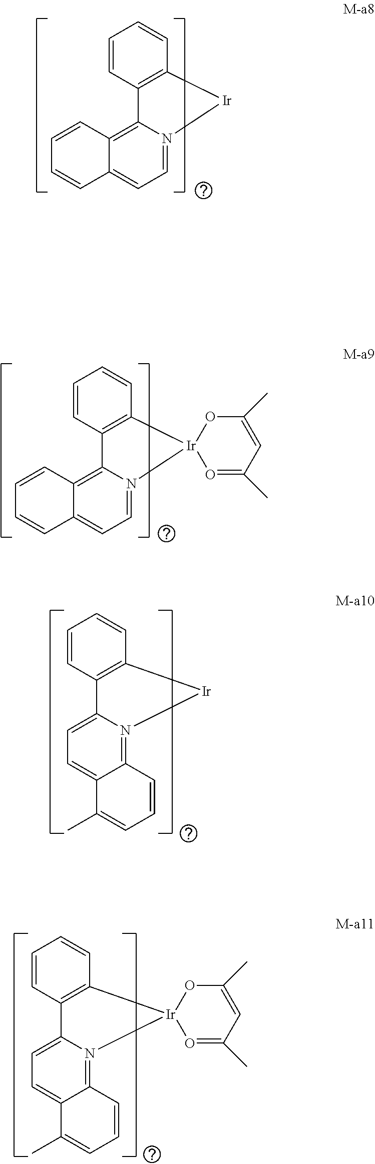

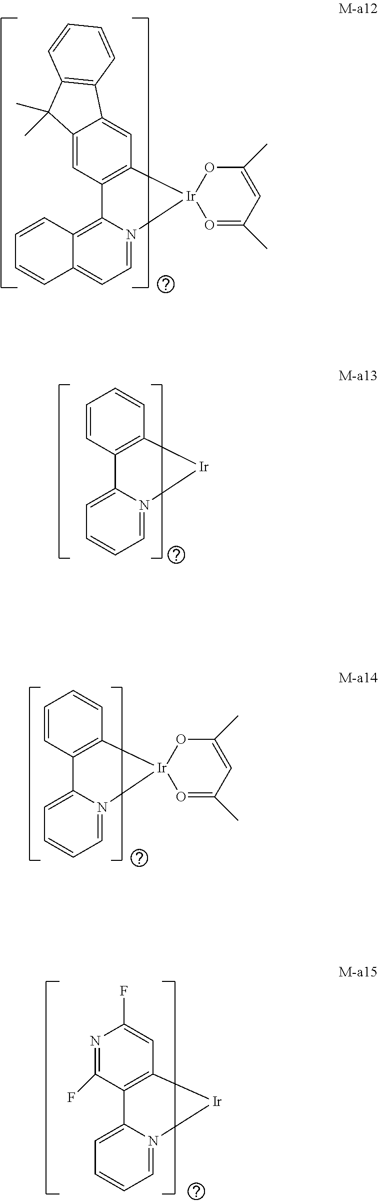

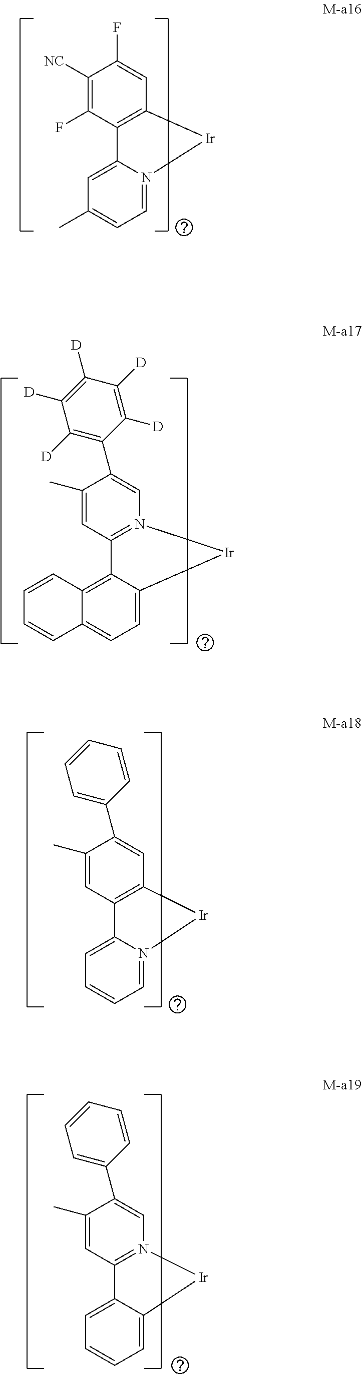

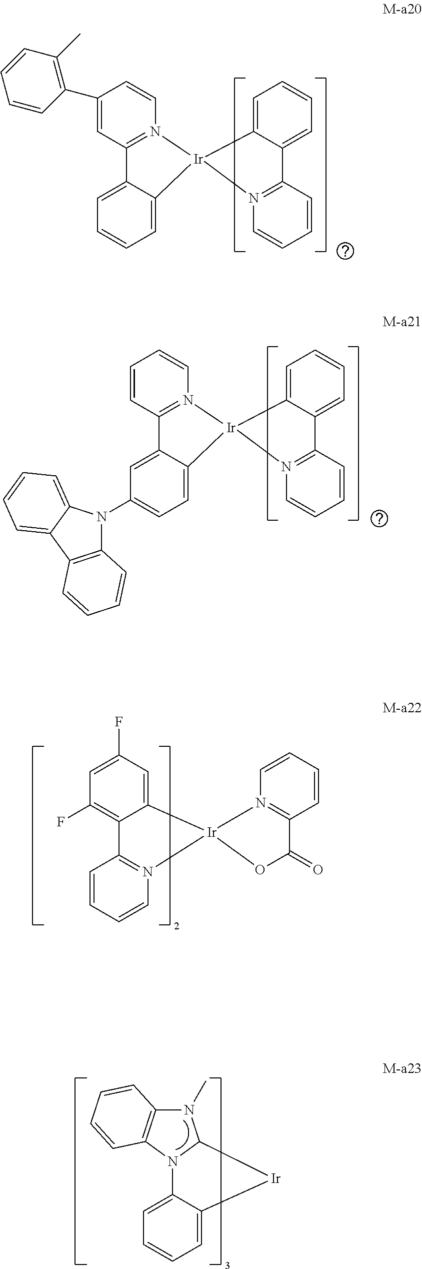

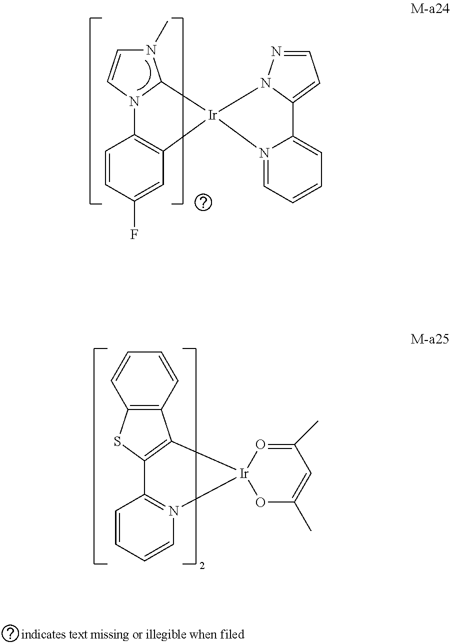

Compound M-a1 and Compound M-a2 may be utilized as red dopant materials, and Compound M-a3 to Compound M-a7 may be utilized as green dopant materials.

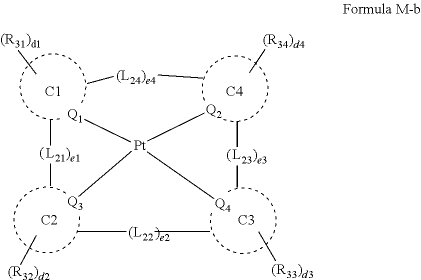

In Formula M-b, Q₁ to Q₄ may each independently be C or N, C1 to C4 may each independently be a substituted or unsubstituted hydrocarbon ring of 5 to 30 ring-forming carbon atoms, or a substituted or unsubstituted heterocycle of 2 to 30 ring-forming carbon atoms. L₂₁ to L₂₄ may each independently be a direct linkage,

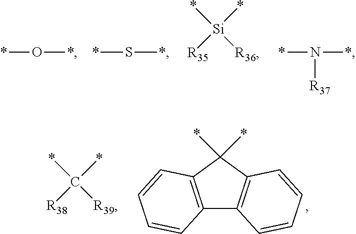

a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms, and e1 to e4 may each independently be 0 or 1. R₃₁ to R₃₉ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring, and d1 to d4 may each independently be an integer of 0 to 4.

The compound represented by Formula M-b may be utilized as a blue phosphorescence dopant or a green phosphorescence dopant.

The compound represented by Formula M-b may be represented by any one selected from among Compound M-b-1 to Compound M-b-11. However, the compounds are illustrations, and the compound represented by Formula M-b is not limited to the compounds M-b-1 to M-b-11.

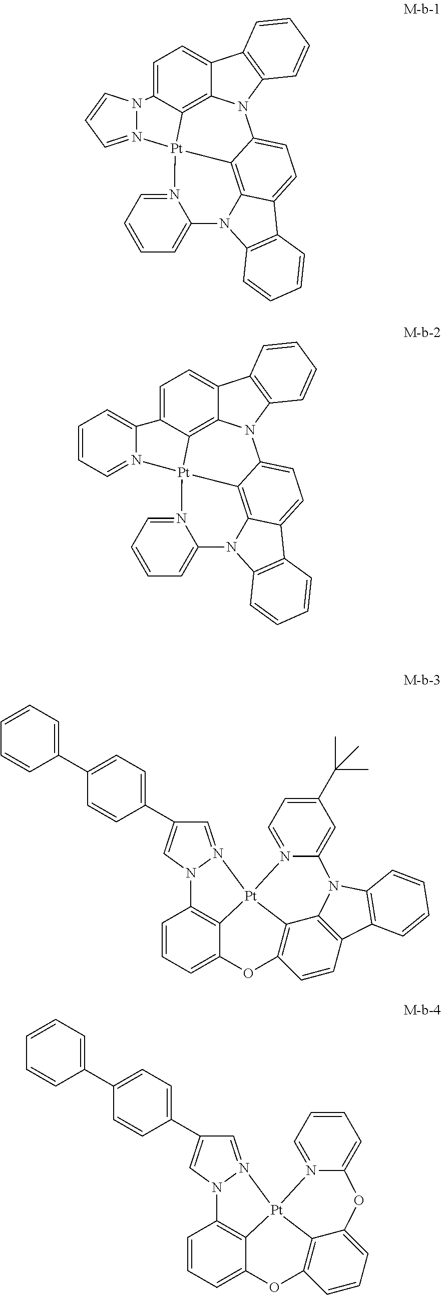

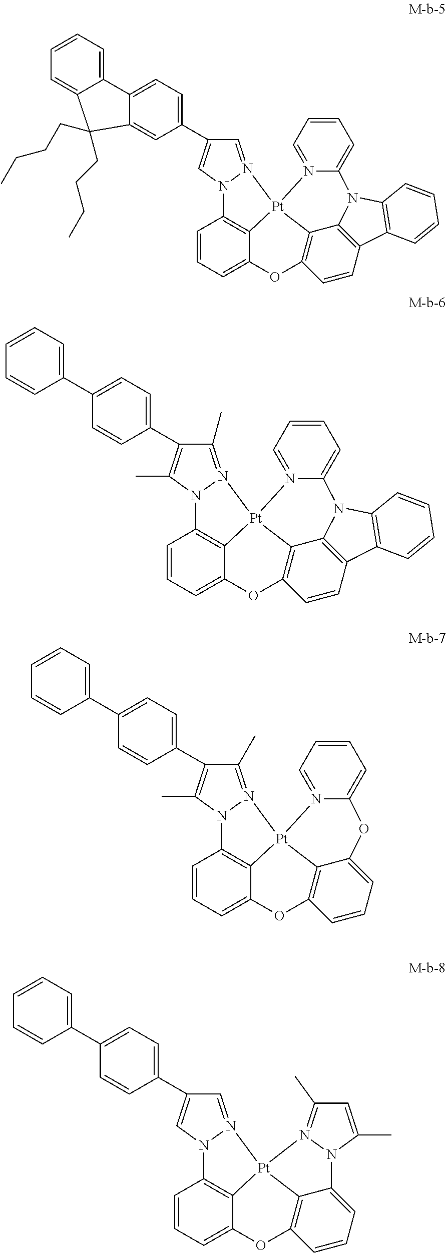

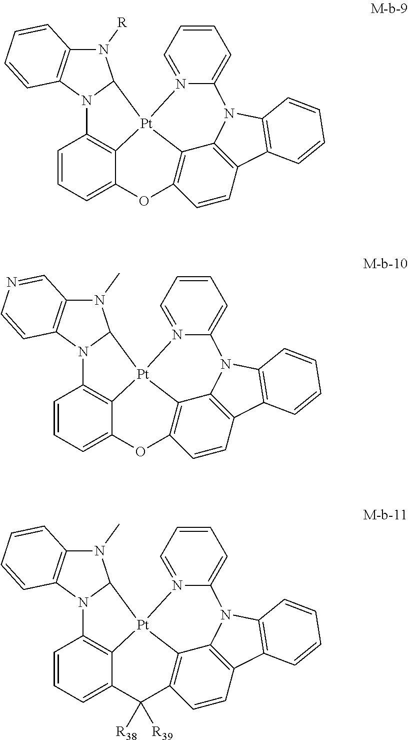

In the compounds M-b-1 to M-b-11, R, R₃₈, and R₃₉ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

The emission layer EML may include any one selected from among Formula F-a to Formula F-c. The compounds represented by Formula F-a to Formula F-c may be utilized as fluorescence dopant materials.

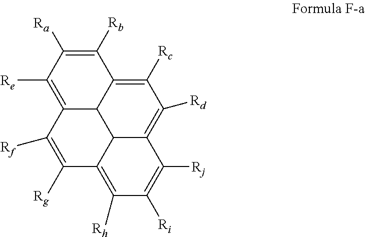

In Formula F-a, two selected from among Rₐ to Rⱼ may each independently be substituted with *—NAr₁Ar₂. The remainder not substituted with *—NAr₁Ar₂ among Rₐ to Rⱼ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In *—NAr₁Ar₂, Ar₁ and Ar₂ may each independently be a substituted or unsubstituted aryl group of 6 to 30 ring-forming carbon atoms, or a substituted or unsubstituted heteroaryl group of 2 to 30 ring-forming carbon atoms. For example, at least one selected from among Ar₁ and Ar₂ may be a heteroaryl group including O or S as a ring-forming atom.

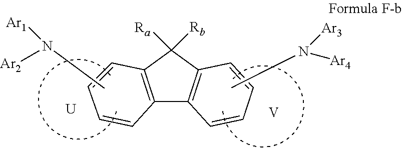

In Formula F-b, Rₐ and R_{b} may each independently be a hydrogen atom, a deuterium atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. Ar₁ to Ar₄ may each independently be a substituted or unsubstituted aryl group of 6 to 30 ring-forming carbon atoms, or a substituted or unsubstituted heteroaryl group of 2 to 30 ring-forming carbon atoms.

In Formula F-b, U and V may each independently be a substituted or unsubstituted hydrocarbon ring of 5 to 30 ring-forming carbon atoms, or a substituted or unsubstituted heterocycle of 2 to 30 ring-forming carbon atoms.

In Formula F-b, the number of rings represented by U and V may each independently be 0 or 1. For example, in Formula F-b, when the number of U or V is 1, one ring forms a fused ring at the designated part by U or V, and when the number of U or V is 0, a ring is not present at the designated part by U or V. For example, when the number of U is 0, and the number of V is 1, or when the number of U is 1, and the number of V is 0, a fused ring having the fluorene core of Formula F-b may be a ring compound with four rings. In some embodiments, when the number of both (e.g., simultaneously) U and V is 0, the fused ring of Formula F-b may be a ring compound with three rings. In some embodiments, when the number of both (e.g., simultaneously) U and V is 1, a fused ring having the fluorene core of Formula F-b may be a ring compound with five rings.

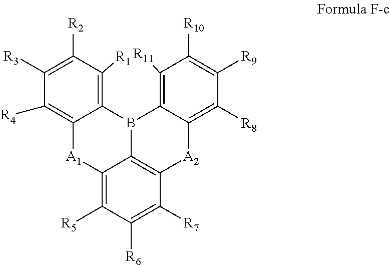

In Formula F-c, A₁ and A₂ may each independently be O, S, Se, or NRₘ, and Rₘ may be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring.

In Formula F-c, A₁ and A₂ may each independently be combined with the substituents of an adjacent ring to form a fused ring. For example, when A₁ and A₂ may each independently be NRₘ, A₁ may be combined with R₄ or R₅ to form a ring. In some embodiments, A₂ may be combined with R₇ or R₈ to form a ring.

In one or more embodiments, the emission layer EML may include as a suitable dopant material, styryl derivatives (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and 4,4′-bis[2-(4-(N,N-diphenylamino)phenyl)vinyl]biphenyl (DPAVBi)), perylene and the derivatives thereof (for example, 2,5,8,11-tetra-t-butylperylene (TBP)), pyrene and the derivatives thereof (for example, 1,1-dipyrene, 1,4-dipyrenylbenzene, and 1,4-bis(N,N-diphenylamino)pyrene), and/or the like. The emission layer EML may include a suitable phosphorescence dopant material. For example, the phosphorescence dopant may utilize a metal complex including iridium (Ir), platinum (Pt), osmium (Os), gold (Au), titanium (Ti), zirconium (Zr), hafnium (Hf), europium (Eu), terbium (Tb) or thulium (Tm). For example, iridium(III) bis(4,6-difluorophenylpyridinato-N, C2′)picolinate (FIrpic), bis(2,4-difluorophenylpyridinato)-tetrakis(1-pyrazolyl)borate iridium(III) (Fir6), or platinum octaethyl porphyrin (PtOEP) may be utilized as the phosphorescence dopant. However, one or more embodiments of the present disclosure is not limited thereto.

The emission layer EML may include a quantum dot material. The core of the quantum dot may be selected from among II-VI group compounds, III-VI group compounds, I-III-VI group compounds, III-V group compounds, III-II-V group compounds, IV-VI group compounds, IV group elements, IV group compounds, and combinations thereof.

The II-VI group compound may be selected from the group consisting of: a binary compound selected from the group consisting of CdSe, CdTe, CdS, ZnS, ZnSe, ZnTe, ZnO, HgS, HgSe, HgTe, MgSe, MgS, and mixtures thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and mixtures thereof; and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, and mixtures thereof.

The III-VI group compound may include a binary compound such as In₂S₃ and/pr In₂Se₃, a ternary compound such as InGaS₃ and/or InGaSe₃, or optional combination(s) thereof.

The I-III-VI group compound may be selected from a ternary compound selected from the group consisting of AgInS, AgInS₂, CuInS, CuInS₂, AgGaS₂, CuGaS₂, CuGaO₂, AgGaO₂, AgAlO₂ and mixtures thereof, or a quaternary compound such as AgInGaS₂ and/or CuInGaS₂.

The III-V group compound may be selected from the group consisting of a binary compound selected from the group consisting of GaN, GaP, GaAs, GaSb, AlN, AlP, AlAs, AlSb, InN, InP, InAs, InSb, and mixtures thereof, a ternary compound selected from the group consisting of GaNP, GaNAs, GaNSb, GaPAs, GaPSb, AlNP, AlNAs, AlNSb, AlPAS, AlPSb, InGaP, InAlP, InNP, InNAs, InNSb, InPAs, InPSb, and mixtures thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and mixtures thereof. In some embodiments, the III-V group compound may further include a II group metal. For example, InZnP, and/or the like may be selected as a III-II-V group compound.

The IV-VI group compound may be selected from the group consisting of a binary compound selected from the group consisting of SnS, SnSe, SnTe, PbS, PbSe, PbTe, and mixtures thereof, a ternary compound selected from the group consisting of SnSeS, SnSeTe, SnSTe, PbSeS, PbSeTe, PbSTe, SnPbS, SnPbSe, SnPbTe, and mixtures thereof, and a quaternary compound selected from the group consisting of SnPbSSe, SnPbSeTe, SnPbSTe, and mixtures thereof. The IV group element may be selected from the group consisting of Si, Ge, and a mixture thereof. The IV group compound may be a binary compound selected from the group consisting of SiC, SiGe, and a mixture thereof.

In some embodiments, the binary compound, the ternary compound and/or the quaternary compound may be present at substantially uniform concentration in a particle or may be present at a partially different concentration distribution state in substantially the same particle. In some embodiments, a core/shell structure in which one quantum dot wraps another quantum dot may be possible. The interface of the core and the shell may have a concentration gradient in which the concentration of an element present in the shell is decreased toward the center.

In some embodiments, the quantum dot may have the described core-shell structure including a core including a nanocrystal and a shell wrapping (e.g., around or surround) the core. The shell of the quantum dot may play the role of a protection layer for preventing or reducing the chemical deformation of the core to maintain semiconductor properties and/or a charging layer for imparting the quantum dot with electrophoretic properties. The shell may have a single layer or a multilayer. Examples of the shell of the quantum dot may include a metal or non-metal oxide, a semiconductor compound, or combinations thereof.

For example, the metal or non-metal oxide may include a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄ and NiO, or a ternary compound such as MgAl₂O₄, CoFe₂O₄, NiFe₂O₄ and CoMn₂O₄, but one or more embodiments of the present disclosure is not limited thereto.

Also, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, and/or the like, but one or more embodiments of the present disclosure is not limited thereto.

The quantum dot may have a full width of half maximum (FWHM) of emission wavelength spectrum of about 45 nm or less, about 40 nm or less, or, about 30 nm or less. Within the range(s), color purity or color reproducibility may be improved. In some embodiments, light emitted via such quantum dot is emitted in all directions, and light view angle properties may be improved (e.g., the size or width of the viewing angle may be enhanced or increased).

In some embodiments, the shape of the quantum dot may be generally utilized shapes in the art, without specific limitation. More particularly, the shape of spherical, pyramidal, multi-arm, or cubic nanoparticle, nanotube, nanowire, nanofiber, nanoplate particle, and/or the like may be utilized.

The quantum dot may control the color of light emitted according to the particle size, and accordingly, the quantum dot may have one or more suitable emission colors such as blue, red and green.

In the light emitting elements ED of embodiments, as shown in FIG. 3 to FIG. 6, the electron transport region ETR is provided on the emission layer EML. The electron transport region ETR may include at least one of a hole blocking layer HBL, an electron transport layer ETL or an electron injection layer EIL. However, one or more embodiments of the present disclosure is not limited thereto.

The electron transport region ETR may have a single layer formed utilizing a single material, a single layer formed utilizing multiple different materials, or a multilayer structure having multiple layers formed utilizing multiple different materials.

For example, the electron transport region ETR may have a single layer structure of an electron injection layer EIL or an electron transport layer ETL, or a single layer structure formed utilizing an electron injection material and an electron transport material. Further, the electron transport region ETR may have a single layer structure formed utilizing multiple different materials, or a structure stacked from the emission layer EML of electron transport layer ETL/electron injection layer EIL, hole blocking layer HBL/electron transport layer ETL/electron injection layer EIL, without limitation. The thickness of the electron transport region ETR may be, for example, from about 1,000 Å to about 1,500 Å.

The electron transport region ETR may be formed utilizing one or more suitable methods such as a vacuum deposition method, a spin coating method, a cast method, a Langmuir-Blodgett (LB) method, an inkjet printing method, a laser printing method, and/or a laser induced thermal imaging (LITI) method.

The electron transport region ETR may include a compound represented by Formula ET-2.

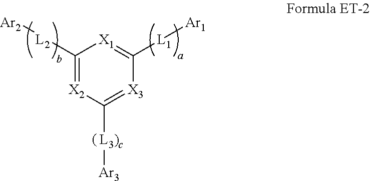

In Formula ET-2, at least one selected from among X₁ to X₃ is N, and the remainder are CRₐ. Each Rₐ may independently be a hydrogen atom, a deuterium atom, a substituted or unsubstituted alkyl of 1 to 20 carbon atoms, a substituted or unsubstituted aryl group of 6 to 30 ring-forming carbon atoms, or a substituted or unsubstituted heteroaryl group of 2 to 30 ring-forming carbon atoms. Ar₁ to Ar₃ may each independently be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula ET-2, “a” to “c” may each independently be an integer of 0 to 10. In Formula ET-2, L₁ to L₃ may each independently be a direct linkage, a substituted or unsubstituted arylene group of 6 to 30 ring-forming carbon atoms, or a substituted or unsubstituted heteroarylene group of 2 to 30 ring-forming carbon atoms. In some embodiments, when “a” to “c” are integers of 2 or more, L₁ to L₃ may each independently be a substituted or unsubstituted arylene group of 6 to 30 ring-forming carbon atoms, or a substituted or unsubstituted heteroarylene group of 2 to 30 ring-forming carbon atoms.

The electron transport region ETR may include an anthracene-based compound. However, one or more embodiments of the present disclosure is not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazolyl-1-ylphenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (Balq), berylliumbis(benzoquinolin-10-olate (Bebq₂), 9,10-di(naphthalene-2-yl)anthracene (and), 1,3-bis[3,5-di(pyridin-3-yl)phenyl]benzene (BmPyPhB), diphenyl(4-(triphenylsilyl)phenyl)phosphine oxide (TSPO1), and/or one or more mixtures thereof, without limitation.

The electron transport region ETR may include at least one selected from among Compounds ET1 to ET36.

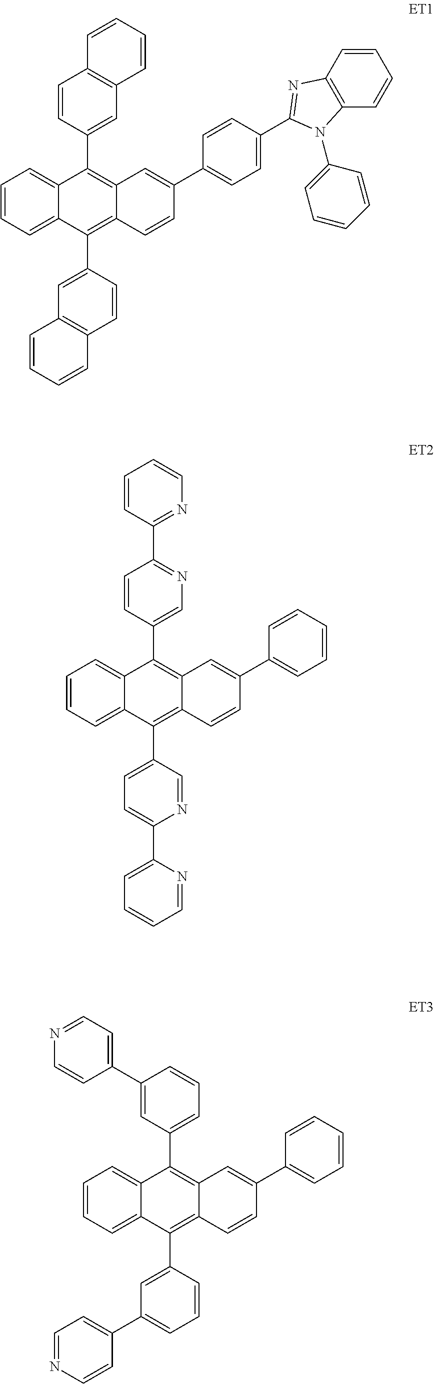 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

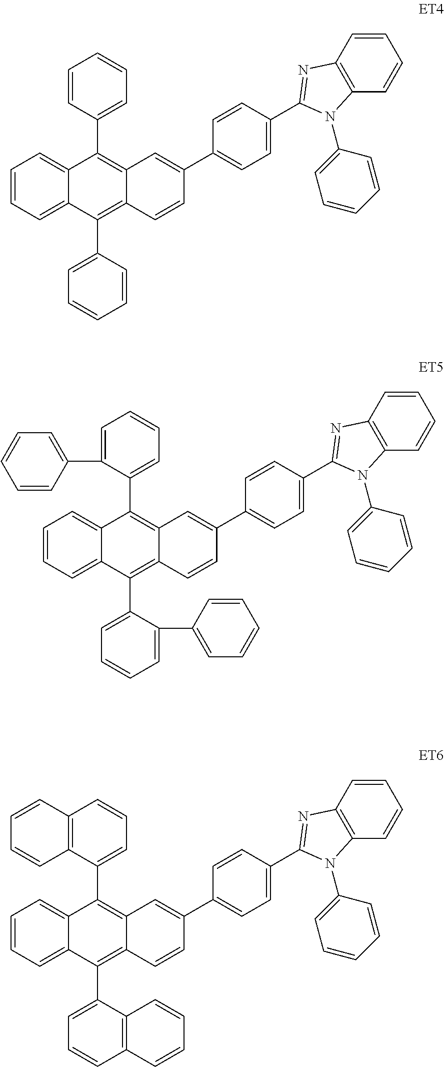 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

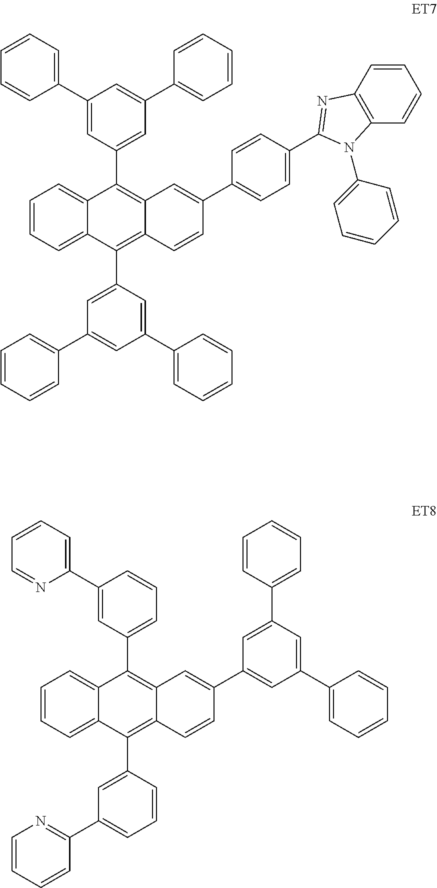 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1]

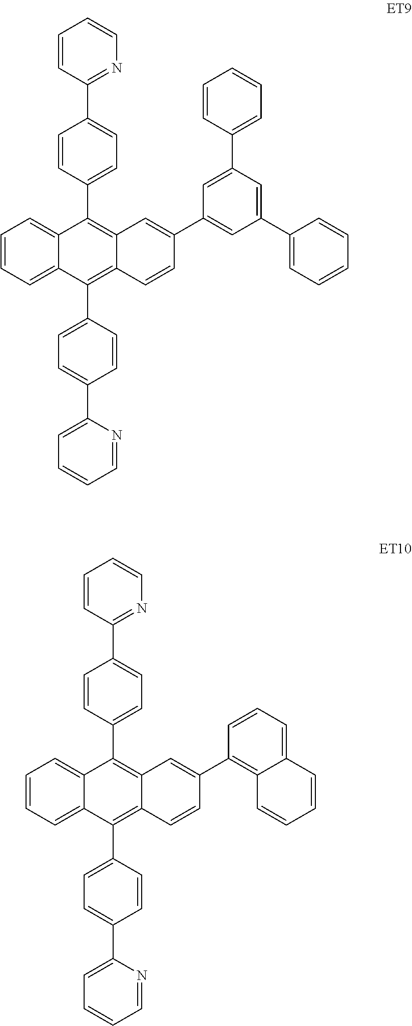 [SMILES: c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

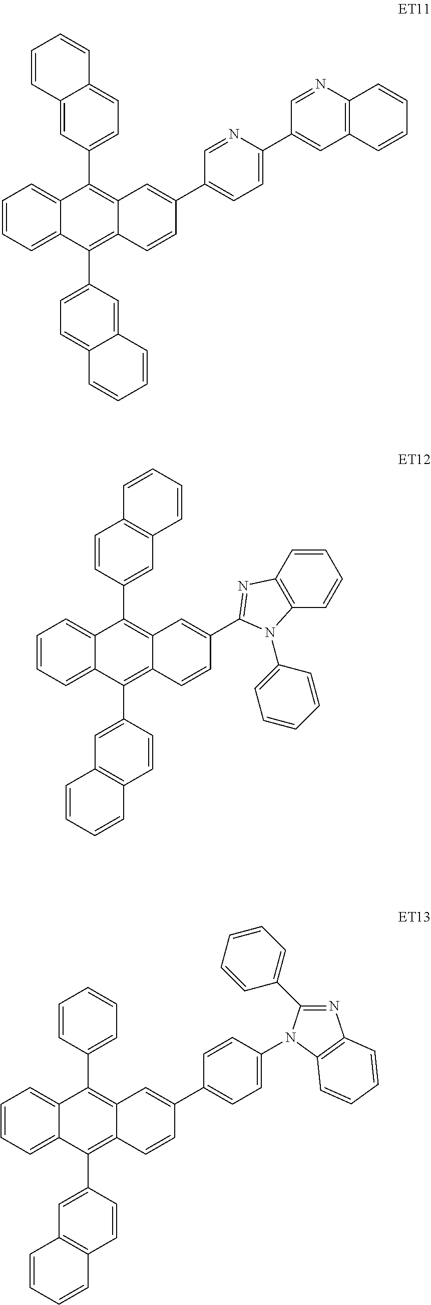 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

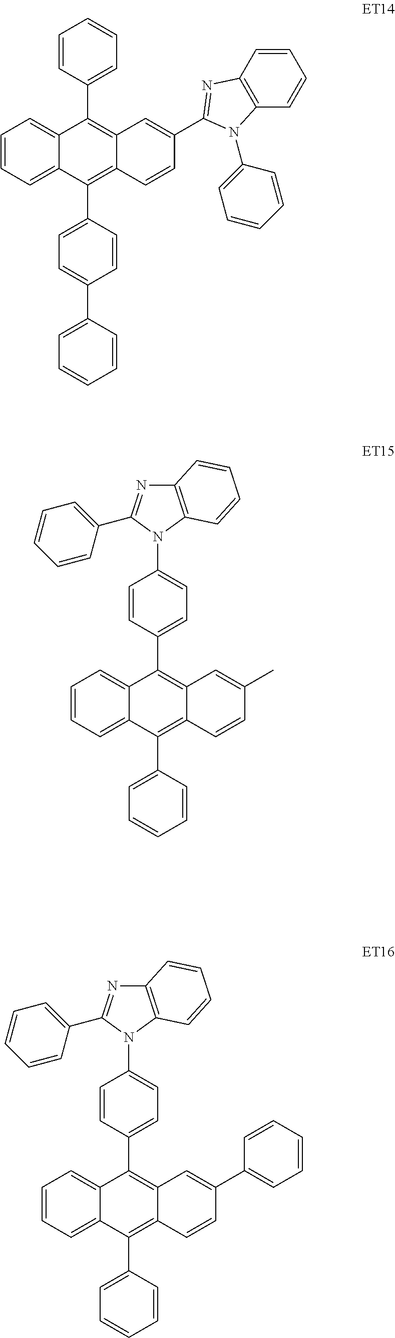 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

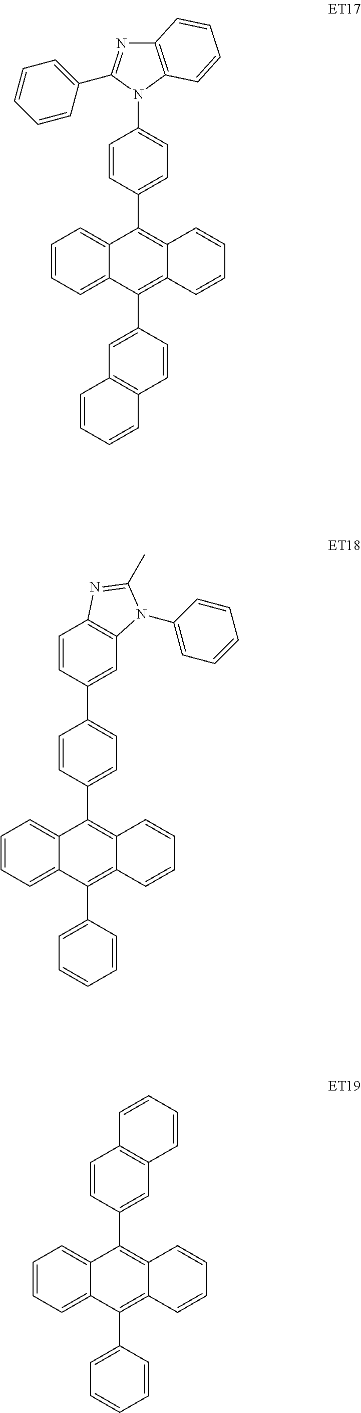

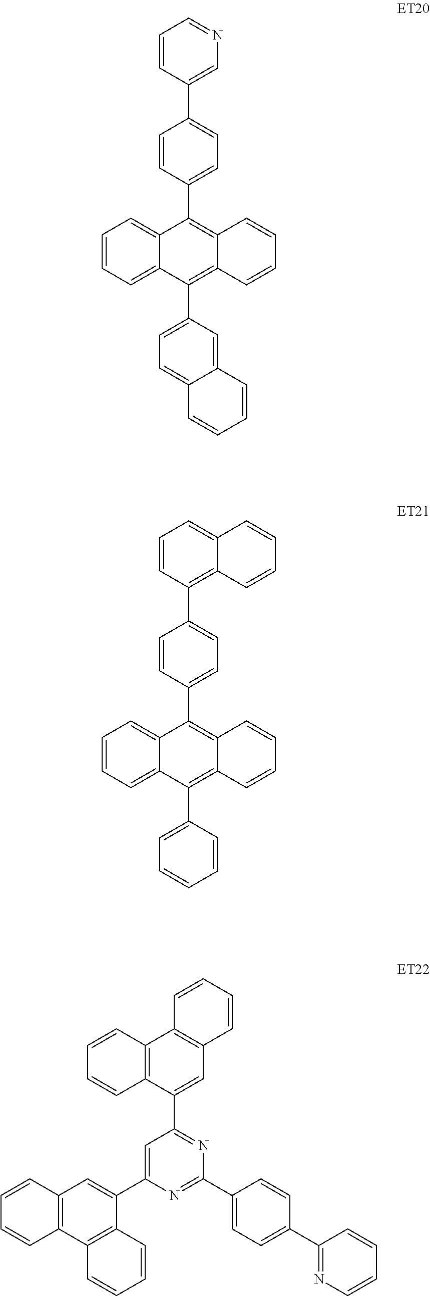

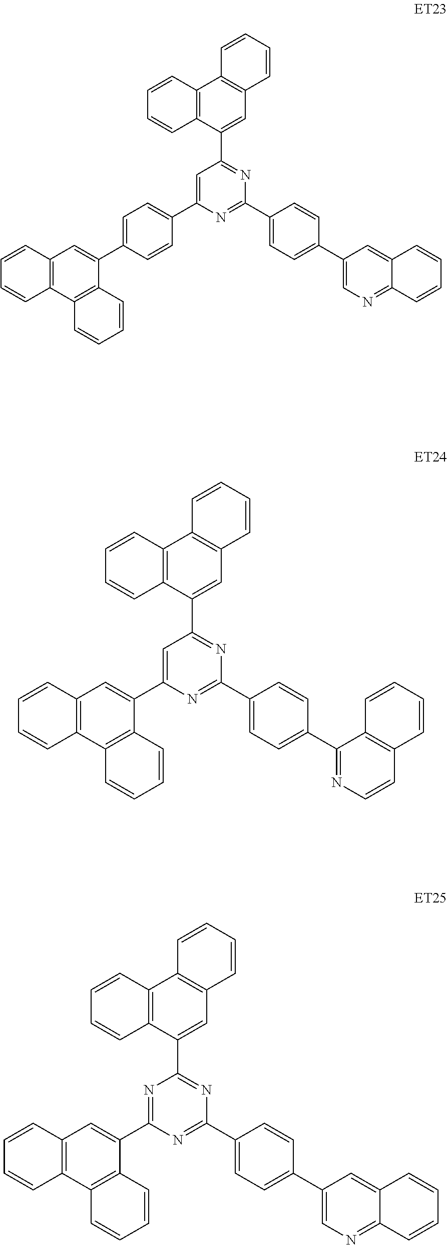 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

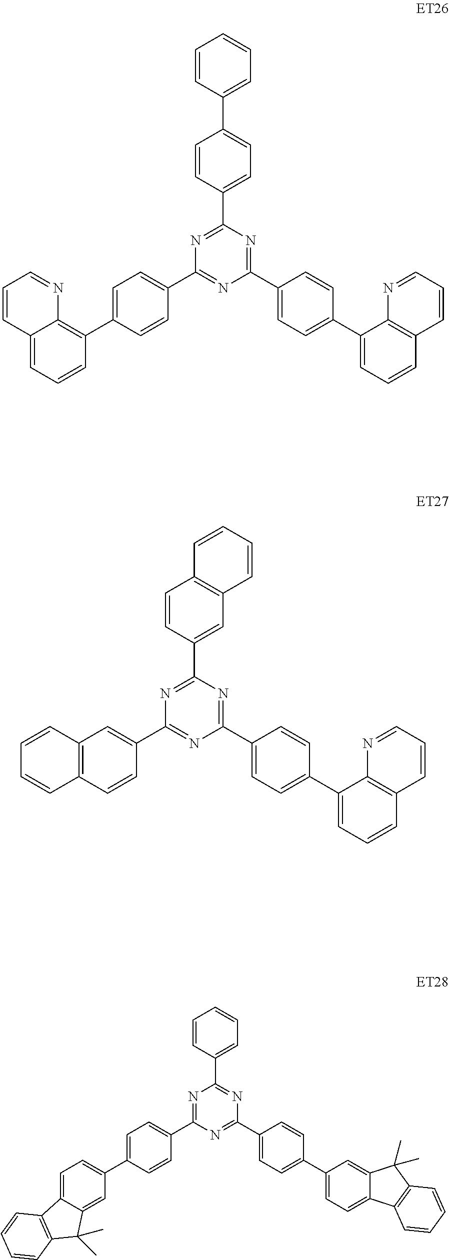 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21]

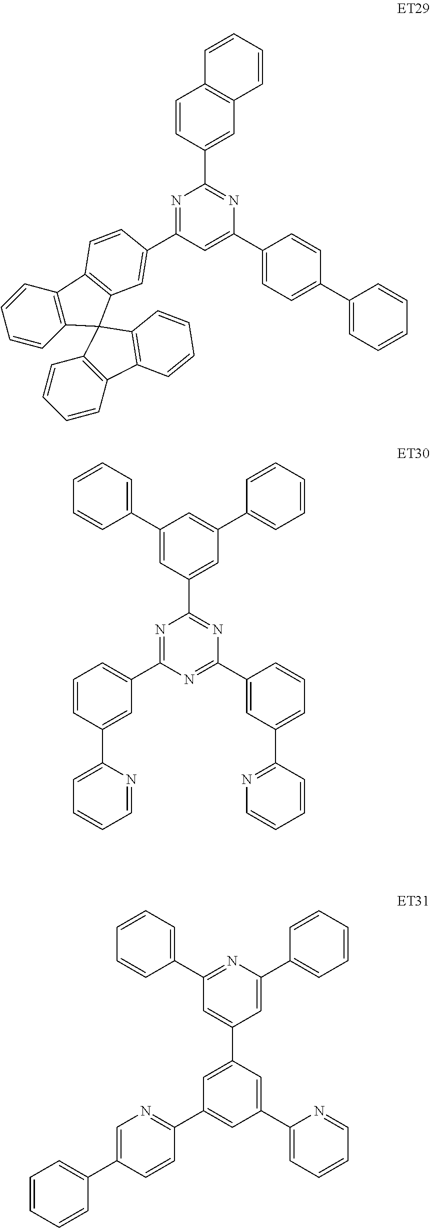 [SMILES: c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1]

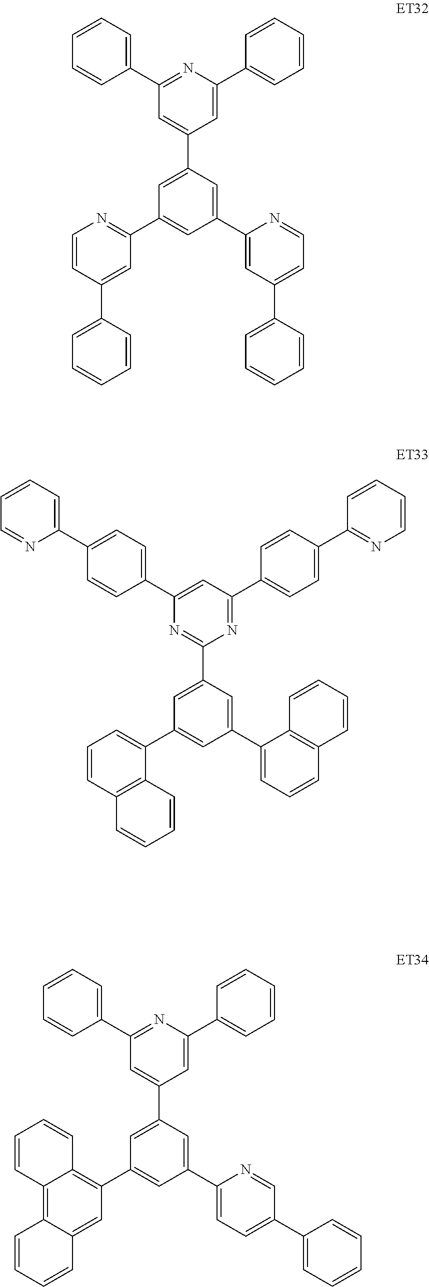 [SMILES: c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1]

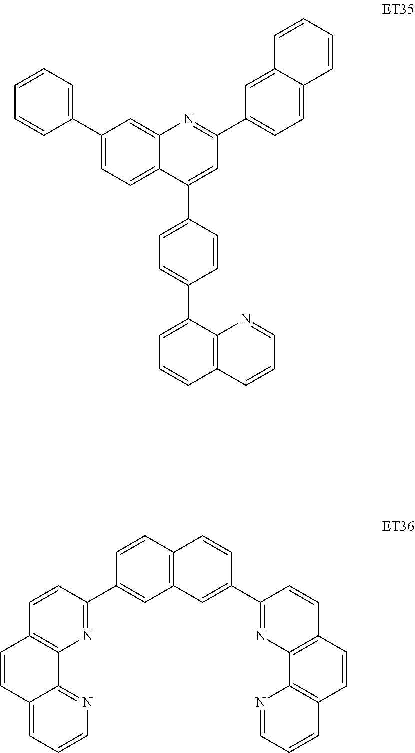 [SMILES: c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In some embodiments, the electron transport region ETR may include a metal halide such as LiF, NaCl, CsF, RbCl, Rbl, Cul and/or Kl, a lanthanide metal such as Yb, or a co-depositing material of the metal halide and the lanthanide metal. For example, the electron transport region ETR may include Kl:Yb, Rbl:Yb, LiF:Yb, and/or the like, as the co-depositing material. In some embodiments, the electron transport region ETR may utilize a metal oxide such as Li₂O and BaO, or 8-hydroxy-lithium quinolate (Liq). However, one or more embodiments of the present disclosure is not limited thereto. The electron transport region ETR also may be formed utilizing a mixture material of an electron transport material and an insulating organo metal salt.

The organo metal salt may be a material having an energy band gap of about 4 electron volt (eV) or more. For example, the organo metal salt may include, for example, metal acetates, metal benzoates, metal acetoacetates, metal acetylacetonates, and/or metal stearates.

The electron transport region ETR may include at least one of 2,9-dimethyl-4,7-diphenyl-1,10-phenanthroline (BCP), diphenyl(4-(triphenylsilyl)phenyl)phosphine oxide (TSPO1) and/or 4,7-diphenyl-1,10-phenanthroline (Bphen) in addition to the aforementioned materials. However, one or more embodiments of the present disclosure is not limited thereto.

The electron transport region ETR may include the compounds of the electron transport region in at least one selected from among an electron injection layer EIL, an electron transport layer ETL, and a hole blocking layer HBL.

When the electron transport region ETR includes the electron transport layer ETL, the thickness of the electron transport layer ETL may be from about 100 Å to about 1,000 Å, for example, from about 150 Å to about 500 Å. When the thickness of the electron transport layer ETL satisfies the described range, satisfactory electron transport properties may be obtained without substantial increase of a driving voltage. When the electron transport region ETR includes the electron injection layer EIL, the thickness of the electron injection layer EIL may be from about 1 Å to about 100 Å, and from about 3 Å to about 90 Å. When the thickness of the electron injection layer EIL satisfies the described range, satisfactory electron injection properties may be obtained without inducing substantial increase of a driving voltage.

The second electrode EL2 is provided on the electron transport region ETR. The second electrode EL2 may be a common electrode. The second electrode EL2 may be a cathode or an anode, but one or more embodiments of the present disclosure is not limited thereto. For example, when the first electrode EL1 is an anode, the second cathode EL2 may be a cathode, and when the first electrode EL1 is a cathode, the second electrode EL2 may be an anode. The second electrode EL2 may include at least one selected among Ag, Mg, Cu, Al, Pt, Pd, Au, Ni, Nd, Ir, Cr, Li, Ca, LiF, Mo, Ti, W, In, Sn, and Zn, compounds of two or more selected therefrom, mixtures of two or more selected therefrom, and/or oxides thereof.

The second electrode EL2 may be a transmissive electrode, a transflective electrode or a reflective electrode. When the second electrode EL2 is the transmissive electrode, the second electrode EL2 may include a transparent metal oxide, for example, ITO, IZO, ZnO, ITZO, and/or the like.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, one or more compounds including thereof, or one or more mixtures thereof (for example, AgMg, AgYb, or MgAg). In some embodiments, the second electrode EL2 may have a multilayered structure including a reflective layer or a transflective layer formed utilizing the described materials and a transparent conductive layer formed utilizing ITO, IZO, ZnO, ITZO, and/or the like. For example, the second electrode EL2 may include one or more of the aforementioned metal materials, combinations of two or more metal materials selected from the aforementioned metal materials, or oxides of the aforementioned metal materials.

In some embodiments, the second electrode EL2 may be connected with an auxiliary electrode. When the second electrode EL2 is connected with the auxiliary electrode, the resistance of the second electrode EL2 may decrease.

In some embodiments, on the second electrode EL2 in the light emitting element ED of one or more embodiments, a capping layer CPL may be further provided. The capping layer CPL may be a multilayer or a single layer.

In one or more embodiments, the capping layer CPL may be an organic layer or an inorganic layer. For example, when the capping layer CPL includes an inorganic material, the inorganic material may include an alkali metal compound such as LiF, an alkaline earth metal compound such as SiON, SiNx, SiOy, and/or the like

For example, when the capping layer CPL includes an organic material, the organic material may include α-NPD, NPB, TPD, m-MTDATA, Alq₃, CuPc, N⁴,N⁴, N⁴′, N⁴′-tetra(biphenyl-4-yl) biphenyl-4,4′-diamine (TPD15), 4,4′,4″-tris(carbazol sol-9-yl) triphenylamine (TCTA), and/or the like, or includes an epoxy resin, or acrylate such as methacrylate. In some embodiments, a capping layer CPL may include at least one selected from among Compounds P1 to P5, but one or more embodiments of the present disclosure is not limited thereto.

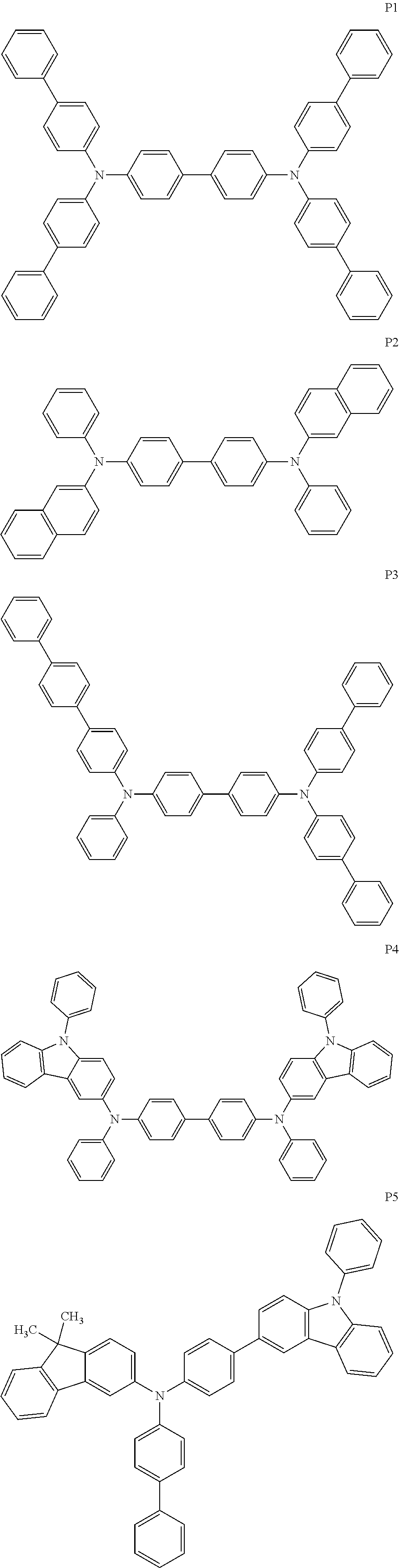

In some embodiments, the refractive index of the capping layer CPL may be about 1.6 or more. For example, the refractive index of the capping layer CPL with respect to light in a wavelength range of about 550 nm to about 660 nm may be about 1.6 or more.

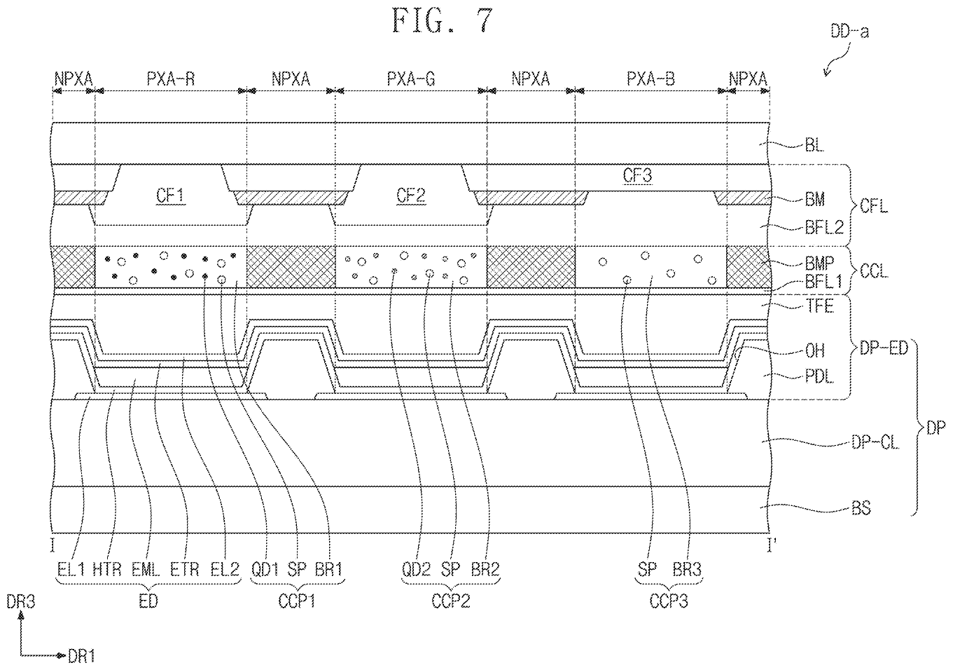

FIG. 7 to FIG. 10 are cross-sectional views on display devices DD-a, DD-TD, DD-b and DD-c, respectively, according to embodiments. Hereinafter, in the explanation on the display devices DD-a, DD-TD, DD-b and DD-c of embodiments, referring to FIG. 7 to FIG. 10, the overlapping parts with the explanation on FIG. 1 to FIG. 6 will not be explained again, and the different features will be explained chiefly (e.g., mainly described).

Referring to FIG. 7, the display device DD-a according to embodiments may include a display panel DP including a display element layer DP-ED, a light controlling layer CCL provided on the display panel DP, and a color filter layer CFL.

In one or more embodiments shown in FIG. 7, the display panel DP includes a base layer BS, a circuit layer DP-CL provided on the base layer BS and a display element layer DP-ED, and the display element layer DP-ED may include a light emitting element ED.

The light emitting element ED may include a first electrode EL1, a hole transport region HTR provided on the first electrode EL1, an emission layer EML provided on the hole transport region HTR, an electron transport region ETR provided on the emission layer EML, and a second electrode EL2 provided on the electron transport region ETR. In some embodiments, the structures of the light emitting elements of FIG. 3 to FIG. 6 may be applied to the structure of the light emitting element ED shown in FIG. 7.

Referring to FIG. 7, the emission layer EML may be provided in an opening part OH defined in a pixel definition layer PDL. For example, the emission layer EML divided by the pixel definition layer PDL and correspondingly provided to each of luminous areas PXA-R, PXA-G and PXA-B may be to emit (e.g., configured to emit) light in substantially the same wavelength region. In the display device DD-a of one or more embodiments, the emission layer EML may be to emit (e.g., configured to emit) blue light. In some embodiments, different from the drawings, in one or more embodiments, the emission layer EML may be provided as a common layer for all luminous areas PXA-R, PXA-G and PXA-B.

The light controlling layer CCL may be provided on the display panel DP. The light controlling layer CCL may include a light converter. The light converter may be a quantum dot or a phosphor. The light converter may transform the wavelength of light incident (e.g., incoming or provided) and then emit. For example, the light controlling layer CCL may be a layer including a quantum dot or a layer including a phosphor.

The light controlling layer CCL may include multiple light controlling parts CCP1, CCP2 and CCP3. The light controlling parts CCP1, CCP2 and CCP3 may be separated from one another.

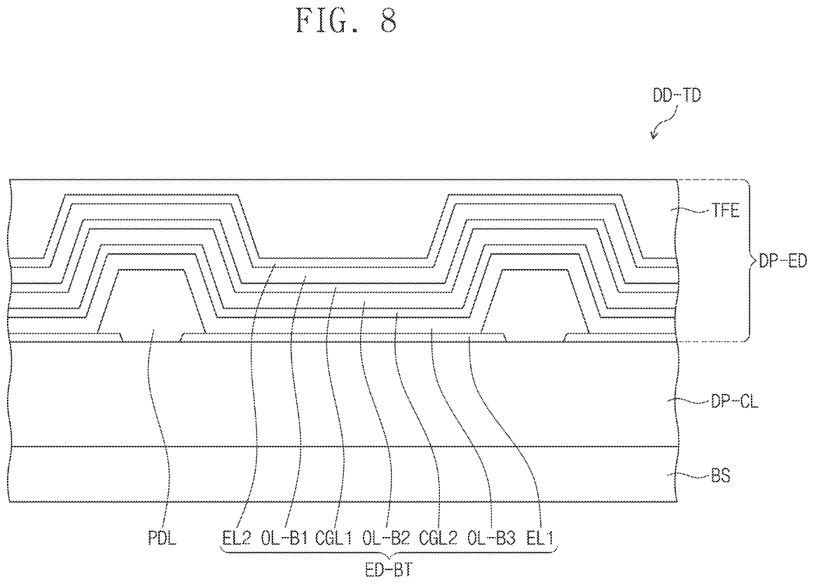

Referring to FIG. 7, a partition pattern BMP may be provided between the separated light controlling parts CCP1, CCP2 and CCP3, but one or more embodiments of the present disclosure is not limited thereto. In FIG. 8, the partition pattern BMP is shown not to be overlapped with the light controlling parts CCP1, CCP2 and CCP3, but at least a portion of the edge of the light controlling parts CCP1, CCP2 and CCP3 may be overlapped with the partition pattern BMP.

The light controlling layer CCL may include a first light controlling part CCP1 including a first quantum dot QD1 converting first color light provided from the light emitting element ED into second color light, a second light controlling part CCP2 including a second quantum dot QD2 converting first color light into third color light, and a third light controlling part CCP3 transmitting first color light.

In one or more embodiments, the first light controlling part CCP1 may provide red light which is the second color light, and the second light controlling part CCP2 may provide green light which is the third color light. The third color controlling part CCP3 may be to transmit and provide blue light which is the first color light provided from the light emitting element ED. For example, the first quantum dot QD1 may be a red quantum dot, and the second quantum dot QD2 may be a green quantum dot. On the quantum dots QD1 and QD2, the same contents as those described herein may be applied.

In some embodiments, the light controlling layer CCL may further include a scatterer SP. The first light controlling part CCP1 may include the first quantum dot QD1 and the scatterer SP, the second light controlling part CCP2 may include the second quantum dot QD2 and the scatterer SP, and the third light controlling part CCP3 may not include (e.g., may exclude) a (e.g., any) quantum dot but include the scatterer SP.

The scatterer SP may be an inorganic particle. For example, the scatterer SP may include at least one selected from among TiO₂, ZnO, Al₂O₃, SiO₂, and hollow silica. The scatterer SP may include at least one selected from among TiO₂, ZnO, Al₂O₃, SiO₂, and hollow silica, or may be a mixture of two or more materials selected among TiO₂, ZnO, Al₂O₃, SiO₂, and hollow silica.

Each of the first light controlling part CCP1, the second light controlling part CCP2, and the third light controlling part CCP3 may include base resins BR1, BR2 and BR3 dispersing the quantum dots QD1 and QD2 and the scatterer SP. In one or more embodiments, the first light controlling part CCP1 may include the first quantum dot QD1 and the scatterer SP dispersed in the first base resin BR1, the second light controlling part CCP2 may include the second quantum dot QD2 and the scatterer SP dispersed in the second base resin BR2, and the third light controlling part CCP3 may include the scatterer particle SP dispersed in the third base resin BR3. The base resins BR1, BR2 and BR3 are each a composition or medium in which the quantum dots QD1 and QD2 and the scatterer SP are dispersed, and may be composed of one or more suitable resin compositions which may be generally referred to as a binder. For example, the base resins BR1, BR2 and BR3 may be acrylic resins, urethane-based resins, silicone-based resins, epoxy-based resins, and/or the like. The base resins BR1, BR2 and BR3 may be transparent resins. In one or more embodiments, the first base resin BR1, the second base resin BR2 and the third base resin BR3 may be the same or different from each other.

The light controlling layer CCL may include a barrier layer BFL1. The barrier layer BFL1 may play the role of blocking the penetration of moisture and/or oxygen (hereinafter, will be referred to as “humidity/oxygen”). The barrier layer BFL1 may be provided on the light controlling parts CCP1, CCP2 and CCP3 and may block or reduce the exposure of the light controlling parts CCP1, CCP2 and CCP3 to humidity/oxygen. In some embodiments, the barrier layer BFL1 may cover the light controlling parts CCP1, CCP2 and CCP3. In some embodiments, the barrier layer BFL2 may be provided between the light controlling parts CCP1, CCP2 and CCP3 and a color filter layer CFL.

The barrier layers BFL1 and BFL2 may include at least one inorganic layer. For example, the barrier layers BFL1 and BFL2 may include (or be formed by including) an inorganic material. For example, the barrier layers BFL1 and BFL2 may be formed by including silicon nitride, aluminum nitride, zirconium nitride, titanium nitride, hafnium nitride, tantalum nitride, silicon oxide, aluminum oxide, titanium oxide, tin oxide, cerium oxide and silicon oxynitride or a metal thin film securing light transmittance. In some embodiments, the barrier layers BFL1 and BFL2 may further include an organic layer. The barrier layers BFL1 and BFL2 may include (or be composed of) a single layer of multiple layers.

In the display device DD-a of one or more embodiments, the color filter layer CFL may be provided on the light controlling layer CCL. For example, the color filter layer CFL may be provided directly on the light controlling layer CCL. In this case, the barrier layer BFL2 may not be provided.

The color filter layer CFL may include a light blocking part BM and filters CF1, CF2 and CF3. The color filter layer CFL may include a first filter CF1 transmitting second color light, a second filter CF2 transmitting third color light, and a third filter CF3 transmitting first color light. For example, the first filter CF1 may be a red filter, the second filter CF2 may be a green filter, and the third filter CF3 may be a blue filter. Each of the filters CF1, CF2 and CF3 may include a polymer photosensitive resin and a pigment or dye. The first filter CF1 may include a red pigment or dye, the second filter CF2 may include a green pigment or dye, and the third filter CF3 may include a blue pigment or dye. In some embodiments, one or more embodiments of the present disclosure is not limited thereto, and the third filter CF3 may not include (e.g., may exclude) the (e.g., any) pigment or dye. The third filter CF3 may include a polymer photosensitive resin and not include a (e.g., any) pigment or dye. The third filter CF3 may be transparent. The third filter CF3 may be formed utilizing a transparent photosensitive resin.

In some embodiments, in one or more embodiments, the first filter CF1 and the second filter CF2 may be yellow filters. The first filter CF1 and the second filter CF2 may be provided in one body without distinction.

The light blocking part BM may be a black matrix. The light blocking part BM may include (or be formed by including) an organic light blocking material or an inorganic light blocking material, including a black pigment or a black dye. The light blocking part BM may prevent or reduce light leakage phenomenon and divide the boundaries among adjacent filters CF1, CF2 and CF3. In some embodiments, in one or more embodiments, the light blocking part BM may include (or be formed as) a blue filter.

The first to third filters CF1, CF2 and CF3 may be provided corresponding to the red luminous area PXA-R, the green luminous area PXA-G and the blue luminous area PXA-B, respectively.

On the color filter layer CFL, a base substrate BL may be provided. The base substrate BL may be a member providing a base surface on which the color filter layer CFL, the light controlling layer CCL, and/or the like are provided. The base substrate BL may be a glass substrate, a metal substrate, a plastic substrate, and/or the like. However, one or more embodiments of the present disclosure is not limited thereto, and the base substrate BL may be an inorganic layer, an organic layer or a composite material layer. In some embodiments, different from the drawing, the base substrate BL may not be provided in one or more embodiments.

FIG. 8 is a cross-sectional view showing a part of the display device DD-TD according to one or more embodiments. In FIG. 8, the cross-sectional view of a part corresponding to the display panel DP of FIG. 7 is shown. In a display device DD-TD of one or more embodiments, the light emitting element ED-BT may include multiple light emitting structures OL-B1, OL-B2 and OL-B3. The light emitting element ED-BT may include oppositely provided first electrode EL1 and second electrode EL2, and the multiple light emitting structures OL-B1, OL-B2 and OL-B3 stacked in order in a thickness direction and provided between the first electrode EL1 and the second electrode EL2. Each of the light emitting structures OL-B1, OL-B2 and OL-B3 may include the emission layer EML (FIG. 7), and a hole transport region HTR and an electron transport region ETR, provided with the emission layer (FIG. 7) therebetween.

For example, the light emitting element ED-BT included in the display device DD-TD of one or more embodiments may be a light emitting element of a tandem structure including multiple emission layers.

In one or more embodiments shown in FIG. 8, light emitted from the light emitting structures OL-B1, OL-B2 and OL-B3 may be all blue light. However, one or more embodiments of the present disclosure is not limited thereto, and the wavelength regions of light emitted from the light emitting structures OL-B1, OL-B2 and OL-B3 may be different from each other. For example, the light emitting element ED-BT including the multiple light emitting structures OL-B1, OL-B2 and OL-B3 emitting light in different wavelength regions may be to emit white light.

Between neighboring light emitting structures OL-B1, OL-B2 and OL-B3, charge generating layers CGL1 and CGL2 may be provided. The charge generating layers CGL1 and CGL2 may include a p-type or kind charge generating layer (e.g., p-charge generating (or generation) layer) and/or an n-type or kind charge generating layer (e.g., n-charge generating (or generation) layer).

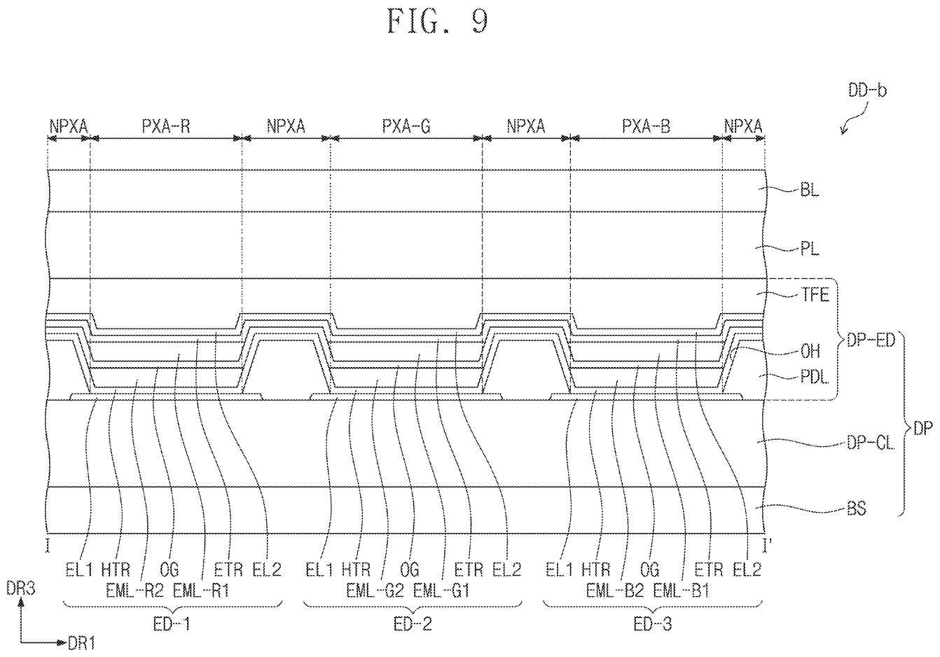

Referring to FIG. 9, a display device DD-b according to one or more embodiments may include light emitting elements ED-1, ED-2 and ED-3, formed by stacking two emission layers. Compared to the display device DD of one or more embodiments, shown in FIG. 2, one or more embodiments shown in FIG. 9 is different in that first to third light emitting elements ED-1, ED-2 and ED-3 include two emission layers stacked in a thickness direction, each. In the first to third light emitting elements ED-1, ED-2 and ED-3, two emission layers may be to emit light in substantially the same wavelength region.

The first light emitting element ED-1 may include a first red emission layer EML-R1 and a second red emission layer EML-R2. The second light emitting element ED-2 may include a first green emission layer EML-G1 and a second green emission layer EML-G2. In some embodiments, the third light emitting element ED-3 may include a first blue emission layer EML-B1 and a second blue emission layer EML-B2. Between the first red emission layer EML-R1 and the second red emission layer EML-R2, between the first green emission layer EML-G1 and the second green emission layer EML-G2, and between the first blue emission layer EML-B1 and the second blue emission layer EML-B2, an emission auxiliary part OG may be provided.

The emission auxiliary part OG may include a single layer or a multilayer. The emission auxiliary part OG may include a charge generating layer. More particularly, the emission auxiliary part OG may include an electron transport region, a charge generating layer, and a hole transport region stacked in order. The emission auxiliary part OG may be provided as a common layer in all of the first to third light emitting elements ED-1, ED-2 and ED-3. However, one or more embodiments of the present disclosure is not limited thereto, and the emission auxiliary part OG may be patterned and provided in an opening part OH defined in a pixel definition layer PDL.

The first red emission layer EML-R1, the first green emission layer EML-G1 and the first blue emission layer EML-B1 may be provided between the electron transport region ETR and the emission auxiliary part OG. The second red emission layer EML-R2, the second green emission layer EML-G2 and the second blue emission layer EML-B2 may be provided between the emission auxiliary part OG and the hole transport region HTR.

For example, the first light emitting element ED-1 may include a first electrode EL1, a hole transport region HTR, a second red emission layer EML-R2, an emission auxiliary part OG, a first red emission layer EML-R1, an electron transport region ETR, and a second electrode EL2, stacked in order. The second light emitting element ED-2 may include a first electrode EL1, a hole transport region HTR, a second green emission layer EML-G2, an emission auxiliary part OG, a first green emission layer EML-G1, an electron transport region ETR, and a second electrode EL2, stacked in order. The third light emitting element ED-3 may include a first electrode EL1, a hole transport region HTR, a second blue emission layer EML-B2, an emission auxiliary part OG, a first blue emission layer EML-B1, an electron transport region ETR, and a second electrode EL2, stacked in order.

In some embodiments, an optical auxiliary layer PL may be provided on a display element layer DP-ED. The optical auxiliary layer PL may include a polarization layer. The optical auxiliary layer PL may be provided on a display panel DP and may control reflected light at the display panel DP by external light. Different from the drawings, the optical auxiliary layer PL may not be provided from the display device according to one or more embodiments.

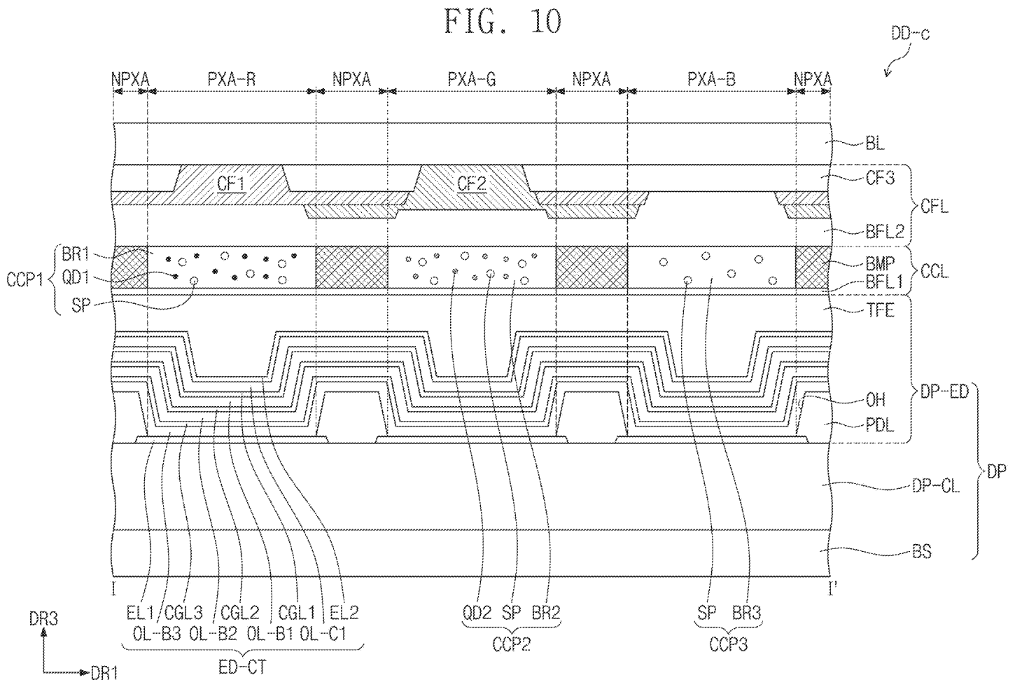

Different from FIG. 8 and FIG. 9, a display device DD-c in FIG. 10 is shown to include four light emitting structures OL-B1, OL-B2, OL-B3 and OL-C1. A light emitting element ED-CT may include oppositely provided first electrode EL1 and second electrode EL2, and first to fourth light emitting structures OL-B1, OL-B2, OL-B3 and OL-C1 stacked in order in a thickness direction between the first electrode EL1 and the second electrode EL2. Between the first to fourth light emitting structures OL-B1, OL-B2, OL-B3 and OL-C1, charge generating layers CGL1, CGL2 and CGL3 may be provided. Among the four light emitting structures, the first to third light emitting structures OL-B1, OL-B2 and OL-B3 may be to emit blue light, and the fourth light emitting structure OL-C1 may be to emit green light. However, one or more embodiments of the present disclosure is not limited thereto, and the first to fourth light emitting structures OL-B1, OL-B2, OL-B3 and OL-C1 may be to emit different wavelengths of light.

Charge generating layers CGL1, CGL2 and CGL3 provided among neighboring light emitting structures OL-B1, OL-B2, OL-B3 and OL-C1 may include a p-type or kind charge generating layer and/or an n-type or kind charge generating layer.

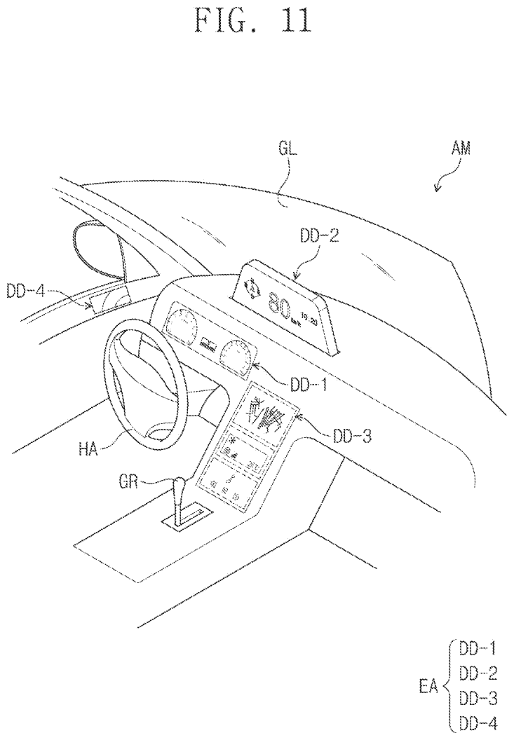

FIG. 11 is a perspective view schematically showing an electronic device including the display device according to one or more embodiments. In FIG. 11, as an electronic device, a part of an automobile AM is shown as an illustration. However, this is an illustration, and one or more suitable transport refers to such as bicycles, motorcycles, trains, ships and airplanes may be included.

Referring to FIG. 11, the automobile AM may include first to fourth display devices DD-1, DD-2, DD-3 and DD-4 for a vehicle. The same contents on the display devices DD, DD-a, DD-TD, DD-b and DD-c, explained referring to FIG. 1, FIG. 2, and FIG. 7 to FIG. 10, may be applied for at least one selected from among the first to fourth display devices DD-1, DD-2, DD-3 and DD-4.

In one or more embodiments, at least one selected from among the first to fourth display devices DD-1, DD-2, DD-3 and DD-4 may include the light emitting elements ED explained referring to FIG. 3 to FIG. 6. The first to fourth display devices DD-1, DD-2, DD-3 and DD-4 may each independently include multiple light emitting elements ED (see FIG. 3 to FIG. 6). Each of the light emitting elements ED may include a first electrode EL1, a hole transport region HTL, an emission layer EML, an electron transport region ETR and a second electrode EL2 (see FIG. 3 to FIG. 6). In some embodiments, the emission layer EML may include the explained amine compound of one or more embodiments. Accordingly, the first to fourth display devices DD-1, DD-2, DD-3 and DD-4 for a vehicle may show improved quality of images.

Referring to FIG. 11, the automobile AM may include a steering wheel HA for the operation of the automobile AM and a gear GR. The automobile AM may include a front window GL provided to face a driver.

A first display device DD-1 may be provided in a first region overlapping with the steering wheel HA. For example, the first display device DD-1 may be a digital cluster displaying the first information of the automobile AM. The first information may include a first graduation showing the running speed of the automobile AM, a second graduation showing the number of revolution of an engine (i.e., revolutions per minute (RPM)), and images showing a fuel state. First graduation and second graduation may be represented by digital images.

A second display device DD-2 may be provided in a second region facing a driver's seat and overlapping with the front window GL. The driver's seat may be a seat where the steering wheel HA is provided. For example, the second display device DD-2 may be a head up display (HUD) showing the second information of the automobile AM. The second display device DD-2 may be optically clear. The second information may include digital numbers showing the running speed of the automobile AM and may further include information including the current time.

A third display device DD-3 may be provided in a third region adjacent to the gear GR. For example, the third display device DD-3 may be a center information display (CID) for an automobile, provided between a driver's seat and a passenger seat and showing third information. The passenger seat may be a seat separated from the driver's seat with the gear GR therebetween. The third information may include information on road conditions (for example, navigation information), on playing music or radio, on playing a dynamic image, on the temperature in the automobile AM, and/or the like.

A fourth display device DD-4 may be provided in a fourth region separated from the steering wheel HA and the gear GR and adjacent to the side of the automobile AM. For example, the fourth display device DD-4 may be a digital wing mirror displaying fourth information. The fourth display device DD-4 may include the external image of the automobile AM, taken by a camera module provided at the outside of the automobile AM.

The described first to fourth information is for illustration, and the first to fourth display devices DD-1, DD-2, DD-3 and DD-4 may further display information on the inside and outside of the automobile. The first to fourth information may include different information from each other. However, one or more embodiments of the present disclosure is not limited thereto, and a portion of the first to fourth information may include the same information.

Terms such as “substantially,” “about,” and “approximately” are used as relative terms and not as terms of degree, and are intended to account for the inherent deviations in measured or calculated values that would be recognized by those of ordinary skill in the art. They may be inclusive of the stated value and an acceptable range of deviation as determined by one of ordinary skill in the art, considering the limitations and error associated with measurement of that quantity. For example, “about” may refer to one or more standard deviations, or ±30%, 20%, 10%, 5% of the stated value.

Numerical ranges disclosed herein include and are intended to disclose all subsumed sub-ranges of the same numerical precision. For example, a range of “1.0 to 10.0” includes all subranges having a minimum value equal to or greater than 1.0 and a maximum value equal to or less than 10.0, such as, for example, 2.4 to 7.6. Applicant therefore reserves the right to amend this specification, including the claims, to expressly recite any sub-range subsumed within the ranges expressly recited herein.

The light emitting element, the display device and/or any other relevant devices or components according to embodiments of the present disclosure described herein may be implemented utilizing any suitable hardware, firmware (e.g., an application-specific integrated circuit), software, or a combination of software, firmware, and hardware. For example, the various components of the light emitting element and/or the display device may be formed on one integrated circuit (IC) chip or on separate IC chips. Further, the various components of the elements and/or devices may be implemented on a flexible printed circuit film, a tape carrier package (TCP), a printed circuit board (PCB), or formed on one substrate. Further, the various components of the elements and/or devices may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

Hereinafter, referring to embodiments and comparative embodiments, the amine compound according to one or more embodiments and the light emitting element according to one or more embodiments of the present disclosure will be explained in particular. In some embodiments, the embodiments are illustrations to assist the understanding of the present disclosure, but the scope of the present disclosure is not limited thereto.

### EXAMPLES

### 1. Synthesis of Amine Compounds

The synthetic methods of amine compounds according to embodiments will be explained by illustrating the synthetic methods of Compounds 1, 10, 43, 52, 151, 410, 411, 418, and 436. The synthetic methods of the amine compounds explained hereinafter are embodiments, and the synthetic method of the amine compound according to one or more embodiments of the present disclosure is not limited to the embodiments.

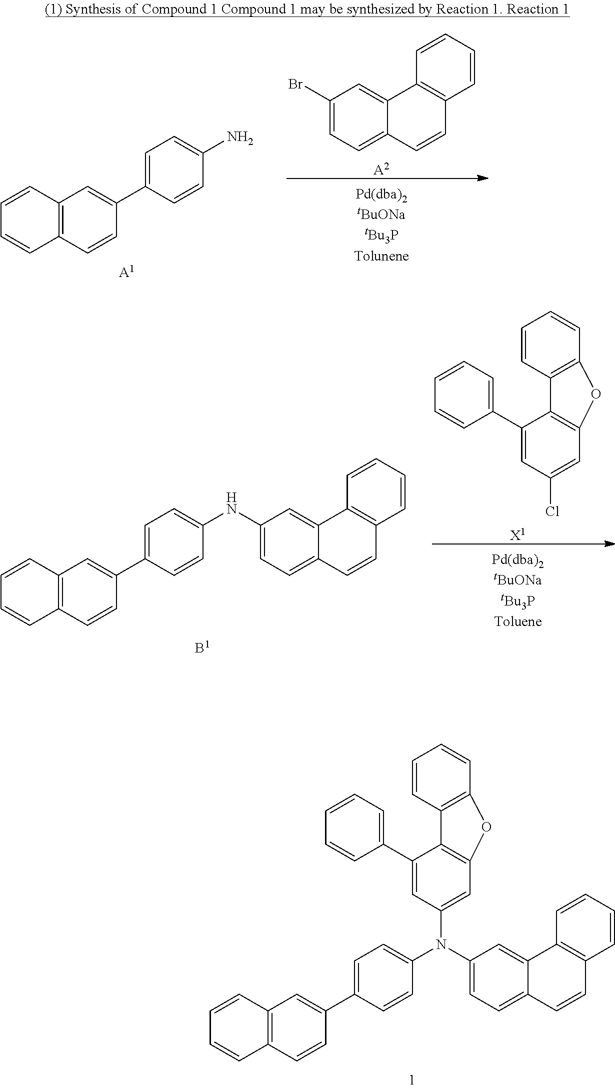

### 1) Synthesis of B¹

To a mixture of A¹ (12.8 g, 58.4 mmol), A² (12.0 g, 46.7 mmol), bis(dibenzilideneacetone)palladium(0) (1.01 g, 17.5 mmol), sodium tert-butoxide (16.8 g, 175 mmol), and toluene (500 mL), under an argon atmosphere, tri-tert-butylphosphine (2 M solution, 1.75 mL, 35.0 mmol) was added dropwise, followed by stirring at about 110° C. for about 8 hours. The reaction mixture thus obtained was cooled, filtered through celite, washed with water and a saturated saline solution and concentrated. The residue thus obtained was purified through column chromatography to obtain B¹ (15.7 g, yield 85%). (FABMS m/z=395.2)

### 2) Synthesis of Compound 1

To a mixture of B¹ (4.13 g, 10.5 mmol), X¹ (3.01 g, 10.5 mmol, [CAS: 2361006-01-9]), bis(dibenzilideneacetone)palladium(0) (180 mg, 0.314 mmol), sodium tert-butoxide (3.02 g, 31.4 mmol), and toluene (200 mL), under an argon atmosphere, tri-tert-butylphosphine (2 M solution, 0.310 mL, 0.628 mmol) was added dropwise, followed by stirring at about 110° C. for about 12 hours. The reaction mixture thus obtained was cooled, filtered through celite, washed with water and a saturated saline solution and concentrated. The residue thus obtained was purified through column chromatography to obtain Compound 1 (15.7 g, yield 78%). (FABMS m/z=637.2) (2) Synthesis of Compound 10

The same method as utilized for the synthesis of Compound 1 was performed except for utilizing X² instead of X¹ in the synthesis of Compound 1 to obtain Compound 10 (yield 70%, FABMS m/z=653.3).

### (3) Synthesis of Compound 43

The same method as utilized for the synthesis of Compound 1 was performed except for utilizing B⁸ instead of B¹ in the synthesis of Compound 1 to obtain Compound 43 (yield 77%, FABMS m/z=663.3).

### (4) Synthesis of Compound 52

The same method as utilized for the synthesis of Compound 1 was performed except for utilizing B⁴ instead of B¹ in the synthesis of Compound 1 to obtain Compound 52 (yield 75%, FABMS m/z=727.3).

### (5) Synthesis of Compound 151

The same method as utilized for the synthesis of Compound 1 was performed except for utilizing B⁶ instead of B¹ in the synthesis of Compound 1 to obtain Compound 151 (yield 65%, FABMS m/z=643.2).

### (6) Synthesis of Compound 410

The same method as utilized for the synthesis of Compound 1 was performed except for utilizing B² instead of B¹ in the synthesis of Compound 1 to obtain Compound 410 (yield 73%, FABMS m/z=627.2).

### (7) Synthesis of Compound 411

The same method as utilized for the synthesis of Compound 1 was performed except for utilizing B³ instead of B¹ in the synthesis of Compound 1 to obtain Compound 411 (yield 81%, FABMS m/z=627.2).

### (8) Synthesis of Compound 418

The same method as utilized for the synthesis of Compound 1 was performed except for utilizing B⁵ instead of B¹ in the synthesis of Compound 1 to obtain Compound 418 (yield 68%, FABMS m/z=643.2).

### (9) Synthesis of Compound 436

The same method as utilized for the synthesis of Compound 1 was performed except for utilizing B⁷ instead of B¹ in the synthesis of Compound 1 to obtain Compound 436 (yield 72%, FABMS m/z=702.3).

In the synthesis of Compounds 10, 43, 52, 151, 410, 411, 418, and 436, the compounds additionally utilized are suitable compounds and as follows.

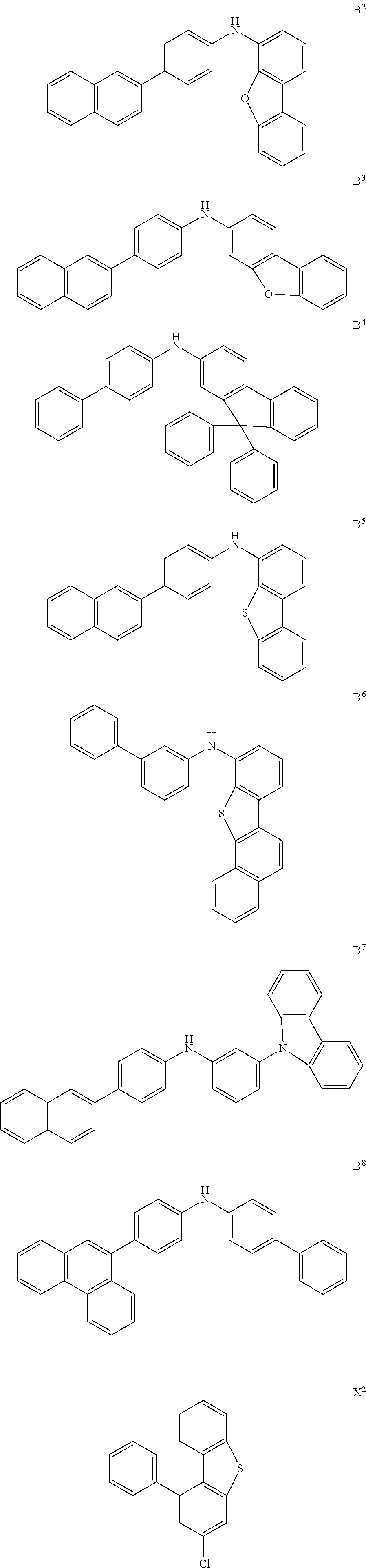

B²: [CAS: 2413378-82-0]
B³: [CAS: 2413378-82-0]
B⁴: [CAS: 1268520-04-2]
B⁵: [CAS: 2413379-18-5]
B⁶: [CAS: 1846603-27-7]
B⁷: [CAS: 2826962-66-5]
B⁸: [CAS: 1918982-76-9]
X²: [CAS: 2839441-16-4].

### 2. Manufacture of Light Emitting Elements Including Amine Compounds

A light emitting element including the amine compound of one or more embodiments in a hole transport region was manufactured by a method described herein. Light emitting elements of Examples 1 to 9 were manufactured utilizing Compounds 1, 10, 43, 52, 151, 410, 411, 418, and 436, which are the amine compounds of embodiments, as the materials of a hole transport layer. Light emitting elements of Comparative Examples 1 to 13 corresponded to light emitting elements manufactured utilizing Comparative Compounds C1 to C13 as the materials of a hole transport layer.

An ITO glass substrate with about 15 ohm per square centimeter (Ω/cm²) (thickness of about 150 nanometer (nm)) of Corning Co. was cut into a size of 50 millimeter (mm)×50 mm×0.7 mm, cleansed by ultrasonic waves utilizing isopropyl alcohol and pure water for about 5 minutes each, exposed to UV for about 30 minutes and treated with ozone. The glass substrate was installed in a vacuum deposition apparatus, and a first electrode was formed.

On the first electrode, a suitable material of 2-TNATA was vacuum deposited to a thickness of about 60 nm to form a hole injection layer, and then, the Example Compound or Comparative Compound was vacuum deposited to a thickness of about 30 nm to form a hole transport layer.

On the hole transport layer, a blue fluorescence host of a suitable material of 9,10-di(naphthalen-2-yl)anthracene (hereinafter, ADN) and a blue fluorescence dopant of a suitable material of 2,5,8,11-tetra-tert-butylperylene (hereinafter, TBP) were co-deposited in a ratio (e.g., amount) of about 97:3 to form an emission layer with a thickness of about 25 nm.

On the emission layer, an electron transport layer was formed to a thickness of about 25 nm by depositing Alq₃, and then, on the hole transport layer, an electron injection layer was formed to a thickness of about 1 nm by depositing an alkali metal halide of LiF. On the electron injection layer, Al was vacuum deposited to a thickness of about 100 nm to form a second electrode, thereby manufacturing a light emitting element.

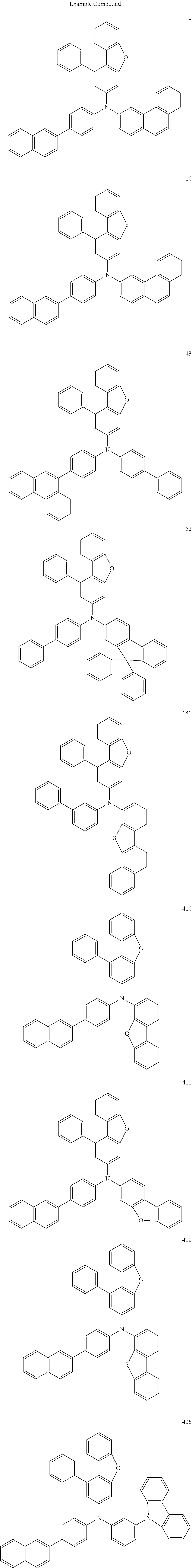

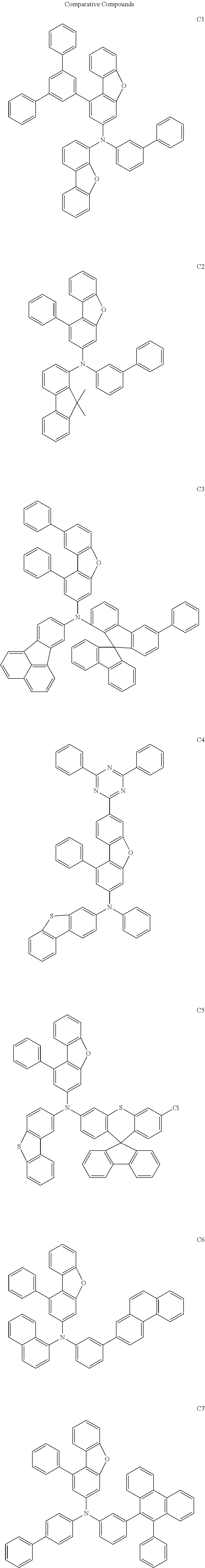

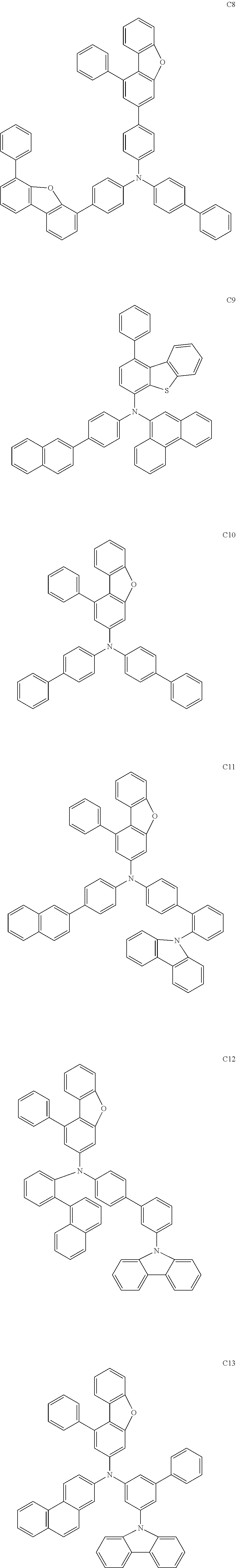

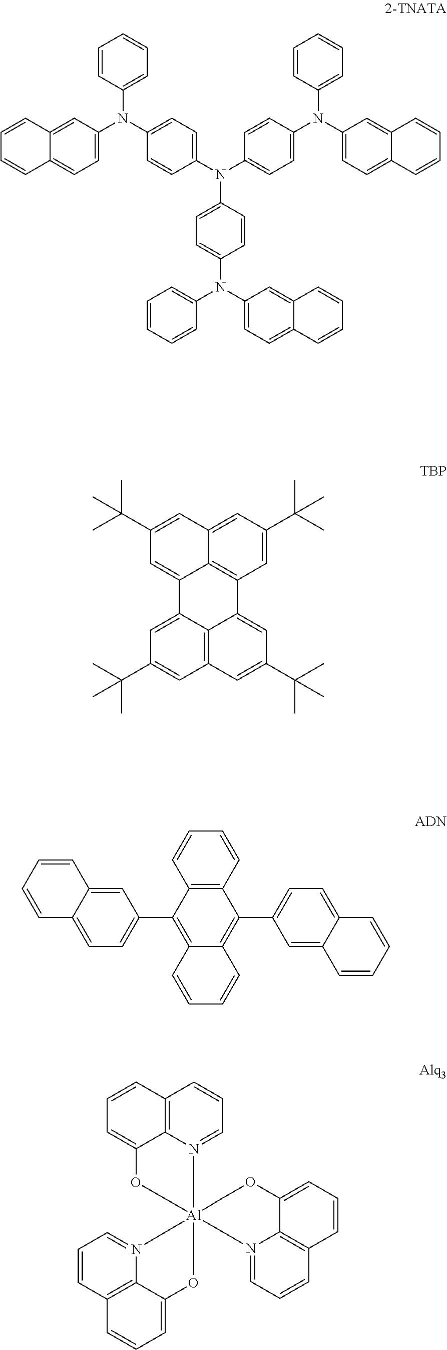

### 3. Evaluation of Light Emitting Elements Including Amine Compounds

The properties of the light emitting elements of Examples 1 to 9 and Comparative Examples 1 to 13 were evaluated. The light emitting elements of Examples 1 to 9 and Comparative Examples 1 to 13 were manufactured according to the described element manufacturing method.

Table 12 shows relative emission efficiency, relative lifetime, and material decomposition ratios. Measurement was conducted utilizing I-V-L Test System Polaronix V7000 (manufacturer: DichloromethaneSience Inc.). The lifetime of the light emitting element was obtained by measuring the time from an initial value to 50% luminance deterioration when driven continuously at a current density of about 10 milliampere per square centimeter (mA/cm²). The relative element lifetime (%) and the relative lifetime (%) were calculated and shown based on Comparative Example 1. The material composition ratio was measured from a difference obtained by subtracting the purity of a remaining material after manufacturing a light emitting element from the purity of a material before manufacturing a light emitting element.

TABLE 12

| | | | | Material |
|---|---|---|---|---|
| | | Relative | | decom- |
| | | emission | Relative | position |
| Element | HTL | efficiency | lifetime | ratio |
| Example 1 | Compound 1 | 110% | 125% | <0.1% |
| Example 2 | Compound 410 | 115% | 120% | <0.1% |
| Example 3 | Compound 411 | 105% | 130% | <0.1% |
| Example 4 | Compound 52 | 110% | 130% | <0.1% |
| Example 5 | Compound 52 | 115% | 120% | <0.1% |
| Example 6 | Compound 151 | 105% | 125% | <0.1% |
| Example 7 | Compound 436 | 110% | 120% | <0.1% |
| Example 8 | Compound 43 | 105% | 130% | <0.1% |
| Example 9 | Compound 10 | 110% | 120% | <0.1% |
| Comparative | Compound C1 | 100% | 100% | 0.1% |
| Example 1 | | | | |
| Comparative | Compound C2 | 95% | 70% | <0.1% |
| Example 2 | | | | |
| Comparative | Compound C3 | 70% | 30% | 0.1% |
| Example 3 | | | | |
| Comparative | Compound C4 | 70% | 10% | <0.1% |
| Example 4 | | | | |
| Comparative | Compound C5 | 80% | 10% | 0.1% |
| Example 5 | | | | |
| Comparative | Compound C6 | 95% | 40% | 0.2% |
| Example 6 | | | | |
| Comparative | Compound C7 | 95% | 60% | 0.1% |
| Example 7 | | | | |
| Comparative | Compound C8 | 85% | 100% | <0.1% |
| Example 8 | | | | |
| Comparative | Compound C9 | 100% | 95% | 0.1% |
| Example 9 | | | | |
| Comparative | Compound C10 | 95% | 100% | <0.1% |
| Example 10 | | | | |
| Comparative | Compound C11 | 95% | 95% | 0.1% |
| Example 11 | | | | |
| Comparative | Compound C12 | 100% | 95% | 0.1% |
| Example 12 | | | | |
| Comparative | Compound C13 | 100% | 80% | 0.1% |
| Example 13 | | | | |

Referring to Table 12, the light emitting elements of Examples 1 to 9, which are light emitting elements in which the amine compound of the present disclosure is applied, showed element properties of higher emission efficiency and longer lifetime, when compared to the light emitting elements of Comparative Examples 1 to 13. In some embodiments, the material decomposition ratios of the light emitting elements of Examples 1 to 9 were less than about 0.1%, which were similar to or less (e.g., smaller) than the material decomposition ratios of the light emitting elements of Comparative Examples 1 to 13.

As described herein, the monoamine compound represented by Formula 1 of the present disclosure includes a 1-aryldibenzofuran-3-yl group or a 1-aryldibenzothiophen-3-yl group connected to the nitrogen atom of an amine (hereinafter, a first substituent). Because an aryl group is bonded at position 1 of dibenzofuran or dibenzothiophene, the hole transport capacity and stability of a compound were improved.

The total carbon number (i.e., number of carbon atoms) of the aryl group bonded at position 1 (i.e., of the dibenzofuran or dibenzothiophene of the first substituent) is 6 to 16. The aryl group having the carbon number in the described range may improve or enhance the stability of the compound.

In some embodiments, position 3 of the dibenzofuran or dibenzothiophene of the first substituent is directly bonded to the nitrogen atom of the amine. Because no linker that may, e.g., inhibit interaction between the dibenzofuran or dibenzothiophene of the first substituent with the nitrogen atom, is provided, that hole transport capacity may be excellent or suitable.

In some embodiments, the second substituent and the third substituent are substituted or unsubstituted aryl groups or heteroaryl groups. The second substituent and the third substituent may be directly bonded to the nitrogen atom of the amine or may be bonded via a linker.

As described herein, the amine compound has excellent or suitable hole transport capacity and excellent or suitable compound stability. Accordingly, a light emitting element including the amine compound of the present disclosure in a hole transport layer may show relatively high emission efficiency and long lifetime.

In some embodiments, Comparative Compound C1 is a compound in which the aryl group bonded at position 1 of the dibenzofuran that is bonded to the nitrogen atom of the amine is a phenyl group substituted with two phenyl groups. For example, the total carbon number of the aryl group is 18, and the steric volume of the aryl group bonded to the dibenzofuran is very large, and molecular distortion occurs. Accordingly, the material stability and hole transport capacity may be deteriorated, and the emission efficiency and lifetime of a light emitting element including the same are deteriorated.

Comparative Compound C2 is a compound in which a 9,9-dimethylfluorenyl group is bonded to the nitrogen atom of the amine, and the chemical stability may be low, accordingly the emission efficiency and lifetime of a light emitting element including the same are deteriorated.

Comparative Compound C3 is a compound in which a fluoranthene group is bonded to the nitrogen atom of the amine, and thus has a low triplet energy level (T1) and insufficient capturing function of energy in an emission layer, produced in the emission layer. Accordingly, the emission efficiency and lifetime of a light emitting element including the same may be degraded.

Comparative Compound C4 is a compound in which a triazine group is bonded to a dibenzofuran. Because a triazine group has a structure having high electron transport capacity, the hole transport capacity of a compound may be degraded. Accordingly, the emission efficiency and lifetime of a light emitting element including the same may be degraded.

Comparative Compound C5 is a compound including a halogen atom as a substituent. Because a halogen atom has low chemical stability, the emission efficiency and lifetime of a light emitting element including the same may be degraded.

Comparative Compound C6 is a compound in which a naphthyl group is directly bonded to the nitrogen atom of the amine. When a naphthyl group is directly bonded to the amine nitrogen, the influence of the amine nitrogen may act largely to the naphthyl group, and the stability of the naphthyl group may be degraded. Accordingly, the emission efficiency and lifetime of a light emitting element including the same may be degraded.

Comparative Compound C7 is a compound in which a 10-arylphenanthrenyl group is bonded at position 9 to the nitrogen atom of the amine through a phenylene linker. In the phenylene linker, the 10-arylphenanthrenyl group and the nitrogen atom of the amine are present at m-positions. In the structure, a large distortion may arise in a molecule, and the stability of a molecule may be degraded. Accordingly, the emission efficiency and lifetime of a light emitting element including the same may be degraded.

Comparative Compound C8 is a compound in which a 1-phenyl-dibenzofuran group is not directly bonded to the nitrogen atom of the amine but bonded via a phenylene linker. Due to the phenylene linker, interaction between the 1-phenyl-dibenzofuran group and the nitrogen atom of the amine is deteriorated, and the emission efficiency and lifetime of a light emitting element including the same may be degraded.

Comparative Compound C9 is a compound in which a 1-phenyl-dibenzothiophene group is bonded at position 4 to the nitrogen atom of the amine. The phenyl group bonded to the dibenzothiophene group has a largely distorted bonding angle with respect to the nitrogen atom of the amine, the HOMO at the nitrogen atom of the amine may not be spread to the phenyl group, and the HOMO may not be sufficiently stabilized. Accordingly, the emission efficiency and lifetime of a light emitting element including the same may be degraded.

Comparative Compound C10 is a compound in which the second and third substituents are biphenyl groups, and the number of ring-forming carbon atoms of the second and third substituents is 6 in each case. Accordingly, the material stability and hole transport capacity are deteriorated, and the emission efficiency and lifetime of a light emitting element including the same may be degraded.

Comparative Compounds C10 to C13 are compounds in which a carbazole group is bonded to the nitrogen atom of the amine via a biphenylene group or a substituted phenylene linker. Accordingly, molecular distortion arises, the material stability and hole transport capacity are deteriorated, and the emission efficiency and lifetime of a light emitting element including the same may be degraded.

The amine compound of one or more embodiments according to the present disclosure may include a 1-aryldibenzofuran-3-yl group or a 1-aryldibenzothiophen-3-yl group (hereinafter, a first substituent). For example, position 3 of the dibenzofuran/dibenzothiophene of the first substituent may be directly bonded to the nitrogen atom of the amine. To the nitrogen atom of the amine, at least one selected from among a substituted or unsubstituted aryl group of 10 to 30 ring-forming carbon atoms, and a substituted or unsubstituted heteroaryl group of 12 to 30 ring-forming carbon atoms may be bonded (second and third substituents). The second and third substituents bonded to the nitrogen atom of the amine do not include an unsubstituted dimethylfluorenyl group, a substituted or unsubstituted fluoranthene group, and/or a halogen atom. When the second and third substituents are substituted or unsubstituted naphthyl groups, they are not directly bonded to the nitrogen atom of the amine, and the 10-arylphenanthre-9-yl groups of the second and third substituents are not connected via a m-phenylene group. When the second and third substituents are carbazole groups, they are directly bonded to the nitrogen atom of the amine or connected via an unsubstituted phenylene group.

The amine compound of one or more embodiments according to the present disclosure may have excellent or suitable hole transport capacity. Accordingly, a light emitting element including the amine compound of one or more embodiments in a hole transport region may have improved charge balance and may show relatively high emission efficiency and long lifetime.

The light emitting element of one or more embodiments may show improved element properties of relatively high emission efficiency and long lifetime.

The amine compound of one or more embodiments may be included in the hole transport region of a light emitting element and may contribute to the improvement of the emission efficiency and lifetime of the light emitting element.

Although the embodiments of the present disclosure have been described, it is understood that the present disclosure should not be limited to these embodiments, but one or more suitable changes and modifications can be made by one ordinary skilled in the art within the spirit and scope of the present disclosure as set forth in the following claims and equivalents thereof.

What is claimed is:

1. A light emitting element, comprising:
a first electrode;
a second electrode on the first electrode;
at least one functional layer comprising an amine compound represented by Formula 1, and between the first electrode and the second electrode: 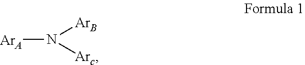
wherein in Formula 1,
Ar_{A} is represented by Formula A,
Ar_{B} is represented by Formula B, and
Ar_{C} is represented by Formula C: 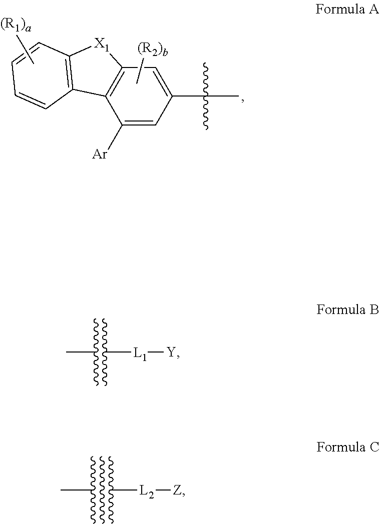
in Formula A to Formula C,
X₁ is O, or S,
Ar is a substituted or unsubstituted aryl group with a total carbon number of 6 to 16,
R₁ and R₂ are each independently a hydrogen atom, a substituted or unsubstituted alkyl group of 1 to 10 carbon atoms, a substituted or unsubstituted aryl group of 6 to 30 ring-forming carbon atoms, or a substituted or unsubstituted heteroaryl group of 2 to 30 ring-forming carbon atoms,
each of R₁ and R₂ does not comprise a substituted or unsubstituted nitrogen-containing six-member heterocycle,
“a” is an integer of 0 to 4,
“b” is an integer of 0 to 2,
L₁ and L₂ are each independently a direct linkage, or a substituted or unsubstituted arylene group of 6 to 30 ring-forming carbon atoms,
Y and Z are each independently a substituted or unsubstituted aryl group of 6 to 30 ring-forming carbon atoms, or a substituted or unsubstituted heteroaryl group of 2 to 30 ring-forming carbon atoms,
at least one selected from among Y and Z is a substituted or unsubstituted aryl group of 10 to 30 ring-forming carbon atoms, or a substituted or unsubstituted heteroaryl group of 12 to 30 ring-forming carbon atoms,
each of Y and Z does not comprise a substituted or unsubstituted dimethylfluorenyl group, a substituted or unsubstituted fluoranthene group, or a halogen atom,
when Y is a substituted or unsubstituted naphthyl group, L₁ is not a direct linkage,
when Z is a substituted or unsubstituted naphthyl group, L₂ is not a direct linkage,
when Y is a substituted or unsubstituted carbazole group, L₁ is a direct linkage or an unsubstituted phenylene group,
when Z is a substituted or unsubstituted carbazole group, L₂ is a direct linkage or an unsubstituted phenylene group,
when L₁ is a m-phenylene group, Y is not a substituted or unsubstituted 10-arylphenanthren-9-yl group,
when L₂ is a m-phenylene group, Z is not a substituted or unsubstituted 10-arylphenanthren-9-yl group, [symbol]
, [symbol] , [symbol] and are positions respectively bonded to the nitrogen atom of Formula 1, and
Formula 1 optionally comprises a deuterium atom.

2. The light emitting element of claim 1, wherein at least one selected from among Y and Z is represented by Formula 1a to Formula 1c: 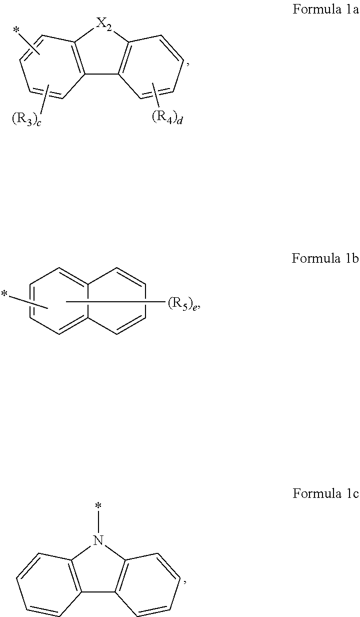
wherein in Formula 1a to Formula 1c,
X₂ is O, S, NRₐ, or CR_{b}R_{c},
R₃ to R₅, and Rₐ to R_{c} are each independently a hydrogen atom, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring,
“c” is an integer of 0 to 3,
“d” is an integer of 0 to 4,
“e” is an integer of 0 to 7,
“*-” is a position where Y is bonded to L₁, or a position where Z is bonded to L₂, and
Formula 1a to Formula 1c each optionally comprise a deuterium atom.

3. The light emitting element of claim 1, wherein the amine compound represented by Formula 1 is a monoamine compound.

4. The light emitting element of claim 1, wherein the amine compound represented by Formula 1 is represented by Formula 2-1 or Formula 2-2: 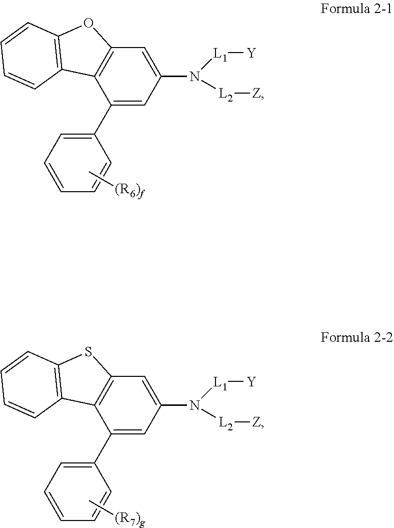
wherein in Formula 2-1 and Formula 2-2,
R₆ and R₇ are each independently a hydrogen atom, a substituted or unsubstituted alkyl group of 1 to 10 carbon atoms, a substituted or unsubstituted alkenyl group of 2 to 10 carbon atoms, or a substituted or unsubstituted aryl group of 6 to 30 ring-forming carbon atoms, and/or combined with an adjacent group to form a ring,
“f” and “g” are each independently an integer of 0 to 5,
L₁, L₂, Y and Z are as defined in Formula 1, and
Formula 2-1 and Formula 2-2 each optionally comprise a deuterium atom.

5. The light emitting element of claim 1, wherein R₁ and R₂ are hydrogen atoms.

6. The light emitting element of claim 1, wherein L₁ and L₂ are each independently a direct linkage, an unsubstituted phenylene group, or an unsubstituted biphenylene group.

7. The light emitting element of claim 1, wherein Ar_{A} is any one selected from among a1 to a9, and b1 to b9: 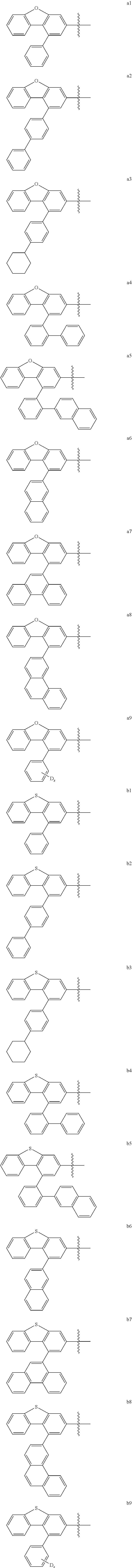
in a1 to a9, and b1 to b9, [symbol]
is a position bonded to the nitrogen atom of Formula 1, and “D” is a deuterium atom.

8. The light emitting element of claim 7, wherein at least one selected from among Ar_{B} and Ar_{C} is selected from among a1 and e1 to e67, and when present a remaining Ar_{B} or Ar_{C} is selected from among d1 to d10: 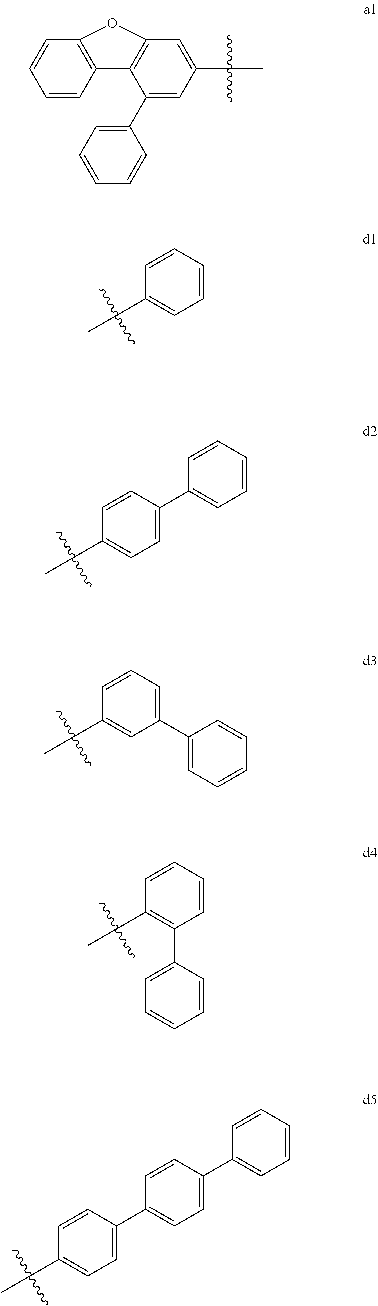 [SMILES: *c1cc(-c2ccccc2)c2c(c1)oc1ccccc12 ; *c1ccccc1 ; *c1ccc(-c2ccccc2)cc1 ; *c1cccc(-c2ccccc2)c1 ; *c1ccccc1-c1ccccc1 ; *c1ccc(-c2ccc(-c3ccccc3)cc2)cc1] 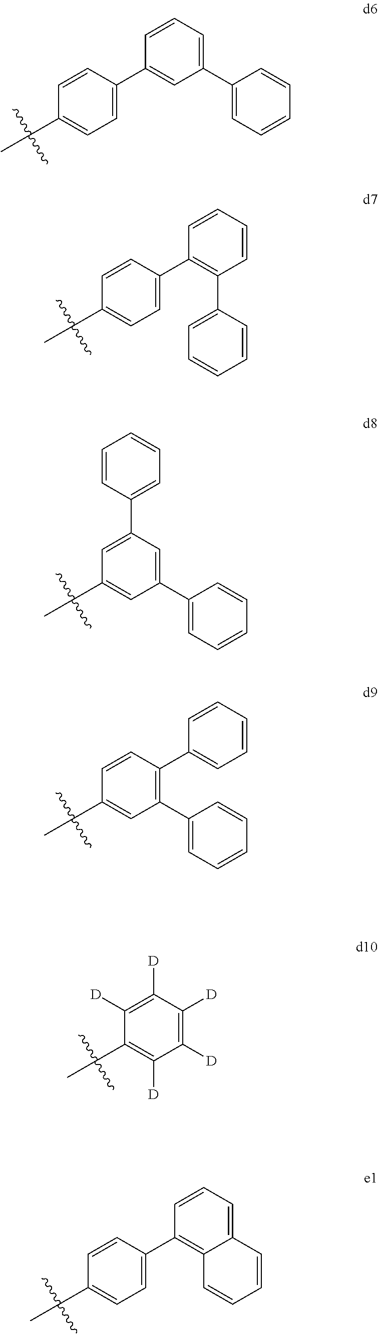 [SMILES: *c1ccc(-c2cccc(-c3ccccc3)c2)cc1 ; *c1ccc(-c2ccccc2-c2ccccc2)cc1 ; *c1cc(-c2ccccc2)cc(-c2ccccc2)c1 ; *c1ccc(-c2ccccc2)c(-c2ccccc2)c1 ; *c1c([2H])c([2H])c([2H])c([2H])c1[2H] ; *c1ccc(-c2cccc3ccccc23)cc1] 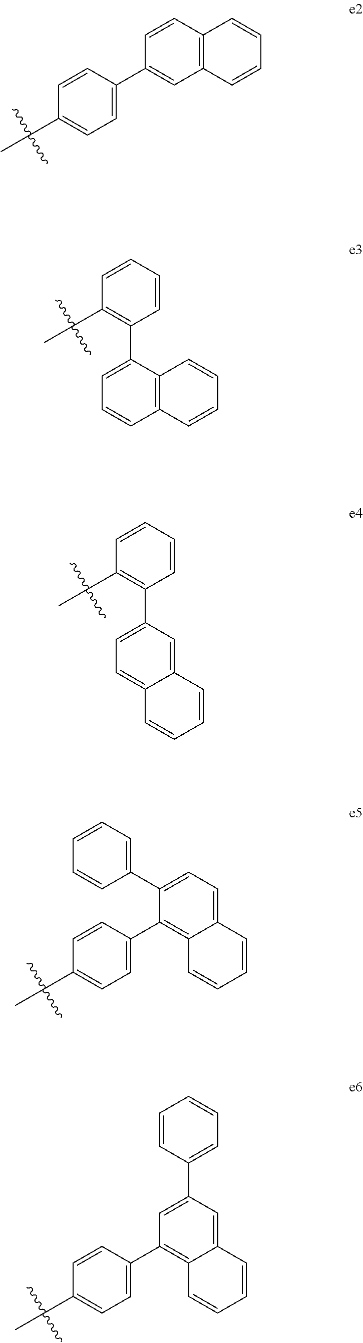 [SMILES: *c1ccc(-c2ccc3ccccc3c2)cc1 ; *c1ccccc1-c1cccc2ccccc12 ; *c1ccccc1-c1ccc2ccccc2c1 ; *c1ccc(-c2c(-c3ccccc3)ccc3ccccc23)cc1 ; *c1ccc(-c2cc(-c3ccccc3)cc3ccccc23)cc1] 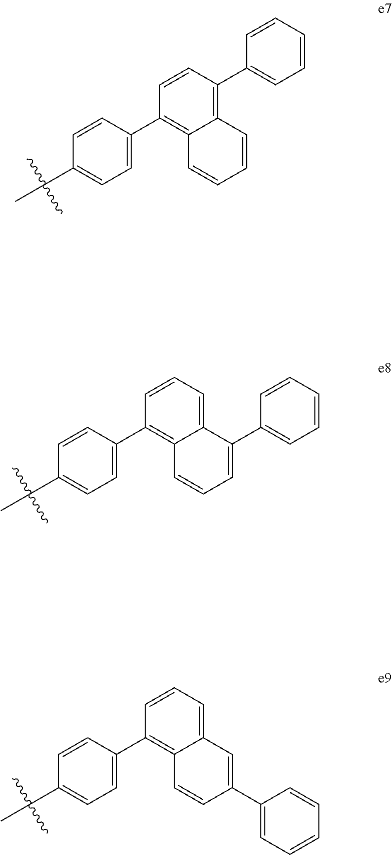 [SMILES: *c1ccc(-c2ccc(-c3ccccc3)c3ccccc23)cc1 ; *c1ccc(-c2cccc3c(-c4ccccc4)cccc23)cc1 ; *c1ccc(-c2cccc3cc(-c4ccccc4)ccc23)cc1] 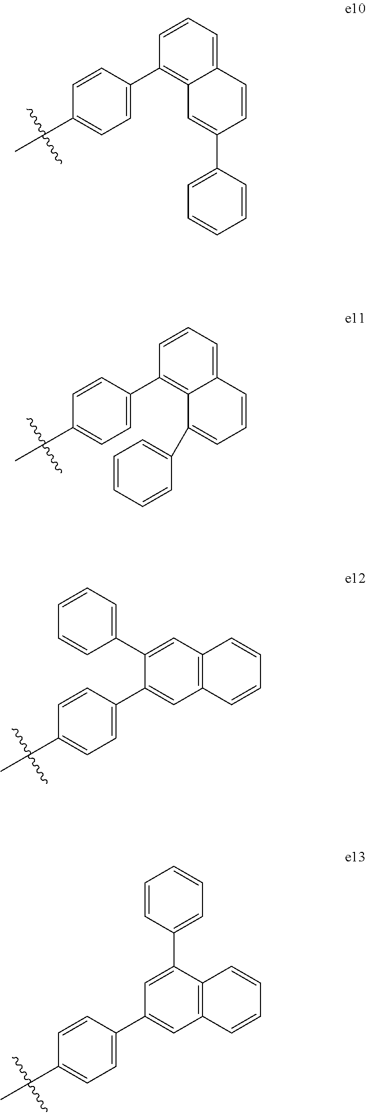 [SMILES: *c1ccc(-c2cccc3ccc(-c4ccccc4)cc23)cc1 ; *c1ccc(-c2cccc3cccc(-c4ccccc4)c23)cc1 ; *c1ccc(-c2cc3ccccc3cc2-c2ccccc2)cc1 ; *c1ccc(-c2cc(-c3ccccc3)c3ccccc3c2)cc1] 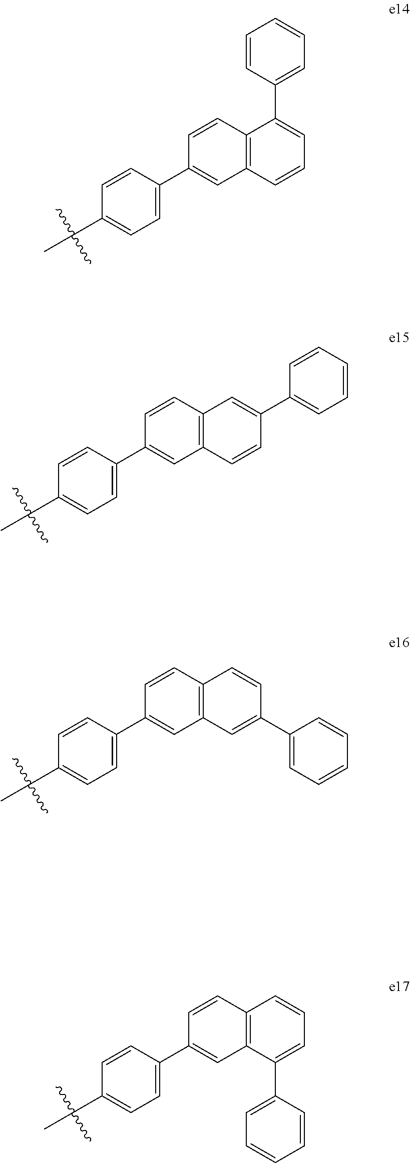 [SMILES: *c1ccc(-c2ccc3c(-c4ccccc4)cccc3c2)cc1 ; *c1ccc(-c2ccc3cc(-c4ccccc4)ccc3c2)cc1 ; *c1ccc(-c2ccc3ccc(-c4ccccc4)cc3c2)cc1 ; *c1ccc(-c2ccc3cccc(-c4ccccc4)c3c2)cc1] 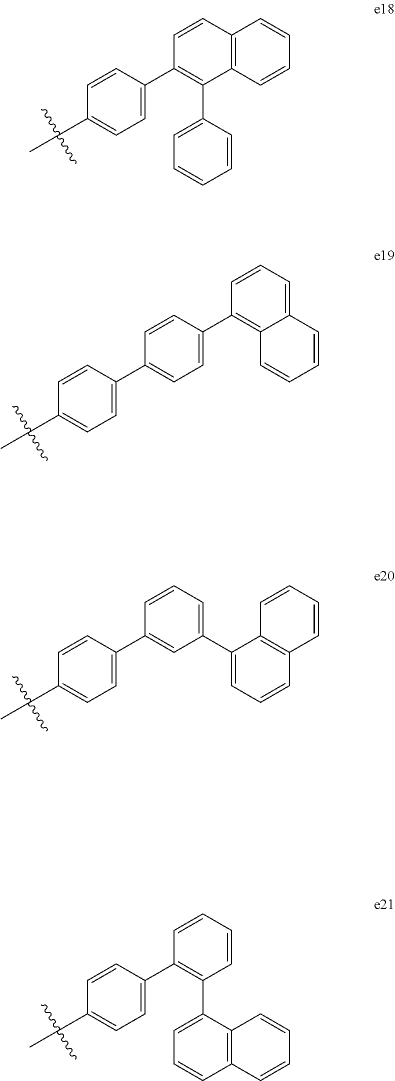 [SMILES: *c1ccc(-c2ccc3ccccc3c2-c2ccccc2)cc1 ; *c1ccc(-c2ccc(-c3cccc4ccccc34)cc2)cc1 ; *c1ccc(-c2cccc(-c3cccc4ccccc34)c2)cc1 ; *c1ccc(-c2ccccc2-c2cccc3ccccc23)cc1] 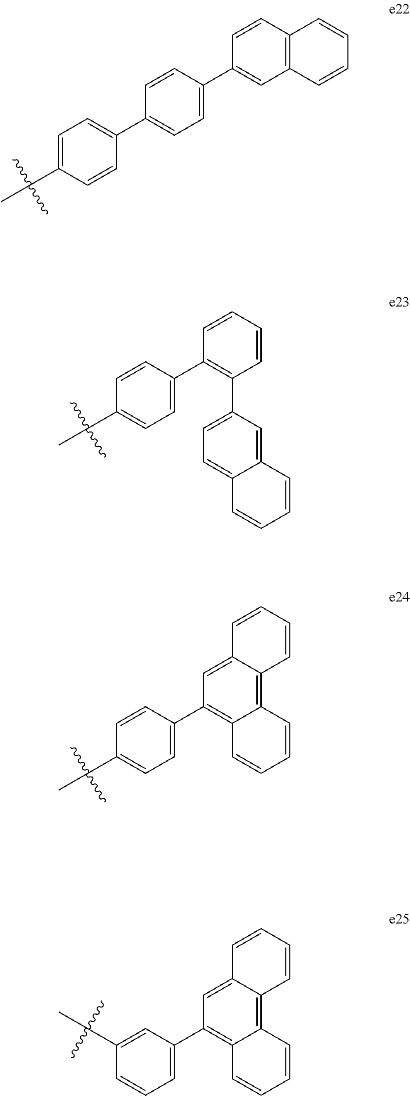 [SMILES: *c1ccc(-c2ccc(-c3ccc4ccccc4c3)cc2)cc1 ; *c1ccc(-c2ccccc2-c2ccc3ccccc3c2)cc1 ; *c1ccc(-c2cc3ccccc3c3ccccc23)cc1 ; *c1cccc(-c2cc3ccccc3c3ccccc23)c1] 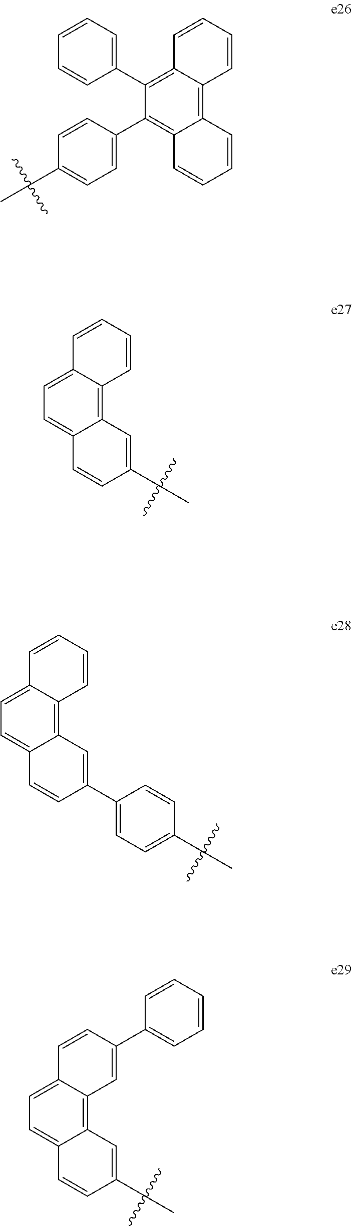 [SMILES: *c1ccc(-c2c(-c3ccccc3)c3ccccc3c3ccccc23)cc1 ; *c1ccc2ccc3ccccc3c2c1 ; *c1ccc(-c2ccc3ccc4ccccc4c3c2)cc1 ; *c1ccc2ccc3ccc(-c4ccccc4)cc3c2c1] 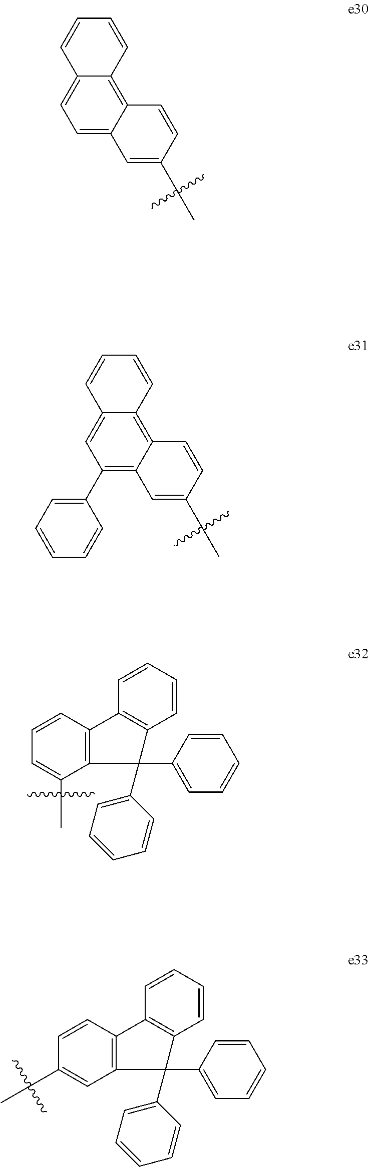 [SMILES: *c1ccc2c(ccc3ccccc32)c1 ; *c1ccc2c(c1)c(-c1ccccc1)cc1ccccc12 ; *c1cccc2c1C(c1ccccc1)(c1ccccc1)c1ccccc1-2 ; *c1ccc2c(c1)C(c1ccccc1)(c1ccccc1)c1ccccc1-2] 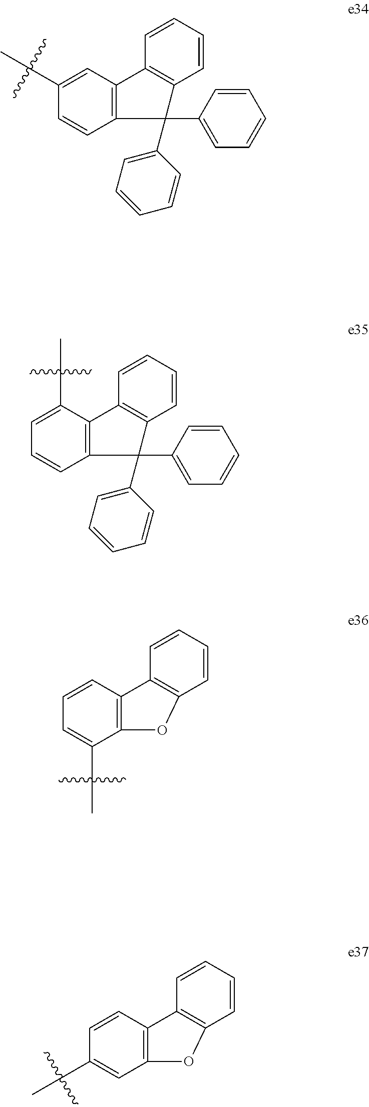 [SMILES: *c1ccc2c(c1)-c1ccccc1C2(c1ccccc1)c1ccccc1 ; *c1cccc2c1-c1ccccc1C2(c1ccccc1)c1ccccc1 ; *c1cccc2c1oc1ccccc12 ; *c1ccc2c(c1)oc1ccccc12] 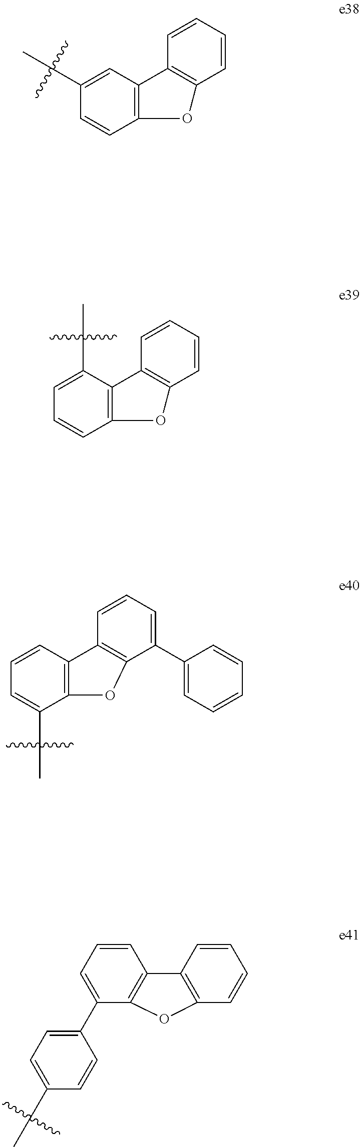 [SMILES: *c1ccc2oc3ccccc3c2c1 ; *c1cccc2oc3ccccc3c12 ; *c1cccc2c1oc1c(-c3ccccc3)cccc12 ; *c1ccc(-c2cccc3c2oc2ccccc23)cc1] 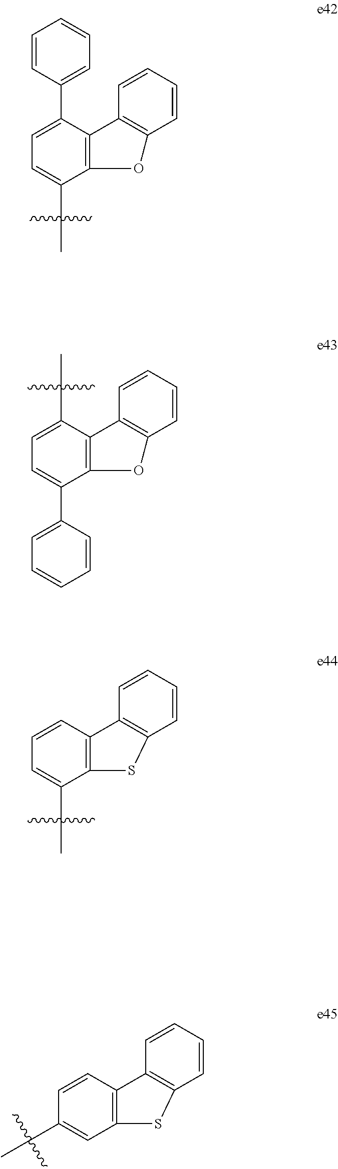 [SMILES: *c1ccc(-c2ccccc2)c2c1oc1ccccc12 ; *c1ccc(-c2ccccc2)c2oc3ccccc3c12 ; *c1cccc2c1sc1ccccc12 ; *c1ccc2c(c1)sc1ccccc12] 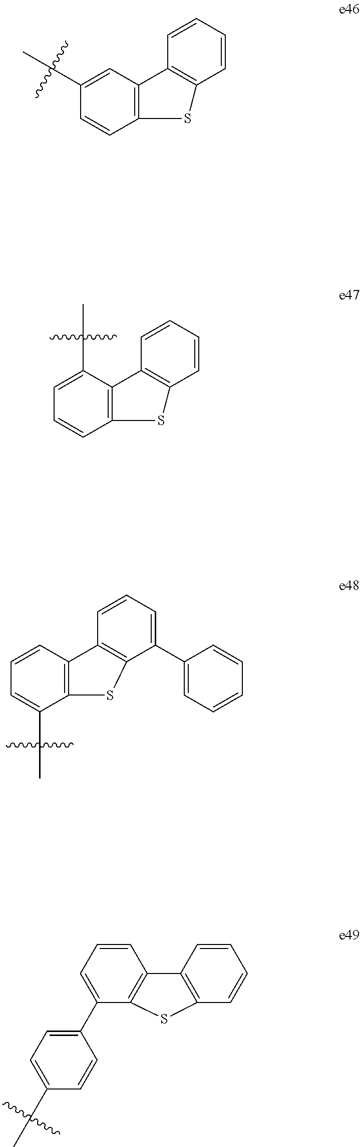 [SMILES: *c1ccc2sc3ccccc3c2c1 ; *c1cccc2sc3ccccc3c12 ; *c1cccc2c1sc1c(-c3ccccc3)cccc12 ; *c1ccc(-c2cccc3c2sc2ccccc23)cc1] 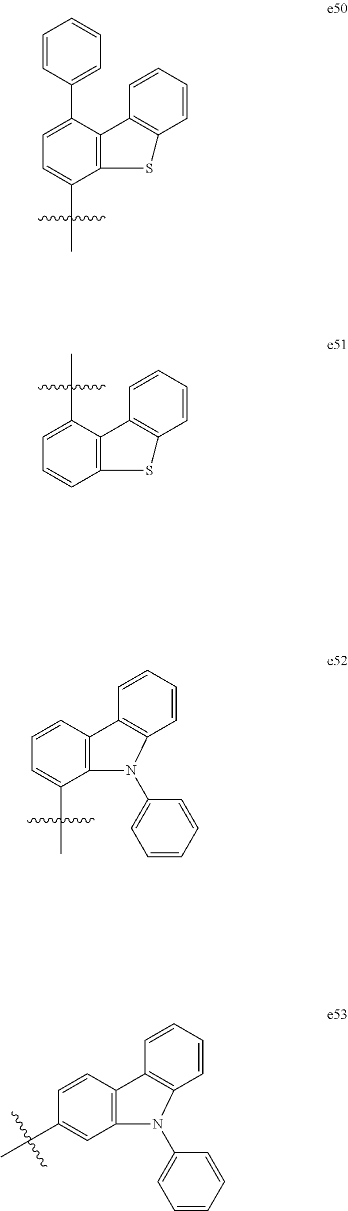 [SMILES: *c1ccc(-c2ccccc2)c2c1sc1ccccc12 ; *c1cccc2sc3ccccc3c12 ; *c1cccc2c3ccccc3n(-c3ccccc3)c12 ; *c1ccc2c3ccccc3n(-c3ccccc3)c2c1] 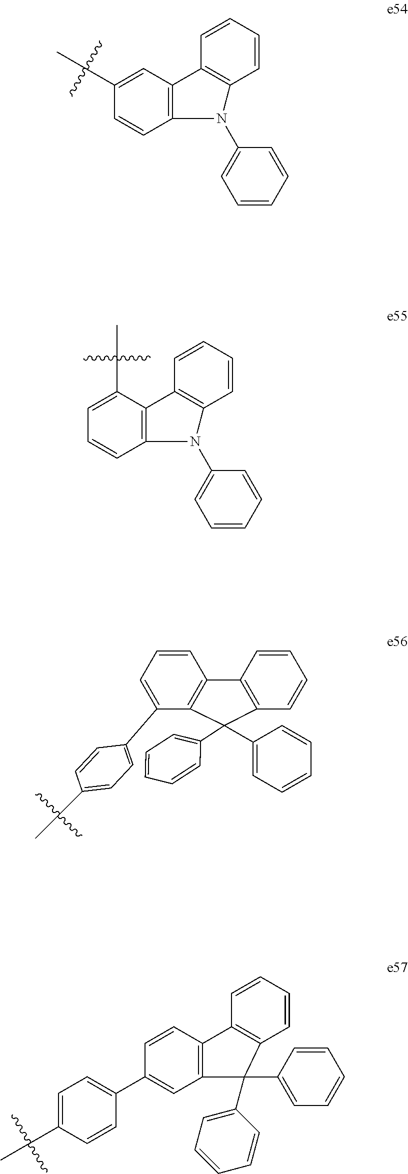 [SMILES: *c1ccc2c(c1)c1ccccc1n2-c1ccccc1 ; *c1cccc2c1c1ccccc1n2-c1ccccc1 ; *c1ccc(-c2cccc3c2C(c2ccccc2)(c2ccccc2)c2ccccc2-3)cc1 ; *c1ccc(-c2ccc3c(c2)C(c2ccccc2)(c2ccccc2)c2ccccc2-3)cc1] 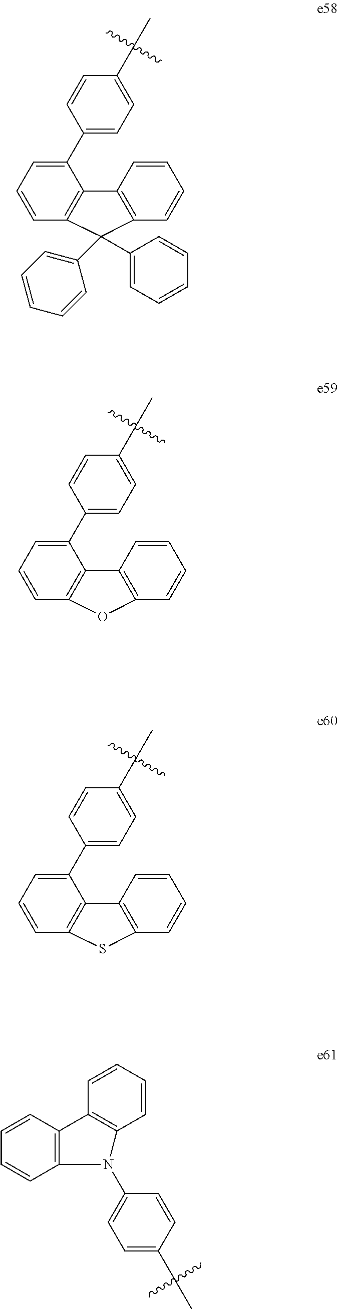 [SMILES: *c1ccc(-c2cccc3c2-c2ccccc2C3(c2ccccc2)c2ccccc2)cc1 ; *c1ccc(-c2cccc3oc4ccccc4c23)cc1 ; *c1ccc(-c2cccc3sc4ccccc4c23)cc1 ; *c1ccc(-n2c3ccccc3c3ccccc32)cc1] 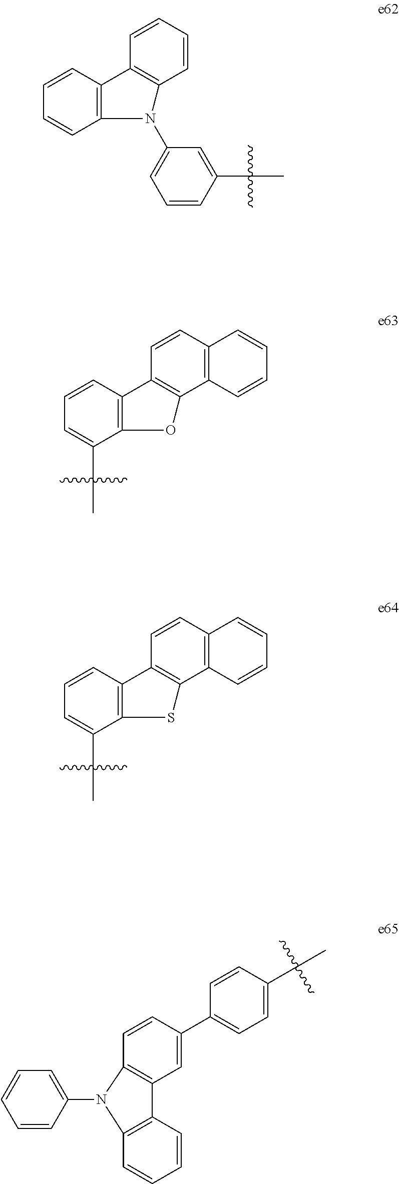 [SMILES: *c1cccc(-n2c3ccccc3c3ccccc32)c1 ; *c1cccc2c1oc1c3ccccc3ccc21 ; *c1cccc2c1sc1c3ccccc3ccc21 ; *c1ccc(-c2ccc3c(c2)c2ccccc2n3-c2ccccc2)cc1] 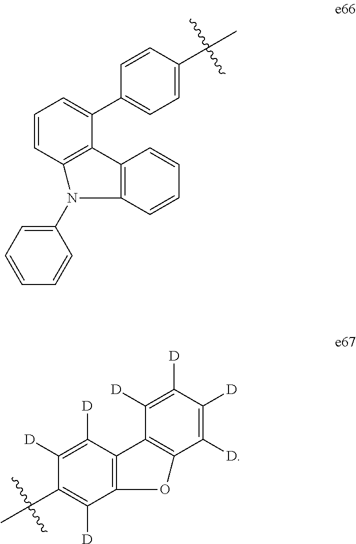
in a1, d1 to d10, and e1 to e67, [symbol]
is a position bonded to the nitrogen atom of Formula 1, and “D” is a deuterium atom.

9. The light emitting element of claim 8, wherein the amine compound represented by Formula 1 is any one selected from among compounds in Compound Group 1:
Compound Group 1

| Compound No. | Ar_{A} | Ar_{B} | Ar_{C} |
|---|---|---|---|
| 1 | a1 | e2 | e27 |
| 2 | a2 | e2 | e27 |
| 3 | a3 | e2 | e27 |
| 4 | a4 | e2 | e27 |
| 5 | a5 | e2 | e27 |
| 6 | a6 | e2 | e27 |
| 7 | a7 | e2 | e27 |
| 8 | a8 | e2 | e27 |
| 9 | a9 | e2 | e27 |
| 10 | b1 | e2 | e27 |
| 11 | b2 | e2 | e27 |
| 12 | b3 | e2 | e27 |
| 13 | b4 | e2 | e27 |
| 14 | b5 | e2 | e27 |
| 15 | b6 | e2 | e27 |
| 16 | b7 | e2 | e27 |
| 17 | b8 | e2 | e27 |
| 18 | b9 | e2 | e27 |
| 19 | a1 | d2 | a1 |
| 20 | a1 | d2 | e1 |
| 21 | a1 | d2 | e2 |
| 22 | a1 | d2 | e3 |
| 23 | a1 | d2 | e4 |
| 24 | a1 | d2 | e5 |
| 25 | a1 | d2 | e6 |
| 26 | a1 | d2 | e7 |
| 27 | a1 | d2 | e8 |
| 28 | a1 | d2 | e9 |
| 29 | a1 | d2 | e10 |
| 30 | a1 | d2 | e11 |
| 31 | a1 | d2 | e12 |
| 32 | a1 | d2 | e13 |
| 33 | a1 | d2 | e14 |
| 34 | a1 | d2 | e15 |
| 35 | a1 | d2 | e16 |
| 36 | a1 | d2 | e17 |
| 37 | a1 | d2 | e18 |
| 38 | a1 | d2 | e19 |
| 39 | a1 | d2 | e20 |
| 40 | a1 | d2 | e21 |
| 41 | a1 | d2 | e22 |
| 42 | a1 | d2 | e23 |
| 43 | a1 | d2 | e24 |
| 44 | a1 | d2 | e25 |
| 45 | a1 | d2 | e26 |
| 46 | a1 | d2 | e27 |
| 47 | a1 | d2 | e28 |
| 48 | a1 | d2 | e29 |
| 49 | a1 | d2 | e30 |
| 50 | a1 | d2 | e31 |
| 51 | a1 | d2 | e32 |
| 52 | a1 | d2 | e33 |
| 53 | a1 | d2 | e34 |
| 54 | a1 | d2 | e35 |
| 55 | a1 | d2 | e36 |
| 56 | a1 | d2 | e37 |
| 57 | a1 | d2 | e38 |
| 58 | a1 | d2 | e39 |
| 59 | a1 | d2 | e40 |
| 60 | a1 | d2 | e41 |
| 61 | a1 | d2 | e42 |
| 62 | a1 | d2 | e43 |
| 63 | a1 | d2 | e44 |
| 64 | a1 | d2 | e45 |
| 65 | a1 | d2 | e46 |
| 66 | a1 | d2 | e47 |
| 67 | a1 | d2 | e48 |
| 68 | a1 | d2 | e49 |
| 69 | a1 | d2 | e50 |
| 70 | a1 | d2 | e51 |
| 71 | a1 | d2 | e52 |
| 72 | a1 | d2 | e53 |
| 73 | a1 | d2 | e54 |
| 74 | a1 | d2 | e55 |
| 75 | a1 | d2 | e56 |
| 76 | a1 | d2 | e57 |
| 77 | a1 | d2 | e58 |
| 78 | a1 | d2 | e59 |
| 79 | a1 | d2 | e60 |
| 80 | a1 | d2 | e61 |
| 81 | a1 | d2 | e62 |
| 82 | a1 | d2 | e63 |
| 83 | a1 | d2 | e64 |
| 84 | a1 | d2 | e65 |
| 85 | a1 | d2 | e66 |
| 86 | a1 | d2 | e67 |
| 87 | a1 | d3 | a1 |
| 88 | a1 | d3 | e1 |
| 89 | a1 | d3 | e2 |
| 90 | a1 | d3 | e3 |
| 91 | a1 | d3 | e4 |
| 92 | a1 | d3 | e5 |
| 93 | a1 | d3 | e6 |
| 94 | a1 | d3 | e7 |
| 95 | a1 | d3 | e8 |
| 96 | a1 | d3 | e9 |
| 97 | a1 | d3 | e10 |
| 98 | a1 | d3 | e11 |
| 99 | a1 | d3 | e12 |
| 100 | a1 | d3 | e13 |
| 101 | a1 | d3 | e14 |
| 102 | a1 | d3 | e15 |
| 103 | a1 | d3 | e16 |
| 104 | a1 | d3 | e17 |
| 105 | a1 | d3 | e18 |
| 106 | a1 | d3 | e19 |
| 107 | a1 | d3 | e20 |
| 108 | a1 | d3 | e21 |
| 109 | a1 | d3 | e22 |
| 110 | a1 | d3 | e23 |
| 111 | a1 | d3 | e24 |
| 112 | a1 | d3 | e25 |
| 113 | a1 | d3 | e26 |
| 114 | a1 | d3 | e27 |
| 115 | a1 | d3 | e28 |
| 116 | a1 | d3 | e29 |
| 117 | a1 | d3 | e30 |
| 118 | a1 | d3 | e31 |
| 119 | a1 | d3 | e32 |
| 120 | a1 | d3 | e33 |
| 121 | a1 | d3 | e34 |
| 122 | a1 | d3 | e35 |
| 123 | a1 | d3 | e36 |
| 124 | a1 | d3 | e37 |
| 125 | a1 | d3 | e38 |
| 126 | a1 | d3 | e39 |
| 127 | a1 | d3 | e40 |
| 128 | a1 | d3 | e41 |
| 129 | a1 | d3 | e42 |
| 130 | a1 | d3 | e43 |
| 131 | a1 | d3 | e44 |
| 132 | a1 | d3 | e45 |
| 133 | a1 | d3 | e46 |
| 134 | a1 | d3 | e47 |
| 135 | a1 | d3 | e48 |
| 136 | a1 | d3 | e49 |
| 137 | a1 | d3 | e50 |
| 138 | a1 | d3 | e51 |
| 139 | a1 | d3 | e52 |
| 140 | a1 | d3 | e53 |
| 141 | a1 | d3 | e54 |
| 142 | a1 | d3 | e55 |
| 143 | a1 | d3 | e56 |
| 144 | a1 | d3 | e57 |
| 145 | a1 | d3 | e58 |
| 146 | a1 | d3 | e59 |
| 147 | a1 | d3 | e60 |
| 148 | a1 | d3 | e61 |
| 149 | a1 | d3 | e62 |
| 150 | a1 | d3 | e63 |
| 151 | a1 | d3 | e64 |
| 152 | a1 | d3 | e65 |
| 153 | a1 | d3 | e66 |
| 154 | a1 | d3 | e67 |
| 155 | a1 | d7 | a1 |
| 156 | a1 | d7 | e1 |
| 157 | a1 | d7 | e1 |
| 158 | a1 | d7 | e2 |
| 159 | a1 | d7 | e3 |
| 160 | a1 | d7 | e4 |
| 161 | a1 | d7 | e5 |
| 162 | a1 | d7 | e6 |
| 163 | a1 | d7 | e7 |
| 164 | a1 | d7 | e8 |
| 165 | a1 | d7 | e9 |
| 166 | a1 | d7 | e10 |
| 167 | a1 | d7 | e11 |
| 168 | a1 | d7 | e12 |
| 169 | a1 | d7 | e13 |
| 170 | a1 | d7 | e14 |
| 171 | a1 | d7 | e15 |
| 172 | a1 | d7 | e16 |
| 173 | a1 | d7 | e17 |
| 174 | a1 | d7 | e18 |
| 175 | a1 | d7 | e19 |
| 176 | a1 | d7 | e20 |
| 177 | a1 | d7 | e21 |
| 178 | a1 | d7 | e22 |
| 179 | a1 | d7 | e23 |
| 180 | a1 | d7 | e24 |
| 181 | a1 | d7 | e25 |
| 182 | a1 | d7 | e26 |
| 183 | a1 | d7 | e27 |
| 184 | a1 | d7 | e28 |
| 185 | a1 | d7 | e29 |
| 186 | a1 | d7 | e30 |
| 187 | a1 | d7 | e31 |
| 188 | a1 | d7 | e32 |
| 189 | a1 | d7 | e33 |
| 190 | a1 | d7 | e34 |
| 191 | a1 | d7 | e35 |
| 192 | a1 | d7 | e36 |
| 193 | a1 | d7 | e37 |
| 194 | a1 | d7 | e38 |
| 195 | a1 | d7 | e39 |
| 196 | a1 | d7 | e40 |
| 197 | a1 | d7 | e41 |
| 198 | a1 | d7 | e42 |
| 199 | a1 | d7 | e43 |
| 200 | a1 | d7 | e44 |
| Compound No. | Ar_{A} | Ar_{B} | Ar_{C} |
|---|---|---|---|
| 201 | a1 | d7 | e45 |
| 202 | a1 | d7 | e46 |
| 203 | a1 | d7 | e47 |
| 204 | a1 | d7 | e48 |
| 205 | a1 | d7 | e49 |
| 206 | a1 | d7 | e50 |
| 207 | a1 | d7 | e51 |
| 208 | a1 | d7 | e52 |
| 209 | a1 | d7 | e53 |
| 210 | a1 | d7 | e54 |
| 211 | a1 | d7 | e55 |
| 212 | a1 | d7 | e56 |
| 213 | a1 | d7 | e57 |
| 214 | a1 | d7 | e58 |
| 215 | a1 | d7 | e59 |
| 216 | a1 | d7 | e60 |
| 217 | a1 | d7 | e61 |
| 218 | a1 | d7 | e62 |
| 219 | a1 | d7 | e63 |
| 220 | a1 | d7 | e64 |
| 221 | a1 | d7 | e65 |
| 222 | a1 | d7 | e66 |
| 223 | a1 | d7 | e67 |
| 224 | a1 | d9 | a1 |
| 225 | a1 | d9 | e1 |
| 226 | a1 | d9 | e1 |
| 227 | a1 | d9 | e2 |
| 228 | a1 | d9 | e3 |
| 229 | a1 | d9 | e4 |
| 230 | a1 | d9 | e5 |
| 231 | a1 | d9 | e6 |
| 232 | a1 | d9 | e7 |
| 233 | a1 | d9 | e8 |
| 234 | a1 | d9 | e9 |
| 235 | a1 | d9 | e10 |
| 236 | a1 | d9 | e11 |
| 237 | a1 | d9 | e12 |
| 238 | a1 | d9 | e13 |
| 239 | a1 | d9 | e14 |
| 240 | a1 | d9 | e15 |
| 241 | a1 | d9 | e16 |
| 242 | a1 | d9 | e17 |
| 243 | a1 | d9 | e18 |
| 244 | a1 | d9 | e19 |
| 245 | a1 | d9 | e20 |
| 246 | a1 | d9 | e21 |
| 247 | a1 | d9 | e22 |
| 248 | a1 | d9 | e23 |
| 249 | a1 | d9 | e24 |
| 250 | a1 | d9 | e25 |
| 251 | a1 | d9 | e26 |
| 252 | a1 | d9 | e27 |
| 253 | a1 | d9 | e28 |
| 254 | a1 | d9 | e29 |
| 255 | a1 | d9 | e30 |
| 256 | a1 | d9 | e31 |
| 257 | a1 | d9 | e32 |
| 258 | a1 | d9 | e33 |
| 259 | a1 | d9 | e34 |
| 260 | a1 | d9 | e35 |
| 261 | a1 | d9 | e36 |
| 262 | a1 | d9 | e37 |
| 263 | a1 | d9 | e38 |
| 264 | a1 | d9 | e39 |
| 265 | a1 | d9 | e40 |
| 266 | a1 | d9 | e41 |
| 267 | a1 | d9 | e42 |
| 268 | a1 | d9 | e43 |
| 269 | a1 | d9 | e44 |
| 270 | a1 | d9 | e45 |
| 271 | a1 | d9 | e46 |
| 272 | a1 | d9 | e47 |
| 273 | a1 | d9 | e48 |
| 274 | a1 | d9 | e49 |
| 275 | a1 | d9 | e50 |
| 276 | a1 | d9 | e51 |
| 277 | a1 | d9 | e52 |
| 278 | a1 | d9 | e53 |
| 279 | a1 | d9 | e54 |
| 280 | a1 | d9 | e55 |
| 281 | a1 | d9 | e56 |
| 282 | a1 | d9 | e57 |
| 283 | a1 | d9 | e58 |
| 284 | a1 | d9 | e59 |
| 285 | a1 | d9 | e60 |
| 286 | a1 | d9 | e61 |
| 287 | a1 | d9 | e62 |
| 288 | a1 | d9 | e63 |
| 289 | a1 | d9 | e64 |
| 290 | a1 | d9 | e65 |
| 291 | a1 | d9 | e66 |
| 292 | a1 | d9 | e67 |
| 293 | a1 | e1 | a1 |
| 294 | a1 | e1 | d1 |
| 295 | a1 | e1 | d1 |
| 296 | a1 | e1 | d4 |
| 297 | a1 | e1 | d5 |
| 298 | a1 | e1 | d6 |
| 299 | a1 | e1 | d8 |
| 300 | a1 | e1 | d10 |
| 301 | a1 | e1 | e1 |
| 302 | a1 | e1 | e1 |
| 303 | a1 | e1 | e2 |
| 304 | a1 | e1 | e3 |
| 305 | a1 | e1 | e4 |
| 306 | a1 | e1 | e5 |
| 307 | a1 | e1 | e6 |
| 308 | a1 | e1 | e7 |
| 309 | a1 | e1 | e8 |
| 310 | a1 | e1 | e9 |
| 311 | a1 | e1 | e10 |
| 312 | a1 | e1 | e11 |
| 313 | a1 | e1 | e12 |
| 314 | a1 | e1 | e13 |
| 315 | a1 | e1 | e14 |
| 316 | a1 | e1 | e15 |
| 317 | a1 | e1 | e16 |
| 318 | a1 | e1 | e17 |
| 319 | a1 | e1 | e18 |
| 320 | a1 | e1 | e19 |
| 321 | a1 | e1 | e20 |
| 322 | a1 | e1 | e21 |
| 323 | a1 | e1 | e22 |
| 324 | a1 | e1 | e23 |
| 325 | a1 | e1 | e24 |
| 326 | a1 | e1 | e25 |
| 327 | a1 | e1 | e26 |
| 328 | a1 | e1 | e27 |
| 329 | a1 | e1 | e28 |
| 330 | a1 | e1 | e29 |
| 331 | a1 | e1 | e30 |
| 332 | a1 | e1 | e31 |
| 333 | a1 | e1 | e32 |
| 334 | a1 | e1 | e33 |
| 335 | a1 | e1 | e34 |
| 336 | a1 | e1 | e35 |
| 337 | a1 | e1 | e36 |
| 338 | a1 | e1 | e37 |
| 339 | a1 | e1 | e38 |
| 340 | a1 | e1 | e39 |
| 341 | a1 | e1 | e40 |
| 342 | a1 | e1 | e41 |
| 343 | a1 | e1 | e42 |
| 344 | a1 | e1 | e43 |
| 345 | a1 | e1 | e44 |
| 346 | a1 | e1 | e45 |
| 347 | a1 | e1 | e46 |
| 348 | a1 | e1 | e47 |
| 349 | a1 | e1 | e48 |
| 350 | a1 | e1 | e49 |
| 351 | a1 | e1 | e50 |
| 352 | a1 | e1 | e51 |
| 353 | a1 | e1 | e52 |
| 354 | a1 | e1 | e53 |
| 355 | a1 | e1 | e54 |
| 356 | a1 | e1 | e55 |
| 357 | a1 | e1 | e56 |
| 358 | a1 | e1 | e57 |
| 359 | a1 | e1 | e58 |
| 360 | a1 | e1 | e59 |
| 361 | a1 | e1 | e60 |
| 362 | a1 | e1 | e61 |
| 363 | a1 | e1 | e62 |
| 364 | a1 | e1 | e63 |
| 365 | a1 | e1 | e64 |
| 366 | a1 | e1 | e65 |
| 367 | a1 | e1 | e66 |
| 368 | a1 | e1 | e67 |
| 369 | a1 | e2 | a1 |
| 370 | a1 | e2 | d1 |
| 371 | a1 | e2 | d1 |
| 372 | a1 | e2 | d4 |
| 373 | a1 | e2 | d5 |
| 374 | a1 | e2 | d6 |
| 375 | a1 | e2 | d8 |
| 376 | a1 | e2 | d10 |
| 377 | a1 | e2 | e2 |
| 378 | a1 | e2 | e3 |
| 379 | a1 | e2 | e4 |
| 380 | a1 | e2 | e5 |
| 381 | a1 | e2 | e6 |
| 382 | a1 | e2 | e7 |
| 383 | a1 | e2 | e8 |
| 384 | a1 | e2 | e9 |
| 385 | a1 | e2 | e10 |
| 386 | a1 | e2 | e11 |
| 387 | a1 | e2 | e12 |
| 388 | a1 | e2 | e13 |
| 389 | a1 | e2 | e14 |
| 390 | a1 | e2 | e15 |
| 391 | a1 | e2 | e16 |
| 392 | a1 | e2 | e17 |
| 393 | a1 | e2 | e18 |
| 394 | a1 | e2 | e19 |
| 395 | a1 | e2 | e20 |
| 396 | a1 | e2 | e21 |
| 397 | a1 | e2 | e22 |
| 398 | a1 | e2 | e23 |
| 399 | a1 | e2 | e24 |
| 400 | a1 | e2 | e25 |
| Compound No. | Ar_{A} | Ar_{B} | Ar_{C} |
|---|---|---|---|
| 401 | a1 | e2 | e26 |
| 402 | a1 | e2 | e28 |
| 403 | a1 | e2 | e29 |
| 404 | a1 | e2 | e30 |
| 405 | a1 | e2 | e31 |
| 406 | a1 | e2 | e32 |
| 407 | a1 | e2 | e33 |
| 408 | a1 | e2 | e34 |
| 409 | a1 | e2 | e35 |
| 410 | a1 | e2 | e36 |
| 411 | a1 | e2 | e37 |
| 412 | a1 | e2 | e38 |
| 413 | a1 | e2 | e39 |
| 414 | a1 | e2 | e40 |
| 415 | a1 | e2 | e41 |
| 416 | a1 | e2 | e42 |
| 417 | a1 | e2 | e43 |
| 418 | a1 | e2 | e44 |
| 419 | a1 | e2 | e45 |
| 420 | a1 | e2 | e46 |
| 421 | a1 | e2 | e47 |
| 422 | a1 | e2 | e48 |
| 423 | a1 | e2 | e49 |
| 424 | a1 | e2 | e50 |
| 425 | a1 | e2 | e51 |
| 426 | a1 | e2 | e52 |
| 427 | a1 | e2 | e53 |
| 428 | a1 | e2 | e54 |
| 429 | a1 | e2 | e55 |
| 430 | a1 | e2 | e56 |
| 431 | a1 | e2 | e57 |
| 432 | a1 | e2 | e58 |
| 433 | a1 | e2 | e59 |
| 434 | a1 | e2 | e60 |
| 435 | a1 | e2 | e61 |
| 436 | a1 | e2 | e62 |
| 437 | a1 | e2 | e63 |
| 438 | a1 | e2 | e64 |
| 439 | a1 | e2 | e65 |
| 440 | a1 | e2 | e66 |
| 441 | a1 | e2 | e67 |
| 442 | a1 | e24 | a1 |
| 443 | a1 | e24 | d1 |
| 444 | a1 | e24 | d1 |
| 445 | a1 | e24 | d4 |
| 446 | a1 | e24 | d5 |
| 447 | a1 | e24 | d6 |
| 448 | a1 | e24 | d8 |
| 449 | a1 | e24 | d10 |
| 450 | a1 | e24 | e3 |
| 451 | a1 | e24 | e4 |
| 452 | a1 | e24 | e5 |
| 453 | a1 | e24 | e6 |
| 454 | a1 | e24 | e7 |
| 455 | a1 | e24 | e8 |
| 456 | a1 | e24 | e9 |
| 457 | a1 | e24 | e10 |
| 458 | a1 | e24 | e11 |
| 459 | a1 | e24 | e12 |
| 460 | a1 | e24 | e13 |
| 461 | a1 | e24 | e14 |
| 462 | a1 | e24 | e15 |
| 463 | a1 | e24 | e16 |
| 464 | a1 | e24 | e17 |
| 465 | a1 | e24 | e18 |
| 466 | a1 | e24 | e19 |
| 467 | a1 | e24 | e20 |
| 468 | a1 | e24 | e21 |
| 469 | a1 | e24 | e22 |
| 470 | a1 | e24 | e23 |
| 471 | a1 | e24 | e24 |
| 472 | a1 | e24 | e25 |
| 473 | a1 | e24 | e26 |
| 474 | a1 | e24 | e27 |
| 475 | a1 | e24 | e28 |
| 476 | a1 | e24 | e29 |
| 477 | a1 | e24 | e30 |
| 478 | a1 | e24 | e31 |
| 479 | a1 | e24 | e32 |
| 480 | a1 | e24 | e33 |
| 481 | a1 | e24 | e34 |
| 482 | a1 | e24 | e35 |
| 483 | a1 | e24 | e36 |
| 484 | a1 | e24 | e37 |
| 485 | a1 | e24 | e38 |
| 486 | a1 | e24 | e39 |
| 487 | a1 | e24 | e40 |
| 488 | a1 | e24 | e41 |
| 489 | a1 | e24 | e42 |
| 490 | a1 | e24 | e43 |
| 491 | a1 | e24 | e44 |
| 492 | a1 | e24 | e45 |
| 493 | a1 | e24 | e46 |
| 494 | a1 | e24 | e47 |
| 495 | a1 | e24 | e48 |
| 496 | a1 | e24 | e49 |
| 497 | a1 | e24 | e50 |
| 498 | a1 | e24 | e51 |
| 499 | a1 | e24 | e52 |
| 500 | a1 | e24 | e53 |
| 501 | a1 | e24 | e54 |
| 502 | a1 | e24 | e55 |
| 503 | a1 | e24 | e56 |
| 504 | a1 | e24 | e57 |
| 505 | a1 | e24 | e58 |
| 506 | a1 | e24 | e59 |
| 507 | a1 | e24 | e60 |
| 508 | a1 | e24 | e61 |
| 509 | a1 | e24 | e62 |
| 510 | a1 | e24 | e63 |
| 511 | a1 | e24 | e64 |
| 512 | a1 | e24 | e65 |
| 513 | a1 | e24 | e66 |
| 514 | a1 | e24 | e67 |
| 515 | a1 | e27 | a1 |
| 516 | a1 | e27 | d1 |
| 517 | a1 | e27 | d1 |
| 518 | a1 | e27 | d4 |
| 519 | a1 | e27 | d5 |
| 520 | a1 | e27 | d6 |
| 521 | a1 | e27 | d8 |
| 522 | a1 | e27 | d10 |
| 523 | a1 | e27 | e3 |
| 524 | a1 | e27 | e4 |
| 525 | a1 | e27 | e5 |
| 526 | a1 | e27 | e6 |
| 527 | a1 | e27 | e7 |
| 528 | a1 | e27 | e8 |
| 529 | a1 | e27 | e9 |
| 530 | a1 | e27 | e10 |
| 531 | a1 | e27 | e11 |
| 532 | a1 | e27 | e12 |
| 533 | a1 | e27 | e13 |
| 534 | a1 | e27 | e14 |
| 535 | a1 | e27 | e15 |
| 536 | a1 | e27 | e16 |
| 537 | a1 | e27 | e17 |
| 538 | a1 | e27 | e18 |
| 539 | a1 | e27 | e19 |
| 540 | a1 | e27 | e20 |
| 541 | a1 | e27 | e21 |
| 542 | a1 | e27 | e22 |
| 543 | a1 | e27 | e23 |
| 544 | a1 | e27 | e25 |
| 545 | a1 | e27 | e26 |
| 546 | a1 | e27 | e27 |
| 547 | a1 | e27 | e28 |
| 548 | a1 | e27 | e29 |
| 549 | a1 | e27 | e30 |
| 550 | a1 | e27 | e31 |
| 551 | a1 | e27 | e32 |
| 552 | a1 | e27 | e33 |
| 553 | a1 | e27 | e34 |
| 554 | a1 | e27 | e35 |
| 555 | a1 | e27 | e36 |
| 556 | a1 | e27 | e37 |
| 557 | a1 | e27 | e38 |
| 558 | a1 | e27 | e39 |
| 559 | a1 | e27 | e40 |
| 560 | a1 | e27 | e41 |
| 561 | a1 | e27 | e42 |
| 562 | a1 | e27 | e43 |
| 563 | a1 | e27 | e44 |
| 564 | a1 | e27 | e45 |
| 565 | a1 | e27 | e46 |
| 566 | a1 | e27 | e47 |
| 567 | a1 | e27 | e48 |
| 568 | a1 | e27 | e49 |
| 569 | a1 | e27 | e50 |
| 570 | a1 | e27 | e51 |
| 571 | a1 | e27 | e52 |
| 572 | a1 | e27 | e53 |
| 573 | a1 | e27 | e54 |
| 574 | a1 | e27 | e55 |
| 575 | a1 | e27 | e56 |
| 576 | a1 | e27 | e57 |
| 577 | a1 | e27 | e58 |
| 578 | a1 | e27 | e59 |
| 579 | a1 | e27 | e60 |
| 580 | a1 | e27 | e61 |
| 581 | a1 | e27 | e62 |
| 582 | a1 | e27 | e63 |
| 583 | a1 | e27 | e64 |
| 584 | a1 | e27 | e65 |
| 585 | a1 | e27 | e66 |
| 586 | a1 | e27 | e67 |
| 587 | a1 | e33 | a1 |
| 588 | a1 | e33 | d1 |
| 589 | a1 | e33 | d1 |
| 590 | a1 | e33 | d4 |
| 591 | a1 | e33 | d5 |
| 592 | a1 | e33 | d6 |
| 593 | a1 | e33 | d8 |
| 594 | a1 | e33 | d10 |
| 595 | a1 | e33 | e3 |
| 596 | a1 | e33 | e4 |
| 597 | a1 | e33 | e5 |
| 598 | a1 | e33 | e6 |
| 599 | a1 | e33 | e7 |
| 600 | a1 | e33 | e8 |
| Compound No. | Ar_{A} | Ar_{B} | Ar_{C} |
|---|---|---|---|
| 801 | a1 | e41 | d4 |
| 802 | a1 | e41 | d5 |
| 803 | a1 | e41 | d6 |
| 804 | a1 | e41 | d8 |
| 805 | a1 | e41 | d10 |
| 806 | a1 | e41 | e3 |
| 807 | a1 | e41 | e4 |
| 808 | a1 | e41 | e5 |
| 809 | a1 | e41 | e6 |
| 810 | a1 | e41 | e7 |
| 811 | a1 | e41 | e8 |
| 812 | a1 | e41 | e9 |
| 813 | a1 | e41 | e10 |
| 814 | a1 | e41 | e11 |
| 815 | a1 | e41 | e12 |
| 816 | a1 | e41 | e13 |
| 817 | a1 | e41 | e14 |
| 818 | a1 | e41 | e15 |
| 819 | a1 | e41 | e16 |
| 820 | a1 | e41 | e17 |
| 821 | a1 | e41 | e18 |
| 822 | a1 | e41 | e19 |
| 823 | a1 | e41 | e20 |
| 824 | a1 | e41 | e21 |
| 825 | a1 | e41 | e22 |
| 826 | a1 | e41 | e23 |
| 827 | a1 | e41 | e25 |
| 828 | a1 | e41 | e26 |
| 829 | a1 | e41 | e28 |
| 830 | a1 | e41 | e29 |
| 831 | a1 | e41 | e30 |
| 832 | a1 | e41 | e31 |
| 833 | a1 | e41 | e32 |
| 834 | a1 | e41 | e34 |
| 835 | a1 | e41 | e35 |
| 836 | a1 | e41 | e38 |
| 837 | a1 | e41 | e39 |
| 838 | a1 | e41 | e40 |
| 839 | a1 | e41 | e41 |
| 840 | a1 | e41 | e42 |
| 841 | a1 | e41 | e43 |
| 842 | a1 | e41 | e44 |
| 843 | a1 | e41 | e45 |
| 844 | a1 | e41 | e46 |
| 845 | a1 | e41 | e47 |
| 846 | a1 | e41 | e48 |
| 847 | a1 | e41 | e49 |
| 848 | a1 | e41 | e50 |
| 849 | a1 | e41 | e51 |
| 850 | a1 | e41 | e52 |
| 851 | a1 | e41 | e53 |
| 852 | a1 | e41 | e54 |
| 853 | a1 | e41 | e55 |
| 854 | a1 | e41 | e56 |
| 855 | a1 | e41 | e57 |
| 856 | a1 | e41 | e58 |
| 857 | a1 | e41 | e59 |
| 858 | a1 | e41 | e60 |
| 859 | a1 | e41 | e61 |
| 860 | a1 | e41 | e62 |
| 861 | a1 | e41 | e63 |
| 862 | a1 | e41 | e64 |
| 863 | a1 | e41 | e65 |
| 864 | a1 | e41 | e66 |
| 865 | a1 | e41 | e67 |
| 866 | a1 | e59 | a1 |
| 867 | a1 | e59 | d1 |
| 868 | a1 | e59 | d1 |
| 869 | a1 | e59 | d4 |
| 870 | a1 | e59 | d5 |
| 871 | a1 | e59 | d6 |
| 872 | a1 | e59 | d8 |
| 873 | a1 | e59 | d10 |
| 874 | a1 | e59 | e3 |
| 875 | a1 | e59 | e4 |
| 876 | a1 | e59 | e5 |
| 877 | a1 | e59 | e6 |
| 878 | a1 | e59 | e7 |
| 879 | a1 | e59 | e8 |
| 880 | a1 | e59 | e9 |
| 881 | a1 | e59 | e10 |
| 882 | a1 | e59 | e11 |
| 883 | a1 | e59 | e12 |
| 884 | a1 | e59 | e13 |
| 885 | a1 | e59 | e14 |
| 886 | a1 | e59 | e15 |
| 887 | a1 | e59 | e16 |
| 888 | a1 | e59 | e17 |
| 889 | a1 | e59 | e18 |
| 890 | a1 | e59 | e19 |
| 891 | a1 | e59 | e20 |
| 892 | a1 | e59 | e21 |
| 893 | a1 | e59 | e22 |
| 894 | a1 | e59 | e23 |
| 895 | a1 | e59 | e25 |
| 896 | a1 | e59 | e26 |
| 897 | a1 | e59 | e28 |
| 898 | a1 | e59 | e29 |
| 899 | a1 | e59 | e30 |
| 900 | a1 | e59 | e31 |
| 901 | a1 | e59 | e32 |
| 902 | a1 | e59 | e34 |
| 903 | a1 | e59 | e35 |
| 904 | a1 | e59 | e38 |
| 905 | a1 | e59 | e39 |
| 906 | a1 | e59 | e40 |
| 907 | a1 | e59 | e42 |
| 908 | a1 | e59 | e43 |
| 909 | a1 | e59 | e44 |
| 910 | a1 | e59 | e45 |
| 911 | a1 | e59 | e46 |
| 912 | a1 | e59 | e47 |
| 913 | a1 | e59 | e48 |
| 914 | a1 | e59 | e49 |
| 915 | a1 | e59 | e50 |
| 916 | a1 | e59 | e51 |
| 917 | a1 | e59 | e52 |
| 918 | a1 | e59 | e53 |
| 919 | a1 | e59 | e54 |
| 920 | a1 | e59 | e55 |
| 921 | a1 | e59 | e56 |
| 922 | a1 | e59 | e57 |
| 923 | a1 | e59 | e58 |
| 924 | a1 | e59 | e59 |
| 925 | a1 | e59 | e60 |
| 926 | a1 | e59 | e61 |
| 927 | a1 | e59 | e62 |
| 928 | a1 | e59 | e63 |
| 929 | a1 | e59 | e64 |
| 930 | a1 | e59 | e65 |
| 931 | a1 | e59 | e66 |
| 932 | a1 | e59 | e67 |
| 933 | a1 | e62 | a1 |
| 934 | a1 | e62 | d1 |
| 935 | a1 | e62 | d1 |
| 936 | a1 | e62 | d5 |
| 937 | a1 | e62 | d6 |
| 938 | a1 | e62 | d8 |
| 939 | a1 | e62 | d10 |
| 940 | a1 | e62 | e5 |
| 941 | a1 | e62 | e6 |
| 942 | a1 | e62 | e7 |
| 943 | a1 | e62 | e8 |
| 944 | a1 | e62 | e9 |
| 945 | a1 | e62 | e10 |
| 946 | a1 | e62 | e11 |
| 947 | a1 | e62 | e12 |
| 948 | a1 | e62 | e13 |
| 949 | a1 | e62 | e14 |
| 950 | a1 | e62 | e15 |
| 951 | a1 | e62 | e16 |
| 952 | a1 | e62 | e17 |
| 953 | a1 | e62 | e18 |
| 954 | a1 | e62 | e19 |
| 955 | a1 | e62 | e20 |
| 956 | a1 | e62 | e21 |
| 957 | a1 | e62 | e22 |
| 958 | a1 | e62 | e23 |
| 959 | a1 | e62 | e25 |
| 960 | a1 | e62 | e26 |
| 961 | a1 | e62 | e28 |
| 962 | a1 | e62 | e29 |
| 963 | a1 | e62 | e30 |
| 964 | a1 | e62 | e31 |
| 965 | a1 | e62 | e32 |
| 966 | a1 | e62 | e34 |
| 967 | a1 | e62 | e35 |
| 968 | a1 | e62 | e38 |
| 969 | a1 | e62 | e39 |
| 970 | a1 | e62 | e40 |
| 971 | a1 | e62 | e42 |
| 972 | a1 | e62 | e43 |
| 973 | a1 | e62 | e44 |
| 974 | a1 | e62 | e45 |
| 975 | a1 | e62 | e46 |
| 976 | a1 | e62 | e47 |
| 977 | a1 | e62 | e48 |
| 978 | a1 | e62 | e49 |
| 979 | a1 | e62 | e50 |
| 980 | a1 | e62 | e51 |
| 981 | a1 | e62 | e52 |
| 982 | a1 | e62 | e53 |
| 983 | a1 | e62 | e54 |
| 984 | a1 | e62 | e55 |
| 985 | a1 | e62 | e56 |
| 986 | a1 | e62 | e57 |
| 987 | a1 | e62 | e58 |
| 988 | a1 | e62 | e60 |
| 989 | a1 | e62 | e61 |
| 990 | a1 | e62 | e62 |
| 991 | a1 | e62 | e63 |
| 992 | a1 | e62 | e64 |
| 993 | a1 | e62 | e65 |
| 994 | a1 | e62 | e66 |
| 995 | a1 | e62 | e67 |
| 996 | a1 | e63 | a1 |
| 997 | a1 | e63 | d1 |
| 998 | a1 | e63 | d1 |
| 999 | a1 | e63 | d4 |
| 1000 | a1 | e63 | d5 |
| Compound No. | Ar_{A} | Ar_{B} | Ar_{C} |
|---|---|---|---|
| 1001 | a1 | e63 | d6 |
| 1002 | a1 | e63 | d8 |
| 1003 | a1 | e63 | d10 |
| 1004 | a1 | e63 | e3 |
| 1005 | a1 | e63 | e4 |
| 1006 | a1 | e63 | e5 |
| 1007 | a1 | e63 | e6 |
| 1008 | a1 | e63 | e7 |
| 1009 | a1 | e63 | e8 |
| 1010 | a1 | e63 | e9 |
| 1011 | a1 | e63 | e10 |
| 1012 | a1 | e63 | e11 |
| 1013 | a1 | e63 | e12 |
| 1014 | a1 | e63 | e13 |
| 1015 | a1 | e63 | e14 |
| 1016 | a1 | e63 | e15 |
| 1017 | a1 | e63 | e16 |
| 1018 | a1 | e63 | e17 |
| 1019 | a1 | e63 | e18 |
| 1020 | a1 | e63 | e19 |
| 1021 | a1 | e63 | e20 |
| 1022 | a1 | e63 | e21 |
| 1023 | a1 | e63 | e22 |
| 1024 | a1 | e63 | e23 |
| 1025 | a1 | e63 | e25 |
| 1026 | a1 | e63 | e26 |
| 1027 | a1 | e63 | e28 |
| 1028 | a1 | e63 | e29 |
| 1029 | a1 | e63 | e30 |
| 1030 | a1 | e63 | e31 |
| 1031 | a1 | e63 | e32 |
| 1032 | a1 | e63 | e34 |
| 1033 | a1 | e63 | e35 |
| 1034 | a1 | e63 | e38 |
| 1035 | a1 | e63 | e39 |
| 1036 | a1 | e63 | e40 |
| 1037 | a1 | e63 | e42 |
| 1038 | a1 | e63 | e43 |
| 1039 | a1 | e63 | e44 |
| 1040 | a1 | e63 | e45 |
| 1041 | a1 | e63 | e46 |
| 1042 | a1 | e63 | e47 |
| 1043 | a1 | e63 | e48 |
| 1044 | a1 | e63 | e49 |
| 1045 | a1 | e63 | e50 |
| 1046 | a1 | e63 | e51 |
| 1047 | a1 | e63 | e52 |
| 1048 | a1 | e63 | e53 |
| 1049 | a1 | e63 | e54 |
| 1050 | a1 | e63 | e55 |
| 1051 | a1 | e63 | e56 |
| 1052 | a1 | e63 | e57 |
| 1053 | a1 | e63 | e58 |
| 1054 | a1 | e63 | e60 |
| 1055 | a1 | e63 | e61 |
| 1056 | a1 | e63 | e63 |
| 1057 | a1 | e63 | e64 |
| 1058 | a1 | e63 | e65 |
| 1059 | a1 | e63 | e66 |
| 1060 | a1 | e63 | e67 |
| 1061 | a1 | e64 | a1 |
| 1062 | a1 | e64 | d1 |
| 1063 | a1 | e64 | d1 |
| 1064 | a1 | e64 | d4 |
| 1065 | a1 | e64 | d5 |
| 1066 | a1 | e64 | d6 |
| 1067 | a1 | e64 | d8 |
| 1068 | a1 | e64 | d10 |
| 1069 | a1 | e64 | e3 |
| 1070 | a1 | e64 | e4 |
| 1071 | a1 | e64 | e5 |
| 1072 | a1 | e64 | e6 |
| 1073 | a1 | e64 | e7 |
| 1074 | a1 | e64 | e8 |
| 1075 | a1 | e64 | e9 |
| 1076 | a1 | e64 | e10 |
| 1077 | a1 | e64 | e11 |
| 1078 | a1 | e64 | e12 |
| 1079 | a1 | e64 | e13 |
| 1080 | a1 | e64 | e14 |
| 1081 | a1 | e64 | e15 |
| 1082 | a1 | e64 | e16 |
| 1083 | a1 | e64 | e17 |
| 1084 | a1 | e64 | e18 |
| 1085 | a1 | e64 | e19 |
| 1086 | a1 | e64 | e20 |
| 1087 | a1 | e64 | e21 |
| 1088 | a1 | e64 | e22 |
| 1089 | a1 | e64 | e23 |
| 1090 | a1 | e64 | e25 |
| 1091 | a1 | e64 | e26 |
| 1092 | a1 | e64 | e28 |
| 1093 | a1 | e64 | e29 |
| 1094 | a1 | e64 | e30 |
| 1095 | a1 | e64 | e31 |
| 1096 | a1 | e64 | e32 |
| 1097 | a1 | e64 | e34 |
| 1098 | a1 | e64 | e35 |
| 1099 | a1 | e64 | e38 |
| 1100 | a1 | e64 | e39 |
| 1101 | a1 | e64 | e40 |
| 1102 | a1 | e64 | e42 |
| 1103 | a1 | e64 | e43 |
| 1104 | a1 | e64 | e44 |
| 1105 | a1 | e64 | e45 |
| 1106 | a1 | e64 | e46 |
| 1107 | a1 | e64 | e47 |
| 1108 | a1 | e64 | e48 |
| 1109 | a1 | e64 | e49 |
| 1110 | a1 | e64 | e50 |
| 1111 | a1 | e64 | e51 |
| 1112 | a1 | e64 | e52 |
| 1113 | a1 | e64 | e53 |
| 1114 | a1 | e64 | e54 |
| 1115 | a1 | e64 | e55 |
| 1116 | a1 | e64 | e56 |
| 1117 | a1 | e64 | e57 |
| 1118 | a1 | e64 | e58 |
| 1119 | a1 | e64 | e60 |
| 1120 | a1 | e64 | e61 |
| 1121 | a1 | e64 | e64 |
| 1122 | a1 | e64 | e65 |
| 1123 | a1 | e64 | e66 |
| 1124 | a1 | e64 | e67 |
| 1125 | a2 | d2 | a1 |
| 1126 | a2 | d2 | e1 |
| 1127 | a2 | d2 | e2 |
| 1128 | a2 | d2 | e3 |
| 1129 | a2 | d2 | e4 |
| 1130 | a2 | d2 | e5 |
| 1131 | a2 | d2 | e6 |
| 1132 | a2 | d2 | e7 |
| 1133 | a2 | d2 | e8 |
| 1134 | a2 | d2 | e9 |
| 1135 | a2 | d2 | e10 |
| 1136 | a2 | d2 | e11 |
| 1137 | a2 | d2 | e12 |
| 1138 | a2 | d2 | e13 |
| 1139 | a2 | d2 | e14 |
| 1140 | a2 | d2 | e15 |
| 1141 | a2 | d2 | e16 |
| 1142 | a2 | d2 | e17 |
| 1143 | a2 | d2 | e18 |
| 1144 | a2 | d2 | e19 |
| 1145 | a2 | d2 | e20 |
| 1146 | a2 | d2 | e21 |
| 1147 | a2 | d2 | e22 |
| 1148 | a2 | d2 | e23 |
| 1149 | a2 | d2 | e24 |
| 1150 | a2 | d2 | e25 |
| 1151 | a2 | d2 | e26 |
| 1152 | a2 | d2 | e27 |
| 1153 | a2 | d2 | e28 |
| 1154 | a2 | d2 | e29 |
| 1155 | a2 | d2 | e30 |
| 1156 | a2 | d2 | e31 |
| 1157 | a2 | d2 | e32 |
| 1158 | a2 | d2 | e33 |
| 1159 | a2 | d2 | e34 |
| 1160 | a2 | d2 | e35 |
| 1161 | a2 | d2 | e36 |
| 1162 | a2 | d2 | e37 |
| 1163 | a2 | d2 | e38 |
| 1164 | a2 | d2 | e39 |
| 1165 | a2 | d2 | e40 |
| 1166 | a2 | d2 | e41 |
| 1167 | a2 | d2 | e42 |
| 1168 | a2 | d2 | e43 |
| 1169 | a2 | d2 | e44 |
| 1170 | a2 | d2 | e45 |
| 1171 | a2 | d2 | e46 |
| 1172 | a2 | d2 | e47 |
| 1173 | a2 | d2 | e48 |
| 1174 | a2 | d2 | e49 |
| 1175 | a2 | d2 | e50 |
| 1176 | a2 | d2 | e51 |
| 1177 | a2 | d2 | e52 |
| 1178 | a2 | d2 | e53 |
| 1179 | a2 | d2 | e54 |
| 1180 | a2 | d2 | e55 |
| 1181 | a2 | d2 | e56 |
| 1182 | a2 | d2 | e57 |
| 1183 | a2 | d2 | e58 |
| 1184 | a2 | d2 | e59 |
| 1185 | a2 | d2 | e60 |
| 1186 | a2 | d2 | e61 |
| 1187 | a2 | d2 | e62 |
| 1188 | a2 | d2 | e63 |
| 1189 | a2 | d2 | e64 |
| 1190 | a2 | d2 | e65 |
| 1191 | a2 | d2 | e66 |
| 1192 | a2 | d2 | e67 |
| 1193 | a2 | d3 | a1 |
| 1194 | a2 | d3 | e1 |
| 1195 | a2 | d3 | e2 |
| 1196 | a2 | d3 | e3 |
| 1197 | a2 | d3 | e4 |
| 1198 | a2 | d3 | e5 |
| 1199 | a2 | d3 | e6 |
| 1200 | a2 | d3 | e7 |
| Compound No. | Ar_{A} | Ar_{B} | Ar_{C} |
|---|---|---|---|
| 1201 | a2 | d3 | e8 |
| 1202 | a2 | d3 | e9 |
| 1203 | a2 | d3 | e10 |
| 1204 | a2 | d3 | e11 |
| 1205 | a2 | d3 | e12 |
| 1206 | a2 | d3 | e13 |
| 1207 | a2 | d3 | e14 |
| 1208 | a2 | d3 | e15 |
| 1209 | a2 | d3 | e16 |
| 1210 | a2 | d3 | e17 |
| 1211 | a2 | d3 | e18 |
| 1212 | a2 | d3 | e19 |
| 1213 | a2 | d3 | e20 |
| 1214 | a2 | d3 | e21 |
| 1215 | a2 | d3 | e22 |
| 1216 | a2 | d3 | e23 |
| 1217 | a2 | d3 | e24 |
| 1218 | a2 | d3 | e25 |
| 1219 | a2 | d3 | e26 |
| 1220 | a2 | d3 | e27 |
| 1221 | a2 | d3 | e28 |
| 1222 | a2 | d3 | e29 |
| 1223 | a2 | d3 | e30 |
| 1224 | a2 | d3 | e31 |
| 1225 | a2 | d3 | e32 |
| 1226 | a2 | d3 | e33 |
| 1227 | a2 | d3 | e34 |
| 1228 | a2 | d3 | e35 |
| 1229 | a2 | d3 | e36 |
| 1230 | a2 | d3 | e37 |
| 1231 | a2 | d3 | e38 |
| 1232 | a2 | d3 | e39 |
| 1233 | a2 | d3 | e40 |
| 1234 | a2 | d3 | e41 |
| 1235 | a2 | d3 | e42 |
| 1236 | a2 | d3 | e43 |
| 1237 | a2 | d3 | e44 |
| 1238 | a2 | d3 | e45 |
| 1239 | a2 | d3 | e46 |
| 1240 | a2 | d3 | e47 |
| 1241 | a2 | d3 | e48 |
| 1242 | a2 | d3 | e49 |
| 1243 | a2 | d3 | e50 |
| 1244 | a2 | d3 | e51 |
| 1245 | a2 | d3 | e52 |
| 1246 | a2 | d3 | e53 |
| 1247 | a2 | d3 | e54 |
| 1248 | a2 | d3 | e55 |
| 1249 | a2 | d3 | e56 |
| 1250 | a2 | d3 | e57 |
| 1251 | a2 | d3 | e58 |
| 1252 | a2 | d3 | e59 |
| 1253 | a2 | d3 | e60 |
| 1254 | a2 | d3 | e61 |
| 1255 | a2 | d3 | e62 |
| 1256 | a2 | d3 | e63 |
| 1257 | a2 | d3 | e64 |
| 1258 | a2 | d3 | e65 |
| 1259 | a2 | d3 | e66 |
| 1260 | a2 | d3 | e67 |
| 1261 | a2 | d7 | a1 |
| 1262 | a2 | d7 | e1 |
| 1263 | a2 | d7 | e1 |
| 1264 | a2 | d7 | e2 |
| 1265 | a2 | d7 | e3 |
| 1266 | a2 | d7 | e4 |
| 1267 | a2 | d7 | e5 |
| 1268 | a2 | d7 | e6 |
| 1269 | a2 | d7 | e7 |
| 1270 | a2 | d7 | e8 |
| 1271 | a2 | d7 | e9 |
| 1272 | a2 | d7 | e10 |
| 1273 | a2 | d7 | e11 |
| 1274 | a2 | d7 | e12 |
| 1275 | a2 | d7 | e13 |
| 1276 | a2 | d7 | e14 |
| 1277 | a2 | d7 | e15 |
| 1278 | a2 | d7 | e16 |
| 1279 | a2 | d7 | e17 |
| 1280 | a2 | d7 | e18 |
| 1281 | a2 | d7 | e19 |
| 1282 | a2 | d7 | e20 |
| 1283 | a2 | d7 | e21 |
| 1284 | a2 | d7 | e22 |
| 1285 | a2 | d7 | e23 |
| 1286 | a2 | d7 | e24 |
| 1287 | a2 | d7 | e25 |
| 1288 | a2 | d7 | e26 |
| 1289 | a2 | d7 | e27 |
| 1290 | a2 | d7 | e28 |
| 1291 | a2 | d7 | e29 |
| 1292 | a2 | d7 | e30 |
| 1293 | a2 | d7 | e31 |
| 1294 | a2 | d7 | e32 |
| 1295 | a2 | d7 | e33 |
| 1296 | a2 | d7 | e34 |
| 1297 | a2 | d7 | e35 |
| 1298 | a2 | d7 | e36 |
| 1299 | a2 | d7 | e37 |
| 1300 | a2 | d7 | e38 |
| 1301 | a2 | d7 | e39 |
| 1302 | a2 | d7 | e40 |
| 1303 | a2 | d7 | e41 |
| 1304 | a2 | d7 | e42 |
| 1305 | a2 | d7 | e43 |
| 1306 | a2 | d7 | e44 |
| 1307 | a2 | d7 | e45 |
| 1308 | a2 | d7 | e46 |
| 1309 | a2 | d7 | e47 |
| 1310 | a2 | d7 | e48 |
| 1311 | a2 | d7 | e49 |
| 1312 | a2 | d7 | e50 |
| 1313 | a2 | d7 | e51 |
| 1314 | a2 | d7 | e52 |
| 1315 | a2 | d7 | e53 |
| 1316 | a2 | d7 | e54 |
| 1317 | a2 | d7 | e55 |
| 1318 | a2 | d7 | e56 |
| 1319 | a2 | d7 | e57 |
| 1320 | a2 | d7 | e58 |
| 1321 | a2 | d7 | e59 |
| 1322 | a2 | d7 | e60 |
| 1323 | a2 | d7 | e61 |
| 1324 | a2 | d7 | e62 |
| 1325 | a2 | d7 | e63 |
| 1326 | a2 | d7 | e64 |
| 1327 | a2 | d7 | e65 |
| 1328 | a2 | d7 | e66 |
| 1329 | a2 | d7 | e67 |
| 1330 | a2 | d9 | a1 |
| 1331 | a2 | d9 | e1 |
| 1332 | a2 | d9 | e1 |
| 1333 | a2 | d9 | e2 |
| 1334 | a2 | d9 | e3 |
| 1335 | a2 | d9 | e4 |
| 1336 | a2 | d9 | e5 |
| 1337 | a2 | d9 | e6 |
| 1338 | a2 | d9 | e7 |
| 1339 | a2 | d9 | e8 |
| 1340 | a2 | d9 | e9 |
| 1341 | a2 | d9 | e10 |
| 1342 | a2 | d9 | e11 |
| 1343 | a2 | d9 | e12 |
| 1344 | a2 | d9 | e13 |
| 1345 | a2 | d9 | e14 |
| 1346 | a2 | d9 | e15 |
| 1347 | a2 | d9 | e16 |
| 1348 | a2 | d9 | e17 |
| 1349 | a2 | d9 | e18 |
| 1350 | a2 | d9 | e19 |
| 1351 | a2 | d9 | e20 |
| 1352 | a2 | d9 | e21 |
| 1353 | a2 | d9 | e22 |
| 1354 | a2 | d9 | e23 |
| 1355 | a2 | d9 | e24 |
| 1356 | a2 | d9 | e25 |
| 1357 | a2 | d9 | e26 |
| 1358 | a2 | d9 | e27 |
| 1359 | a2 | d9 | e28 |
| 1360 | a2 | d9 | e29 |
| 1361 | a2 | d9 | e30 |
| 1362 | a2 | d9 | e31 |
| 1363 | a2 | d9 | e32 |
| 1364 | a2 | d9 | e33 |
| 1365 | a2 | d9 | e34 |
| 1366 | a2 | d9 | e35 |
| 1367 | a2 | d9 | e36 |
| 1368 | a2 | d9 | e37 |
| 1369 | a2 | d9 | e38 |
| 1370 | a2 | d9 | e39 |
| 1371 | a2 | d9 | e40 |
| 1372 | a2 | d9 | e41 |
| 1373 | a2 | d9 | e42 |
| 1374 | a2 | d9 | e43 |
| 1375 | a2 | d9 | e44 |
| 1376 | a2 | d9 | e45 |
| 1377 | a2 | d9 | e46 |
| 1378 | a2 | d9 | e47 |
| 1379 | a2 | d9 | e48 |
| 1380 | a2 | d9 | e49 |
| 1381 | a2 | d9 | e50 |
| 1382 | a2 | d9 | e51 |
| 1383 | a2 | d9 | e52 |
| 1384 | a2 | d9 | e53 |
| 1385 | a2 | d9 | e54 |
| 1386 | a2 | d9 | e55 |
| 1387 | a2 | d9 | e56 |
| 1388 | a2 | d9 | e57 |
| 1389 | a2 | d9 | e58 |
| 1390 | a2 | d9 | e59 |
| 1391 | a2 | d9 | e60 |
| 1392 | a2 | d9 | e61 |
| 1393 | a2 | d9 | e62 |
| 1394 | a2 | d9 | e63 |
| 1395 | a2 | d9 | e64 |
| 1396 | a2 | d9 | e65 |
| 1397 | a2 | d9 | e66 |
| 1398 | a2 | d9 | e67 |
| 1399 | a2 | e1 | a1 |
| 1400 | a2 | e1 | d1 |
| Compound No. | Ar_{A} | Ar_{B} | Ar_{C} |
|---|---|---|---|
| 1401 | a2 | e1 | d1 |
| 1402 | a2 | e1 | d4 |
| 1403 | a2 | e1 | d5 |
| 1404 | a2 | e1 | d6 |
| 1405 | a2 | e1 | d8 |
| 1406 | a2 | e1 | d10 |
| 1407 | a2 | e1 | e1 |
| 1408 | a2 | e1 | e1 |
| 1409 | a2 | e1 | e2 |
| 1410 | a2 | e1 | e3 |
| 1411 | a2 | e1 | e4 |
| 1412 | a2 | e1 | e5 |
| 1413 | a2 | e1 | e6 |
| 1414 | a2 | e1 | e7 |
| 1415 | a2 | e1 | e8 |
| 1416 | a2 | e1 | e9 |
| 1417 | a2 | e1 | e10 |
| 1418 | a2 | e1 | e11 |
| 1419 | a2 | e1 | e12 |
| 1420 | a2 | e1 | e13 |
| 1421 | a2 | e1 | e14 |
| 1422 | a2 | e1 | e15 |
| 1423 | a2 | e1 | e16 |
| 1424 | a2 | e1 | e17 |
| 1425 | a2 | e1 | e18 |
| 1426 | a2 | e1 | e19 |
| 1427 | a2 | e1 | e20 |
| 1428 | a2 | e1 | e21 |
| 1429 | a2 | e1 | e22 |
| 1430 | a2 | e1 | e23 |
| 1431 | a2 | e1 | e24 |
| 1432 | a2 | e1 | e25 |
| 1433 | a2 | e1 | e26 |
| 1434 | a2 | e1 | e27 |
| 1435 | a2 | e1 | e28 |
| 1436 | a2 | e1 | e29 |
| 1437 | a2 | e1 | e30 |
| 1438 | a2 | e1 | e31 |
| 1439 | a2 | e1 | e32 |
| 1440 | a2 | e1 | e33 |
| 1441 | a2 | e1 | e34 |
| 1442 | a2 | e1 | e35 |
| 1443 | a2 | e1 | e36 |
| 1444 | a2 | e1 | e37 |
| 1445 | a2 | e1 | e38 |
| 1446 | a2 | e1 | e39 |
| 1447 | a2 | e1 | e40 |
| 1448 | a2 | e1 | e41 |
| 1449 | a2 | e1 | e42 |
| 1450 | a2 | e1 | e43 |
| 1451 | a2 | e1 | e44 |
| 1452 | a2 | e1 | e45 |
| 1453 | a2 | e1 | e46 |
| 1454 | a2 | e1 | e47 |
| 1455 | a2 | e1 | e48 |
| 1456 | a2 | e1 | e49 |
| 1457 | a2 | e1 | e50 |
| 1458 | a2 | e1 | e51 |
| 1459 | a2 | e1 | e52 |
| 1460 | a2 | e1 | e53 |
| 1461 | a2 | e1 | e54 |
| 1462 | a2 | e1 | e55 |
| 1463 | a2 | e1 | e56 |
| 1464 | a2 | e1 | e57 |
| 1465 | a2 | e1 | e58 |
| 1466 | a2 | e1 | e59 |
| 1467 | a2 | e1 | e60 |
| 1468 | a2 | e1 | e61 |
| 1469 | a2 | e1 | e62 |
| 1470 | a2 | e1 | e63 |
| 1471 | a2 | e1 | e64 |
| 1472 | a2 | e1 | e65 |
| 1473 | a2 | e1 | e66 |
| 1474 | a2 | e1 | e67 |
| 1475 | a2 | e2 | a1 |
| 1476 | a2 | e2 | d1 |
| 1477 | a2 | e2 | d1 |
| 1478 | a2 | e2 | d4 |
| 1479 | a2 | e2 | d5 |
| 1480 | a2 | e2 | d6 |
| 1481 | a2 | e2 | d8 |
| 1482 | a2 | e2 | d10 |
| 1483 | a2 | e2 | e2 |
| 1484 | a2 | e2 | e3 |
| 1485 | a2 | e2 | e4 |
| 1486 | a2 | e2 | e5 |
| 1487 | a2 | e2 | e6 |
| 1488 | a2 | e2 | e7 |
| 1489 | a2 | e2 | e8 |
| 1490 | a2 | e2 | e9 |
| 1491 | a2 | e2 | e10 |
| 1492 | a2 | e2 | e11 |
| 1493 | a2 | e2 | e12 |
| 1494 | a2 | e2 | e13 |
| 1495 | a2 | e2 | e14 |
| 1496 | a2 | e2 | e15 |
| 1497 | a2 | e2 | e16 |
| 1498 | a2 | e2 | e17 |
| 1499 | a2 | e2 | e18 |
| 1500 | a2 | e2 | e19 |
| 1501 | a2 | e2 | e20 |
| 1502 | a2 | e2 | e21 |
| 1503 | a2 | e2 | e22 |
| 1504 | a2 | e2 | e23 |
| 1505 | a2 | e2 | e24 |
| 1506 | a2 | e2 | e25 |
| 1507 | a2 | e2 | e26 |
| 1508 | a2 | e2 | e28 |
| 1509 | a2 | e2 | e29 |
| 1510 | a2 | e2 | e30 |
| 1511 | a2 | e2 | e31 |
| 1512 | a2 | e2 | e32 |
| 1513 | a2 | e2 | e33 |
| 1514 | a2 | e2 | e34 |
| 1515 | a2 | e2 | e35 |
| 1516 | a2 | e2 | e36 |
| 1517 | a2 | e2 | e37 |
| 1518 | a2 | e2 | e38 |
| 1519 | a2 | e2 | e39 |
| 1520 | a2 | e2 | e40 |
| 1521 | a2 | e2 | e41 |
| 1522 | a2 | e2 | e42 |
| 1523 | a2 | e2 | e43 |
| 1524 | a2 | e2 | e44 |
| 1525 | a2 | e2 | e45 |
| 1526 | a2 | e2 | e46 |
| 1527 | a2 | e2 | e47 |
| 1528 | a2 | e2 | e48 |
| 1529 | a2 | e2 | e49 |
| 1530 | a2 | e2 | e50 |
| 1531 | a2 | e2 | e51 |
| 1532 | a2 | e2 | e52 |
| 1533 | a2 | e2 | e53 |
| 1534 | a2 | e2 | e54 |
| 1535 | a2 | e2 | e55 |
| 1536 | a2 | e2 | e56 |
| 1537 | a2 | e2 | e57 |
| 1538 | a2 | e2 | e58 |
| 1539 | a2 | e2 | e59 |
| 1540 | a2 | e2 | e60 |
| 1541 | a2 | e2 | e61 |
| 1542 | a2 | e2 | e62 |
| 1543 | a2 | e2 | e63 |
| 1544 | a2 | e2 | e64 |
| 1545 | a2 | e2 | e65 |
| 1546 | a2 | e2 | e66 |
| 1547 | a2 | e2 | e67 |
| 1548 | a2 | e24 | a1 |
| 1549 | a2 | e24 | d1 |
| 1550 | a2 | e24 | d1 |
| 1551 | a2 | e24 | d4 |
| 1552 | a2 | e24 | d5 |
| 1553 | a2 | e24 | d6 |
| 1554 | a2 | e24 | d8 |
| 1555 | a2 | e24 | d10 |
| 1556 | a2 | e24 | e3 |
| 1557 | a2 | e24 | e4 |
| 1558 | a2 | e24 | e5 |
| 1559 | a2 | e24 | e6 |
| 1560 | a2 | e24 | e7 |
| 1561 | a2 | e24 | e8 |
| 1562 | a2 | e24 | e9 |
| 1563 | a2 | e24 | e10 |
| 1564 | a2 | e24 | e11 |
| 1565 | a2 | e24 | e12 |
| 1566 | a2 | e24 | e13 |
| 1567 | a2 | e24 | e14 |
| 1568 | a2 | e24 | e15 |
| 1569 | a2 | e24 | e16 |
| 1570 | a2 | e24 | e17 |
| 1571 | a2 | e24 | e18 |
| 1572 | a2 | e24 | e19 |
| 1573 | a2 | e24 | e20 |
| 1574 | a2 | e24 | e21 |
| 1575 | a2 | e24 | e22 |
| 1576 | a2 | e24 | e23 |
| 1577 | a2 | e24 | e24 |
| 1578 | a2 | e24 | e25 |
| 1579 | a2 | e24 | e26 |
| 1580 | a2 | e24 | e27 |
| 1581 | a2 | e24 | e28 |
| 1582 | a2 | e24 | e29 |
| 1583 | a2 | e24 | e30 |
| 1584 | a2 | e24 | e31 |
| 1585 | a2 | e24 | e32 |
| 1586 | a2 | e24 | e33 |
| 1587 | a2 | e24 | e34 |
| 1588 | a2 | e24 | e35 |
| 1589 | a2 | e24 | e36 |
| 1590 | a2 | e24 | e37 |
| 1591 | a2 | e24 | e38 |
| 1592 | a2 | e24 | e39 |
| 1593 | a2 | e24 | e40 |
| 1594 | a2 | e24 | e41 |
| 1595 | a2 | e24 | e42 |
| 1596 | a2 | e24 | e43 |
| 1597 | a2 | e24 | e44 |
| 1598 | a2 | e24 | e45 |
| 1599 | a2 | e24 | e46 |
| 1600 | a2 | e24 | e47 |
| Compound No. | Ar_{A} | Ar_{B} | Ar_{C} |
|---|---|---|---|
| 1601 | a2 | e24 | e48 |
| 1602 | a2 | e24 | e49 |
| 1603 | a2 | e24 | e50 |
| 1604 | a2 | e24 | e51 |
| 1605 | a2 | e24 | e52 |
| 1606 | a2 | e24 | e53 |
| 1607 | a2 | e24 | e54 |
| 1608 | a2 | e24 | e55 |
| 1609 | a2 | e24 | e56 |
| 1610 | a2 | e24 | e57 |
| 1611 | a2 | e24 | e58 |
| 1612 | a2 | e24 | e59 |
| 1613 | a2 | e24 | e60 |
| 1614 | a2 | e24 | e61 |
| 1615 | a2 | e24 | e62 |
| 1616 | a2 | e24 | e63 |
| 1617 | a2 | e24 | e64 |
| 1618 | a2 | e24 | e65 |
| 1619 | a2 | e24 | e66 |
| 1620 | a2 | e24 | e67 |
| 1621 | a2 | e27 | a1 |
| 1622 | a2 | e27 | d1 |
| 1623 | a2 | e27 | d1 |
| 1624 | a2 | e27 | d4 |
| 1625 | a2 | e27 | d5 |
| 1626 | a2 | e27 | d6 |
| 1627 | a2 | e27 | d8 |
| 1628 | a2 | e27 | d10 |
| 1629 | a2 | e27 | e3 |
| 1630 | a2 | e27 | e4 |
| 1631 | a2 | e27 | e5 |
| 1632 | a2 | e27 | e6 |
| 1633 | a2 | e27 | e7 |
| 1634 | a2 | e27 | e8 |
| 1635 | a2 | e27 | e9 |
| 1636 | a2 | e27 | e10 |
| 1637 | a2 | e27 | e11 |
| 1638 | a2 | e27 | e12 |
| 1639 | a2 | e27 | e13 |
| 1640 | a2 | e27 | e14 |
| 1641 | a2 | e27 | e15 |
| 1642 | a2 | e27 | e16 |
| 1643 | a2 | e27 | e17 |
| 1644 | a2 | e27 | e18 |
| 1645 | a2 | e27 | e19 |
| 1646 | a2 | e27 | e20 |
| 1647 | a2 | e27 | e21 |
| 1648 | a2 | e27 | e22 |
| 1649 | a2 | e27 | e23 |
| 1650 | a2 | e27 | e25 |
| 1651 | a2 | e27 | e26 |
| 1652 | a2 | e27 | e27 |
| 1653 | a2 | e27 | e28 |
| 1654 | a2 | e27 | e29 |
| 1655 | a2 | e27 | e30 |
| 1656 | a2 | e27 | e31 |
| 1657 | a2 | e27 | e32 |
| 1658 | a2 | e27 | e33 |
| 1659 | a2 | e27 | e34 |
| 1660 | a2 | e27 | e35 |
| 1661 | a2 | e27 | e36 |
| 1662 | a2 | e27 | e37 |
| 1663 | a2 | e27 | e38 |
| 1664 | a2 | e27 | e39 |
| 1665 | a2 | e27 | e40 |
| 1666 | a2 | e27 | e41 |
| 1667 | a2 | e27 | e42 |
| 1668 | a2 | e27 | e43 |
| 1669 | a2 | e27 | e44 |
| 1670 | a2 | e27 | e45 |
| 1671 | a2 | e27 | e46 |
| 1672 | a2 | e27 | e47 |
| 1673 | a2 | e27 | e48 |
| 1674 | a2 | e27 | e49 |
| 1675 | a2 | e27 | e50 |
| 1676 | a2 | e27 | e51 |
| 1677 | a2 | e27 | e52 |
| 1678 | a2 | e27 | e53 |
| 1679 | a2 | e27 | e54 |
| 1680 | a2 | e27 | e55 |
| 1681 | a2 | e27 | e56 |
| 1682 | a2 | e27 | e57 |
| 1683 | a2 | e27 | e58 |
| 1684 | a2 | e27 | e59 |
| 1685 | a2 | e27 | e60 |
| 1686 | a2 | e27 | e61 |
| 1687 | a2 | e27 | e62 |
| 1688 | a2 | e27 | e63 |
| 1689 | a2 | e27 | e64 |
| 1690 | a2 | e27 | e65 |
| 1691 | a2 | e27 | e66 |
| 1692 | a2 | e27 | e67 |
| 1693 | a2 | e33 | a1 |
| 1694 | a2 | e33 | d1 |
| 1695 | a2 | e33 | d1 |
| 1696 | a2 | e33 | d4 |
| 1697 | a2 | e33 | d5 |
| 1698 | a2 | e33 | d6 |
| 1699 | a2 | e33 | d8 |
| 1700 | a2 | e33 | d10 |
| 1701 | a2 | e33 | e3 |
| 1702 | a2 | e33 | e4 |
| 1703 | a2 | e33 | e5 |
| 1704 | a2 | e33 | e6 |
| 1705 | a2 | e33 | e7 |
| 1706 | a2 | e33 | e8 |
| 1707 | a2 | e33 | e9 |
| 1708 | a2 | e33 | e10 |
| 1709 | a2 | e33 | e11 |
| 1710 | a2 | e33 | e12 |
| 1711 | a2 | e33 | e13 |
| 1712 | a2 | e33 | e14 |
| 1713 | a2 | e33 | e15 |
| 1714 | a2 | e33 | e16 |
| 1715 | a2 | e33 | e17 |
| 1716 | a2 | e33 | e18 |
| 1717 | a2 | e33 | e19 |
| 1718 | a2 | e33 | e20 |
| 1719 | a2 | e33 | e21 |
| 1720 | a2 | e33 | e22 |
| 1721 | a2 | e33 | e23 |
| 1722 | a2 | e33 | e25 |
| 1723 | a2 | e33 | e26 |
| 1724 | a2 | e33 | e27 |
| 1725 | a2 | e33 | e28 |
| 1726 | a2 | e33 | e29 |
| 1727 | a2 | e33 | e30 |
| 1728 | a2 | e33 | e31 |
| 1729 | a2 | e33 | e32 |
| 1730 | a2 | e33 | e33 |
| 1731 | a2 | e33 | e34 |
| 1732 | a2 | e33 | e35 |
| 1733 | a2 | e33 | e36 |
| 1734 | a2 | e33 | e37 |
| 1735 | a2 | e33 | e38 |
| 1736 | a2 | e33 | e39 |
| 1737 | a2 | e33 | e40 |
| 1738 | a2 | e33 | e41 |
| 1739 | a2 | e33 | e42 |
| 1740 | a2 | e33 | e43 |
| 1741 | a2 | e33 | e44 |
| 1742 | a2 | e33 | e45 |
| 1743 | a2 | e33 | e46 |
| 1744 | a2 | e33 | e47 |
| 1745 | a2 | e33 | e48 |
| 1746 | a2 | e33 | e49 |
| 1747 | a2 | e33 | e50 |
| 1748 | a2 | e33 | e51 |
| 1749 | a2 | e33 | e52 |
| 1750 | a2 | e33 | e53 |
| 1751 | a2 | e33 | e54 |
| 1752 | a2 | e33 | e55 |
| 1753 | a2 | e33 | e56 |
| 1754 | a2 | e33 | e57 |
| 1755 | a2 | e33 | e58 |
| 1756 | a2 | e33 | e59 |
| 1757 | a2 | e33 | e60 |
| 1758 | a2 | e33 | e61 |
| 1759 | a2 | e33 | e62 |
| 1760 | a2 | e33 | e63 |
| 1761 | a2 | e33 | e64 |
| 1762 | a2 | e33 | e65 |
| 1763 | a2 | e33 | e66 |
| 1764 | a2 | e33 | e67 |
| 1765 | a2 | e36 | a1 |
| 1766 | a2 | e36 | d1 |
| 1767 | a2 | e36 | d1 |
| 1768 | a2 | e36 | d4 |
| 1769 | a2 | e36 | d5 |
| 1770 | a2 | e36 | d6 |
| 1771 | a2 | e36 | d8 |
| 1772 | a2 | e36 | d10 |
| 1773 | a2 | e36 | e3 |
| 1774 | a2 | e36 | e4 |
| 1775 | a2 | e36 | e5 |
| 1776 | a2 | e36 | e6 |
| 1777 | a2 | e36 | e7 |
| 1778 | a2 | e36 | e8 |
| 1779 | a2 | e36 | e9 |
| 1780 | a2 | e36 | e10 |
| 1781 | a2 | e36 | e11 |
| 1782 | a2 | e36 | e12 |
| 1783 | a2 | e36 | e13 |
| 1784 | a2 | e36 | e14 |
| 1785 | a2 | e36 | e15 |
| 1786 | a2 | e36 | e16 |
| 1787 | a2 | e36 | e17 |
| 1788 | a2 | e36 | e18 |
| 1789 | a2 | e36 | e19 |
| 1790 | a2 | e36 | e20 |
| 1791 | a2 | e36 | e21 |
| 1792 | a2 | e36 | e22 |
| 1793 | a2 | e36 | e23 |
| 1794 | a2 | e36 | e25 |
| 1795 | a2 | e36 | e26 |
| 1796 | a2 | e36 | e28 |
| 1797 | a2 | e36 | e29 |
| 1798 | a2 | e36 | e30 |
| 1799 | a2 | e36 | e31 |
| 1800 | a2 | e36 | e32 |
| Compound No. | Ar_{A} | Ar_{B} | Ar_{C} |
|---|---|---|---|
| 1801 | a2 | e36 | e34 |
| 1802 | a2 | e36 | e35 |
| 1803 | a2 | e36 | e36 |
| 1804 | a2 | e36 | e37 |
| 1805 | a2 | e36 | e38 |
| 1806 | a2 | e36 | e39 |
| 1807 | a2 | e36 | e40 |
| 1808 | a2 | e36 | e41 |
| 1809 | a2 | e36 | e42 |
| 1810 | a2 | e36 | e43 |
| 1811 | a2 | e36 | e44 |
| 1812 | a2 | e36 | e45 |
| 1813 | a2 | e36 | e46 |
| 1814 | a2 | e36 | e47 |
| 1815 | a2 | e36 | e48 |
| 1816 | a2 | e36 | e49 |
| 1817 | a2 | e36 | e50 |
| 1818 | a2 | e36 | e51 |
| 1819 | a2 | e36 | e52 |
| 1820 | a2 | e36 | e53 |
| 1821 | a2 | e36 | e54 |
| 1822 | a2 | e36 | e55 |
| 1823 | a2 | e36 | e56 |
| 1824 | a2 | e36 | e57 |
| 1825 | a2 | e36 | e58 |
| 1826 | a2 | e36 | e59 |
| 1827 | a2 | e36 | e60 |
| 1828 | a2 | e36 | e61 |
| 1829 | a2 | e36 | e62 |
| 1830 | a2 | e36 | e63 |
| 1831 | a2 | e36 | e64 |
| 1832 | a2 | e36 | e65 |
| 1833 | a2 | e36 | e66 |
| 1834 | a2 | e36 | e67 |
| 1835 | a2 | e37 | a1 |
| 1836 | a2 | e37 | d1 |
| 1837 | a2 | e37 | d1 |
| 1838 | a2 | e37 | d4 |
| 1839 | a2 | e37 | d5 |
| 1840 | a2 | e37 | d6 |
| 1841 | a2 | e37 | d8 |
| 1842 | a2 | e37 | d10 |
| 1843 | a2 | e37 | e3 |
| 1844 | a2 | e37 | e4 |
| 1845 | a2 | e37 | e5 |
| 1846 | a2 | e37 | e6 |
| 1847 | a2 | e37 | e7 |
| 1848 | a2 | e37 | e8 |
| 1849 | a2 | e37 | e9 |
| 1850 | a2 | e37 | e10 |
| 1851 | a2 | e37 | e11 |
| 1852 | a2 | e37 | e12 |
| 1853 | a2 | e37 | e13 |
| 1854 | a2 | e37 | e14 |
| 1855 | a2 | e37 | e15 |
| 1856 | a2 | e37 | e16 |
| 1857 | a2 | e37 | e17 |
| 1858 | a2 | e37 | e18 |
| 1859 | a2 | e37 | e19 |
| 1860 | a2 | e37 | e20 |
| 1861 | a2 | e37 | e21 |
| 1862 | a2 | e37 | e22 |
| 1863 | a2 | e37 | e23 |
| 1864 | a2 | e37 | e25 |
| 1865 | a2 | e37 | e26 |
| 1866 | a2 | e37 | e28 |
| 1867 | a2 | e37 | e29 |
| 1868 | a2 | e37 | e30 |
| 1869 | a2 | e37 | e31 |
| 1870 | a2 | e37 | e32 |
| 1871 | a2 | e37 | e34 |
| 1872 | a2 | e37 | e35 |
| 1873 | a2 | e37 | e37 |
| 1874 | a2 | e37 | e38 |
| 1875 | a2 | e37 | e39 |
| 1876 | a2 | e37 | e40 |
| 1877 | a2 | e37 | e41 |
| 1878 | a2 | e37 | e42 |
| 1879 | a2 | e37 | e43 |
| 1880 | a2 | e37 | e44 |
| 1881 | a2 | e37 | e45 |
| 1882 | a2 | e37 | e46 |
| 1883 | a2 | e37 | e47 |
| 1884 | a2 | e37 | e48 |
| 1885 | a2 | e37 | e49 |
| 1886 | a2 | e37 | e50 |
| 1887 | a2 | e37 | e51 |
| 1888 | a2 | e37 | e52 |
| 1889 | a2 | e37 | e53 |
| 1890 | a2 | e37 | e54 |
| 1891 | a2 | e37 | e55 |
| 1892 | a2 | e37 | e56 |
| 1893 | a2 | e37 | e57 |
| 1894 | a2 | e37 | e58 |
| 1895 | a2 | e37 | e59 |
| 1896 | a2 | e37 | e60 |
| 1897 | a2 | e37 | e61 |
| 1898 | a2 | e37 | e62 |
| 1899 | a2 | e37 | e63 |
| 1900 | a2 | e37 | e64 |
| 1901 | a2 | e37 | e65 |
| 1902 | a2 | e37 | e66 |
| 1903 | a2 | e37 | e67 |
| 1904 | a2 | e41 | a1 |
| 1905 | a2 | e41 | d1 |
| 1906 | a2 | e41 | d1 |
| 1907 | a2 | e41 | d4 |
| 1908 | a2 | e41 | d5 |
| 1909 | a2 | e41 | d6 |
| 1910 | a2 | e41 | d8 |
| 1911 | a2 | e41 | d10 |
| 1912 | a2 | e41 | e3 |
| 1913 | a2 | e41 | e4 |
| 1914 | a2 | e41 | e5 |
| 1915 | a2 | e41 | e6 |
| 1916 | a2 | e41 | e7 |
| 1917 | a2 | e41 | e8 |
| 1918 | a2 | e41 | e9 |
| 1919 | a2 | e41 | e10 |
| 1920 | a2 | e41 | e11 |
| 1921 | a2 | e41 | e12 |
| 1922 | a2 | e41 | e13 |
| 1923 | a2 | e41 | e14 |
| 1924 | a2 | e41 | e15 |
| 1925 | a2 | e41 | e16 |
| 1926 | a2 | e41 | e17 |
| 1927 | a2 | e41 | e18 |
| 1928 | a2 | e41 | e19 |
| 1929 | a2 | e41 | e20 |
| 1930 | a2 | e41 | e21 |
| 1931 | a2 | e41 | e22 |
| 1932 | a2 | e41 | e23 |
| 1933 | a2 | e41 | e25 |
| 1934 | a2 | e41 | e26 |
| 1935 | a2 | e41 | e28 |
| 1936 | a2 | e41 | e29 |
| 1937 | a2 | e41 | e30 |
| 1938 | a2 | e41 | e31 |
| 1939 | a2 | e41 | e32 |
| 1940 | a2 | e41 | e34 |
| 1941 | a2 | e41 | e35 |
| 1942 | a2 | e41 | e38 |
| 1943 | a2 | e41 | e39 |
| 1944 | a2 | e41 | e40 |
| 1945 | a2 | e41 | e41 |
| 1946 | a2 | e41 | e42 |
| 1947 | a2 | e41 | e43 |
| 1948 | a2 | e41 | e44 |
| 1949 | a2 | e41 | e45 |
| 1950 | a2 | e41 | e46 |
| 1951 | a2 | e41 | e47 |
| 1952 | a2 | e41 | e48 |
| 1953 | a2 | e41 | e49 |
| 1954 | a2 | e41 | e50 |
| 1955 | a2 | e41 | e51 |
| 1956 | a2 | e41 | e52 |
| 1957 | a2 | e41 | e53 |
| 1958 | a2 | e41 | e54 |
| 1959 | a2 | e41 | e55 |
| 1960 | a2 | e41 | e56 |
| 1961 | a2 | e41 | e57 |
| 1962 | a2 | e41 | e58 |
| 1963 | a2 | e41 | e59 |
| 1964 | a2 | e41 | e60 |
| 1965 | a2 | e41 | e61 |
| 1966 | a2 | e41 | e62 |
| 1967 | a2 | e41 | e63 |
| 1968 | a2 | e41 | e64 |
| 1969 | a2 | e41 | e65 |
| 1970 | a2 | e41 | e66 |
| 1971 | a2 | e41 | e67 |
| 1972 | a2 | e59 | a1 |
| 1973 | a2 | e59 | d1 |
| 1974 | a2 | e59 | d1 |
| 1975 | a2 | e59 | d4 |
| 1976 | a2 | e59 | d5 |
| 1977 | a2 | e59 | d6 |
| 1978 | a2 | e59 | d8 |
| 1979 | a2 | e59 | d10 |
| 1980 | a2 | e59 | e3 |
| 1981 | a2 | e59 | e4 |
| 1982 | a2 | e59 | e5 |
| 1983 | a2 | e59 | e6 |
| 1984 | a2 | e59 | e7 |
| 1985 | a2 | e59 | e8 |
| 1986 | a2 | e59 | e9 |
| 1987 | a2 | e59 | e10 |
| 1988 | a2 | e59 | e11 |
| 1989 | a2 | e59 | e12 |
| 1990 | a2 | e59 | e13 |
| 1991 | a2 | e59 | e14 |
| 1992 | a2 | e59 | e15 |
| 1993 | a2 | e59 | e16 |
| 1994 | a2 | e59 | e17 |
| 1995 | a2 | e59 | e18 |
| 1996 | a2 | e59 | e19 |
| 1997 | a2 | e59 | e20 |
| 1998 | a2 | e59 | e21 |
| 1999 | a2 | e59 | e22 |
| 2000 | a2 | e59 | e23 |
| Compound No. | Ar_{A} | Ar_{B} | Ar_{C} |
|---|---|---|---|
| 2001 | a2 | e59 | e25 |
| 2002 | a2 | e59 | e26 |
| 2003 | a2 | e59 | e28 |
| 2004 | a2 | e59 | e29 |
| 2005 | a2 | e59 | e30 |
| 2006 | a2 | e59 | e31 |
| 2007 | a2 | e59 | e32 |
| 2008 | a2 | e59 | e34 |
| 2009 | a2 | e59 | e35 |
| 2010 | a2 | e59 | e38 |
| 2011 | a2 | e59 | e39 |
| 2012 | a2 | e59 | e40 |
| 2013 | a2 | e59 | e42 |
| 2014 | a2 | e59 | e43 |
| 2015 | a2 | e59 | e44 |
| 2016 | a2 | e59 | e45 |
| 2017 | a2 | e59 | e46 |
| 2018 | a2 | e59 | e47 |
| 2019 | a2 | e59 | e48 |
| 2020 | a2 | e59 | e49 |
| 2021 | a2 | e59 | e50 |
| 2022 | a2 | e59 | e51 |
| 2023 | a2 | e59 | e52 |
| 2024 | a2 | e59 | e53 |
| 2025 | a2 | e59 | e54 |
| 2026 | a2 | e59 | e55 |
| 2027 | a2 | e59 | e56 |
| 2028 | a2 | e59 | e57 |
| 2029 | a2 | e59 | e58 |
| 2030 | a2 | e59 | e59 |
| 2031 | a2 | e59 | e60 |
| 2032 | a2 | e59 | e61 |
| 2033 | a2 | e59 | e62 |
| 2034 | a2 | e59 | e63 |
| 2035 | a2 | e59 | e64 |
| 2036 | a2 | e59 | e65 |
| 2037 | a2 | e59 | e66 |
| 2038 | a2 | e59 | e67 |
| 2039 | a2 | e62 | a1 |
| 2040 | a2 | e62 | d1 |
| 2041 | a2 | e62 | d1 |
| 2042 | a2 | e62 | d5 |
| 2043 | a2 | e62 | d6 |
| 2044 | a2 | e62 | d8 |
| 2045 | a2 | e62 | d10 |
| 2046 | a2 | e62 | e5 |
| 2047 | a2 | e62 | e6 |
| 2048 | a2 | e62 | e7 |
| 2049 | a2 | e62 | e8 |
| 2050 | a2 | e62 | e9 |
| 2051 | a2 | e62 | e10 |
| 2052 | a2 | e62 | e11 |
| 2053 | a2 | e62 | e12 |
| 2054 | a2 | e62 | e13 |
| 2055 | a2 | e62 | e14 |
| 2056 | a2 | e62 | e15 |
| 2057 | a2 | e62 | e16 |
| 2058 | a2 | e62 | e17 |
| 2059 | a2 | e62 | e18 |
| 2060 | a2 | e62 | e19 |
| 2061 | a2 | e62 | e20 |
| 2062 | a2 | e62 | e21 |
| 2063 | a2 | e62 | e22 |
| 2064 | a2 | e62 | e23 |
| 2065 | a2 | e62 | e25 |
| 2066 | a2 | e62 | e26 |
| 2067 | a2 | e62 | e28 |
| 2068 | a2 | e62 | e29 |
| 2069 | a2 | e62 | e30 |
| 2070 | a2 | e62 | e31 |
| 2071 | a2 | e62 | e32 |
| 2072 | a2 | e62 | e34 |
| 2073 | a2 | e62 | e35 |
| 2074 | a2 | e62 | e38 |
| 2075 | a2 | e62 | e39 |
| 2076 | a2 | e62 | e40 |
| 2077 | a2 | e62 | e42 |
| 2078 | a2 | e62 | e43 |
| 2079 | a2 | e62 | e44 |
| 2080 | a2 | e62 | e45 |
| 2081 | a2 | e62 | e46 |
| 2082 | a2 | e62 | e47 |
| 2083 | a2 | e62 | e48 |
| 2084 | a2 | e62 | e49 |
| 2085 | a2 | e62 | e50 |
| 2086 | a2 | e62 | e51 |
| 2087 | a2 | e62 | e52 |
| 2088 | a2 | e62 | e53 |
| 2089 | a2 | e62 | e54 |
| 2090 | a2 | e62 | e55 |
| 2091 | a2 | e62 | e56 |
| 2092 | a2 | e62 | e57 |
| 2093 | a2 | e62 | e58 |
| 2094 | a2 | e62 | e60 |
| 2095 | a2 | e62 | e61 |
| 2096 | a2 | e62 | e62 |
| 2097 | a2 | e62 | e63 |
| 2098 | a2 | e62 | e64 |
| 2099 | a2 | e62 | e65 |
| 2100 | a2 | e62 | e66 |
| 2101 | a2 | e62 | e67 |
| 2102 | a2 | e63 | a1 |
| 2103 | a2 | e63 | d1 |
| 2104 | a2 | e63 | d1 |
| 2105 | a2 | e63 | d4 |
| 2106 | a2 | e63 | d5 |
| 2107 | a2 | e63 | d6 |
| 2108 | a2 | e63 | d8 |
| 2109 | a2 | e63 | d10 |
| 2110 | a2 | e63 | e3 |
| 2111 | a2 | e63 | e4 |
| 2112 | a2 | e63 | e5 |
| 2113 | a2 | e63 | e6 |
| 2114 | a2 | e63 | e7 |
| 2115 | a2 | e63 | e8 |
| 2116 | a2 | e63 | e9 |
| 2117 | a2 | e63 | e10 |
| 2118 | a2 | e63 | e11 |
| 2119 | a2 | e63 | e12 |
| 2120 | a2 | e63 | e13 |
| 2121 | a2 | e63 | e14 |
| 2122 | a2 | e63 | e15 |
| 2123 | a2 | e63 | e16 |
| 2124 | a2 | e63 | e17 |
| 2125 | a2 | e63 | e18 |
| 2126 | a2 | e63 | e19 |
| 2127 | a2 | e63 | e20 |
| 2128 | a2 | e63 | e21 |
| 2129 | a2 | e63 | e22 |
| 2130 | a2 | e63 | e23 |
| 2131 | a2 | e63 | e25 |
| 2132 | a2 | e63 | e26 |
| 2133 | a2 | e63 | e28 |
| 2134 | a2 | e63 | e29 |
| 2135 | a2 | e63 | e30 |
| 2136 | a2 | e63 | e31 |
| 2137 | a2 | e63 | e32 |
| 2138 | a2 | e63 | e34 |
| 2139 | a2 | e63 | e35 |
| 2140 | a2 | e63 | e38 |
| 2141 | a2 | e63 | e39 |
| 2142 | a2 | e63 | e40 |
| 2143 | a2 | e63 | e42 |
| 2144 | a2 | e63 | e43 |
| 2145 | a2 | e63 | e44 |
| 2146 | a2 | e63 | e45 |
| 2147 | a2 | e63 | e46 |
| 2148 | a2 | e63 | e47 |
| 2149 | a2 | e63 | e48 |
| 2150 | a2 | e63 | e49 |
| 2151 | a2 | e63 | e50 |
| 2152 | a2 | e63 | e51 |
| 2153 | a2 | e63 | e52 |
| 2154 | a2 | e63 | e53 |
| 2155 | a2 | e63 | e54 |
| 2156 | a2 | e63 | e55 |
| 2157 | a2 | e63 | e56 |
| 2158 | a2 | e63 | e57 |
| 2159 | a2 | e63 | e58 |
| 2160 | a2 | e63 | e60 |
| 2161 | a2 | e63 | e61 |
| 2162 | a2 | e63 | e63 |
| 2163 | a2 | e63 | e64 |
| 2164 | a2 | e63 | e65 |
| 2165 | a2 | e63 | e66 |
| 2166 | a2 | e63 | e67 |
| 2167 | a2 | e64 | a1 |
| 2168 | a2 | e64 | d1 |
| 2169 | a2 | e64 | d1 |
| 2170 | a2 | e64 | d4 |
| 2171 | a2 | e64 | d5 |
| 2172 | a2 | e64 | d6 |
| 2173 | a2 | e64 | d8 |
| 2174 | a2 | e64 | d10 |
| 2175 | a2 | e64 | e3 |
| 2176 | a2 | e64 | e4 |
| 2177 | a2 | e64 | e5 |
| 2178 | a2 | e64 | e6 |
| 2179 | a2 | e64 | e7 |
| 2180 | a2 | e64 | e8 |
| 2181 | a2 | e64 | e9 |
| 2182 | a2 | e64 | e10 |
| 2183 | a2 | e64 | e11 |
| 2184 | a2 | e64 | e12 |
| 2185 | a2 | e64 | e13 |
| 2186 | a2 | e64 | e14 |
| 2187 | a2 | e64 | e15 |
| 2188 | a2 | e64 | e16 |
| 2189 | a2 | e64 | e17 |
| 2190 | a2 | e64 | e18 |
| 2191 | a2 | e64 | e19 |
| 2192 | a2 | e64 | e20 |
| 2193 | a2 | e64 | e21 |
| 2194 | a2 | e64 | e22 |
| 2195 | a2 | e64 | e23 |
| 2196 | a2 | e64 | e25 |
| 2197 | a2 | e64 | e26 |
| 2198 | a2 | e64 | e28 |
| 2199 | a2 | e64 | e29 |
| 2200 | a2 | e64 | e30 |
| Compound No. | Ar_{A} | Ar_{B} | Ar_{C} |
|---|---|---|---|
| 2201 | a2 | e64 | e31 |
| 2202 | a2 | e64 | e32 |
| 2203 | a2 | e64 | e34 |
| 2204 | a2 | e64 | e35 |
| 2205 | a2 | e64 | e38 |
| 2206 | a2 | e64 | e39 |
| 2207 | a2 | e64 | e40 |
| 2208 | a2 | e64 | e42 |
| 2209 | a2 | e64 | e43 |
| 2210 | a2 | e64 | e44 |
| 2211 | a2 | e64 | e45 |
| 2212 | a2 | e64 | e46 |
| 2213 | a2 | e64 | e47 |
| 2214 | a2 | e64 | e48 |
| 2215 | a2 | e64 | e49 |
| 2216 | a2 | e64 | e50 |
| 2217 | a2 | e64 | e51 |
| 2218 | a2 | e64 | e52 |
| 2219 | a2 | e64 | e53 |
| 2220 | a2 | e64 | e54 |
| 2221 | a2 | e64 | e55 |
| 2222 | a2 | e64 | e56 |
| 2223 | a2 | e64 | e57 |
| 2224 | a2 | e64 | e58 |
| 2225 | a2 | e64 | e60 |
| 2226 | a2 | e64 | e61 |
| 2227 | a2 | e64 | e64 |
| 2228 | a2 | e64 | e65 |
| 2229 | a2 | e64 | e66 |
| 2230 | a2 | e64 | e67 |

10. The light emitting element of claim 1, wherein the at least one function layer comprises:
an emission layer; and
a hole transport region between the first electrode and the emission layer, and
the hole transport region comprises the amine compound represented by Formula 1.

11. The light emitting element of claim 10, wherein the hole transport region comprises:
a hole injection layer on the first electrode; and
a hole transport layer on the hole injection layer, and
the hole transport layer comprises the amine compound represented by Formula 1.

12. An amine compound represented by Formula 1: 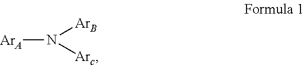
wherein in Formula 1,
Ar_{A} is represented by Formula A,
Ar_{B} is represented by Formula B, and
Ar_{C} is represented by Formula C: 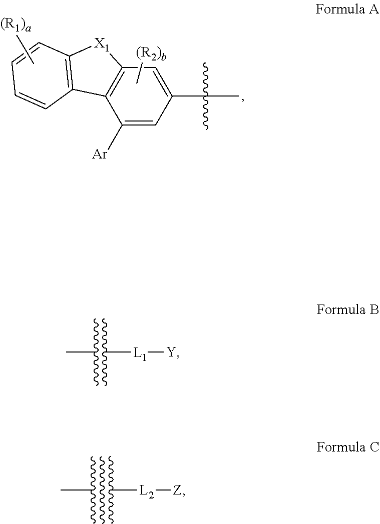
in Formula A to Formula C,
X₁ is O, or S,
Ar is a substituted or unsubstituted aryl group with a total carbon number of 6 to 16,
R₁ and R₂ are each independently a hydrogen atom, a substituted or unsubstituted alkyl group of 1 to 10 carbon atoms, a substituted or unsubstituted aryl group of 6 to 30 ring-forming carbon atoms, or a substituted or unsubstituted heteroaryl group of 2 to 30 ring-forming carbon atoms,
each of R₁ and R₂ does not comprise a substituted or unsubstituted, nitrogen-containing six-member heterocycle,
“a” is an integer of 0 to 4,
“b” is an integer of 0 to 2,
L₁ and L₂ are each independently a direct linkage, or a substituted or unsubstituted arylene group of 6 to 30 ring-forming carbon atoms,
Y and Z are each independently a substituted or unsubstituted aryl group of 6 to 30 ring-forming carbon atoms, or a substituted or unsubstituted heteroaryl group of 2 to 30 ring-forming carbon atoms,
at least one selected from among Y and Z is a substituted or unsubstituted aryl group of 10 to 30 ring-forming carbon atoms, or a substituted or unsubstituted heteroaryl group of 12 to 30 ring-forming carbon atoms,
each of Y and Z does not comprise a substituted or unsubstituted dimethylfluorenyl group, a substituted or unsubstituted fluoranthene group, or a halogen atom,
when Y is a substituted or unsubstituted naphthyl group, L₁ is not a direct linkage,
when Z is a substituted or unsubstituted naphthyl group, L₂ is not a direct linkage,
when Y is a substituted or unsubstituted carbazole group, L₁ is a direct linkage or an unsubstituted phenylene group,
when Z is a substituted or unsubstituted carbazole group, L₂ is a direct linkage or an unsubstituted phenylene group,
when L₁ is a m-phenylene group, Y is not a substituted or unsubstituted 10-arylphenanthren-9-yl group,
when L₂ is a m-phenylene group, Z is not a substituted or unsubstituted 10-arylphenanthren-9-yl group, [symbol]
, [symbol] , [symbol] and are positions respectively bonded to the nitrogen atom of Formula 1, and
Formula 1 optionally comprises a deuterium atom.

13. The amine compound of claim 12, wherein at least one selected from among Y and Z is represented by Formula 1a to Formula 1c: 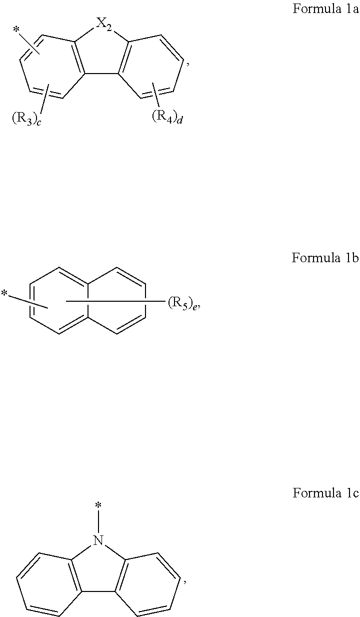
wherein in Formula 1a to Formula 1c,
X₂ is O, S, NRₐ, or CR_{b}R_{c},
R₃ to R₅, and Rₐ to R_{c} are each independently a hydrogen atom, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring,
“c” is an integer of 0 to 3,
“d” is an integer of 0 to 4,
“e” is an integer of 0 to 7,
“*-” is a position where Y is bonded to L₁, or a position where Z is bonded to L₂, and
Formula 1a to Formula 1c each optionally comprise a deuterium atom.

14. The amine compound of claim 12, wherein the amine compound represented by Formula 1 is a monoamine compound.

15. The amine compound of claim 12, wherein the amine compound represented by Formula 1 is represented by Formula 2-1 or Formula 2-2: 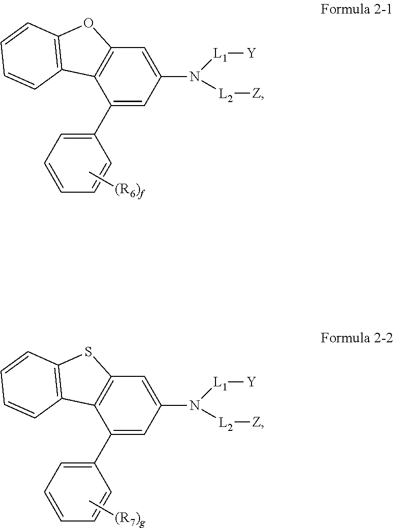
wherein in Formula 2-1 and Formula 2-2,
R₆ and R₇ are each independently a hydrogen atom, a substituted or unsubstituted alkyl group of 1 to 10 carbon atoms, a substituted or unsubstituted alkenyl group of 2 to 10 carbon atoms, or a substituted or unsubstituted aryl group of 6 to 30 ring-forming carbon atoms, and/or combined with an adjacent group to form a ring,
“f” and “g” are each independently an integer of 0 to 5,
L₁, L₂, Y and Z are as defined in Formula 1, and
Formula 2-1 and Formula 2-2 each optionally comprise a deuterium atom.

16. The amine compound of claim 12, wherein R₁ and R₂ are hydrogen atoms.

17. The amine compound of claim 12, wherein L₁ and L₂ are each independently a direct linkage, an unsubstituted phenylene group, or an unsubstituted biphenylene group.

18. The amine compound of claim 12, wherein Ar_{A} is any one selected from among a1 to a9, and b1 to b9: 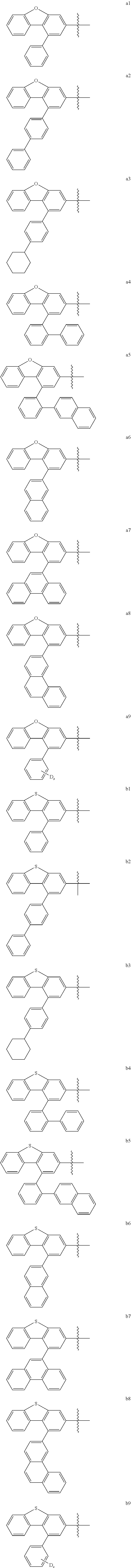
in a1 to a9, and b1 to b9, [symbol]
is a position bonded to the nitrogen atom of Formula 1, and “D” is a deuterium atom.

19. The amine compound of claim 18, wherein at least one selected from among Ar_{B} and Ar_{C} is selected from among a1 and e1 to e67, and when present a remaining Ar_{B} or Ar_{C} is selected from among d1 to d10: 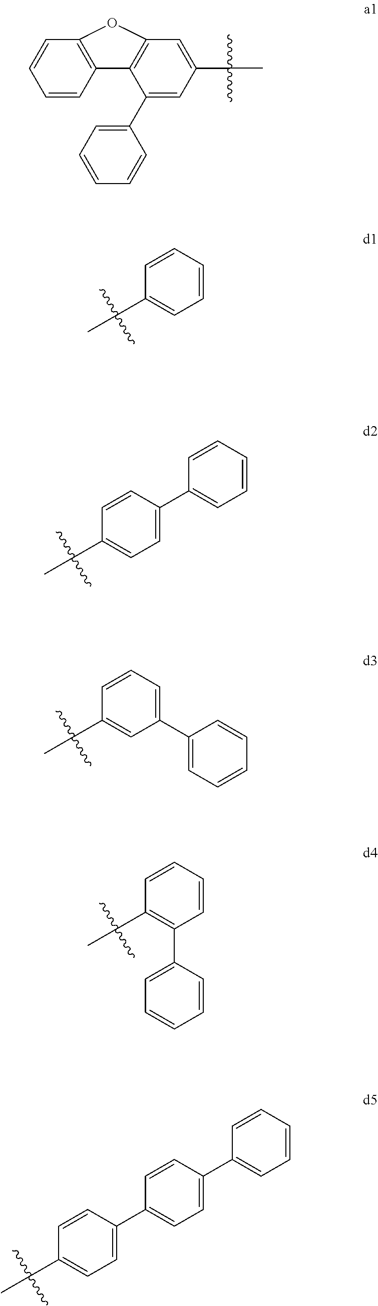 [SMILES: *c1cc(-c2ccccc2)c2c(c1)oc1ccccc12 ; *c1ccccc1 ; *c1ccc(-c2ccccc2)cc1 ; *c1cccc(-c2ccccc2)c1 ; *c1ccccc1-c1ccccc1 ; *c1ccc(-c2ccc(-c3ccccc3)cc2)cc1] 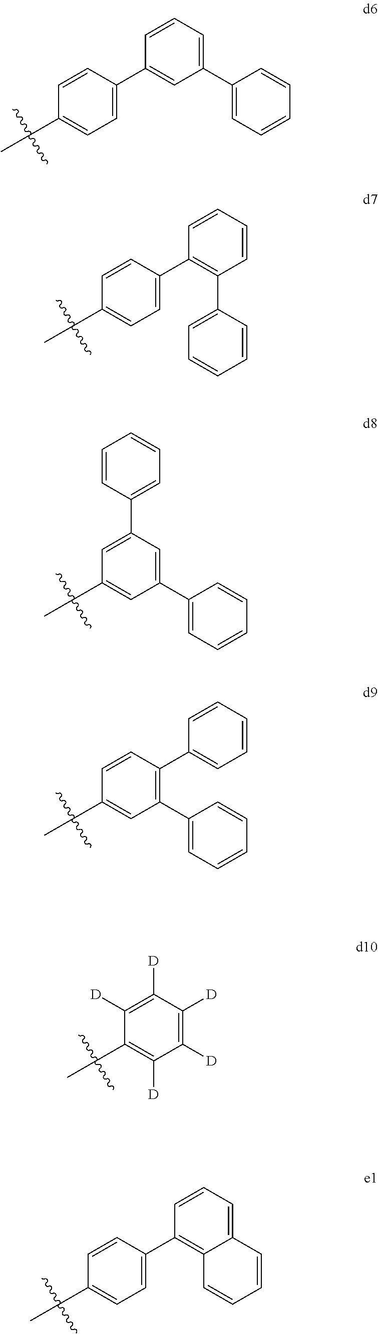 [SMILES: *c1ccc(-c2cccc(-c3ccccc3)c2)cc1 ; *c1ccc(-c2ccccc2-c2ccccc2)cc1 ; *c1cc(-c2ccccc2)cc(-c2ccccc2)c1 ; *c1ccc(-c2ccccc2)c(-c2ccccc2)c1 ; *c1c([2H])c([2H])c([2H])c([2H])c1[2H] ; *c1ccc(-c2cccc3ccccc23)cc1] 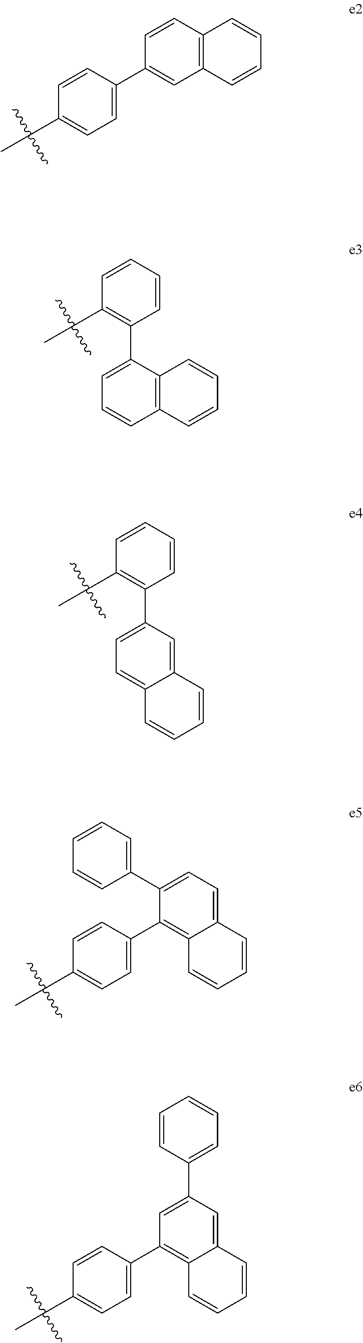 [SMILES: *c1ccc(-c2ccc3ccccc3c2)cc1 ; *c1ccccc1-c1cccc2ccccc12 ; *c1ccccc1-c1ccc2ccccc2c1 ; *c1ccc(-c2c(-c3ccccc3)ccc3ccccc23)cc1 ; *c1ccc(-c2cc(-c3ccccc3)cc3ccccc23)cc1] 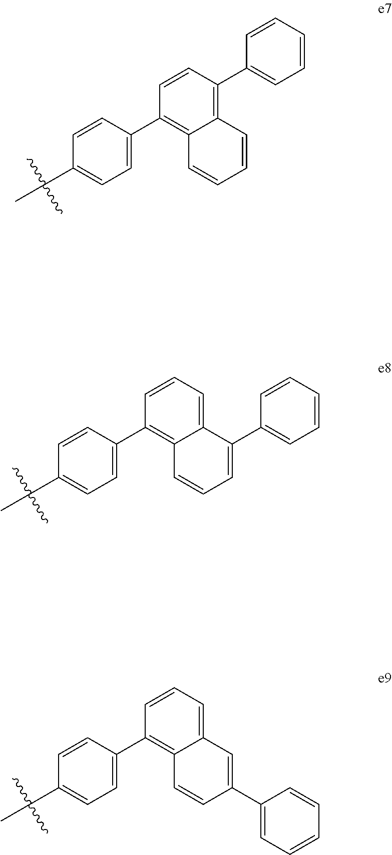 [SMILES: *c1ccc(-c2ccc(-c3ccccc3)c3ccccc23)cc1 ; *c1ccc(-c2cccc3c(-c4ccccc4)cccc23)cc1 ; *c1ccc(-c2cccc3cc(-c4ccccc4)ccc23)cc1] 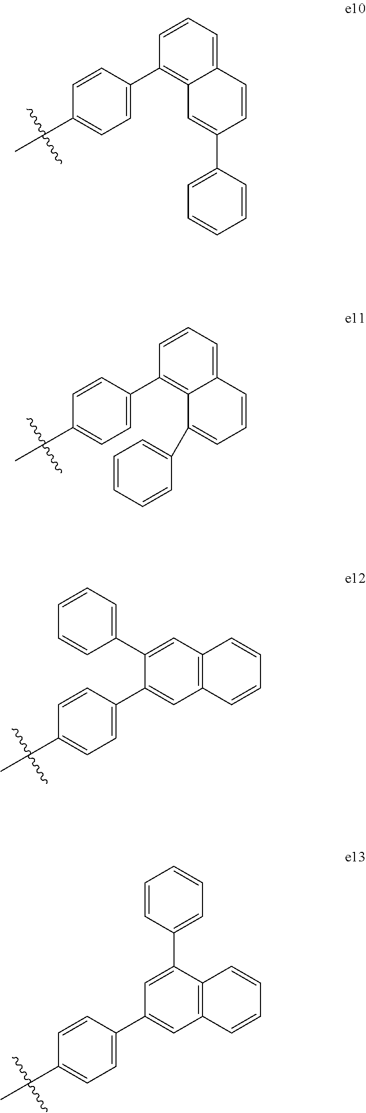 [SMILES: *c1ccc(-c2cccc3ccc(-c4ccccc4)cc23)cc1 ; *c1ccc(-c2cccc3cccc(-c4ccccc4)c23)cc1 ; *c1ccc(-c2cc3ccccc3cc2-c2ccccc2)cc1 ; *c1ccc(-c2cc(-c3ccccc3)c3ccccc3c2)cc1] 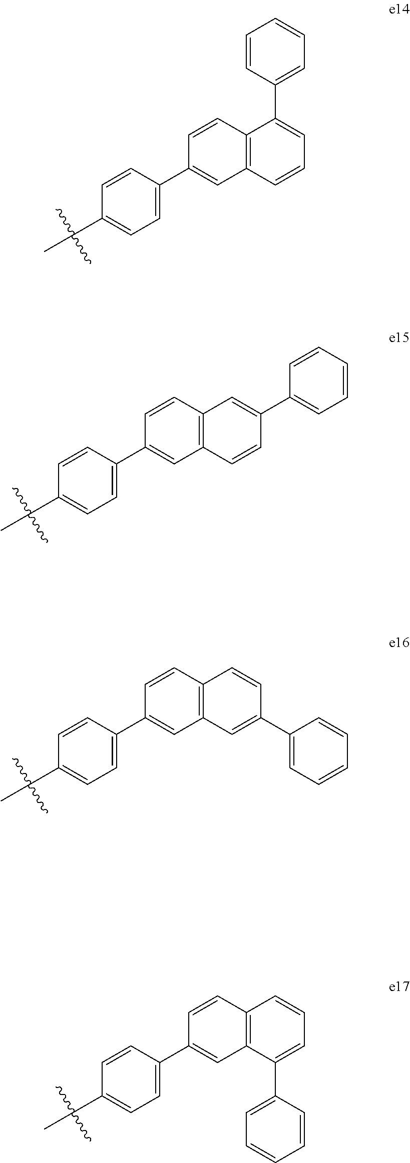 [SMILES: *c1ccc(-c2ccc3c(-c4ccccc4)cccc3c2)cc1 ; *c1ccc(-c2ccc3cc(-c4ccccc4)ccc3c2)cc1 ; *c1ccc(-c2ccc3ccc(-c4ccccc4)cc3c2)cc1 ; *c1ccc(-c2ccc3cccc(-c4ccccc4)c3c2)cc1] 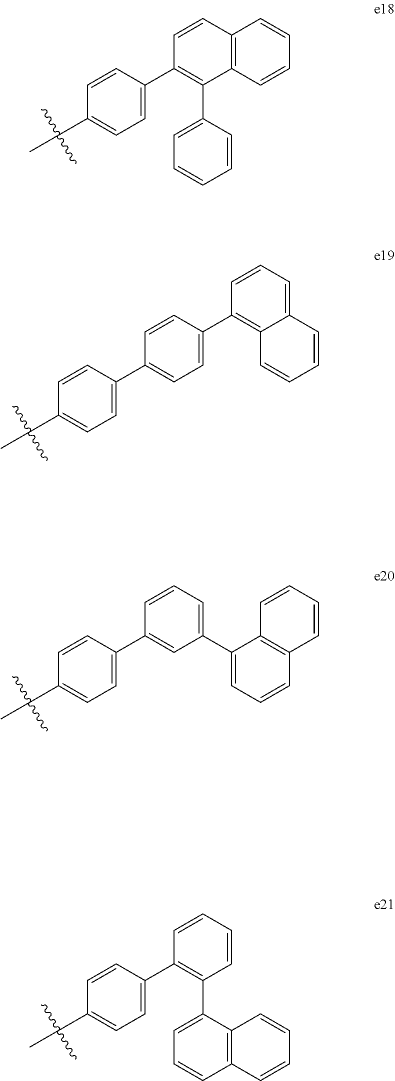 [SMILES: *c1ccc(-c2ccc3ccccc3c2-c2ccccc2)cc1 ; *c1ccc(-c2ccc(-c3cccc4ccccc34)cc2)cc1 ; *c1ccc(-c2cccc(-c3cccc4ccccc34)c2)cc1 ; *c1ccc(-c2ccccc2-c2cccc3ccccc23)cc1] 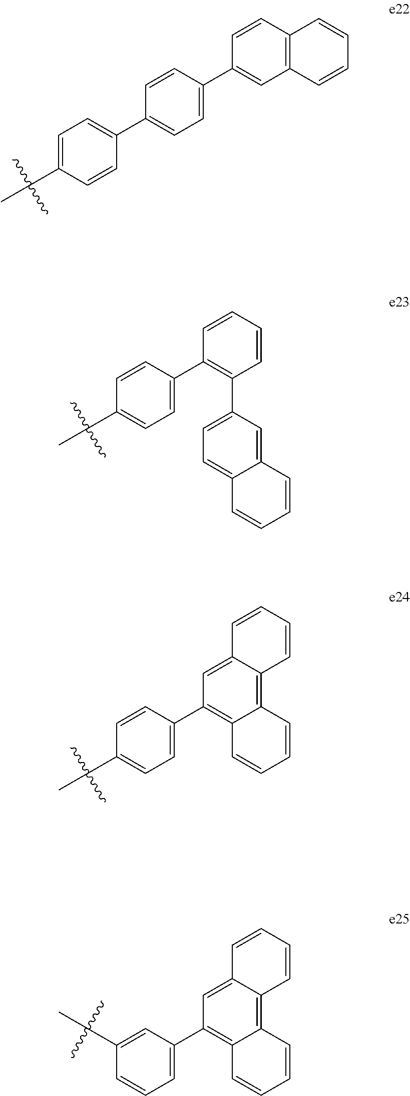 [SMILES: *c1ccc(-c2ccc(-c3ccc4ccccc4c3)cc2)cc1 ; *c1ccc(-c2ccccc2-c2ccc3ccccc3c2)cc1 ; *c1ccc(-c2cc3ccccc3c3ccccc23)cc1 ; *c1cccc(-c2cc3ccccc3c3ccccc23)c1] 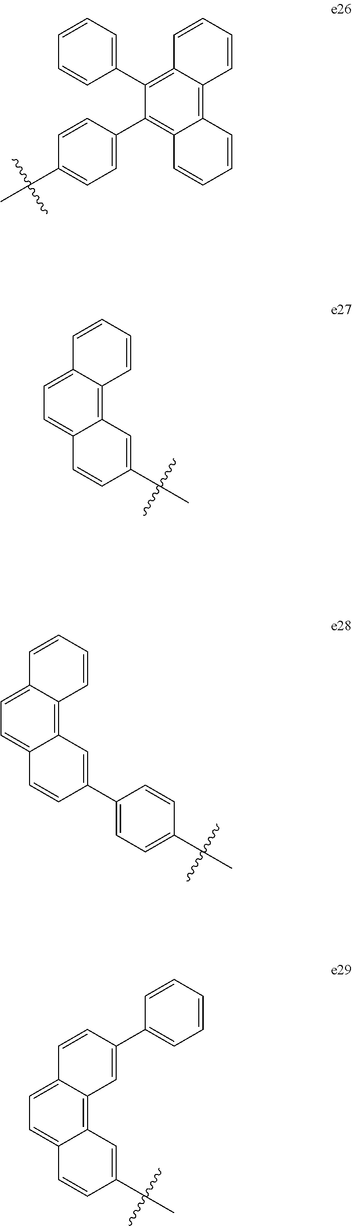 [SMILES: *c1ccc(-c2c(-c3ccccc3)c3ccccc3c3ccccc23)cc1 ; *c1ccc2ccc3ccccc3c2c1 ; *c1ccc(-c2ccc3ccc4ccccc4c3c2)cc1 ; *c1ccc2ccc3ccc(-c4ccccc4)cc3c2c1] 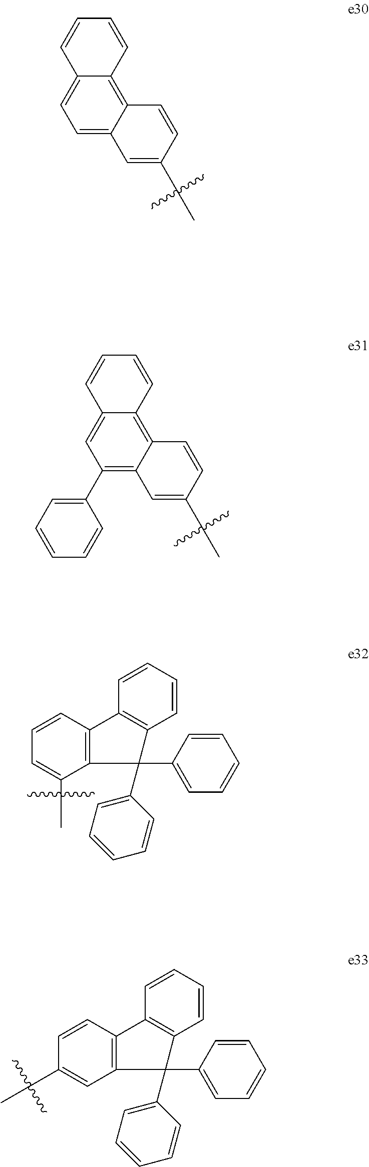 [SMILES: *c1ccc2c(ccc3ccccc32)c1 ; *c1ccc2c(c1)c(-c1ccccc1)cc1ccccc12 ; *c1cccc2c1C(c1ccccc1)(c1ccccc1)c1ccccc1-2 ; *c1ccc2c(c1)C(c1ccccc1)(c1ccccc1)c1ccccc1-2] 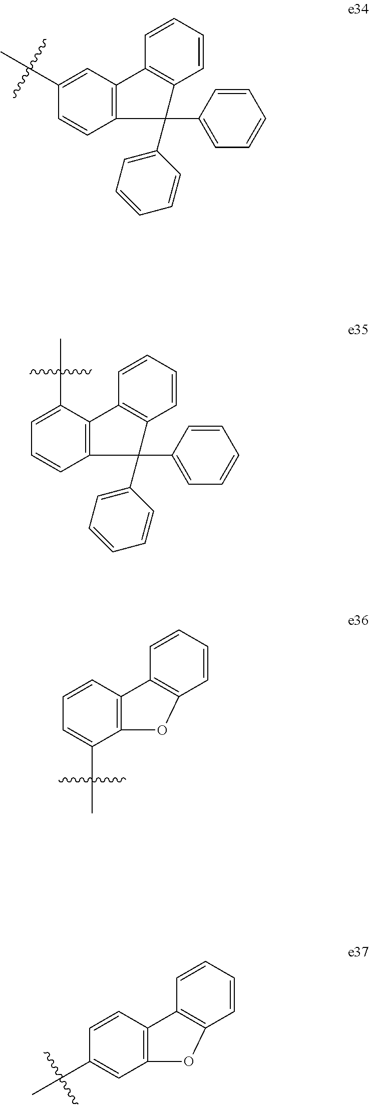 [SMILES: *c1ccc2c(c1)-c1ccccc1C2(c1ccccc1)c1ccccc1 ; *c1cccc2c1-c1ccccc1C2(c1ccccc1)c1ccccc1 ; *c1cccc2c1oc1ccccc12 ; *c1ccc2c(c1)oc1ccccc12] 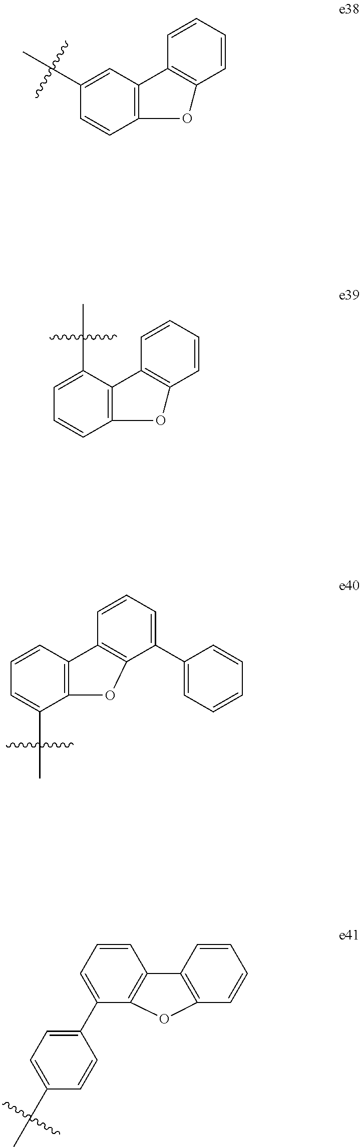 [SMILES: *c1ccc2oc3ccccc3c2c1 ; *c1cccc2oc3ccccc3c12 ; *c1cccc2c1oc1c(-c3ccccc3)cccc12 ; *c1ccc(-c2cccc3c2oc2ccccc23)cc1] 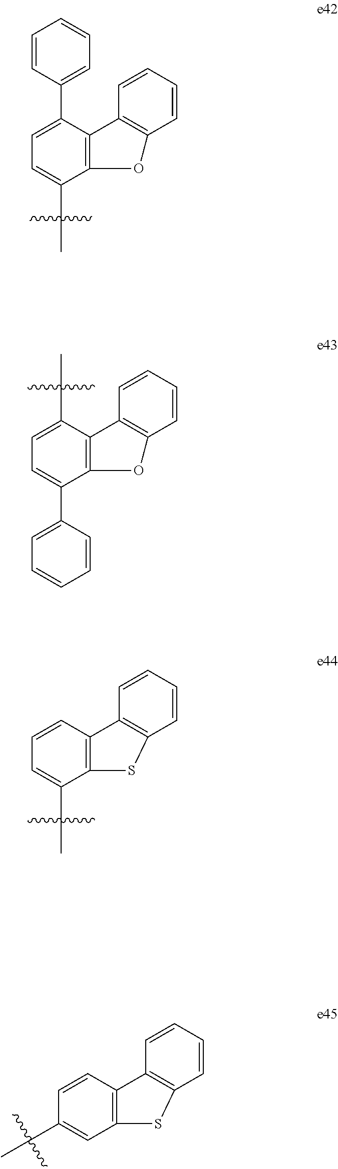 [SMILES: *c1ccc(-c2ccccc2)c2c1oc1ccccc12 ; *c1ccc(-c2ccccc2)c2oc3ccccc3c12 ; *c1cccc2c1sc1ccccc12 ; *c1ccc2c(c1)sc1ccccc12] 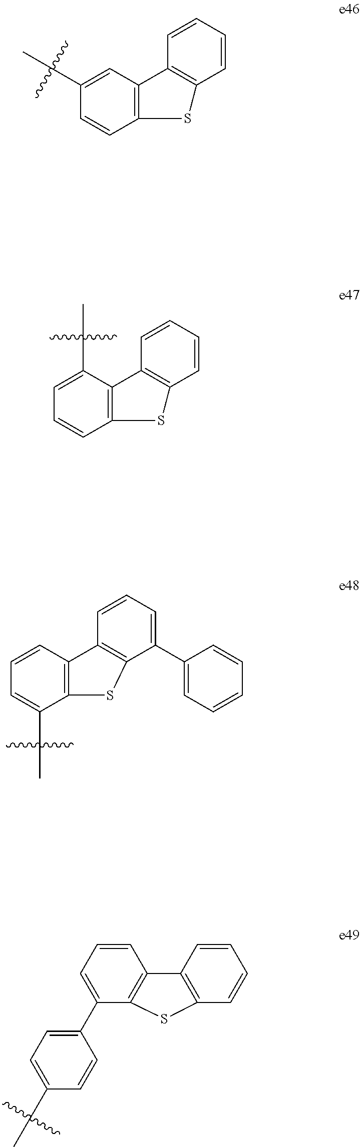 [SMILES: *c1ccc2sc3ccccc3c2c1 ; *c1cccc2sc3ccccc3c12 ; *c1cccc2c1sc1c(-c3ccccc3)cccc12 ; *c1ccc(-c2cccc3c2sc2ccccc23)cc1] 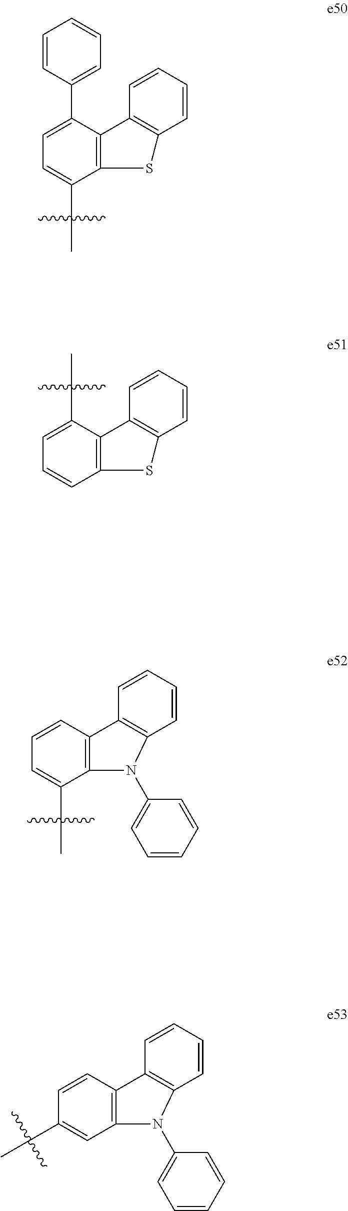 [SMILES: *c1ccc(-c2ccccc2)c2c1sc1ccccc12 ; *c1cccc2sc3ccccc3c12 ; *c1cccc2c3ccccc3n(-c3ccccc3)c12 ; *c1ccc2c3ccccc3n(-c3ccccc3)c2c1] 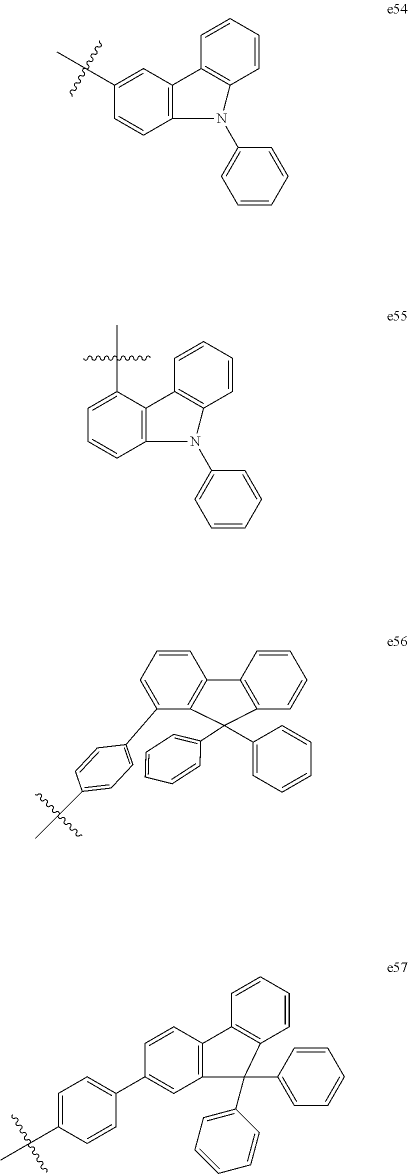 [SMILES: *c1ccc2c(c1)c1ccccc1n2-c1ccccc1 ; *c1cccc2c1c1ccccc1n2-c1ccccc1 ; *c1ccc(-c2cccc3c2C(c2ccccc2)(c2ccccc2)c2ccccc2-3)cc1 ; *c1ccc(-c2ccc3c(c2)C(c2ccccc2)(c2ccccc2)c2ccccc2-3)cc1] 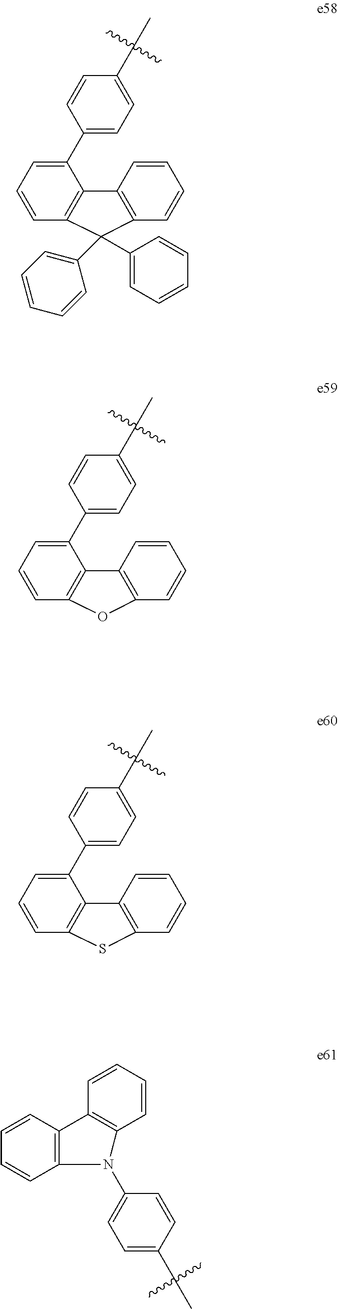 [SMILES: *c1ccc(-c2cccc3c2-c2ccccc2C3(c2ccccc2)c2ccccc2)cc1 ; *c1ccc(-c2cccc3oc4ccccc4c23)cc1 ; *c1ccc(-c2cccc3sc4ccccc4c23)cc1 ; *c1ccc(-n2c3ccccc3c3ccccc32)cc1] 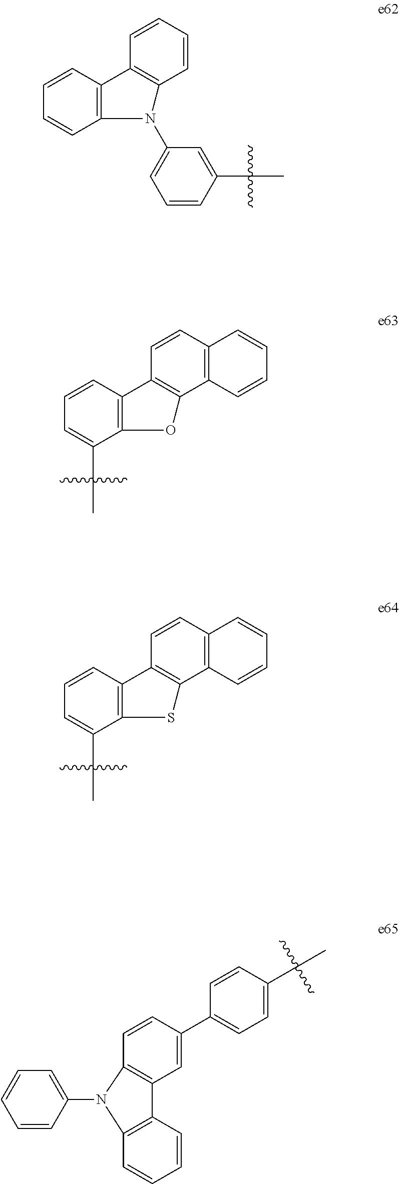 [SMILES: *c1cccc(-n2c3ccccc3c3ccccc32)c1 ; *c1cccc2c1oc1c3ccccc3ccc21 ; *c1cccc2c1sc1c3ccccc3ccc21 ; *c1ccc(-c2ccc3c(c2)c2ccccc2n3-c2ccccc2)cc1] 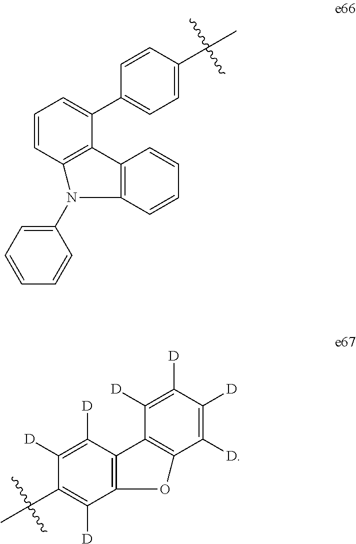
in a1, d1 to d10, and e1 to e67, [symbol]
is a position bonded to the nitrogen atom of Formula 1, and “D” is a deuterium atom.

20. The amine compound of claim 19, wherein the amine compound represented by Formula 1 is any one selected from among compounds in Compound Group 1:
Compound Group 1

| Compound No. | Ar_{A} | Ar_{B} | Ar_{C} |
|---|---|---|---|
| 1 | a1 | e2 | e27 |
| 2 | a2 | e2 | e27 |
| 3 | a3 | e2 | e27 |
| 4 | a4 | e2 | e27 |
| 5 | a5 | e2 | e27 |
| 6 | a6 | e2 | e27 |
| 7 | a7 | e2 | e27 |
| 8 | a8 | e2 | e27 |
| 9 | a9 | e2 | e27 |
| 10 | b1 | e2 | e27 |
| 11 | b2 | e2 | e27 |
| 12 | b3 | e2 | e27 |
| 13 | b4 | e2 | e27 |
| 14 | b5 | e2 | e27 |
| 15 | b6 | e2 | e27 |
| 16 | b7 | e2 | e27 |
| 17 | b8 | e2 | e27 |
| 18 | b9 | e2 | e27 |
| 19 | a1 | d2 | a1 |
| 20 | a1 | d2 | e1 |
| 21 | a1 | d2 | e2 |
| 22 | a1 | d2 | e3 |
| 23 | a1 | d2 | e4 |
| 24 | a1 | d2 | e5 |
| 25 | a1 | d2 | e6 |
| 26 | a1 | d2 | e7 |
| 27 | a1 | d2 | e8 |
| 28 | a1 | d2 | e9 |
| 29 | a1 | d2 | e10 |
| 30 | a1 | d2 | e11 |
| 31 | a1 | d2 | e12 |
| 32 | a1 | d2 | e13 |
| 33 | a1 | d2 | e14 |
| 34 | a1 | d2 | e15 |
| 35 | a1 | d2 | e16 |
| 36 | a1 | d2 | e17 |
| 37 | a1 | d2 | e18 |
| 38 | a1 | d2 | e19 |
| 39 | a1 | d2 | e20 |
| 40 | a1 | d2 | e21 |
| 41 | a1 | d2 | e22 |
| 42 | a1 | d2 | e23 |
| 43 | a1 | d2 | e24 |
| 44 | a1 | d2 | e25 |
| 45 | a1 | d2 | e26 |
| 46 | a1 | d2 | e27 |
| 47 | a1 | d2 | e28 |
| 48 | a1 | d2 | e29 |
| 49 | a1 | d2 | e30 |
| 50 | a1 | d2 | e31 |
| 51 | a1 | d2 | e32 |
| 52 | a1 | d2 | e33 |
| 53 | a1 | d2 | e34 |
| 54 | a1 | d2 | e35 |
| 55 | a1 | d2 | e36 |
| 56 | a1 | d2 | e37 |
| 57 | a1 | d2 | e38 |
| 58 | a1 | d2 | e39 |
| 59 | a1 | d2 | e40 |
| 60 | a1 | d2 | e41 |
| 61 | a1 | d2 | e42 |
| 62 | a1 | d2 | e43 |
| 63 | a1 | d2 | e44 |
| 64 | a1 | d2 | e45 |
| 65 | a1 | d2 | e46 |
| 66 | a1 | d2 | e47 |
| 67 | a1 | d2 | e48 |
| 68 | a1 | d2 | e49 |
| 69 | a1 | d2 | e50 |
| 70 | a1 | d2 | e51 |
| 71 | a1 | d2 | e52 |
| 72 | a1 | d2 | e53 |
| 73 | a1 | d2 | e54 |
| 74 | a1 | d2 | e55 |
| 75 | a1 | d2 | e56 |
| 76 | a1 | d2 | e57 |
| 77 | a1 | d2 | e58 |
| 78 | a1 | d2 | e59 |
| 79 | a1 | d2 | e60 |
| 80 | a1 | d2 | e61 |
| 81 | a1 | d2 | e62 |
| 82 | a1 | d2 | e63 |
| 83 | a1 | d2 | e64 |
| 84 | a1 | d2 | e65 |
| 85 | a1 | d2 | e66 |
| 86 | a1 | d2 | e67 |
| 87 | a1 | d3 | a1 |
| 88 | a1 | d3 | e1 |
| 89 | a1 | d3 | e2 |
| 90 | a1 | d3 | e3 |
| 91 | a1 | d3 | e4 |
| 92 | a1 | d3 | e5 |
| 93 | a1 | d3 | e6 |
| 94 | a1 | d3 | e7 |
| 95 | a1 | d3 | e8 |
| 96 | a1 | d3 | e9 |
| 97 | a1 | d3 | e10 |
| 98 | a1 | d3 | e11 |
| 99 | a1 | d3 | e12 |
| 100 | a1 | d3 | e13 |
| 101 | a1 | d3 | e14 |
| 102 | a1 | d3 | e15 |
| 103 | a1 | d3 | e16 |
| 104 | a1 | d3 | e17 |
| 105 | a1 | d3 | e18 |
| 106 | a1 | d3 | e19 |
| 107 | a1 | d3 | e20 |
| 108 | a1 | d3 | e21 |
| 109 | a1 | d3 | e22 |
| 110 | a1 | d3 | e23 |
| 111 | a1 | d3 | e24 |
| 112 | a1 | d3 | e25 |
| 113 | a1 | d3 | e26 |
| 114 | a1 | d3 | e27 |
| 115 | a1 | d3 | e28 |
| 116 | a1 | d3 | e29 |
| 117 | a1 | d3 | e30 |
| 118 | a1 | d3 | e31 |
| 119 | a1 | d3 | e32 |
| 120 | a1 | d3 | e33 |
| 121 | a1 | d3 | e34 |
| 122 | a1 | d3 | e35 |
| 123 | a1 | d3 | e36 |
| 124 | a1 | d3 | e37 |
| 125 | a1 | d3 | e38 |
| 126 | a1 | d3 | e39 |
| 127 | a1 | d3 | e40 |
| 128 | a1 | d3 | e41 |
| 129 | a1 | d3 | e42 |
| 130 | a1 | d3 | e43 |
| 131 | a1 | d3 | e44 |
| 132 | a1 | d3 | e45 |
| 133 | a1 | d3 | e46 |
| 134 | a1 | d3 | e47 |
| 135 | a1 | d3 | e48 |
| 136 | a1 | d3 | e49 |
| 137 | a1 | d3 | e50 |
| 138 | a1 | d3 | e51 |
| 139 | a1 | d3 | e52 |
| 140 | a1 | d3 | e53 |
| 141 | a1 | d3 | e54 |
| 142 | a1 | d3 | e55 |
| 143 | a1 | d3 | e56 |
| 144 | a1 | d3 | e57 |
| 145 | a1 | d3 | e58 |
| 146 | a1 | d3 | e59 |
| 147 | a1 | d3 | e60 |
| 148 | a1 | d3 | e61 |
| 149 | a1 | d3 | e62 |
| 150 | a1 | d3 | e63 |
| 151 | a1 | d3 | e64 |
| 152 | a1 | d3 | e65 |
| 153 | a1 | d3 | e66 |
| 154 | a1 | d3 | e67 |
| 155 | a1 | d7 | a1 |
| 156 | a1 | d7 | e1 |
| 157 | a1 | d7 | e1 |
| 158 | a1 | d7 | e2 |
| 159 | a1 | d7 | e3 |
| 160 | a1 | d7 | e4 |
| 161 | a1 | d7 | e5 |
| 162 | a1 | d7 | e6 |
| 163 | a1 | d7 | e7 |
| 164 | a1 | d7 | e8 |
| 165 | a1 | d7 | e9 |
| 166 | a1 | d7 | e10 |
| 167 | a1 | d7 | e11 |
| 168 | a1 | d7 | e12 |
| 169 | a1 | d7 | e13 |
| 170 | a1 | d7 | e14 |
| 171 | a1 | d7 | e15 |
| 172 | a1 | d7 | e16 |
| 173 | a1 | d7 | e17 |
| 174 | a1 | d7 | e18 |
| 175 | a1 | d7 | e19 |
| 176 | a1 | d7 | e20 |
| 177 | a1 | d7 | e21 |
| 178 | a1 | d7 | e22 |
| 179 | a1 | d7 | e23 |
| 180 | a1 | d7 | e24 |
| 181 | a1 | d7 | e25 |
| 182 | a1 | d7 | e26 |
| 183 | a1 | d7 | e27 |
| 184 | a1 | d7 | e28 |
| 185 | a1 | d7 | e29 |
| 186 | a1 | d7 | e30 |
| 187 | a1 | d7 | e31 |
| 188 | a1 | d7 | e32 |
| 189 | a1 | d7 | e33 |
| 190 | a1 | d7 | e34 |
| 191 | a1 | d7 | e35 |
| 192 | a1 | d7 | e36 |
| 193 | a1 | d7 | e37 |
| 194 | a1 | d7 | e38 |
| 195 | a1 | d7 | e39 |
| 196 | a1 | d7 | e40 |
| 197 | a1 | d7 | e41 |
| 198 | a1 | d7 | e42 |
| 199 | a1 | d7 | e43 |
| 200 | a1 | d7 | e44 |
| Compound No. | Ar_{A} | Ar_{B} | Ar_{C} |
|---|---|---|---|
| 201 | a1 | d7 | e45 |
| 202 | a1 | d7 | e46 |
| 203 | a1 | d7 | e47 |
| 204 | a1 | d7 | e48 |
| 205 | a1 | d7 | e49 |
| 206 | a1 | d7 | e50 |
| 207 | a1 | d7 | e51 |
| 208 | a1 | d7 | e52 |
| 209 | a1 | d7 | e53 |
| 210 | a1 | d7 | e54 |
| 211 | a1 | d7 | e55 |
| 212 | a1 | d7 | e56 |
| 213 | a1 | d7 | e57 |
| 214 | a1 | d7 | e58 |
| 215 | a1 | d7 | e59 |
| 216 | a1 | d7 | e60 |
| 217 | a1 | d7 | e61 |
| 218 | a1 | d7 | e62 |
| 219 | a1 | d7 | e63 |
| 220 | a1 | d7 | e64 |
| 221 | a1 | d7 | e65 |
| 222 | a1 | d7 | e66 |
| 223 | a1 | d7 | e67 |
| 224 | a1 | d9 | a1 |
| 225 | a1 | d9 | e1 |
| 226 | a1 | d9 | e1 |
| 227 | a1 | d9 | e2 |
| 228 | a1 | d9 | e3 |
| 229 | a1 | d9 | e4 |
| 230 | a1 | d9 | e5 |
| 231 | a1 | d9 | e6 |
| 232 | a1 | d9 | e7 |
| 233 | a1 | d9 | e8 |
| 234 | a1 | d9 | e9 |
| 235 | a1 | d9 | e10 |
| 236 | a1 | d9 | e11 |
| 237 | a1 | d9 | e12 |
| 238 | a1 | d9 | e13 |
| 239 | a1 | d9 | e14 |
| 240 | a1 | d9 | e15 |
| 241 | a1 | d9 | e16 |
| 242 | a1 | d9 | e17 |
| 243 | a1 | d9 | e18 |
| 244 | a1 | d9 | e19 |
| 245 | a1 | d9 | e20 |
| 246 | a1 | d9 | e21 |
| 247 | a1 | d9 | e22 |
| 248 | a1 | d9 | e23 |
| 249 | a1 | d9 | e24 |
| 250 | a1 | d9 | e25 |
| 251 | a1 | d9 | e26 |
| 252 | a1 | d9 | e27 |
| 253 | a1 | d9 | e28 |
| 254 | a1 | d9 | e29 |
| 255 | a1 | d9 | e30 |
| 256 | a1 | d9 | e31 |
| 257 | a1 | d9 | e32 |
| 258 | a1 | d9 | e33 |
| 259 | a1 | d9 | e34 |
| 260 | a1 | d9 | e35 |
| 261 | a1 | d9 | e36 |
| 262 | a1 | d9 | e37 |
| 263 | a1 | d9 | e38 |
| 264 | a1 | d9 | e39 |
| 265 | a1 | d9 | e40 |
| 266 | a1 | d9 | e41 |
| 267 | a1 | d9 | e42 |
| 268 | a1 | d9 | e43 |
| 269 | a1 | d9 | e44 |
| 270 | a1 | d9 | e45 |
| 271 | a1 | d9 | e46 |
| 272 | a1 | d9 | e47 |
| 273 | a1 | d9 | e48 |
| 274 | a1 | d9 | e49 |
| 275 | a1 | d9 | e50 |
| 276 | a1 | d9 | e51 |
| 277 | a1 | d9 | e52 |
| 278 | a1 | d9 | e53 |
| 279 | a1 | d9 | e54 |
| 280 | a1 | d9 | e55 |
| 281 | a1 | d9 | e56 |
| 282 | a1 | d9 | e57 |
| 283 | a1 | d9 | e58 |
| 284 | a1 | d9 | e59 |
| 285 | a1 | d9 | e60 |
| 286 | a1 | d9 | e61 |
| 287 | a1 | d9 | e62 |
| 288 | a1 | d9 | e63 |
| 289 | a1 | d9 | e64 |
| 290 | a1 | d9 | e65 |
| 291 | a1 | d9 | e66 |
| 292 | a1 | d9 | e67 |
| 293 | a1 | e1 | a1 |
| 294 | a1 | e1 | d1 |
| 295 | a1 | e1 | d1 |
| 296 | a1 | e1 | d4 |
| 297 | a1 | e1 | d5 |
| 298 | a1 | e1 | d6 |
| 299 | a1 | e1 | d8 |
| 300 | a1 | e1 | d10 |
| 301 | a1 | e1 | e1 |
| 302 | a1 | e1 | e1 |
| 303 | a1 | e1 | e2 |
| 304 | a1 | e1 | e3 |
| 305 | a1 | e1 | e4 |
| 306 | a1 | e1 | e5 |
| 307 | a1 | e1 | e6 |
| 308 | a1 | e1 | e7 |
| 309 | a1 | e1 | e8 |
| 310 | a1 | e1 | e9 |
| 311 | a1 | e1 | e10 |
| 312 | a1 | e1 | e11 |
| 313 | a1 | e1 | e12 |
| 314 | a1 | e1 | e13 |
| 315 | a1 | e1 | e14 |
| 316 | a1 | e1 | e15 |
| 317 | a1 | e1 | e16 |
| 318 | a1 | e1 | e17 |
| 319 | a1 | e1 | e18 |
| 320 | a1 | e1 | e19 |
| 321 | a1 | e1 | e20 |
| 322 | a1 | e1 | e21 |
| 323 | a1 | e1 | e22 |
| 324 | a1 | e1 | e23 |
| 325 | a1 | e1 | e24 |
| 326 | a1 | e1 | e25 |
| 327 | a1 | e1 | e26 |
| 328 | a1 | e1 | e27 |
| 329 | a1 | e1 | e28 |
| 330 | a1 | e1 | e29 |
| 331 | a1 | e1 | e30 |
| 332 | a1 | e1 | e31 |
| 333 | a1 | e1 | e32 |
| 334 | a1 | e1 | e33 |
| 335 | a1 | e1 | e34 |
| 336 | a1 | e1 | e35 |
| 337 | a1 | e1 | e36 |
| 338 | a1 | e1 | e37 |
| 339 | a1 | e1 | e38 |
| 340 | a1 | e1 | e39 |
| 341 | a1 | e1 | e40 |
| 342 | a1 | e1 | e41 |
| 343 | a1 | e1 | e42 |
| 344 | a1 | e1 | e43 |
| 345 | a1 | e1 | e44 |
| 346 | a1 | e1 | e45 |
| 347 | a1 | e1 | e46 |
| 348 | a1 | e1 | e47 |
| 349 | a1 | e1 | e48 |
| 350 | a1 | e1 | e49 |
| 351 | a1 | e1 | e50 |
| 352 | a1 | e1 | e51 |
| 353 | a1 | e1 | e52 |
| 354 | a1 | e1 | e53 |
| 355 | a1 | e1 | e54 |
| 356 | a1 | e1 | e55 |
| 357 | a1 | e1 | e56 |
| 358 | a1 | e1 | e57 |
| 359 | a1 | e1 | e58 |
| 360 | a1 | e1 | e59 |
| 361 | a1 | e1 | e60 |
| 362 | a1 | e1 | e61 |
| 363 | a1 | e1 | e62 |
| 364 | a1 | e1 | e63 |
| 365 | a1 | e1 | e64 |
| 366 | a1 | e1 | e65 |
| 367 | a1 | e1 | e66 |
| 368 | a1 | e1 | e67 |
| 369 | a1 | e2 | a1 |
| 370 | a1 | e2 | d1 |
| 371 | a1 | e2 | d1 |
| 372 | a1 | e2 | d4 |
| 373 | a1 | e2 | d5 |
| 374 | a1 | e2 | d6 |
| 375 | a1 | e2 | d8 |
| 376 | a1 | e2 | d10 |
| 377 | a1 | e2 | e2 |
| 378 | a1 | e2 | e3 |
| 379 | a1 | e2 | e4 |
| 380 | a1 | e2 | e5 |
| 381 | a1 | e2 | e6 |
| 382 | a1 | e2 | e7 |
| 383 | a1 | e2 | e8 |
| 384 | a1 | e2 | e9 |
| 385 | a1 | e2 | e10 |
| 386 | a1 | e2 | e11 |
| 387 | a1 | e2 | e12 |
| 388 | a1 | e2 | e13 |
| 389 | a1 | e2 | e14 |
| 390 | a1 | e2 | e15 |
| 391 | a1 | e2 | e16 |
| 392 | a1 | e2 | e17 |
| 393 | a1 | e2 | e18 |
| 394 | a1 | e2 | e19 |
| 395 | a1 | e2 | e20 |
| 396 | a1 | e2 | e21 |
| 397 | a1 | e2 | e22 |
| 398 | a1 | e2 | e23 |
| 399 | a1 | e2 | e24 |
| 400 | a1 | e2 | e25 |
| Compound No. | Ar_{A} | Ar_{B} | Ar_{C} |
|---|---|---|---|
| 401 | a1 | e2 | e26 |
| 402 | a1 | e2 | e28 |
| 403 | a1 | e2 | e29 |
| 404 | a1 | e2 | e30 |
| 405 | a1 | e2 | e31 |
| 406 | a1 | e2 | e32 |
| 407 | a1 | e2 | e33 |
| 408 | a1 | e2 | e34 |
| 409 | a1 | e2 | e35 |
| 410 | a1 | e2 | e36 |
| 411 | a1 | e2 | e37 |
| 412 | a1 | e2 | e38 |
| 413 | a1 | e2 | e39 |
| 414 | a1 | e2 | e40 |
| 415 | a1 | e2 | e41 |
| 416 | a1 | e2 | e42 |
| 417 | a1 | e2 | e43 |
| 418 | a1 | e2 | e44 |
| 419 | a1 | e2 | e45 |
| 420 | a1 | e2 | e46 |
| 421 | a1 | e2 | e47 |
| 422 | a1 | e2 | e48 |
| 423 | a1 | e2 | e49 |
| 424 | a1 | e2 | e50 |
| 425 | a1 | e2 | e51 |
| 426 | a1 | e2 | e52 |
| 427 | a1 | e2 | e53 |
| 428 | a1 | e2 | e54 |
| 429 | a1 | e2 | e55 |
| 430 | a1 | e2 | e56 |
| 431 | a1 | e2 | e57 |
| 432 | a1 | e2 | e58 |
| 433 | a1 | e2 | e59 |
| 434 | a1 | e2 | e60 |
| 435 | a1 | e2 | e61 |
| 436 | a1 | e2 | e62 |
| 437 | a1 | e2 | e63 |
| 438 | a1 | e2 | e64 |
| 439 | a1 | e2 | e65 |
| 440 | a1 | e2 | e66 |
| 441 | a1 | e2 | e67 |
| 442 | a1 | e24 | a1 |
| 443 | a1 | e24 | d1 |
| 444 | a1 | e24 | d1 |
| 445 | a1 | e24 | d4 |
| 446 | a1 | e24 | d5 |
| 447 | a1 | e24 | d6 |
| 448 | a1 | e24 | d8 |
| 449 | a1 | e24 | d10 |
| 450 | a1 | e24 | e3 |
| 451 | a1 | e24 | e4 |
| 452 | a1 | e24 | e5 |
| 453 | a1 | e24 | e6 |
| 454 | a1 | e24 | e7 |
| 455 | a1 | e24 | e8 |
| 456 | a1 | e24 | e9 |
| 457 | a1 | e24 | e10 |
| 458 | a1 | e24 | e11 |
| 459 | a1 | e24 | e12 |
| 460 | a1 | e24 | e13 |
| 461 | a1 | e24 | e14 |
| 462 | a1 | e24 | e15 |
| 463 | a1 | e24 | e16 |
| 464 | a1 | e24 | e17 |
| 465 | a1 | e24 | e18 |
| 466 | a1 | e24 | e19 |
| 467 | a1 | e24 | e20 |
| 468 | a1 | e24 | e21 |
| 469 | a1 | e24 | e22 |
| 470 | a1 | e24 | e23 |
| 471 | a1 | e24 | e24 |
| 472 | a1 | e24 | e25 |
| 473 | a1 | e24 | e26 |
| 474 | a1 | e24 | e27 |
| 475 | a1 | e24 | e28 |
| 476 | a1 | e24 | e29 |
| 477 | a1 | e24 | e30 |
| 478 | a1 | e24 | e31 |
| 479 | a1 | e24 | e32 |
| 480 | a1 | e24 | e33 |
| 481 | a1 | e24 | e34 |
| 482 | a1 | e24 | e35 |
| 483 | a1 | e24 | e36 |
| 484 | a1 | e24 | e37 |
| 485 | a1 | e24 | e38 |
| 486 | a1 | e24 | e39 |
| 487 | a1 | e24 | e40 |
| 488 | a1 | e24 | e41 |
| 489 | a1 | e24 | e42 |
| 490 | a1 | e24 | e43 |
| 491 | a1 | e24 | e44 |
| 492 | a1 | e24 | e45 |
| 493 | a1 | e24 | e46 |
| 494 | a1 | e24 | e47 |
| 495 | a1 | e24 | e48 |
| 496 | a1 | e24 | e49 |
| 497 | a1 | e24 | e50 |
| 498 | a1 | e24 | e51 |
| 499 | a1 | e24 | e52 |
| 500 | a1 | e24 | e53 |
| 501 | a1 | e24 | e54 |
| 502 | a1 | e24 | e55 |
| 503 | a1 | e24 | e56 |
| 504 | a1 | e24 | e57 |
| 505 | a1 | e24 | e58 |
| 506 | a1 | e24 | e59 |
| 507 | a1 | e24 | e60 |
| 508 | a1 | e24 | e61 |
| 509 | a1 | e24 | e62 |
| 510 | a1 | e24 | e63 |
| 511 | a1 | e24 | e64 |
| 512 | a1 | e24 | e65 |
| 513 | a1 | e24 | e66 |
| 514 | a1 | e24 | e67 |
| 515 | a1 | e27 | a1 |
| 516 | a1 | e27 | d1 |
| 517 | a1 | e27 | d1 |
| 518 | a1 | e27 | d4 |
| 519 | a1 | e27 | d5 |
| 520 | a1 | e27 | d6 |
| 521 | a1 | e27 | d8 |
| 522 | a1 | e27 | d10 |
| 523 | a1 | e27 | e3 |
| 524 | a1 | e27 | e4 |
| 525 | a1 | e27 | e5 |
| 526 | a1 | e27 | e6 |
| 527 | a1 | e27 | e7 |
| 528 | a1 | e27 | e8 |
| 529 | a1 | e27 | e9 |
| 530 | a1 | e27 | e10 |
| 531 | a1 | e27 | e11 |
| 532 | a1 | e27 | e12 |
| 533 | a1 | e27 | e13 |
| 534 | a1 | e27 | e14 |
| 535 | a1 | e27 | e15 |
| 536 | a1 | e27 | e16 |
| 537 | a1 | e27 | e17 |
| 538 | a1 | e27 | e18 |
| 539 | a1 | e27 | e19 |
| 540 | a1 | e27 | e20 |
| 541 | a1 | e27 | e21 |
| 542 | a1 | e27 | e22 |
| 543 | a1 | e27 | e23 |
| 544 | a1 | e27 | e25 |
| 545 | a1 | e27 | e26 |
| 546 | a1 | e27 | e27 |
| 547 | a1 | e27 | e28 |
| 548 | a1 | e27 | e29 |
| 549 | a1 | e27 | e30 |
| 550 | a1 | e27 | e31 |
| 551 | a1 | e27 | e32 |
| 552 | a1 | e27 | e33 |
| 553 | a1 | e27 | e34 |
| 554 | a1 | e27 | e35 |
| 555 | a1 | e27 | e36 |
| 556 | a1 | e27 | e37 |
| 557 | a1 | e27 | e38 |
| 558 | a1 | e27 | e39 |
| 559 | a1 | e27 | e40 |
| 560 | a1 | e27 | e41 |
| 561 | a1 | e27 | e42 |
| 562 | a1 | e27 | e43 |
| 563 | a1 | e27 | e44 |
| 564 | a1 | e27 | e45 |
| 565 | a1 | e27 | e46 |
| 566 | a1 | e27 | e47 |
| 567 | a1 | e27 | e48 |
| 568 | a1 | e27 | e49 |
| 569 | a1 | e27 | e50 |
| 570 | a1 | e27 | e51 |
| 571 | a1 | e27 | e52 |
| 572 | a1 | e27 | e53 |
| 573 | a1 | e27 | e54 |
| 574 | a1 | e27 | e55 |
| 575 | a1 | e27 | e56 |
| 576 | a1 | e27 | e57 |
| 577 | a1 | e27 | e58 |
| 578 | a1 | e27 | e59 |
| 579 | a1 | e27 | e60 |
| 580 | a1 | e27 | e61 |
| 581 | a1 | e27 | e62 |
| 582 | a1 | e27 | e63 |
| 583 | a1 | e27 | e64 |
| 584 | a1 | e27 | e65 |
| 585 | a1 | e27 | e66 |
| 586 | a1 | e27 | e67 |
| 587 | a1 | e33 | a1 |
| 588 | a1 | e33 | d1 |
| 589 | a1 | e33 | d1 |
| 590 | a1 | e33 | d4 |
| 591 | a1 | e33 | d5 |
| 592 | a1 | e33 | d6 |
| 593 | a1 | e33 | d8 |
| 594 | a1 | e33 | d10 |
| 595 | a1 | e33 | e3 |
| 596 | a1 | e33 | e4 |
| 597 | a1 | e33 | e5 |
| 598 | a1 | e33 | e6 |
| 599 | a1 | e33 | e7 |
| 600 | a1 | e33 | e8 |
| Compound No. | Ar_{A} | Ar_{B} | Ar_{C} |
|---|---|---|---|
| 801 | a1 | e41 | d4 |
| 802 | a1 | e41 | d5 |
| 803 | a1 | e41 | d6 |
| 804 | a1 | e41 | d8 |
| 805 | a1 | e41 | d10 |
| 806 | a1 | e41 | e3 |
| 807 | a1 | e41 | e4 |
| 808 | a1 | e41 | e5 |
| 809 | a1 | e41 | e6 |
| 810 | a1 | e41 | e7 |
| 811 | a1 | e41 | e8 |
| 812 | a1 | e41 | e9 |
| 813 | a1 | e41 | e10 |
| 814 | a1 | e41 | e11 |
| 815 | a1 | e41 | e12 |
| 816 | a1 | e41 | e13 |
| 817 | a1 | e41 | e14 |
| 818 | a1 | e41 | e15 |
| 819 | a1 | e41 | e16 |
| 820 | a1 | e41 | e17 |
| 821 | a1 | e41 | e18 |
| 822 | a1 | e41 | e19 |
| 823 | a1 | e41 | e20 |
| 824 | a1 | e41 | e21 |
| 825 | a1 | e41 | e22 |
| 826 | a1 | e41 | e23 |
| 827 | a1 | e41 | e25 |
| 828 | a1 | e41 | e26 |
| 829 | a1 | e41 | e28 |
| 830 | a1 | e41 | e29 |
| 831 | a1 | e41 | e30 |
| 832 | a1 | e41 | e31 |
| 833 | a1 | e41 | e32 |
| 834 | a1 | e41 | e34 |
| 835 | a1 | e41 | e35 |
| 836 | a1 | e41 | e38 |
| 837 | a1 | e41 | e39 |
| 838 | a1 | e41 | e40 |
| 839 | a1 | e41 | e41 |
| 840 | a1 | e41 | e42 |
| 841 | a1 | e41 | e43 |
| 842 | a1 | e41 | e44 |
| 843 | a1 | e41 | e45 |
| 844 | a1 | e41 | e46 |
| 845 | a1 | e41 | e47 |
| 846 | a1 | e41 | e48 |
| 847 | a1 | e41 | e49 |
| 848 | a1 | e41 | e50 |
| 849 | a1 | e41 | e51 |
| 850 | a1 | e41 | e52 |
| 851 | a1 | e41 | e53 |
| 852 | a1 | e41 | e54 |
| 853 | a1 | e41 | e55 |
| 854 | a1 | e41 | e56 |
| 855 | a1 | e41 | e57 |
| 856 | a1 | e41 | e58 |
| 857 | a1 | e41 | e59 |
| 858 | a1 | e41 | e60 |
| 859 | a1 | e41 | e61 |
| 860 | a1 | e41 | e62 |
| 861 | a1 | e41 | e63 |
| 862 | a1 | e41 | e64 |
| 863 | a1 | e41 | e65 |
| 864 | a1 | e41 | e66 |
| 865 | a1 | e41 | e67 |
| 866 | a1 | e59 | a1 |
| 867 | a1 | e59 | d1 |
| 868 | a1 | e59 | d1 |
| 869 | a1 | e59 | d4 |
| 870 | a1 | e59 | d5 |
| 871 | a1 | e59 | d6 |
| 872 | a1 | e59 | d8 |
| 873 | a1 | e59 | d10 |
| 874 | a1 | e59 | e3 |
| 875 | a1 | e59 | e4 |
| 876 | a1 | e59 | e5 |
| 877 | a1 | e59 | e6 |
| 878 | a1 | e59 | e7 |
| 879 | a1 | e59 | e8 |
| 880 | a1 | e59 | e9 |
| 881 | a1 | e59 | e10 |
| 882 | a1 | e59 | e11 |
| 883 | a1 | e59 | e12 |
| 884 | a1 | e59 | e13 |
| 885 | a1 | e59 | e14 |
| 886 | a1 | e59 | e15 |
| 887 | a1 | e59 | e16 |
| 888 | a1 | e59 | e17 |
| 889 | a1 | e59 | e18 |
| 890 | a1 | e59 | e19 |
| 891 | a1 | e59 | e20 |
| 892 | a1 | e59 | e21 |
| 893 | a1 | e59 | e22 |
| 894 | a1 | e59 | e23 |
| 895 | a1 | e59 | e25 |
| 896 | a1 | e59 | e26 |
| 897 | a1 | e59 | e28 |
| 898 | a1 | e59 | e29 |
| 899 | a1 | e59 | e30 |
| 900 | a1 | e59 | e31 |
| 901 | a1 | e59 | e32 |
| 902 | a1 | e59 | e34 |
| 903 | a1 | e59 | e35 |
| 904 | a1 | e59 | e38 |
| 905 | a1 | e59 | e39 |
| 906 | a1 | e59 | e40 |
| 907 | a1 | e59 | e42 |
| 908 | a1 | e59 | e43 |
| 909 | a1 | e59 | e44 |
| 910 | a1 | e59 | e45 |
| 911 | a1 | e59 | e46 |
| 912 | a1 | e59 | e47 |
| 913 | a1 | e59 | e48 |
| 914 | a1 | e59 | e49 |
| 915 | a1 | e59 | e50 |
| 916 | a1 | e59 | e51 |
| 917 | a1 | e59 | e52 |
| 918 | a1 | e59 | e53 |
| 919 | a1 | e59 | e54 |
| 920 | a1 | e59 | e55 |
| 921 | a1 | e59 | e56 |
| 922 | a1 | e59 | e57 |
| 923 | a1 | e59 | e58 |
| 924 | a1 | e59 | e59 |
| 925 | a1 | e59 | e60 |
| 926 | a1 | e59 | e61 |
| 927 | a1 | e59 | e62 |
| 928 | a1 | e59 | e63 |
| 929 | a1 | e59 | e64 |
| 930 | a1 | e59 | e65 |
| 931 | a1 | e59 | e66 |
| 932 | a1 | e59 | e67 |
| 933 | a1 | e62 | a1 |
| 934 | a1 | e62 | d1 |
| 935 | a1 | e62 | d1 |
| 936 | a1 | e62 | d5 |
| 937 | a1 | e62 | d6 |
| 938 | a1 | e62 | d8 |
| 939 | a1 | e62 | d10 |
| 940 | a1 | e62 | e5 |
| 941 | a1 | e62 | e6 |
| 942 | a1 | e62 | e7 |
| 943 | a1 | e62 | e8 |
| 944 | a1 | e62 | e9 |
| 945 | a1 | e62 | e10 |
| 946 | a1 | e62 | e11 |
| 947 | a1 | e62 | e12 |
| 948 | a1 | e62 | e13 |
| 949 | a1 | e62 | e14 |
| 950 | a1 | e62 | e15 |
| 951 | a1 | e62 | e16 |
| 952 | a1 | e62 | e17 |
| 953 | a1 | e62 | e18 |
| 954 | a1 | e62 | e19 |
| 955 | a1 | e62 | e20 |
| 956 | a1 | e62 | e21 |
| 957 | a1 | e62 | e22 |
| 958 | a1 | e62 | e23 |
| 959 | a1 | e62 | e25 |
| 960 | a1 | e62 | e26 |
| 961 | a1 | e62 | e28 |
| 962 | a1 | e62 | e29 |
| 963 | a1 | e62 | e30 |
| 964 | a1 | e62 | e31 |
| 965 | a1 | e62 | e32 |
| 966 | a1 | e62 | e34 |
| 967 | a1 | e62 | e35 |
| 968 | a1 | e62 | e38 |
| 969 | a1 | e62 | e39 |
| 970 | a1 | e62 | e40 |
| 971 | a1 | e62 | e42 |
| 972 | a1 | e62 | e43 |
| 973 | a1 | e62 | e44 |
| 974 | a1 | e62 | e45 |
| 975 | a1 | e62 | e46 |
| 976 | a1 | e62 | e47 |
| 977 | a1 | e62 | e48 |
| 978 | a1 | e62 | e49 |
| 979 | a1 | e62 | e50 |
| 980 | a1 | e62 | e51 |
| 981 | a1 | e62 | e52 |
| 982 | a1 | e62 | e53 |
| 983 | a1 | e62 | e54 |
| 984 | a1 | e62 | e55 |
| 985 | a1 | e62 | e56 |
| 986 | a1 | e62 | e57 |
| 987 | a1 | e62 | e58 |
| 988 | a1 | e62 | e60 |
| 989 | a1 | e62 | e61 |
| 990 | a1 | e62 | e62 |
| 991 | a1 | e62 | e63 |
| 992 | a1 | e62 | e64 |
| 993 | a1 | e62 | e65 |
| 994 | a1 | e62 | e66 |
| 995 | a1 | e62 | e67 |
| 996 | a1 | e63 | a1 |
| 997 | a1 | e63 | d1 |
| 998 | a1 | e63 | d1 |
| 999 | a1 | e63 | d4 |
| 1000 | a1 | e63 | d5 |
| Compound No. | Ar_{A} | Ar_{B} | Ar_{C} |
|---|---|---|---|
| 1001 | a1 | e63 | d6 |
| 1002 | a1 | e63 | d8 |
| 1003 | a1 | e63 | d10 |
| 1004 | a1 | e63 | e3 |
| 1005 | a1 | e63 | e4 |
| 1006 | a1 | e63 | e5 |
| 1007 | a1 | e63 | e6 |
| 1008 | a1 | e63 | e7 |
| 1009 | a1 | e63 | e8 |
| 1010 | a1 | e63 | e9 |
| 1011 | a1 | e63 | e10 |
| 1012 | a1 | e63 | e11 |
| 1013 | a1 | e63 | e12 |
| 1014 | a1 | e63 | e13 |
| 1015 | a1 | e63 | e14 |
| 1016 | a1 | e63 | e15 |
| 1017 | a1 | e63 | e16 |
| 1018 | a1 | e63 | e17 |
| 1019 | a1 | e63 | e18 |
| 1020 | a1 | e63 | e19 |
| 1021 | a1 | e63 | e20 |
| 1022 | a1 | e63 | e21 |
| 1023 | a1 | e63 | e22 |
| 1024 | a1 | e63 | e23 |
| 1025 | a1 | e63 | e25 |
| 1026 | a1 | e63 | e26 |
| 1027 | a1 | e63 | e28 |
| 1028 | a1 | e63 | e29 |
| 1029 | a1 | e63 | e30 |
| 1030 | a1 | e63 | e31 |
| 1031 | a1 | e63 | e32 |
| 1032 | a1 | e63 | e34 |
| 1033 | a1 | e63 | e35 |
| 1034 | a1 | e63 | e38 |
| 1035 | a1 | e63 | e39 |
| 1036 | a1 | e63 | e40 |
| 1037 | a1 | e63 | e42 |
| 1038 | a1 | e63 | e43 |
| 1039 | a1 | e63 | e44 |
| 1040 | a1 | e63 | e45 |
| 1041 | a1 | e63 | e46 |
| 1042 | a1 | e63 | e47 |
| 1043 | a1 | e63 | e48 |
| 1044 | a1 | e63 | e49 |
| 1045 | a1 | e63 | e50 |
| 1046 | a1 | e63 | e51 |
| 1047 | a1 | e63 | e52 |
| 1048 | a1 | e63 | e53 |
| 1049 | a1 | e63 | e54 |
| 1050 | a1 | e63 | e55 |
| 1051 | a1 | e63 | e56 |
| 1052 | a1 | e63 | e57 |
| 1053 | a1 | e63 | e58 |
| 1054 | a1 | e63 | e60 |
| 1055 | a1 | e63 | e61 |
| 1056 | a1 | e63 | e63 |
| 1057 | a1 | e63 | e64 |
| 1058 | a1 | e63 | e65 |
| 1059 | a1 | e63 | e66 |
| 1060 | a1 | e63 | e67 |
| 1061 | a1 | e64 | a1 |
| 1062 | a1 | e64 | d1 |
| 1063 | a1 | e64 | d1 |
| 1064 | a1 | e64 | d4 |
| 1065 | a1 | e64 | d5 |
| 1066 | a1 | e64 | d6 |
| 1067 | a1 | e64 | d8 |
| 1068 | a1 | e64 | d10 |
| 1069 | a1 | e64 | e3 |
| 1070 | a1 | e64 | e4 |
| 1071 | a1 | e64 | e5 |
| 1072 | a1 | e64 | e6 |
| 1073 | a1 | e64 | e7 |
| 1074 | a1 | e64 | e8 |
| 1075 | a1 | e64 | e9 |
| 1076 | a1 | e64 | e10 |
| 1077 | a1 | e64 | e11 |
| 1078 | a1 | e64 | e12 |
| 1079 | a1 | e64 | e13 |
| 1080 | a1 | e64 | e14 |
| 1081 | a1 | e64 | e15 |
| 1082 | a1 | e64 | e16 |
| 1083 | a1 | e64 | e17 |
| 1084 | a1 | e64 | e18 |
| 1085 | a1 | e64 | e19 |
| 1086 | a1 | e64 | e20 |
| 1087 | a1 | e64 | e21 |
| 1088 | a1 | e64 | e22 |
| 1089 | a1 | e64 | e23 |
| 1090 | a1 | e64 | e25 |
| 1091 | a1 | e64 | e26 |
| 1092 | a1 | e64 | e28 |
| 1093 | a1 | e64 | e29 |
| 1094 | a1 | e64 | e30 |
| 1095 | a1 | e64 | e31 |
| 1096 | a1 | e64 | e32 |
| 1097 | a1 | e64 | e34 |
| 1098 | a1 | e64 | e35 |
| 1099 | a1 | e64 | e38 |
| 1100 | a1 | e64 | e39 |
| 1101 | a1 | e64 | e40 |
| 1102 | a1 | e64 | e42 |
| 1103 | a1 | e64 | e43 |
| 1104 | a1 | e64 | e44 |
| 1105 | a1 | e64 | e45 |
| 1106 | a1 | e64 | e46 |
| 1107 | a1 | e64 | e47 |
| 1108 | a1 | e64 | e48 |
| 1109 | a1 | e64 | e49 |
| 1110 | a1 | e64 | e50 |
| 1111 | a1 | e64 | e51 |
| 1112 | a1 | e64 | e52 |
| 1113 | a1 | e64 | e53 |
| 1114 | a1 | e64 | e54 |
| 1115 | a1 | e64 | e55 |
| 1116 | a1 | e64 | e56 |
| 1117 | a1 | e64 | e57 |
| 1118 | a1 | e64 | e58 |
| 1119 | a1 | e64 | e60 |
| 1120 | a1 | e64 | e61 |
| 1121 | a1 | e64 | e64 |
| 1122 | a1 | e64 | e65 |
| 1123 | a1 | e64 | e66 |
| 1124 | a1 | e64 | e67 |
| 1125 | a2 | d2 | a1 |
| 1126 | a2 | d2 | e1 |
| 1127 | a2 | d2 | e2 |
| 1128 | a2 | d2 | e3 |
| 1129 | a2 | d2 | e4 |
| 1130 | a2 | d2 | e5 |
| 1131 | a2 | d2 | e6 |
| 1132 | a2 | d2 | e7 |
| 1133 | a2 | d2 | e8 |
| 1134 | a2 | d2 | e9 |
| 1135 | a2 | d2 | e10 |
| 1136 | a2 | d2 | e11 |
| 1137 | a2 | d2 | e12 |
| 1138 | a2 | d2 | e13 |
| 1139 | a2 | d2 | e14 |
| 1140 | a2 | d2 | e15 |
| 1141 | a2 | d2 | e16 |
| 1142 | a2 | d2 | e17 |
| 1143 | a2 | d2 | e18 |
| 1144 | a2 | d2 | e19 |
| 1145 | a2 | d2 | e20 |
| 1146 | a2 | d2 | e21 |
| 1147 | a2 | d2 | e22 |
| 1148 | a2 | d2 | e23 |
| 1149 | a2 | d2 | e24 |
| 1150 | a2 | d2 | e25 |
| 1151 | a2 | d2 | e26 |
| 1152 | a2 | d2 | e27 |
| 1153 | a2 | d2 | e28 |
| 1154 | a2 | d2 | e29 |
| 1155 | a2 | d2 | e30 |
| 1156 | a2 | d2 | e31 |
| 1157 | a2 | d2 | e32 |
| 1158 | a2 | d2 | e33 |
| 1159 | a2 | d2 | e34 |
| 1160 | a2 | d2 | e35 |
| 1161 | a2 | d2 | e36 |
| 1162 | a2 | d2 | e37 |
| 1163 | a2 | d2 | e38 |
| 1164 | a2 | d2 | e39 |
| 1165 | a2 | d2 | e40 |
| 1166 | a2 | d2 | e41 |
| 1167 | a2 | d2 | e42 |
| 1168 | a2 | d2 | e43 |
| 1169 | a2 | d2 | e44 |
| 1170 | a2 | d2 | e45 |
| 1171 | a2 | d2 | e46 |
| 1172 | a2 | d2 | e47 |
| 1173 | a2 | d2 | e48 |
| 1174 | a2 | d2 | e49 |
| 1175 | a2 | d2 | e50 |
| 1176 | a2 | d2 | e51 |
| 1177 | a2 | d2 | e52 |
| 1178 | a2 | d2 | e53 |
| 1179 | a2 | d2 | e54 |
| 1180 | a2 | d2 | e55 |
| 1181 | a2 | d2 | e56 |
| 1182 | a2 | d2 | e57 |
| 1183 | a2 | d2 | e58 |
| 1184 | a2 | d2 | e59 |
| 1185 | a2 | d2 | e60 |
| 1186 | a2 | d2 | e61 |
| 1187 | a2 | d2 | e62 |
| 1188 | a2 | d2 | e63 |
| 1189 | a2 | d2 | e64 |
| 1190 | a2 | d2 | e65 |
| 1191 | a2 | d2 | e66 |
| 1192 | a2 | d2 | e67 |
| 1193 | a2 | d3 | a1 |
| 1194 | a2 | d3 | e1 |
| 1195 | a2 | d3 | e2 |
| 1196 | a2 | d3 | e3 |
| 1197 | a2 | d3 | e4 |
| 1198 | a2 | d3 | e5 |
| 1199 | a2 | d3 | e6 |
| 1200 | a2 | d3 | e7 |
| Compound No. | Ar_{A} | Ar_{B} | Ar_{C} |
|---|---|---|---|
| 1201 | a2 | d3 | e8 |
| 1202 | a2 | d3 | e9 |
| 1203 | a2 | d3 | e10 |
| 1204 | a2 | d3 | e11 |
| 1205 | a2 | d3 | e12 |
| 1206 | a2 | d3 | e13 |
| 1207 | a2 | d3 | e14 |
| 1208 | a2 | d3 | e15 |
| 1209 | a2 | d3 | e16 |
| 1210 | a2 | d3 | e17 |
| 1211 | a2 | d3 | e18 |
| 1212 | a2 | d3 | e19 |
| 1213 | a2 | d3 | e20 |
| 1214 | a2 | d3 | e21 |
| 1215 | a2 | d3 | e22 |
| 1216 | a2 | d3 | e23 |
| 1217 | a2 | d3 | e24 |
| 1218 | a2 | d3 | e25 |
| 1219 | a2 | d3 | e26 |
| 1220 | a2 | d3 | e27 |
| 1221 | a2 | d3 | e28 |
| 1222 | a2 | d3 | e29 |
| 1223 | a2 | d3 | e30 |
| 1224 | a2 | d3 | e31 |
| 1225 | a2 | d3 | e32 |
| 1226 | a2 | d3 | e33 |
| 1227 | a2 | d3 | e34 |
| 1228 | a2 | d3 | e35 |
| 1229 | a2 | d3 | e36 |
| 1230 | a2 | d3 | e37 |
| 1231 | a2 | d3 | e38 |
| 1232 | a2 | d3 | e39 |
| 1233 | a2 | d3 | e40 |
| 1234 | a2 | d3 | e41 |
| 1235 | a2 | d3 | e42 |
| 1236 | a2 | d3 | e43 |
| 1237 | a2 | d3 | e44 |
| 1238 | a2 | d3 | e45 |
| 1239 | a2 | d3 | e46 |
| 1240 | a2 | d3 | e47 |
| 1241 | a2 | d3 | e48 |
| 1242 | a2 | d3 | e49 |
| 1243 | a2 | d3 | e50 |
| 1244 | a2 | d3 | e51 |
| 1245 | a2 | d3 | e52 |
| 1246 | a2 | d3 | e53 |
| 1247 | a2 | d3 | e54 |
| 1248 | a2 | d3 | e55 |
| 1249 | a2 | d3 | e56 |
| 1250 | a2 | d3 | e57 |
| 1251 | a2 | d3 | e58 |
| 1252 | a2 | d3 | e59 |
| 1253 | a2 | d3 | e60 |
| 1254 | a2 | d3 | e61 |
| 1255 | a2 | d3 | e62 |
| 1256 | a2 | d3 | e63 |
| 1257 | a2 | d3 | e64 |
| 1258 | a2 | d3 | e65 |
| 1259 | a2 | d3 | e66 |
| 1260 | a2 | d3 | e67 |
| 1261 | a2 | d7 | a1 |
| 1262 | a2 | d7 | e1 |
| 1263 | a2 | d7 | e1 |
| 1264 | a2 | d7 | e2 |
| 1265 | a2 | d7 | e3 |
| 1266 | a2 | d7 | e4 |
| 1267 | a2 | d7 | e5 |
| 1268 | a2 | d7 | e6 |
| 1269 | a2 | d7 | e7 |
| 1270 | a2 | d7 | e8 |
| 1271 | a2 | d7 | e9 |
| 1272 | a2 | d7 | e10 |
| 1273 | a2 | d7 | e11 |
| 1274 | a2 | d7 | e12 |
| 1275 | a2 | d7 | e13 |
| 1276 | a2 | d7 | e14 |
| 1277 | a2 | d7 | e15 |
| 1278 | a2 | d7 | e16 |
| 1279 | a2 | d7 | e17 |
| 1280 | a2 | d7 | e18 |
| 1281 | a2 | d7 | e19 |
| 1282 | a2 | d7 | e20 |
| 1283 | a2 | d7 | e21 |
| 1284 | a2 | d7 | e22 |
| 1285 | a2 | d7 | e23 |
| 1286 | a2 | d7 | e24 |
| 1287 | a2 | d7 | e25 |
| 1288 | a2 | d7 | e26 |
| 1289 | a2 | d7 | e27 |
| 1290 | a2 | d7 | e28 |
| 1291 | a2 | d7 | e29 |
| 1292 | a2 | d7 | e30 |
| 1293 | a2 | d7 | e31 |
| 1294 | a2 | d7 | e32 |
| 1295 | a2 | d7 | e33 |
| 1296 | a2 | d7 | e34 |
| 1297 | a2 | d7 | e35 |
| 1298 | a2 | d7 | e36 |
| 1299 | a2 | d7 | e37 |
| 1300 | a2 | d7 | e38 |
| 1301 | a2 | d7 | e39 |
| 1302 | a2 | d7 | e40 |
| 1303 | a2 | d7 | e41 |
| 1304 | a2 | d7 | e42 |
| 1305 | a2 | d7 | e43 |
| 1306 | a2 | d7 | e44 |
| 1307 | a2 | d7 | e45 |
| 1308 | a2 | d7 | e46 |
| 1309 | a2 | d7 | e47 |
| 1310 | a2 | d7 | e48 |
| 1311 | a2 | d7 | e49 |
| 1312 | a2 | d7 | e50 |
| 1313 | a2 | d7 | e51 |
| 1314 | a2 | d7 | e52 |
| 1315 | a2 | d7 | e53 |
| 1316 | a2 | d7 | e54 |
| 1317 | a2 | d7 | e55 |
| 1318 | a2 | d7 | e56 |
| 1319 | a2 | d7 | e57 |
| 1320 | a2 | d7 | e58 |
| 1321 | a2 | d7 | e59 |
| 1322 | a2 | d7 | e60 |
| 1323 | a2 | d7 | e61 |
| 1324 | a2 | d7 | e62 |
| 1325 | a2 | d7 | e63 |
| 1326 | a2 | d7 | e64 |
| 1327 | a2 | d7 | e65 |
| 1328 | a2 | d7 | e66 |
| 1329 | a2 | d7 | e67 |
| 1330 | a2 | d9 | a1 |
| 1331 | a2 | d9 | e1 |
| 1332 | a2 | d9 | e1 |
| 1333 | a2 | d9 | e2 |
| 1334 | a2 | d9 | e3 |
| 1335 | a2 | d9 | e4 |
| 1336 | a2 | d9 | e5 |
| 1337 | a2 | d9 | e6 |
| 1338 | a2 | d9 | e7 |
| 1339 | a2 | d9 | e8 |
| 1340 | a2 | d9 | e9 |
| 1341 | a2 | d9 | e10 |
| 1342 | a2 | d9 | e11 |
| 1343 | a2 | d9 | e12 |
| 1344 | a2 | d9 | e13 |
| 1345 | a2 | d9 | e14 |
| 1346 | a2 | d9 | e15 |
| 1347 | a2 | d9 | e16 |
| 1348 | a2 | d9 | e17 |
| 1349 | a2 | d9 | e18 |
| 1350 | a2 | d9 | e19 |
| 1351 | a2 | d9 | e20 |
| 1352 | a2 | d9 | e21 |
| 1353 | a2 | d9 | e22 |
| 1354 | a2 | d9 | e23 |
| 1355 | a2 | d9 | e24 |
| 1356 | a2 | d9 | e25 |
| 1357 | a2 | d9 | e26 |
| 1358 | a2 | d9 | e27 |
| 1359 | a2 | d9 | e28 |
| 1360 | a2 | d9 | e29 |
| 1361 | a2 | d9 | e30 |
| 1362 | a2 | d9 | e31 |
| 1363 | a2 | d9 | e32 |
| 1364 | a2 | d9 | e33 |
| 1365 | a2 | d9 | e34 |
| 1366 | a2 | d9 | e35 |
| 1367 | a2 | d9 | e36 |
| 1368 | a2 | d9 | e37 |
| 1369 | a2 | d9 | e38 |
| 1370 | a2 | d9 | e39 |
| 1371 | a2 | d9 | e40 |
| 1372 | a2 | d9 | e41 |
| 1373 | a2 | d9 | e42 |
| 1374 | a2 | d9 | e43 |
| 1375 | a2 | d9 | e44 |
| 1376 | a2 | d9 | e45 |
| 1377 | a2 | d9 | e46 |
| 1378 | a2 | d9 | e47 |
| 1379 | a2 | d9 | e48 |
| 1380 | a2 | d9 | e49 |
| 1381 | a2 | d9 | e50 |
| 1382 | a2 | d9 | e51 |
| 1383 | a2 | d9 | e52 |
| 1384 | a2 | d9 | e53 |
| 1385 | a2 | d9 | e54 |
| 1386 | a2 | d9 | e55 |
| 1387 | a2 | d9 | e56 |
| 1388 | a2 | d9 | e57 |
| 1389 | a2 | d9 | e58 |
| 1390 | a2 | d9 | e59 |
| 1391 | a2 | d9 | e60 |
| 1392 | a2 | d9 | e61 |
| 1393 | a2 | d9 | e62 |
| 1394 | a2 | d9 | e63 |
| 1395 | a2 | d9 | e64 |
| 1396 | a2 | d9 | e65 |
| 1397 | a2 | d9 | e66 |
| 1398 | a2 | d9 | e67 |
| 1399 | a2 | e1 | a1 |
| 1400 | a2 | e1 | d1 |
| Compound No. | Ar_{A} | Ar_{B} | Ar_{C} |
|---|---|---|---|
| 1401 | a2 | e1 | d1 |
| 1402 | a2 | e1 | d4 |
| 1403 | a2 | e1 | d5 |
| 1404 | a2 | e1 | d6 |
| 1405 | a2 | e1 | d8 |
| 1406 | a2 | e1 | d10 |
| 1407 | a2 | e1 | e1 |
| 1408 | a2 | e1 | e1 |
| 1409 | a2 | e1 | e2 |
| 1410 | a2 | e1 | e3 |
| 1411 | a2 | e1 | e4 |
| 1412 | a2 | e1 | e5 |
| 1413 | a2 | e1 | e6 |
| 1414 | a2 | e1 | e7 |
| 1415 | a2 | e1 | e8 |
| 1416 | a2 | e1 | e9 |
| 1417 | a2 | e1 | e10 |
| 1418 | a2 | e1 | e11 |
| 1419 | a2 | e1 | e12 |
| 1420 | a2 | e1 | e13 |
| 1421 | a2 | e1 | e14 |
| 1422 | a2 | e1 | e15 |
| 1423 | a2 | e1 | e16 |
| 1424 | a2 | e1 | e17 |
| 1425 | a2 | e1 | e18 |
| 1426 | a2 | e1 | e19 |
| 1427 | a2 | e1 | e20 |
| 1428 | a2 | e1 | e21 |
| 1429 | a2 | e1 | e22 |
| 1430 | a2 | e1 | e23 |
| 1431 | a2 | e1 | e24 |
| 1432 | a2 | e1 | e25 |
| 1433 | a2 | e1 | e26 |
| 1434 | a2 | e1 | e27 |
| 1435 | a2 | e1 | e28 |
| 1436 | a2 | e1 | e29 |
| 1437 | a2 | e1 | e30 |
| 1438 | a2 | e1 | e31 |
| 1439 | a2 | e1 | e32 |
| 1440 | a2 | e1 | e33 |
| 1441 | a2 | e1 | e34 |
| 1442 | a2 | e1 | e35 |
| 1443 | a2 | e1 | e36 |
| 1444 | a2 | e1 | e37 |
| 1445 | a2 | e1 | e38 |
| 1446 | a2 | e1 | e39 |
| 1447 | a2 | e1 | e40 |
| 1448 | a2 | e1 | e41 |
| 1449 | a2 | e1 | e42 |
| 1450 | a2 | e1 | e43 |
| 1451 | a2 | e1 | e44 |
| 1452 | a2 | e1 | e45 |
| 1453 | a2 | e1 | e46 |
| 1454 | a2 | e1 | e47 |
| 1455 | a2 | e1 | e48 |
| 1456 | a2 | e1 | e49 |
| 1457 | a2 | e1 | e50 |
| 1458 | a2 | e1 | e51 |
| 1459 | a2 | e1 | e52 |
| 1460 | a2 | e1 | e53 |
| 1461 | a2 | e1 | e54 |
| 1462 | a2 | e1 | e55 |
| 1463 | a2 | e1 | e56 |
| 1464 | a2 | e1 | e57 |
| 1465 | a2 | e1 | e58 |
| 1466 | a2 | e1 | e59 |
| 1467 | a2 | e1 | e60 |
| 1468 | a2 | e1 | e61 |
| 1469 | a2 | e1 | e62 |
| 1470 | a2 | e1 | e63 |
| 1471 | a2 | e1 | e64 |
| 1472 | a2 | e1 | e65 |
| 1473 | a2 | e1 | e66 |
| 1474 | a2 | e1 | e67 |
| 1475 | a2 | e2 | a1 |
| 1476 | a2 | e2 | d1 |
| 1477 | a2 | e2 | d1 |
| 1478 | a2 | e2 | d4 |
| 1479 | a2 | e2 | d5 |
| 1480 | a2 | e2 | d6 |
| 1481 | a2 | e2 | d8 |
| 1482 | a2 | e2 | d10 |
| 1483 | a2 | e2 | e2 |
| 1484 | a2 | e2 | e3 |
| 1485 | a2 | e2 | e4 |
| 1486 | a2 | e2 | e5 |
| 1487 | a2 | e2 | e6 |
| 1488 | a2 | e2 | e7 |
| 1489 | a2 | e2 | e8 |
| 1490 | a2 | e2 | e9 |
| 1491 | a2 | e2 | e10 |
| 1492 | a2 | e2 | e11 |
| 1493 | a2 | e2 | e12 |
| 1494 | a2 | e2 | e13 |
| 1495 | a2 | e2 | e14 |
| 1496 | a2 | e2 | e15 |
| 1497 | a2 | e2 | e16 |
| 1498 | a2 | e2 | e17 |
| 1499 | a2 | e2 | e18 |
| 1500 | a2 | e2 | e19 |
| 1501 | a2 | e2 | e20 |
| 1502 | a2 | e2 | e21 |
| 1503 | a2 | e2 | e22 |
| 1504 | a2 | e2 | e23 |
| 1505 | a2 | e2 | e24 |
| 1506 | a2 | e2 | e25 |
| 1507 | a2 | e2 | e26 |
| 1508 | a2 | e2 | e28 |
| 1509 | a2 | e2 | e29 |
| 1510 | a2 | e2 | e30 |
| 1511 | a2 | e2 | e31 |
| 1512 | a2 | e2 | e32 |
| 1513 | a2 | e2 | e33 |
| 1514 | a2 | e2 | e34 |
| 1515 | a2 | e2 | e35 |
| 1516 | a2 | e2 | e36 |
| 1517 | a2 | e2 | e37 |
| 1518 | a2 | e2 | e38 |
| 1519 | a2 | e2 | e39 |
| 1520 | a2 | e2 | e40 |
| 1521 | a2 | e2 | e41 |
| 1522 | a2 | e2 | e42 |
| 1523 | a2 | e2 | e43 |
| 1524 | a2 | e2 | e44 |
| 1525 | a2 | e2 | e45 |
| 1526 | a2 | e2 | e46 |
| 1527 | a2 | e2 | e47 |
| 1528 | a2 | e2 | e48 |
| 1529 | a2 | e2 | e49 |
| 1530 | a2 | e2 | e50 |
| 1531 | a2 | e2 | e51 |
| 1532 | a2 | e2 | e52 |
| 1533 | a2 | e2 | e53 |
| 1534 | a2 | e2 | e54 |
| 1535 | a2 | e2 | e55 |
| 1536 | a2 | e2 | e56 |
| 1537 | a2 | e2 | e57 |
| 1538 | a2 | e2 | e58 |
| 1539 | a2 | e2 | e59 |
| 1540 | a2 | e2 | e60 |
| 1541 | a2 | e2 | e61 |
| 1542 | a2 | e2 | e62 |
| 1543 | a2 | e2 | e63 |
| 1544 | a2 | e2 | e64 |
| 1545 | a2 | e2 | e65 |
| 1546 | a2 | e2 | e66 |
| 1547 | a2 | e2 | e67 |
| 1548 | a2 | e24 | a1 |
| 1549 | a2 | e24 | d1 |
| 1550 | a2 | e24 | d1 |
| 1551 | a2 | e24 | d4 |
| 1552 | a2 | e24 | d5 |
| 1553 | a2 | e24 | d6 |
| 1554 | a2 | e24 | d8 |
| 1555 | a2 | e24 | d10 |
| 1556 | a2 | e24 | e3 |
| 1557 | a2 | e24 | e4 |
| 1558 | a2 | e24 | e5 |
| 1559 | a2 | e24 | e6 |
| 1560 | a2 | e24 | e7 |
| 1561 | a2 | e24 | e8 |
| 1562 | a2 | e24 | e9 |
| 1563 | a2 | e24 | e10 |
| 1564 | a2 | e24 | e11 |
| 1565 | a2 | e24 | e12 |
| 1566 | a2 | e24 | e13 |
| 1567 | a2 | e24 | e14 |
| 1568 | a2 | e24 | e15 |
| 1569 | a2 | e24 | e16 |
| 1570 | a2 | e24 | e17 |
| 1571 | a2 | e24 | e18 |
| 1572 | a2 | e24 | e19 |
| 1573 | a2 | e24 | e20 |
| 1574 | a2 | e24 | e21 |
| 1575 | a2 | e24 | e22 |
| 1576 | a2 | e24 | e23 |
| 1577 | a2 | e24 | e24 |
| 1578 | a2 | e24 | e25 |
| 1579 | a2 | e24 | e26 |
| 1580 | a2 | e24 | e27 |
| 1581 | a2 | e24 | e28 |
| 1582 | a2 | e24 | e29 |
| 1583 | a2 | e24 | e30 |
| 1584 | a2 | e24 | e31 |
| 1585 | a2 | e24 | e32 |
| 1586 | a2 | e24 | e33 |
| 1587 | a2 | e24 | e34 |
| 1588 | a2 | e24 | e35 |
| 1589 | a2 | e24 | e36 |
| 1590 | a2 | e24 | e37 |
| 1591 | a2 | e24 | e38 |
| 1592 | a2 | e24 | e39 |
| 1593 | a2 | e24 | e40 |
| 1594 | a2 | e24 | e41 |
| 1595 | a2 | e24 | e42 |
| 1596 | a2 | e24 | e43 |
| 1597 | a2 | e24 | e44 |
| 1598 | a2 | e24 | e45 |
| 1599 | a2 | e24 | e46 |
| 1600 | a2 | e24 | e47 |
| Compound No. | Ar_{A} | Ar_{B} | Ar_{C} |
|---|---|---|---|
| 1601 | a2 | e24 | e48 |
| 1602 | a2 | e24 | e49 |
| 1603 | a2 | e24 | e50 |
| 1604 | a2 | e24 | e51 |
| 1605 | a2 | e24 | e52 |
| 1606 | a2 | e24 | e53 |
| 1607 | a2 | e24 | e54 |
| 1608 | a2 | e24 | e55 |
| 1609 | a2 | e24 | e56 |
| 1610 | a2 | e24 | e57 |
| 1611 | a2 | e24 | e58 |
| 1612 | a2 | e24 | e59 |
| 1613 | a2 | e24 | e60 |
| 1614 | a2 | e24 | e61 |
| 1615 | a2 | e24 | e62 |
| 1616 | a2 | e24 | e63 |
| 1617 | a2 | e24 | e64 |
| 1618 | a2 | e24 | e65 |
| 1619 | a2 | e24 | e66 |
| 1620 | a2 | e24 | e67 |
| 1621 | a2 | e27 | a1 |
| 1622 | a2 | e27 | d1 |
| 1623 | a2 | e27 | d1 |
| 1624 | a2 | e27 | d4 |
| 1625 | a2 | e27 | d5 |
| 1626 | a2 | e27 | d6 |
| 1627 | a2 | e27 | d8 |
| 1628 | a2 | e27 | d10 |
| 1629 | a2 | e27 | e3 |
| 1630 | a2 | e27 | e4 |
| 1631 | a2 | e27 | e5 |
| 1632 | a2 | e27 | e6 |
| 1633 | a2 | e27 | e7 |
| 1634 | a2 | e27 | e8 |
| 1635 | a2 | e27 | e9 |
| 1636 | a2 | e27 | e10 |
| 1637 | a2 | e27 | e11 |
| 1638 | a2 | e27 | e12 |
| 1639 | a2 | e27 | e13 |
| 1640 | a2 | e27 | e14 |
| 1641 | a2 | e27 | e15 |
| 1642 | a2 | e27 | e16 |
| 1643 | a2 | e27 | e17 |
| 1644 | a2 | e27 | e18 |
| 1645 | a2 | e27 | e19 |
| 1646 | a2 | e27 | e20 |
| 1647 | a2 | e27 | e21 |
| 1648 | a2 | e27 | e22 |
| 1649 | a2 | e27 | e23 |
| 1650 | a2 | e27 | e25 |
| 1651 | a2 | e27 | e26 |
| 1652 | a2 | e27 | e27 |
| 1653 | a2 | e27 | e28 |
| 1654 | a2 | e27 | e29 |
| 1655 | a2 | e27 | e30 |
| 1656 | a2 | e27 | e31 |
| 1657 | a2 | e27 | e32 |
| 1658 | a2 | e27 | e33 |
| 1659 | a2 | e27 | e34 |
| 1660 | a2 | e27 | e35 |
| 1661 | a2 | e27 | e36 |
| 1662 | a2 | e27 | e37 |
| 1663 | a2 | e27 | e38 |
| 1664 | a2 | e27 | e39 |
| 1665 | a2 | e27 | e40 |
| 1666 | a2 | e27 | e41 |
| 1667 | a2 | e27 | e42 |
| 1668 | a2 | e27 | e43 |
| 1669 | a2 | e27 | e44 |
| 1670 | a2 | e27 | e45 |
| 1671 | a2 | e27 | e46 |
| 1672 | a2 | e27 | e47 |
| 1673 | a2 | e27 | e48 |
| 1674 | a2 | e27 | e49 |
| 1675 | a2 | e27 | e50 |
| 1676 | a2 | e27 | e51 |
| 1677 | a2 | e27 | e52 |
| 1678 | a2 | e27 | e53 |
| 1679 | a2 | e27 | e54 |
| 1680 | a2 | e27 | e55 |
| 1681 | a2 | e27 | e56 |
| 1682 | a2 | e27 | e57 |
| 1683 | a2 | e27 | e58 |
| 1684 | a2 | e27 | e59 |
| 1685 | a2 | e27 | e60 |
| 1686 | a2 | e27 | e61 |
| 1687 | a2 | e27 | e62 |
| 1688 | a2 | e27 | e63 |
| 1689 | a2 | e27 | e64 |
| 1690 | a2 | e27 | e65 |
| 1691 | a2 | e27 | e66 |
| 1692 | a2 | e27 | e67 |
| 1693 | a2 | e33 | a1 |
| 1694 | a2 | e33 | d1 |
| 1695 | a2 | e33 | d1 |
| 1696 | a2 | e33 | d4 |
| 1697 | a2 | e33 | d5 |
| 1698 | a2 | e33 | d6 |
| 1699 | a2 | e33 | d8 |
| 1700 | a2 | e33 | d10 |
| 1701 | a2 | e33 | e3 |
| 1702 | a2 | e33 | e4 |
| 1703 | a2 | e33 | e5 |
| 1704 | a2 | e33 | e6 |
| 1705 | a2 | e33 | e7 |
| 1706 | a2 | e33 | e8 |
| 1707 | a2 | e33 | e9 |
| 1708 | a2 | e33 | e10 |
| 1709 | a2 | e33 | e11 |
| 1710 | a2 | e33 | e12 |
| 1711 | a2 | e33 | e13 |
| 1712 | a2 | e33 | e14 |
| 1713 | a2 | e33 | e15 |
| 1714 | a2 | e33 | e16 |
| 1715 | a2 | e33 | e17 |
| 1716 | a2 | e33 | e18 |
| 1717 | a2 | e33 | e19 |
| 1718 | a2 | e33 | e20 |
| 1719 | a2 | e33 | e21 |
| 1720 | a2 | e33 | e22 |
| 1721 | a2 | e33 | e23 |
| 1722 | a2 | e33 | e25 |
| 1723 | a2 | e33 | e26 |
| 1724 | a2 | e33 | e27 |
| 1725 | a2 | e33 | e28 |
| 1726 | a2 | e33 | e29 |
| 1727 | a2 | e33 | e30 |
| 1728 | a2 | e33 | e31 |
| 1729 | a2 | e33 | e32 |
| 1730 | a2 | e33 | e33 |
| 1731 | a2 | e33 | e34 |
| 1732 | a2 | e33 | e35 |
| 1733 | a2 | e33 | e36 |
| 1734 | a2 | e33 | e37 |
| 1735 | a2 | e33 | e38 |
| 1736 | a2 | e33 | e39 |
| 1737 | a2 | e33 | e40 |
| 1738 | a2 | e33 | e41 |
| 1739 | a2 | e33 | e42 |
| 1740 | a2 | e33 | e43 |
| 1741 | a2 | e33 | e44 |
| 1742 | a2 | e33 | e45 |
| 1743 | a2 | e33 | e46 |
| 1744 | a2 | e33 | e47 |
| 1745 | a2 | e33 | e48 |
| 1746 | a2 | e33 | e49 |
| 1747 | a2 | e33 | e50 |
| 1748 | a2 | e33 | e51 |
| 1749 | a2 | e33 | e52 |
| 1750 | a2 | e33 | e53 |
| 1751 | a2 | e33 | e54 |
| 1752 | a2 | e33 | e55 |
| 1753 | a2 | e33 | e56 |
| 1754 | a2 | e33 | e57 |
| 1755 | a2 | e33 | e58 |
| 1756 | a2 | e33 | e59 |
| 1757 | a2 | e33 | e60 |
| 1758 | a2 | e33 | e61 |
| 1759 | a2 | e33 | e62 |
| 1760 | a2 | e33 | e63 |
| 1761 | a2 | e33 | e64 |
| 1762 | a2 | e33 | e65 |
| 1763 | a2 | e33 | e66 |
| 1764 | a2 | e33 | e67 |
| 1765 | a2 | e36 | a1 |
| 1766 | a2 | e36 | d1 |
| 1767 | a2 | e36 | d1 |
| 1768 | a2 | e36 | d4 |
| 1769 | a2 | e36 | d5 |
| 1770 | a2 | e36 | d6 |
| 1771 | a2 | e36 | d8 |
| 1772 | a2 | e36 | d10 |
| 1773 | a2 | e36 | e3 |
| 1774 | a2 | e36 | e4 |
| 1775 | a2 | e36 | e5 |
| 1776 | a2 | e36 | e6 |
| 1777 | a2 | e36 | e7 |
| 1778 | a2 | e36 | e8 |
| 1779 | a2 | e36 | e9 |
| 1780 | a2 | e36 | e10 |
| 1781 | a2 | e36 | e11 |
| 1782 | a2 | e36 | e12 |
| 1783 | a2 | e36 | e13 |
| 1784 | a2 | e36 | e14 |
| 1785 | a2 | e36 | e15 |
| 1786 | a2 | e36 | e16 |
| 1787 | a2 | e36 | e17 |
| 1788 | a2 | e36 | e18 |
| 1789 | a2 | e36 | e19 |
| 1790 | a2 | e36 | e20 |
| 1791 | a2 | e36 | e21 |
| 1792 | a2 | e36 | e22 |
| 1793 | a2 | e36 | e23 |
| 1794 | a2 | e36 | e25 |
| 1795 | a2 | e36 | e26 |
| 1796 | a2 | e36 | e28 |
| 1797 | a2 | e36 | e29 |
| 1798 | a2 | e36 | e30 |
| 1799 | a2 | e36 | e31 |
| 1800 | a2 | e36 | e32 |
| Compound No. | Ar_{A} | Ar_{B} | Ar_{C} |
|---|---|---|---|
| 1801 | a2 | e36 | e34 |
| 1802 | a2 | e36 | e35 |
| 1803 | a2 | e36 | e36 |
| 1804 | a2 | e36 | e37 |
| 1805 | a2 | e36 | e38 |
| 1806 | a2 | e36 | e39 |
| 1807 | a2 | e36 | e40 |
| 1808 | a2 | e36 | e4 |
| 1809 | a2 | e36 | e42 |
| 1810 | a2 | e36 | e43 |
| 1811 | a2 | e36 | e44 |
| 1812 | a2 | e36 | e45 |
| 1813 | a2 | e36 | e46 |
| 1814 | a2 | e36 | e47 |
| 1815 | a2 | e36 | e48 |
| 1816 | a2 | e36 | e49 |
| 1817 | a2 | e36 | e50 |
| 1818 | a2 | e36 | e51 |
| 1819 | a2 | e36 | e52 |
| 1820 | a2 | e36 | e53 |
| 1821 | a2 | e36 | e54 |
| 1822 | a2 | e36 | e55 |
| 1823 | a2 | e36 | e56 |
| 1824 | a2 | e36 | e57 |
| 1825 | a2 | e36 | e58 |
| 1826 | a2 | e36 | e59 |
| 1827 | a2 | e36 | e60 |
| 1828 | a2 | e36 | e61 |
| 1829 | a2 | e36 | e62 |
| 1830 | a2 | e36 | e63 |
| 1831 | a2 | e36 | e64 |
| 1832 | a2 | e36 | e65 |
| 1833 | a2 | e36 | e66 |
| 1834 | a2 | e36 | e67 |
| 1835 | a2 | e37 | a1 |
| 1836 | a2 | e37 | d1 |
| 1837 | a2 | e37 | d1 |
| 1838 | a2 | e37 | d4 |
| 1839 | a2 | e37 | d5 |
| 1840 | a2 | e37 | d6 |
| 1841 | a2 | e37 | d8 |
| 1842 | a2 | e37 | d10 |
| 1843 | a2 | e37 | e3 |
| 1844 | a2 | e37 | e4 |
| 1845 | a2 | e37 | e5 |
| 1846 | a2 | e37 | e6 |
| 1847 | a2 | e37 | e7 |
| 1848 | a2 | e37 | e8 |
| 1849 | a2 | e37 | e9 |
| 1850 | a2 | e37 | e10 |
| 1851 | a2 | e37 | e11 |
| 1852 | a2 | e37 | e12 |
| 1853 | a2 | e37 | e13 |
| 1854 | a2 | e37 | e14 |
| 1855 | a2 | e37 | e15 |
| 1856 | a2 | e37 | e16 |
| 1857 | a2 | e37 | e17 |
| 1858 | a2 | e37 | e18 |
| 1859 | a2 | e37 | e19 |
| 1860 | a2 | e37 | e20 |
| 1861 | a2 | e37 | e21 |
| 1862 | a2 | e37 | e22 |
| 1863 | a2 | e37 | e23 |
| 1864 | a2 | e37 | e25 |
| 1865 | a2 | e37 | e26 |
| 1866 | a2 | e37 | e28 |
| 1867 | a2 | e37 | e29 |
| 1868 | a2 | e37 | e30 |
| 1869 | a2 | e37 | e31 |
| 1870 | a2 | e37 | e32 |
| 1871 | a2 | e37 | e34 |
| 1872 | a2 | e37 | e35 |
| 1873 | a2 | e37 | e37 |
| 1874 | a2 | e37 | e38 |
| 1875 | a2 | e37 | e39 |
| 1876 | a2 | e37 | e40 |
| 1877 | a2 | e37 | e41 |
| 1878 | a2 | e37 | e42 |
| 1879 | a2 | e37 | e43 |
| 1880 | a2 | e37 | e44 |
| 1881 | a2 | e37 | e45 |
| 1882 | a2 | e37 | e46 |
| 1883 | a2 | e37 | e47 |
| 1884 | a2 | e37 | e48 |
| 1885 | a2 | e37 | e49 |
| 1886 | a2 | e37 | e50 |
| 1887 | a2 | e37 | e51 |
| 1888 | a2 | e37 | e52 |
| 1889 | a2 | e37 | e53 |
| 1890 | a2 | e37 | e54 |
| 1891 | a2 | e37 | e55 |
| 1892 | a2 | e37 | e56 |
| 1893 | a2 | e37 | e57 |
| 1894 | a2 | e37 | e58 |
| 1895 | a2 | e37 | e59 |
| 1896 | a2 | e37 | e60 |
| 1897 | a2 | e37 | e61 |
| 1898 | a2 | e37 | e62 |
| 1899 | a2 | e37 | e63 |
| 1900 | a2 | e37 | e64 |
| 1901 | a2 | e37 | e65 |
| 1902 | a2 | e37 | e66 |
| 1903 | a2 | e37 | e67 |
| 1904 | a2 | e41 | a1 |
| 1905 | a2 | e41 | d1 |
| 1906 | a2 | e41 | d1 |
| 1907 | a2 | e41 | d4 |
| 1908 | a2 | e41 | d5 |
| 1909 | a2 | e41 | d6 |
| 1910 | a2 | e41 | d8 |
| 1911 | a2 | e41 | d10 |
| 1912 | a2 | e41 | e3 |
| 1913 | a2 | e41 | e4 |
| 1914 | a2 | e41 | e5 |
| 1915 | a2 | e41 | e6 |
| 1916 | a2 | e41 | e7 |
| 1917 | a2 | e41 | e8 |
| 1918 | a2 | e41 | e9 |
| 1919 | a2 | e41 | e10 |
| 1920 | a2 | e41 | e11 |
| 1921 | a2 | e41 | e12 |
| 1922 | a2 | e41 | e13 |
| 1923 | a2 | e41 | e14 |
| 1924 | a2 | e41 | e15 |
| 1925 | a2 | e41 | e16 |
| 1926 | a2 | e41 | e17 |
| 1927 | a2 | e41 | e18 |
| 1928 | a2 | e41 | e19 |
| 1929 | a2 | e41 | e20 |
| 1930 | a2 | e41 | e21 |
| 1931 | a2 | e41 | e22 |
| 1932 | a2 | e41 | e23 |
| 1933 | a2 | e41 | e25 |
| 1934 | a2 | e41 | e26 |
| 1935 | a2 | e41 | e28 |
| 1936 | a2 | e41 | e29 |
| 1937 | a2 | e41 | e30 |
| 1938 | a2 | e41 | e31 |
| 1939 | a2 | e41 | e32 |
| 1940 | a2 | e41 | e34 |
| 1941 | a2 | e41 | e35 |
| 1942 | a2 | e41 | e38 |
| 1943 | a2 | e41 | e39 |
| 1944 | a2 | e41 | e40 |
| 1945 | a2 | e41 | e41 |
| 1946 | a2 | e41 | e42 |
| 1947 | a2 | e41 | e43 |
| 1948 | a2 | e41 | e44 |
| 1949 | a2 | e41 | e45 |
| 1950 | a2 | e41 | e46 |
| 1951 | a2 | e41 | e47 |
| 1952 | a2 | e41 | e48 |
| 1953 | a2 | e41 | e49 |
| 1954 | a2 | e41 | e50 |
| 1955 | a2 | e41 | e51 |
| 1956 | a2 | e41 | e52 |
| 1957 | a2 | e41 | e53 |
| 1958 | a2 | e41 | e54 |
| 1959 | a2 | e41 | e55 |
| 1960 | a2 | e41 | e56 |
| 1961 | a2 | e41 | e57 |
| 1962 | a2 | e41 | e58 |
| 1963 | a2 | e41 | e59 |
| 1964 | a2 | e41 | e60 |
| 1965 | a2 | e41 | e61 |
| 1966 | a2 | e41 | e62 |
| 1967 | a2 | e41 | e63 |
| 1968 | a2 | e41 | e64 |
| 1969 | a2 | e41 | e65 |
| 1970 | a2 | e41 | e66 |
| 1971 | a2 | e41 | e67 |
| 1972 | a2 | e59 | a1 |
| 1973 | a2 | e59 | d1 |
| 1974 | a2 | e59 | d1 |
| 1975 | a2 | e59 | d4 |
| 1976 | a2 | e59 | d5 |
| 1977 | a2 | e59 | d6 |
| 1978 | a2 | e59 | d8 |
| 1979 | a2 | e59 | d10 |
| 1980 | a2 | e59 | e3 |
| 1981 | a2 | e59 | e4 |
| 1982 | a2 | e59 | e5 |
| 1983 | a2 | e59 | e6 |
| 1984 | a2 | e59 | e7 |
| 1985 | a2 | e59 | e8 |
| 1986 | a2 | e59 | e9 |
| 1987 | a2 | e59 | e10 |
| 1988 | a2 | e59 | e11 |
| 1989 | a2 | e59 | e12 |
| 1990 | a2 | e59 | e13 |
| 1991 | a2 | e59 | e14 |
| 1992 | a2 | e59 | e15 |
| 1993 | a2 | e59 | e16 |
| 1994 | a2 | e59 | e17 |
| 1995 | a2 | e59 | e18 |
| 1996 | a2 | e59 | e19 |
| 1997 | a2 | e59 | e20 |
| 1998 | a2 | e59 | e21 |
| 1999 | a2 | e59 | e22 |
| 2000 | a2 | e59 | e23 |
| Compound No. | Ar_{A} | Ar_{B} | Ar_{C} |
|---|---|---|---|
| 2001 | a2 | e59 | e25 |
| 2002 | a2 | e59 | e26 |
| 2003 | a2 | e59 | e28 |
| 2004 | a2 | e59 | e29 |
| 2005 | a2 | e59 | e30 |
| 2006 | a2 | e59 | e31 |
| 2007 | a2 | e59 | e32 |
| 2008 | a2 | e59 | e34 |
| 2009 | a2 | e59 | e35 |
| 2010 | a2 | e59 | e38 |
| 2011 | a2 | e59 | e39 |
| 2012 | a2 | e59 | e40 |
| 2013 | a2 | e59 | e42 |
| 2014 | a2 | e59 | e43 |
| 2015 | a2 | e59 | e44 |
| 2016 | a2 | e59 | e45 |
| 2017 | a2 | e59 | e46 |
| 2018 | a2 | e59 | e47 |
| 2019 | a2 | e59 | e48 |
| 2020 | a2 | e59 | e49 |
| 2021 | a2 | e59 | e50 |
| 2022 | a2 | e59 | e51 |
| 2023 | a2 | e59 | e52 |
| 2024 | a2 | e59 | e53 |
| 2025 | a2 | e59 | e54 |
| 2026 | a2 | e59 | e55 |
| 2027 | a2 | e59 | e56 |
| 2028 | a2 | e59 | e57 |
| 2029 | a2 | e59 | e58 |
| 2030 | a2 | e59 | e59 |
| 2031 | a2 | e59 | e60 |
| 2032 | a2 | e59 | e61 |
| 2033 | a2 | e59 | e62 |
| 2034 | a2 | e59 | e63 |
| 2035 | a2 | e59 | e64 |
| 2036 | a2 | e59 | e65 |
| 2037 | a2 | e59 | e66 |
| 2038 | a2 | e59 | e67 |
| 2039 | a2 | e62 | a1 |
| 2040 | a2 | e62 | d1 |
| 2041 | a2 | e62 | d1 |
| 2042 | a2 | e62 | d5 |
| 2043 | a2 | e62 | d6 |
| 2044 | a2 | e62 | d8 |
| 2045 | a2 | e62 | d10 |
| 2046 | a2 | e62 | e5 |
| 2047 | a2 | e62 | e6 |
| 2048 | a2 | e62 | e7 |
| 2049 | a2 | e62 | e8 |
| 2050 | a2 | e62 | e9 |
| 2051 | a2 | e62 | e10 |
| 2052 | a2 | e62 | e11 |
| 2053 | a2 | e62 | e12 |
| 2054 | a2 | e62 | e13 |
| 2055 | a2 | e62 | e14 |
| 2056 | a2 | e62 | e15 |
| 2057 | a2 | e62 | e16 |
| 2058 | a2 | e62 | e17 |
| 2059 | a2 | e62 | e18 |
| 2060 | a2 | e62 | e19 |
| 2061 | a2 | e62 | e20 |
| 2062 | a2 | e62 | e21 |
| 2063 | a2 | e62 | e22 |
| 2064 | a2 | e62 | e23 |
| 2065 | a2 | e62 | e25 |
| 2066 | a2 | e62 | e26 |
| 2067 | a2 | e62 | e28 |
| 2068 | a2 | e62 | e29 |
| 2069 | a2 | e62 | e30 |
| 2070 | a2 | e62 | e31 |
| 2071 | a2 | e62 | e32 |
| 2072 | a2 | e62 | e34 |
| 2073 | a2 | e62 | e35 |
| 2074 | a2 | e62 | e38 |
| 2075 | a2 | e62 | e39 |
| 2076 | a2 | e62 | e40 |
| 2077 | a2 | e62 | e42 |
| 2078 | a2 | e62 | e43 |
| 2079 | a2 | e62 | e44 |
| 2080 | a2 | e62 | e45 |
| 2081 | a2 | e62 | e46 |
| 2082 | a2 | e62 | e47 |
| 2083 | a2 | e62 | e48 |
| 2084 | a2 | e62 | e49 |
| 2085 | a2 | e62 | e50 |
| 2086 | a2 | e62 | e51 |
| 2087 | a2 | e62 | e52 |
| 2088 | a2 | e62 | e53 |
| 2089 | a2 | e62 | e54 |
| 2090 | a2 | e62 | e55 |
| 2091 | a2 | e62 | e56 |
| 2092 | a2 | e62 | e57 |
| 2093 | a2 | e62 | e58 |
| 2094 | a2 | e62 | e60 |
| 2095 | a2 | e62 | e61 |
| 2096 | a2 | e62 | e62 |
| 2097 | a2 | e62 | e63 |
| 2098 | a2 | e62 | e64 |
| 2099 | a2 | e62 | e65 |
| 2100 | a2 | e62 | e66 |
| 2101 | a2 | e62 | e67 |
| 2102 | a2 | e63 | a1 |
| 2103 | a2 | e63 | d1 |
| 2104 | a2 | e63 | d1 |
| 2105 | a2 | e63 | d4 |
| 2106 | a2 | e63 | d5 |
| 2107 | a2 | e63 | d6 |
| 2108 | a2 | e63 | d8 |
| 2109 | a2 | e63 | d10 |
| 2110 | a2 | e63 | e3 |
| 2111 | a2 | e63 | e4 |
| 2112 | a2 | e63 | e5 |
| 2113 | a2 | e63 | e6 |
| 2114 | a2 | e63 | e7 |
| 2115 | a2 | e63 | e8 |
| 2116 | a2 | e63 | e9 |
| 2117 | a2 | e63 | e10 |
| 2118 | a2 | e63 | e11 |
| 2119 | a2 | e63 | e12 |
| 2120 | a2 | e63 | e13 |
| 2121 | a2 | e63 | e14 |
| 2122 | a2 | e63 | e15 |
| 2123 | a2 | e63 | e16 |
| 2124 | a2 | e63 | e17 |
| 2125 | a2 | e63 | e18 |
| 2126 | a2 | e63 | e19 |
| 2127 | a2 | e63 | e20 |
| 2128 | a2 | e63 | e21 |
| 2129 | a2 | e63 | e22 |
| 2130 | a2 | e63 | e23 |
| 2131 | a2 | e63 | e25 |
| 2132 | a2 | e63 | e26 |
| 2133 | a2 | e63 | e28 |
| 2134 | a2 | e63 | e29 |
| 2135 | a2 | e63 | e30 |
| 2136 | a2 | e63 | e31 |
| 2137 | a2 | e63 | e32 |
| 2138 | a2 | e63 | e34 |
| 2139 | a2 | e63 | e35 |
| 2140 | a2 | e63 | e38 |
| 2141 | a2 | e63 | e39 |
| 2142 | a2 | e63 | e40 |
| 2143 | a2 | e63 | e42 |
| 2144 | a2 | e63 | e43 |
| 2145 | a2 | e63 | e44 |
| 2146 | a2 | e63 | e45 |
| 2147 | a2 | e63 | e46 |
| 2148 | a2 | e63 | e47 |
| 2149 | a2 | e63 | e48 |
| 2150 | a2 | e63 | e49 |
| 2151 | a2 | e63 | e50 |
| 2152 | a2 | e63 | e51 |
| 2153 | a2 | e63 | e52 |
| 2154 | a2 | e63 | e53 |
| 2155 | a2 | e63 | e54 |
| 2156 | a2 | e63 | e55 |
| 2157 | a2 | e63 | e56 |
| 2158 | a2 | e63 | e57 |
| 2159 | a2 | e63 | e58 |
| 2160 | a2 | e63 | e60 |
| 2161 | a2 | e63 | e61 |
| 2162 | a2 | e63 | e63 |
| 2163 | a2 | e63 | e64 |
| 2164 | a2 | e63 | e65 |
| 2165 | a2 | e63 | e66 |
| 2166 | a2 | e63 | e67 |
| 2167 | a2 | e64 | a1 |
| 2168 | a2 | e64 | d1 |
| 2169 | a2 | e64 | d1 |
| 2170 | a2 | e64 | d4 |
| 2171 | a2 | e64 | d5 |
| 2172 | a2 | e64 | d6 |
| 2173 | a2 | e64 | d8 |
| 2174 | a2 | e64 | d10 |
| 2175 | a2 | e64 | e3 |
| 2176 | a2 | e64 | e4 |
| 2177 | a2 | e64 | e5 |
| 2178 | a2 | e64 | e6 |
| 2179 | a2 | e64 | e7 |
| 2180 | a2 | e64 | e8 |
| 2181 | a2 | e64 | e9 |
| 2182 | a2 | e64 | e10 |
| 2183 | a2 | e64 | e11 |
| 2184 | a2 | e64 | e12 |
| 2185 | a2 | e64 | e13 |
| 2186 | a2 | e64 | e14 |
| 2187 | a2 | e64 | e15 |
| 2188 | a2 | e64 | e16 |
| 2189 | a2 | e64 | e17 |
| 2190 | a2 | e64 | e18 |
| 2191 | a2 | e64 | e19 |
| 2192 | a2 | e64 | e20 |
| 2193 | a2 | e64 | e21 |
| 2194 | a2 | e64 | e22 |
| 2195 | a2 | e64 | e23 |
| 2196 | a2 | e64 | e25 |
| 2197 | a2 | e64 | e26 |
| 2198 | a2 | e64 | e28 |
| 2199 | a2 | e64 | e29 |
| 2200 | a2 | e64 | e30 |
| Compound No. | Ar_{A} | Ar_{B} | Ar_{C} |
|---|---|---|---|
| 2201 | a2 | e64 | e31 |
| 2202 | a2 | e64 | e32 |
| 2203 | a2 | e64 | e34 |
| 2204 | a2 | e64 | e35 |
| 2205 | a2 | e64 | e38 |
| 2206 | a2 | e64 | e39 |
| 2207 | a2 | e64 | e40 |
| 2208 | a2 | e64 | e42 |
| 2209 | a2 | e64 | e43 |
| 2210 | a2 | e64 | e44 |
| 2211 | a2 | e64 | e45 |
| 2212 | a2 | e64 | e46 |
| 2213 | a2 | e64 | e47 |
| 2214 | a2 | e64 | e48 |
| 2215 | a2 | e64 | e49 |
| 2216 | a2 | e64 | e50 |
| 2217 | a2 | e64 | e51 |
| 2218 | a2 | e64 | e52 |
| 2219 | a2 | e64 | e53 |
| 2220 | a2 | e64 | e54 |
| 2221 | a2 | e64 | e55 |
| 2222 | a2 | e64 | e56 |
| 2223 | a2 | e64 | e57 |
| 2224 | a2 | e64 | e58 |
| 2225 | a2 | e64 | e60 |
| 2226 | a2 | e64 | e61 |
| 2227 | a2 | e64 | e64 |
| 2228 | a2 | e64 | e65 |
| 2229 | a2 | e64 | e66 |
| 2230 | a2 | e64 | e67 |
